(19) United States
(12) Patent Application Publication
(10) Pub. No.: US 2024/0309348 A1
(43) Pub. Date: Sep. 19, 2024

(54) SYSTEMS, METHODS, AND COMPOSITIONS COMPRISING MINIATURE CRISPR NUCLEASES FOR GENE EDITING AND PROGRAMMABLE GENE ACTIVATION AND INHIBITION

(71) Applicant: MASSACHUSETTS INSTITUTE OF TECHNOLOGY, CAMBRIDGE, MA (US)

(72) Inventors: KAIYI JIANG, CAMBRIDGE, MA (US); LUKAS VILLIGER, CAMBRIDGE, MA (US); OMAR OSAMA ABUDAYYEH, CAMBRIDGE, MA (US); JONATHAN S. GOOTENBERG, CAMBRIDGE, MA (US)

(21) Appl. No.: 18/571,014

(22) PCT Filed: Jun. 16, 2022
(86) PCT No.: PCT/US2022/033749

§ 371 Date: Dec. 15, 2023

Related U.S. Application Data
(60) Provisional application No. 63/211,610, filed on Jun. 17, 2021.

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80

(57) ABSTRACT

This disclosure provides systems, methods, and compositions comprising miniature CRISPR. nucleases for gene editing and programmable gene activation and inhibition. The miniature CRISPR nuclease is a target specific nuclease having a compact structure with a small number of amino acids. The target specific nuclease targets DNA and is directed to a target nucleic acid sequence from the DNA by a guide RNA. In some embodiments, the target specific nuclease exhibits DNA cleavage activity and is directed by a gRNA to a target nucleic acid sequence from a DNA. In some embodiments, the target specific nuclease does not exhibit DNA cleavage activity and is directed by a gRNA to a target nucleic acid sequence from a DNA.

### CROSS REFERENCE TO RELATED APPLICATIONS

This application is a U.S. National Stage filing under 35 U.S.C. § 371 of International Patent Application No. PCT/US2022/033749, filed Jun. 16, 2022, which claims the priority benefit of U.S. Provisional Patent Application Ser. No. 63/211,610, filed Jun. 17, 2021. The entirety of this application is hereby incorporated by reference.

### SEQUENCE LISTING

The instant application contains a Sequence Listing which has been submitted electronically in ASCII format and is hereby incorporated by reference in its entirety. Said ASCII copy, created on Jul. 19, 2022, is named 727972_083474-017PC_SL.txt and is 391,702 bytes in size.

### FIELD OF INVENTION

The subject matter disclosed herein is generally directed to systems, methods, and compositions comprising miniature CRISPR nucleases for gene editing and programmable gene activation and inhibition.

### BACKGROUND

Cluster Regularly Interspaced Short Palindromic Repeat (CRISPR)-associated (Cas) nuclease systems are widely used as genome editing tools. Cas9 and Cas12 are two examples of nucleases that are often used in CRISPR-Cas system to edit genomes. These nucleases are generally more than 1000 amino acids long and can be guided by a guide RNA to edit a single stranded or double-stranded DNA target near a short sequence called protospacer adjacent motif (PAM). However, while these nucleases offer great flexibility, their size remains a significant barrier to their use. For example, gene editing and programmable gene activation and inhibition technologies based on these nucleases can generally not be delivered in mouse models using common methods such as adeno-associated vectors (AAV) because of the large size of the nuclease. Furthermore, development of effective gene and cell therapies requires genome editing tools that can meet the demands for reduced payload sizes and efficient integration of diverse and large sequences, regardless of cell type or active repair pathways. CRISPR associated transposases, such as Cas12k or type I-F directed Tn7 systems, allow for programmable integration in bacteria without the need for repair-pathway dependent editing, but have yet to be reconstituted in eukaryotic cells for mammalian genome editing. The difficulty in reconstitution of these systems can be due to the sheer number of proteins (4-7 proteins) that must be properly expressed and delivered to the nucleus for proper assembly and DNA targeting. Prime editing was also reported for programmable gene editing independent of DNA repair pathways but is limited to base substitutions or small deletions and insertions (about <50 bp).

Thus, there is a need for smaller and more compact CRISPR nucleases for gene editing, programmable gene activation and inhibition, and new applications. Smaller and more compact CRISPR nucleases can simplify delivery and extend application, and the additional space on such nucleases can enable fusion with effector domains.

### SUMMARY

The present disclosure provides systems, methods, and compositions comprising miniature CRISPR nucleases for gene editing and programmable gene activation and inhibition.

In one aspect, this disclosure pertains to a composition comprising a target specific nuclease comprising an amino acid sequence 70% identical to an amino acid sequence selected from the group consisting of SEQ ID NOs: 1-19, and a guide RNA (gRNA), wherein a target comprises a DNA target. In some embodiments, the DNA target can be a single stranded DNA. In some embodiments, the DNA target can be a double stranded DNA. In some embodiments, the target specific nuclease can have a length less than about 1000 amino acids. In some embodiments, the target specific nuclease can have a length less than about 900 amino acids. In some embodiments, the target specific nuclease can have a length less than about 800 amino acids. In some embodiments, the amino acid sequence can be SEQ ID NO: 1. In some embodiments, the target specific nuclease can comprise an amino acid sequence 90% identical to the amino acid sequence of SEQ ID NO: 1, or an amino acid sequence 95% identical to the amino acid sequence of SEQ ID NO: 1, or an amino acid sequence 98% identical to the amino acid sequence of SEQ ID NO: 1, an amino acid sequence 99% identical to the amino acid sequence of SEQ ID NO: 1. In some embodiments, the nuclease can be the amino acid sequence of SEQ ID NO: 1.

In some embodiments, the target specific nuclease can be selected from the group consisting of Cas12m, Cas12f, and any variants thereof, and optionally the target specific nuclease can be PsaCas12f.

In some embodiments, the gRNA can be a single guide RNA (sgRNA) or a dual guide (dgRNA). In some embodiments, the gRNA can be a sgRNA and the sgRNA can comprise a nucleic acid sequence 75% identical to a nucleic acid sequence selected from the group consisting of SEQ ID NOs: 20-43 and 61-79. In some embodiments, the gRNA can have a spacer region with a sequence comprising a length of about 17 to about 53 nucleotides (nt), optionally the sequence can comprise a length of about 29 to about 53 nt, optionally the sequence can comprise a length of about 40 to about 50 nt, or optionally the sequence can comprise a length of about 22 nt. In some embodiments, the gRNA can have a direct repeat region with a sequence having a length of from about 20 to about 29 nt. In some embodiments, the gRNA can have a tracrRNA region with a sequence having a length of from about 27 to about 35 nt.

In some embodiments, the DNA target can be in a cell. In some embodiments, the cell can be a prokaryotic cell. In some embodiments, the cell can be a eukaryotic cell. In some embodiments, the eukaryotic cell can be a mammalian cell. In some embodiments, the mammalian cell can be a human cell.

In some embodiments, the amino acid sequence can specifically bind to a protospacer-adjacent motif (PAM). In some embodiments, the PAM can be selected from the group consisting of NNNNGATT, NNNNGNNN, NNG, NG, NGAN, NGNG, NGAG, NGCG, NAAG, NGN, NRN, NNGRRN, NNNRRT, TTTN, TTTV, TYCV, TATV, TYCV, TATV, TTN, KYTV, TYCV, TATV, TBN, any variants thereof, and any combinations thereof.

In another aspect, a nucleic acid molecule encoding a target specific nuclease is discussed.

In another aspect, a nucleic acid molecule encoding a guide RNA is discussed.

In another aspect, one or more vectors comprising a nucleic acid molecule encoding a target specific nuclease and/or a guide RNA is discussed.

In another aspect, a cell comprising a composition comprising a target specific nuclease comprising an amino acid sequence 70% identical to an amino acid sequence selected from the group consisting of SEQ ID NOs: 1-19, a target comprises a DNA, and a guide RNA; or a cell comprising a nucleic acid molecule encoding the target specific nuclease; or a cell comprising a nucleic acid molecule encoding the gRNA; or a cell comprising one or more vectors comprising a nucleic acid molecule encoding the target specific nuclease and/or the guide RNA is discussed. In some embodiments, the cell can be a prokaryotic cell. In some embodiments, the cell can be a eukaryotic cell. In some embodiments, the eukaryotic cell can be a mammalian cell. In some embodiments, the mammalian cell can be a human cell.

In another aspect, a method of inserting or deleting one or more base pairs in a DNA is discussed, the method comprising cleaving the DNA at a target site with a target specific nuclease, the cleavage results in overhangs on both DNA ends, inserting a nucleotide complementary to the overhanging nucleotide on both of the dsDNA ends, or removing the overhanging nucleotide on both of the DNA ends, and ligating the dsDNA ends together, thereby inserting or deleting one or more base pairs in the dsDNA, the nuclease comprising an amino acid sequence 70% identical to an amino acid sequence selected from the group consisting of SEQ ID NOs: 1-19, and the target specificity of the target specific nuclease is provided by a guide RNA (gRNA). In some embodiments, the target specific nuclease can have a length less than about 1000 amino acids. In some embodiments, the target specific nuclease can have a length less than about 900 amino acids. In some embodiments, the target specific nuclease can have a length less than about 800 amino acids. In some embodiments, the amino acid sequence can be SEQ ID NO: 1.

In some embodiments, the target specific nuclease can comprise an amino acid sequence 90% identical to the amino acid sequence of SEQ ID NO: 1. In some embodiments, the target specific nuclease can comprise an amino acid sequence 95% identical to the amino acid sequence of SEQ ID NO: 1. In some embodiments, the target specific nuclease can comprise an amino acid sequence 98% identical to the amino acid sequence of SEQ ID NO: 1. In some embodiments, the target specific nuclease can comprise an amino acid sequence 99% identical to the amino acid sequence of SEQ ID NO: 1. In some embodiments, the nuclease can be the amino acid sequence of SEQ ID NO: 1.

In some embodiments, the target specific nuclease can be selected from the group consisting of Cas12f, Cas12m, and any variants thereof, and optionally the target specific nuclease can be PsaCas12f.

In some embodiments, the gRNA can be a single guide RNA (sgRNA) or a dual guide RNA (dgRNA). In some embodiments, the gRNA can be a sgRNA comprising a nucleic acid sequence 70% identical to a nucleic acid sequence selected from the group consisting of SEQ ID NOs: 20-43 and 61-79. In some embodiments, the gRNA comprises a spacer region with a sequence having a length of from about 20 to about 30 nucleotides (nt), about 22 nt; or the gRNA comprises a spacer region with sequence having a length of from about 20 to about 53 nt, or from about 29 to about 53 nt or from about 40 to about 50 nt.

In some embodiments, the DNA target can be in a cell. In some embodiments, the cell can be a prokaryotic cell. In some embodiments, the cell can be a eukaryotic cell. In some embodiments, the eukaryotic cell can be a mammalian cell. In some embodiments, the mammalian cell can be a human cell.

In some embodiments, the amino acid sequence can specifically bind to a protospacer-adjacent motif (PAM). In some embodiments, the PAM can be selected from the group consisting of NNNNGATT, NNNNGNNN, NNG, NG, NGAN, NGNG, NGAG, NGCG, NAAG, NGN, NRN, NNGRRN, NNNRRT, TTTN, TTTV, TYCV, TATV, TYCV, TATV, TTN, KYTV, TYCV, TATV, TBN, any variants thereof, and any combinations thereof.

In another aspect, a method of detecting a DNA target is discussed, the method comprising coupling the DNA target with a reporter to form a DNA-reporter complex, mixing the DNA-reporter complex with a target specific nuclease and a guide RNA (gRNA), cleaving the DNA-reporter complex, and measuring a signal from the reporter, thereby detecting the DNA target. In some embodiments, the target specific nuclease can be selected from the group consisting of Cas12f, Cas12m, and any variants thereof, and optionally the target specific nuclease can be PsaCas12f. In some embodiments, the target specific nuclease can be complexed with a crRNA. In some embodiments, the reporter can be a fluorescent reporter.

In another aspect, a method for activating or inhibiting the expression of a gene is discussed, the method comprising mixing a composition with one or more transcription factors, the composition comprising a target specific nuclease comprising an amino acid sequence 70% identical to an amino acid sequence selected from the group consisting of SEQ ID NOs: 1-19, a DNA target, and a guide RNA (gRNA), the target specific nuclease lacks endonuclease ability, and the target DNA comprises the gene, thereby activating the gene.

In another aspect, a method for nucleic acid base editing is discussed, the method comprising mixing a composition, the composition comprising a target specific nuclease comprising an amino acid sequence 70% identical to an amino acid sequence selected from the group consisting of SEQ ID NOs: 1-19, a DNA target, and a guide RNA (gRNA), the target specific nuclease is a nickase or a nuclease coupled to a deaminase, thereby editing the nucleic acid base from the target DNA.

In another aspect, a method for activating or inhibiting the expression of a gene is discussed, the method comprising mixing a composition comprising a target specific nuclease comprising an amino acid sequence 70% identical to an amino acid sequence selected from the group consisting of SEQ ID NOs: 1-19, and a guide RNA (gRNA), a target comprises a DNA target, with one or more epigenetic modifiers, the target specific nuclease lacks endonuclease activity, the target DNA comprises the gene, and modifying the target DNA or one or more histones associated to the target DNA, thereby activating or inhibiting the gene. In some embodiments, the epigenetic modifier can comprise KRAB, DNMT3a, DNMT1, DNMT3b, DNMT3L, TET1, p300, any variants thereof, or any combinations thereof.

These aspects and embodiments, as well as others, are disclosed in further detail herein.

### BRIEF DESCRIPTION OF THE DRAWINGS

Aspects, features, benefits, and advantages of the embodiments described herein will be apparent with regard to the following description, appended claims, and accompanying drawings where:

FIG. 1A shows a schematic diagram illustrating the computational identification of novel miniature CRISPR nucleases from metagenomic samples according to embodiments of the present teachings;

FIG. 1B shows a simulated tree of Cas orthologs according to embodiments of the present teachings;

FIG. 1C shows the size distribution of Cas12a ortholog according to embodiments of the present teachings;

FIG. 1D shows the size distribution of CasM ortholog according to embodiments of the present teachings;

FIG. 1E shows the secondary structure prediction of PasCas12f direct repeat according to embodiments of the present teachings;

FIG. 1F shows the secondary structure prediction of putative PasCas12 tracrRNA according to embodiments of the present teachings;

FIG. 2 shows a schematic diagram illustrating the screening of smaller CRISPR nucleases for functional activity via LASSO and TXTL according to embodiments of the present teachings;

FIG. 3A shows a vector map depicting single-vector activators, base editors, or homology directed repair (HDR) enabled by smaller CRISPR nucleases according to embodiments of the present teachings;

FIG. 3B shows a schematic diagram illustrating in vivo modification via single-vector activators, base editors, or HDR with AAV according to embodiments of the present teachings;

FIG. 3C shows the optimization of small CRISPR effectors for mammalian single-vector delivery according to embodiments of the present teachings;

FIG. 4 shows the testing of PsaCas12f sgRNA constructs in human mammalian cells according to embodiments of the present teachings;

FIG. 5A shows the testing of PsaCas12f NLS constructs according to embodiments of the present teachings;

FIG. 5B shows the editing with PsaCas12f (NLS14) with sgRNA 13 according to embodiments of the present teachings;

FIG. 5C shows the editing with PsaCas12f (NLS14) with non-targeting guide according to embodiments of the present teachings;

FIG. 5D shows the editing with PsaCas12f (no NLS) with sgRNA 14 according to embodiments of the present teachings;

FIG. 5E shows the editing with PsaCas12f (no NLS) with non-targeting guide according to embodiments of the present teachings;

FIG. 6A shows a process for optimal guide RNA prediction according to embodiments of the present teachings;

FIG. 6B shows predicted energy landscape for different RNA designs according to embodiments of the present teachings;

FIG. 6C shows in vitro cleavage with PsaCas12f using different sgRNA scaffolds generated by in silico optimization according to embodiments of the present teachings;

FIG. 7A shows a diagram of luciferase indel reporter for engineering novel CRISPR effectors like PsaCas12f for mammalian genome editing according to embodiments of the present teachings;

FIG. 7B shows genome editing data with PasCas12f in HEK293FT cells showing about 0.05% indel activity that is 100 times higher than background detection, wherein activity is detected with N-terminal NLS Cas12f expression and natural guide scaffold according to embodiments of the present teachings;

FIG. 7C shows a bar graph of gene editing with PasCas12f in HEK293FT cells according to embodiments of the present teachings (Figure discloses SEQ ID NOS 289-290, 290-313, respectively, in order of appearance);

FIG. 7D shows allele plot of Cas12f EMX1 cleavage showing indels at target according to embodiments of the present teachings;

FIG. 7E shows a bar graph of the sgRNA and DR/tracr optimization for Cas12f, wherein the luciferase reporter for indels reveals key sgRNA and tracrRNA/DR combos that have indel activity in HEK293FT cells according to embodiments of the present teachings;

FIG. 8A shows a schematic of PsaCas12f expression locus according to embodiments of the present teachings;

FIG. 8B shows the PasCas12f PAM determined by in vitro cleavage according to embodiments of the present teachings;

FIG. 8C shows the putative crRNA determined by small RNA sequencing according to embodiments of the present teachings;

FIG. 8D shows the validation of PasCas12f PAM in vitro cleavage with recombinant protein according to embodiments of the present teachings;

FIG. 9A shows PsaCas12f coupled to MiniVPR for CRISPR activation (CRISPRa) using dead PsaCas12f according to embodiments of the present teachings;

FIG. 9B shows a bar graph of the RLU for PsaCas12f coupled to VPR and MiniVPR, demonstrating that gene activation using MiniVPR and VPR can be achieved with catalytically dead PsaCas12f, wherein pDF235 and EMX1v2 reporters are different luciferase reporters for measuring gene activation according to embodiments of the present teachings;

FIG. 9C shows a bar graph of the RLU of PsaCas12f coupled with small linker sequences (5-10aa) at 6 different positions according to embodiments of the present teachings; and

FIG. 9D shows a bar graph of the fluorescence for PasCas12f based on target specific collateral activity, which can be used for diagnostics according to embodiments of the present teachings.

FIG. 10A illustrates the resulting sgRNA secondary structure derived from an in silico secondary structure determination with stem loop 1-3 boxed (SL1-3) predicted using via http://rna.tbi.univie.ac.at/. Stem loop 4 (SL4, interacts with crRNA) and stem loop 5 (SL5) were informed by Takeda et al., Mol Cell, 81(3):558-570 (2021). Figure discloses SEQ ID NO: 314.

FIG. 10B displays the annotated stem-loop sequence for the sgRNA stem-loop variants which were mutated to analyze the impact of gene editing efficiencies. Red denotes nucleobase changes that were introduced, orange denotes nucleobases that form stems, and violet denotes loops that were added to allow recruitment of MS2 coat/proteins. Figure discloses SEQ ID NOS 95-144, respectively, in order of appearance.

FIG. 10C shows a bar graph of the RLU using PsaCas12f with the different sgRNA stem-loop variants demonstrating that modifications to the secondary structure of the sgRNA impacts gene editing efficiencies.

FIG. 11A shows a bar graph of the RLU using PsaCas12f with a panel of sgRNA variants which each have a combination of the modifications derived from single modification sgRNA stem-loop variants.

FIG. 11B shows a bar graph of the percent indel formation at the EMX1 genomic locus using PsaCas12f with a panel of sgRNA variants which each have a combination of modifications derived from the single sgRNA stem-loop variants (4× combinations, left panel and 2× combinations, right panel).

FIG. 11C shows a bar graph of the RLU using a panel of thirty mutant PsaCas12f with the two best sgRNA combination stem-loop variants (named scaffold version 3.1 and scaffold version 3.2) demonstrating the robustness of the sgRNA scaffold version 3.2.

FIG. 12A is a schematic of the sgRNA scaffold named version 3.2 which highlights the position of the spacer sequence at the 3′-end. Figure discloses SEQ ID NOS 315-316 and 318, respectively, in order of appearance.

FIG. 12B shows a bar graph of the RLU using PsaCas12f with a panel of version 3.2 sgRNA scaffolds which have varying spacer lengths (2, 3, 18, 19, 20, 21, 22, 23, 24, and 25 base pairs).

FIG. 13 shows the percent indel formation at two different positions within the HBB and the RNF genomic loci (HBB g1, HBB h2, RNF g4, and RNF g6) using either the PsaCas12f with the sgRNA scaffold version 3.2 or the Un1Cas12f1 with nbt scaffold.

FIG. 14 shows a bar graph of the percent indel formation at the EMX genomic locus using a panel of PsaCas12 variants (intra-protein NLS constructs 1-6) where the NLS sequence derived from SV40 was fused at random positions in the PsaCas12f sequence (as shown in bottom schematic).

FIG. 15 shows a bar graph of the percent indel formation at the RUNX1 genomic locus using a PsaCas12f with a sgRNA scaffold (has a flanking SV40 NLS) which was delivered to cells via AAV particles.

FIG. 16A shows a bar graph of the RLU using a panel of 12 circular permutated PsaCas12f mutants (named cpPsaCas12_1-12). The bottom schematic depicts how the PsaCas12f sequence can be split at different positions to create new N- and C-termini by inserting a (GGS)₆ peptide linker. (SEQ ID NO: 286).

FIG. 16B shows a bar graph of the percent indel formation at the RUNX1 genomic locus using a panel of 12 circular permutated PsaCas12f mutants (cpPsaCas12_1-12).

FIG. 17 shows a bar graph of the percent indel formation at the RNF2 genomic locus using a panel of PsaCas12f mutants obtained from a machine learning model which predicted point mutations which could result in higher gene editing efficiencies. PsaCas12f variant with a point mutation at position 333 dramatically increased cleavage efficiency.

### DETAILED DESCRIPTION

It will be appreciated that for clarity, the following disclosure will describe various aspects of embodiments. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

### Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an,” and “the” include both singular and plural referents unless the context clearly dictates otherwise. Thus, for example, reference to “a cell” includes a plurality of such cells.

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, the term “about” or “approximately” refers to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosed invention. It is to be understood that the value to which the modifier “about” or “approximately” refers is itself disclosed.

As used herein, the term “polypeptide” and the likes refer to an amino acid sequence including a plurality of consecutive polymerized amino acid residues (e.g., at least about 2 consecutive polymerized amino acid residues). “Polypeptide” refers to an amino acid sequence, oligopeptide, peptide, protein, enzyme, nuclease, or portions thereof, and the terms “polypeptide,” “oligopeptide,” “peptide,” “protein,” “enzyme,” and “nuclease,” are used interchangeably.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

The term “variant” as used herein means a polypeptide or nucleotide sequence that differs from a given polypeptide or nucleotide sequence in amino acid or nucleic acid sequence by the addition (e.g., insertion), deletion, or conservative substitution of amino acids or nucleotides, but that retains some or all the biological activity of the given polypeptide (e.g., a variant nucleic acid could still encode the same or a similar amino acid sequence). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al., J. Mol. Biol., 157: 105-132 (1982)). The hydropathic index of an amino acid is based on a consideration of its hydrophobicity and charge. It is known in the art that amino acids of similar hydropathic indexes can be substituted and still retain protein function. The present disclosure provides amino acids having hydropathic indexes of 2 that can be substituted. The hydrophilicity of amino acids also can be used to reveal substitutions that would result in proteins retaining some or all biological functions. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining some or all biological activities, for example immunogenicity, as is understood in the art. The present disclosure provides substitutions that can be performed with amino acids having hydrophilicity values within f2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

The term “variant” also can be used to describe a polypeptide or fragment thereof that has been differentially processed, such as by proteolysis, phosphorylation, or other post-translational modification, yet retains some or all its biological and/or antigen reactivities. Use of “variant” herein is intended to encompass fragments of a variant unless otherwise contradicted by context. The term “protospacer-adjacent motif” as used herein refers to a DNA sequence immediately following a DNA sequence targeted by a nuclease. Examples of protospacer-adjacent motif include, without limitation, NNNNGATT, NNNNGNNN, NNG, NG, NGAN, NGNG, NGAG, NGCG, NAAG, NGN, NRN, NNGRRN, NNNRRT, TTTN, TTTV, TYCV, TATV, TYCV, TATV, TTN, KYTV, TYCV, TATV, TBN, any variants thereof, and any combinations thereof.

Alternatively, or additionally, a “variant” is to be understood as a polynucleotide or protein which differs in comparison to the polynucleotide or protein from which it is derived by one or more changes in its length or sequence. The polypeptide or polynucleotide from which a protein or nucleic acid variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed modified molecules such as but not limited to post-translationally modified proteins (e.g., glycosylated, biotinylated, phosphorylated, ubiquitinated, palmitoylated, or proteolytically cleaved proteins) and modified nucleic acids such as methylated DNA. Also, mixtures of different molecules such as but not limited to RNA-DNA hybrids, are encompassed by the term “variant”. Typically, a variant is constructed artificially, by gene-technological means whilst the parent polypeptide or polynucleotide is a wild-type protein or polynucleotide. However, also naturally occurring variants are to be understood to be encompassed by the term “variant” as used herein. Further, the variants usable in the present disclosure may also be derived from homologs, orthologs, or paralogs of the parent molecule or from artificially constructed variant, provided that the variant exhibits at least one biological activity of the parent molecule, i.e., is functionally active.

Alternatively, or additionally, a “variant” as used herein can be characterized by a certain degree of sequence identity to the parent polypeptide or parent polynucleotide from which it is derived. More precisely, a protein variant in the context of the present disclosure exhibits at least 80% sequence identity to its parent polypeptide. A polynucleotide variant in the context of the present disclosure exhibits at least 70% sequence identity to its parent polynucleotide. The term “at least 70% sequence identity” or the like is used throughout the specification with regard to polypeptide and polynucleotide sequence comparisons. This expression refers to a sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The similarity of nucleotide and amino acid sequences, i.e., the percentage of sequence identity, can be determined via sequence alignments. Such alignments can be carried out with several art-known algorithms, with the mathematical algorithm of Karlin and Altschul (Karlin & Altschul (1993) Proc. Natl. Acad. Sci. USA 90: 5873-5877), with hmmalign (HMMER package, hmmer.wustl.edu/) or with the CLUSTAL algorithm (Thompson, J. D., Higgins, D. G. & Gibson, T. J. (1994) Nucleic Acids Res. 22, 4673-80) available e.g. on www.ebi.ac.uk/Tools/clustalw/or on www.ebi.ac.uk/Tools/clustalw2/index.html or on npsa-pbil.ibcp.fr/cgi-bin/npsa_automat.pl?page=/NPSA/npsa_clustalw.html. Some parameters used are the default parameters as they are set on www.ebi.ac.uk/Tools/clustalw/or www.ebi.ac.uk/Tools/clustalw2/index.html. The grade of sequence identity (sequence matching) may be calculated using e.g., BLAST, BLAT or BlastZ (or BlastX). A similar algorithm is incorporated into the BLASTN and BLASTP programs of Altschul et al. (1990) J. Mol. Biol. 215: 403-410. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs can be used. Sequence matching analysis may be supplemented by established homology mapping techniques like Shuffle-LAGAN (Brudno M., Bioinformatics 2003b, 19 Suppl 1:I54-I62) or Markov random fields. When percentages of sequence identity are referred to in the present application, these percentages are calculated in relation to the full length of the longer sequence, if not specifically indicated otherwise.

As used herein, the term “miniature CRISPR nuclease” and the like refer to a “target specific nuclease” having a compact structure with a small number of amino acids.

As used herein, the term “target specific nuclease” and the like refer to a nuclease that targets DNA and is directed to a target nucleic acid sequence from the DNA by a guide RNA (gRNA). The DNA can be a single stranded DNA or a double stranded DNA.

As used herein, the term “guide RNA” (gRNA) and the like refer to an RNA that guides the editing, activation or inhibition of one or more genes of interest or one or more nucleic acid sequences of interest into a target genome. A gRNA is capable of targeting a nuclease to a target nucleic acid or sequence in a genome. The gRNA can also refer to a prime editing guide RNA (pegRNA), a nicking guide RNA (ngRNA), a single guide RNA (sgRNA), i.e., a fusion of two noncoding RNAs, a synthetic CRISPR RNA (crRNA) and a trans-activating CRISPR RNA (tracrRNA), and a dual guide RNA (dgRNA). In some embodiments, the term “gRNA molecule” or the like refer to a nucleic acid encoding a gRNA. In some embodiments, a gRNA molecule is non-naturally occurring. In some embodiments, a gRNA molecule is a synthetic gRNA molecule.

As used herein, the term “target” or the like refer to a polynucleotide or polypeptide that is targeted. In some embodiments, the target is a DNA target. In some embodiments, the DNA target is associated with one or more histones. In some embodiments, the DNA target is a double-stranded DNA target. In other embodiments, the DNA target is a single-stranded DNA target.

As used herein, the terms “circular permutation,” “circularly permuted,” and “(CP),” refer to the conceptual process of taking a linear protein, or its cognate nucleic acid sequence, and fusing the native N- and C-termini (directly or through a linker, using protein or recombinant DNA methodologies) to form a circular molecule, and then cutting the circular molecule at a different location to form a new linear protein, or cognate nucleic acid molecule, with termini different from the termini in the original molecule. Circular permutation thus preserves the sequence, structure, and function of a protein (other than the optional linker), while generating new C- and N-termini at different locations that, in accordance with one aspect of the invention, results in an improved orientation for fusing a desired polypeptide fusion partner as compared to the original ligand. Circular permutation also includes any process that results in a circularly permutated straight-chain molecule, as defined herein. In general, a circularly permuted molecule is de novo expressed as a linear molecule and does not formally go through the circularization and opening steps.

It is noted that all publications and references cited herein are expressly incorporated herein by reference in their entirety. The publications discussed herein are provided solely for their disclosure prior to the filing date of the present application. Nothing herein is to be construed as an admission that the present invention is not entitled to antedate such publication. Further, the dates of publication provided may be different from the actual publication dates which may need to be independently confirmed.

### Overview

The embodiments disclosed herein provide non-naturally occurring or engineered systems, methods, and compositions comprising miniature CRISPR nucleases for gene editing and programmable gene activation and inhibition. The miniature CRISPR nuclease is a target specific nuclease having a compact structure with a small number of amino acids. The target specific nuclease targets single stranded or double stranded DNA and is directed to a target nucleic acid sequence from the DNA by a guide RNA (gRNA). The gRNA can be a single-guide RNA, i.e., a fusion of two non-coding RNA: a synthetic CRISPR RNA (crRNA) and a trans-activating CRISPR RNA (tracrRNA). The crRNA and tracrRNA aid in directing the target specific nuclease to a target nucleic acid sequence, and these RNA molecules can be specifically engineered to target specific nucleic acid sequences. Certain aspects of the present teachings involve a target specific nuclease that exhibits DNA cleavage activity and is directed to a target nucleic acid sequence from a DNA by a gRNA. Certain aspects of the present teachings involve a target specific nuclease that does not exhibit DNA cleavage activity and is directed to a target nucleic acid sequence from a DNA by a gRNA molecule. Certain aspects of the present teachings involve a target specific nuclease for diagnostic applications.

### Miniature CRISPR Nucleases

Some embodiments disclosed herein are directed to non-naturally occurring or engineered CRISPR-Cas (clustered regularly interspaced short palindromic repeats associated proteins) systems. In the conflict between bacterial hosts and their associated viruses, CRISPR-Cas systems provide an adaptive defense mechanism that utilizes programmed immune memory. CRISPR-Cas systems provide their defense through three stages: adaptation, the integration of short nucleic acid sequences into the CRISPR array that serves as memory of past infections; expression, the transcription of the CRISPR array into a pre-crRNA (CRISPR RNA) transcript and processing of the pre-crRNA into functional crRNA species targeting foreign nucleic acids; and interference, the programming of CRISPR effectors by crRNA to cleave nucleic acid of foreign threats. Across all CRISPR-Cas systems, these fundamental stages display enormous variation, including the identity of the target nucleic acid (either RNA, DNA, or both) and the diverse domains and proteins involved in the effector ribonucleoprotein complex of the system.

CRISPR-Cas systems can be broadly split into two classes based on the architecture of the effector modules involved in pre-crRNA processing and interference. Class 1 systems have multi-subunit effector complexes composed of many proteins, whereas Class 2 systems rely on single-effector proteins with multi-domain capabilities for crRNA binding and interference; Class 2 effectors often provide pre-crRNA processing activity as well. Class 1 systems contain 3 types (type I, III, and IV) and 33 subtypes, including the RNA and DNA targeting type III-systems. Class 2 CRISPR families encompass 3 types (type IL, V, and VI) and 17 subtypes of systems, including the RNA-guided DNases Cas9 and Cas12 and the RNA-guided RNase Cas13. Continual sequencing of novel bacterial genomes and metagenomes uncovers new diversity of CRISPR-Cas systems and their evolutionary relationships, necessitating experimental work that reveals the function of these systems and develops them into new tools.

The CRISPR-Cas systems disclosed herein comprise a miniature CRISPR nuclease. The miniature CRISPR nuclease is a target specific nuclease that has a compact structure with a small number of amino acids and targets DNA. The target specific nuclease disclosed herein can be for example, without limitation, Cas12f, Cas12m, and any variants thereof, and optionally the target specific nuclease can be PsaCas12f. In some embodiments, the target specific nuclease is a nuclease that edits a single stranded or double stranded DNA. In some embodiments, the target specific nuclease is a nuclease that edits a single-stranded DNA (ssDNA). In some embodiments, a target specific nuclease is a nuclease that edits a double-stranded DNA. In some embodiments, the target specific nuclease is a nuclease that edits DNA in the genome of a cell.

The CRISPR-Cas systems disclosed herein can comprise one or more epigenetic modifiers. Examples of epigenetic modifiers include, without limitation, KRAB, DNMT3a, DNMT1, DNMT3b, DNMT3L, TET1, p300, any variants thereof, and any combinations thereof.

The target specific nuclease can comprise an amino acid sequence selected from the group consisting of SEQ ID NOs: 1-19. For example, the target specific nuclease comprises an amino acid sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from the group consisting of SEQ ID NOs: 1-19.

In some embodiments, the target specific nucleases include tags such as for example, without limitation, 3×Flag, nuclear localization sequence (NLS), and the combination of 3×Flag and NLS.

The CRISPR-Cas systems disclosed herein comprise a guide RNA (gRNA). The gRNA directs the target specific nuclease to a target nucleic acid sequence from a single stranded or double stranded DNA targeted by the nuclease. In some embodiments, the gRNA is a single-guide RNA (sgRNA). In some embodiments, the gRNA comprises a CRISPR RNA (crRNA), a trans-activating CRISPR RNA (tracrRNA), or a combination thereof. The crRNA and tracrRNA aid in directing the target specific nuclease to a target nucleic acid sequence, and these RNA molecules can be specifically engineered to target specific nucleic acid sequences.

In general, a guide sequence from the gRNA is any polynucleotide sequence having sufficient complementarity with a target polynucleotide sequence to hybridize with the target sequence and direct sequence-specific binding of a target specific nuclease to the target sequence. In some embodiments, the degree of complementarity between a guide sequence and its corresponding target sequence, when optimally aligned using a suitable alignment algorithm, is about or more than about 50%, 52%, 54%, 56%, 58%, 60%, 62%, 64%, 66%, 68%, 70%, 72%, 74%, 76%, 78%, 80%, 82%, 84%, 86%, 88%, 90%, 91%, 92%, 93%, 94%, 95%, 96%, 97%, 98%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, ClustalX, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, or more nucleotides in length. In some embodiments, a guide sequence is less than about 75, 50, 45, 40, 35, 30, 25, 20, 15, 12, or fewer nucleotides in length. In some embodiments, the guide RNA has a spacer region with a sequence having a length of from about 17 to about 53 nucleotides (nt), from about 25 to about 53 nt, from about 29 to about 53 nt or from about 40 to about 50 nt. In some embodiments, the guide RNA has a spacer region with a sequence having a length of about 20 nt, about 21 nt, about 22 nt, about 23 nt, about 24 nt, about 25 nt, about 26 nt, about 27 nt, about 28 nt, about 29 nt, about 30 nt, about 31 nt, about 32 nt, about 33 nt, about 34 nt, about 35 nt, about 36 nt, about 37 nt, about 38 nt, about 39 nt, about 40 nt, about 41 nt, about 42 nt, about 43 nt, about 44 nt, about 45 nt, about 46 nt, about 47 nt, about 48 nt, about 49 nt, about 50 nt, or within any ranges that are made of any two or more points in the above list. In some embodiments, the guide RNA has a direct repeat region with a sequence having a length of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, about 29 nt, about 30 nt, about 31 nt, about 32 nt, about 33 nt, about 34 nt, about 35 nt, about 36 nt, about 37 nt, about 38 nt, about 39 nt, about 40 nt, about 41 nt, about 42 nt, about 43 nt, about 44 nt, about 45 nt, about 46 nt, about 47 nt, about 48 nt, about 49 nt, about 50 nt, or within any ranges that are made of any two or more points in the above list. In some embodiments, the guide RNA has a tracrRNA region having a sequence with a length of about 15 nt, about 16 nt, about 17 nt, about 18 nt, about 19 nt, about 20 nt, about 21 nt, about 22 nt, about 23 nt, about 24 nt, about 25 nt, about 26 nt, about 27 nt, about 28 nt, about 29 nt, about 30 nt, about 31 nt, about 32 nt, about 33 nt, about 34 nt, about 35 nt, about 36 nt, about 37 nt, about 38 nt, about 39 nt, about 40 nt, about 41 nt, about 42 nt, about 43 nt, about 44 nt, about 45 nt, about 46 nt, about 47 nt, about 48 nt, about 49 nt, about 50 nt, or within any ranges that are made of any two or more points in the above list. The ability of a guide sequence to direct sequence-specific binding of a target specific nuclease to a target sequence may be assessed by any suitable assay.

In some embodiments, the gRNA comprises a nucleic acid sequence selected from the group consisting of SEQ ID NOs: 20-43 and 61-79. For example, the sgRNA can comprise a nucleic acid sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 20-43 and 61-79.

### Discovery of Miniature CRISPR Nucleases

A major challenge for in vivo genome engineering is the size of tools, which are prohibitive for viral delivery, especially with applications such as base editing, activation, inhibition, and HDR. The most commonly used Cas9 ortholog is Streptococcus pyogenes SpCas9, a large, 1368 amino acid length protein. Smaller CRISPR nucleases with lengths less than about 1000 amino acids can result in base editors and transcriptional activators that can fit within the 4.7 kb limit of AAV vectors. Smaller CRISPR nucleases can be discovered through metagenomic mining and innovative screening methods. Protein and guide RNA engineering can be used to boost the activity of these smaller nucleases for robust mammalian cell applications.

Cas12f and Cas12h nucleases are among the smallest DNA-targeting Cas12 families characterized to date, with Cas12f having between about 400 and about 700 residues and Cas12h having between about 870 and about 933 residues. However, these enzymes have not been engineered for high efficiency genome editing, with unquantified editing rates by Cas12f in mammalian cells and genome editing not yet demonstrated with Cas12h.

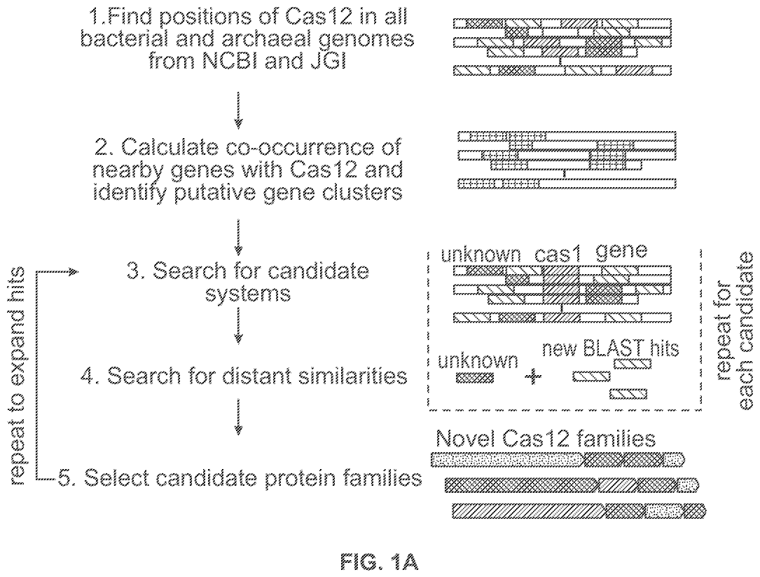

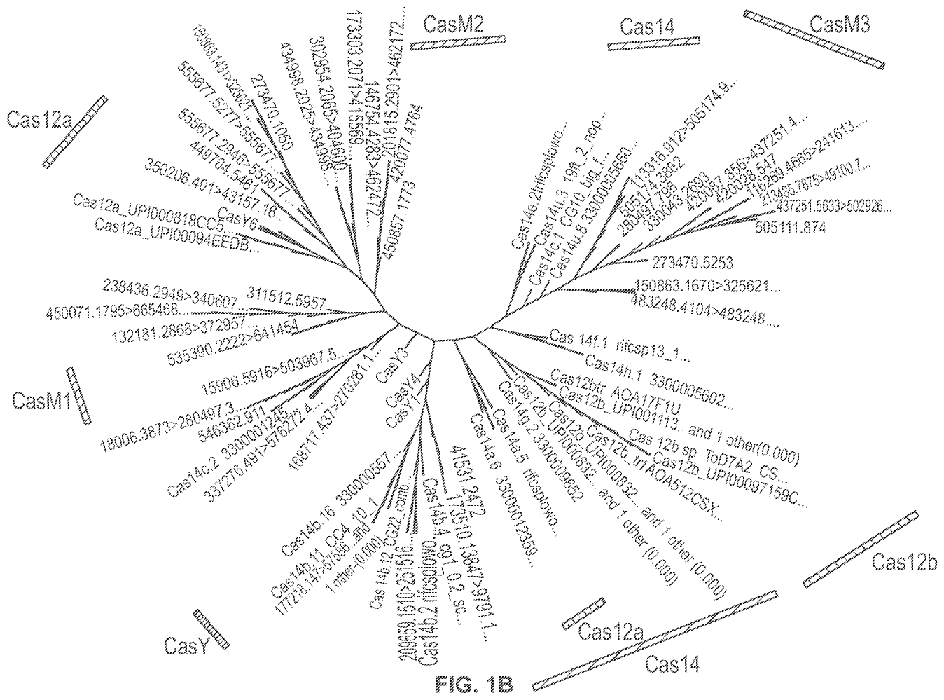

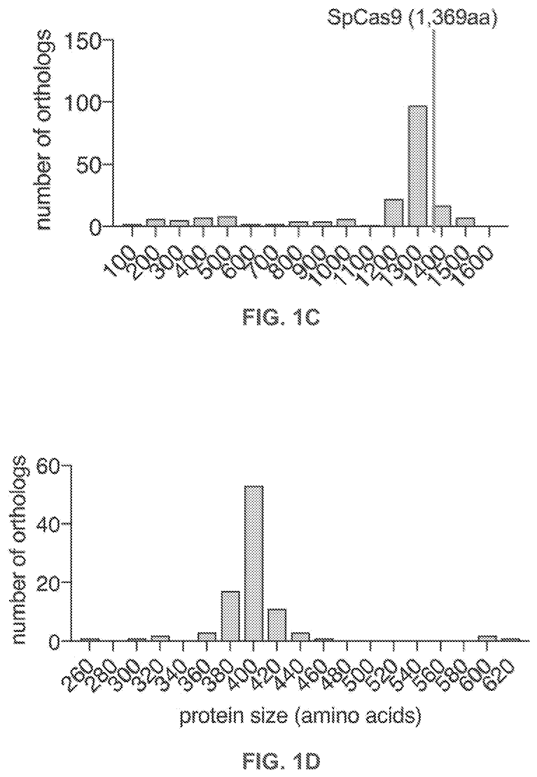

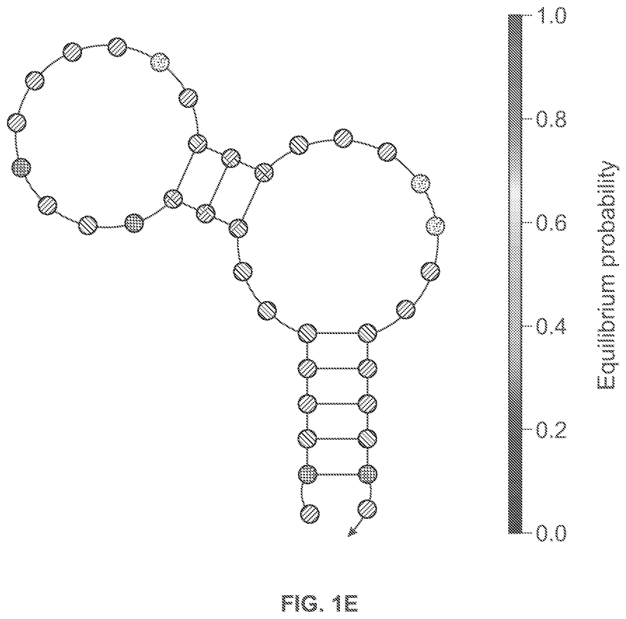

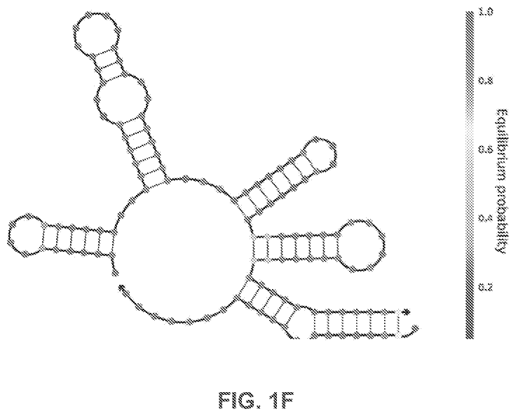

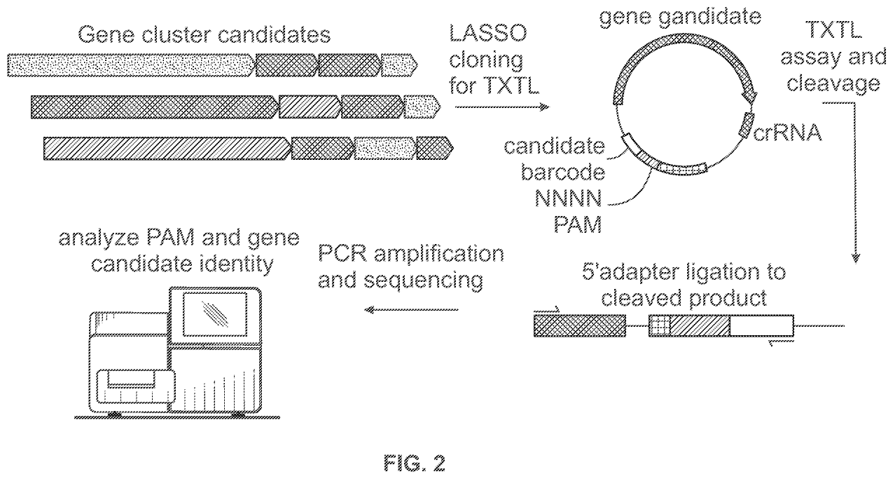

Cas12f, Cas12h and novel Cas12 systems can be mined across diverse prokaryotic genomes to identify shorter proteins. Using families of known Cas12f/h orthologs to seed hidden Markov model (HMM) alignment algorithms, NCBI and JGI databases of prokaryotic genomes and metagenomes can be searched to discovered new enzymes. The computational identification of novel miniature CRISPR nucleases from metagenomic samples is illustrated in FIG. 1A. The JGI database is particularly suitable for this search because it contains more than about 100,000 genomes and metagenomes and over about 54 billion protein coding genes, with continual rapid growth.

Single-effector CRISPR enzyme families lacking homology to classified enzymes can be found by searching for CRISPR arrays across aggregated genomes and CRISPR selecting nearby single-effector proteins, which can be putative new subtypes of Class 2 CRISPR systems. Additional sources of data from novel metagenomic sources can be used to supplement this approach, including urban-sampled metagenomes from diverse subways and microbiomes from non-western cohorts, which have been demonstrated to possess numerous additional uncharacterized genes.

CRISPR arrays as seed markers can be used to select genes within the proximity of these arrays and to develop neighborhoods of CRISPR-associated genes. HMM profiles for CRISPR-associated proteins can be generated from the literature and these profiles can be applied to filter out known systems. All remaining genes in the dataset can be clustered with linear-time clustering algorithms, such as LinClust. To select single effectors, the co-association of different protein clusters with each other can be investigated and filtered for clusters that either associate only with CRISPR arrays, or with known CRISPR adaptation machinery such as for example, without limitation, Cas1, Cas2, and Cas4. These putative single effector clusters can then be annotated for function via HMM-based alignment to assembled pfams. Clusters can be initially selected based on the presence or similarity to known nuclease domains such as for example, without limitation, RuvC and HNH, and if they are below about 800 residues in length. These candidates can be iteratively searched in a unified dataset to guarantee that “shorter” CRISPR nucleases are not misannotated truncations of larger nucleases due to loss of coverage in sequencing or homologs of larger nucleases that were truncated and inactivated. Results from panning for small CRISPR nucleases are shown in FIGS. 1B-1D and describe in Example 1 below.

### Characterization of Miniature CRISPR Nucleases

Small CRISPR nuclease systems found during computational discovery can be screened in vitro and in vivo. DNA synthesis can allow the large-scale synthesis of primers to clone gene clusters from metagenomic samples. For select candidates, the corresponding CRISPR effector gene and any accessory RNAs for testing activity can be synthesized. Although this approach can scale to tens of orthologs, complementary approaches are necessary for screening hundreds to thousands of potential orthologs for screening. Next generation DNA synthesis can allow large scale synthesis of primers to clone gene clusters from metagenomic samples. Small CRISPR nucleases can be amplified from urban sample metagenomes, either in isolation or in context of their neighboring genes and cloned into plasmids for biochemical sampling in bulk using transcription-translation (TXTL) in microfluidic droplets. Biochemical assays can profile sequence constraints or cleavage activity of the CRISPR enzymes. Profiling can enable the engineering of these qualities for subsequent use in mammalian cells.

Small CRISPR nucleases can be cloned using covalently-linked primers (Long Adapter Single-Stranded Oligonucleotide or LASSO) generated via pooled DNA synthesis, allowing cloning of hundreds of thousands of gene candidates. Because these enzymes are selected to be small, they can easily be reconstituted in TXTL systems, allowing for rapid screening of millions of candidates in a controlled biochemical setting with no purification. When small RNAs can be expressed in TXTL system, as crRNA directionality needs to be determined for each CRISPR system, the pooled candidate library can be initially express via RNA sequencing to determine crRNA direction and processing. A second set of LASSO primers that amplify the candidate systems can then be synthesized and a synthetic CRISPR array targeting a synthetic target site can be appended on the plasmid along with a gene specific barcode. Pools of these constructs can be cloned into vectors containing the target site for the synthetic CRISPR array flanked by randomized sequences to accommodate all possible PAMs. In the TXTL system, successful cleavage events can result in a double-stranded break next to the PAM sequence, which can be captured by ligation of an adaptor. Subsequent PCR amplification can produce amplicons containing both the cleaved PAM sequence and the gene-specific barcode. Pooled sequencing of this library can reveal top candidates capable of cleavage and their corresponding sequence preferences. Additionally, the pooled TXTL assay can be performed at different timepoints to profile cleavage kinetics and select orthologs with highest activity. Once top candidates are identified, each of the enzymes can be individually cloned and the cleavage activity can be tested in individual TXTL reactions on fixed PAM targets. The candidates that are the most active and have optimal PAMs that are not too restrictive can then be confirmed.

Existing orthologs of Cas12f/h can also be screened to maximize successful identification of smaller nucleases for genome editing. This may result in issues with expression of candidate nucleases in TXTL systems. For example, base sequence biases can limit expression. If unsatisfactory results in TXTL assays are found, pooled LASSO can be used for assaying constructs heterologously in E. coli cells. Candidates can be screened targeting the synthetic guides towards a ccdB toxin plasmid with a degenerate PAM library, allowing positive selection of gene candidates with activity and facile sequencing of the candidate barcode and PAM sequence by picking surviving clones. Examples of protospacer-adjacent motif include, without limitation, NNNNGATT, NNNNGNNN, NNG, NG, NGAN, NGNG, NGAG, NGCG, NAAG, NGN, NRN, NNGRRN, NNNRRT, TTTN, TTTV, TYCV, TATV, TYCV, TATV, TTN, KYTV, TYCV, TATV, TBN, any variants thereof, and any combinations thereof.

### Guide RNA Discovery for Miniature CRISPR Nucleases

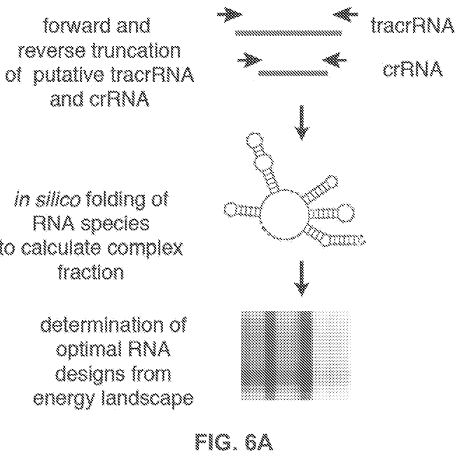

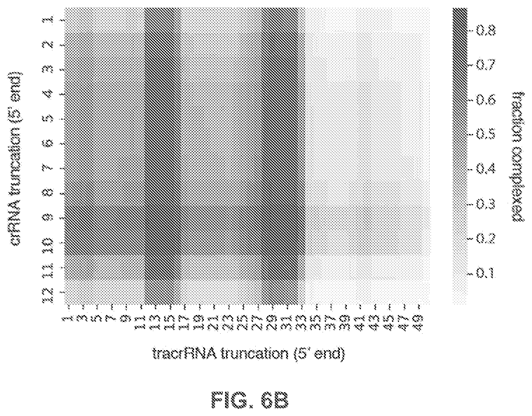

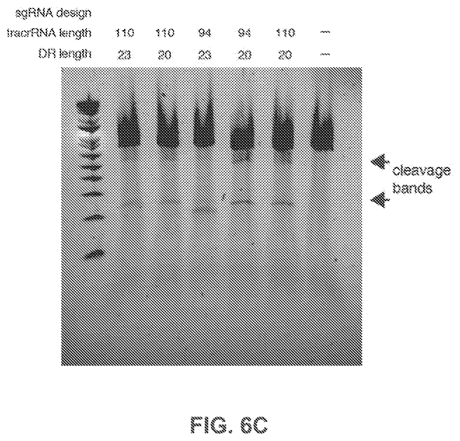

Some embodiments disclosed herein requires a gRNA comprising a tracrRNA. Small RNA sequencing studies can be performed to determine the molecular identity of the tracrRNA and associated crRNAs. However, further optimization of small RNAs is often necessary to reach levels of activity required for DNA cleavage and genome editing in mammalian cells. These designs can be informed by secondary structure algorithms to predict both optimal hybridization and tracrRNA structures with ideal hairpins for protein binding. In vitro cleavage assays can be performed with both panels of crRNAs carrying varying DR and spacer lengths as well as tracrRNAs with different architectures. These models can be further optimized across the design space in silico by progressive truncations of putative tracrRNA or crRNA and simulations of folding, resulting in an energy landscape that can be validated with in vitro cleavage reactions (FIG. 6A and FIG. 6B). Upon finding good candidates, crRNAs and tracrRNAs can then be combined into single-guide RNAs (sgRNAs) using a combination of potential loops and linkers to find the optimal sgRNA design. For Cas12 orthologs without tracrRNAs, crRNA designs can just be screened to find the optimal design. As an example, PsaCas12f was tested with different crRNA/tracrRNA designs as disclosed in Example 4 and FIG. 6C.

With optimal crRNA and sgRNA designs, mutagenesis studies can be performed to find mutations that can optimally stabilize the protein and boost cleavage activity. It was found that mutations, insertions, and deletions can drastically change the editing activity of a CRISPR enzyme. In vitro cleavage screens can be performed to find optimal sgRNA and crRNA mutants for efficient enzymatic activity. Top designs can then be tested in bacteria for confirmation of cellular DNA cleavage activity by these top orthologs.

### Characterization of Genome Editing by Miniature CRISPR Nucleases

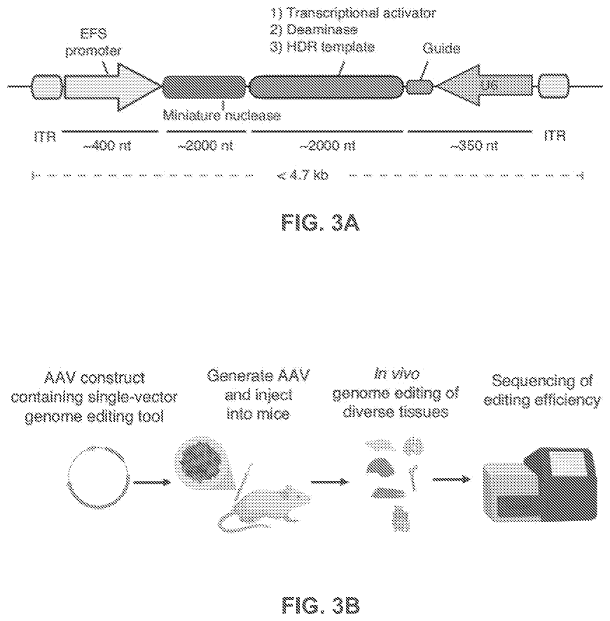

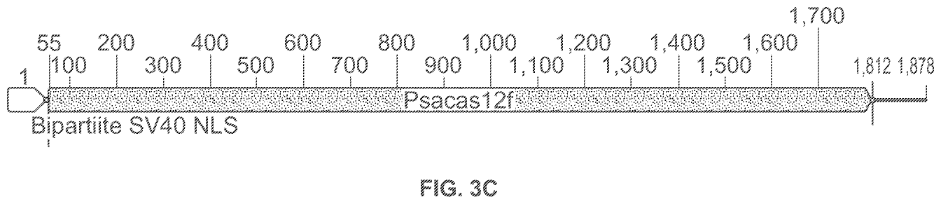

Miniature CRISPR nucleases can serve as a rich base for a new toolbox of easily-deliverable genome engineering tools. As their small size permits delivery with AAV, they can be used for genome editing in vivo. Furthermore, the additional space that is allowed by these miniature proteins can enable fusion with numerous effector domains, including transcriptional activators, repressors, and deaminases, and single vector HDR delivery (FIG. 3A). Miniature CRISPR nucleases can be engineered for mammalian genome editing and editing efficiency can be improved through multiple optimizations of the proteins. The small editors can be fused with transcriptional activators to create miniature, programmable activators capable of in vivo delivery with AAV constructs. These miniature activators can be used to demonstrate selective gene activation to activate the Pdx1 gene in vivo and treat a mouse model of Type I diabetes.

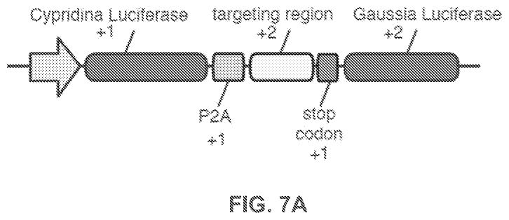

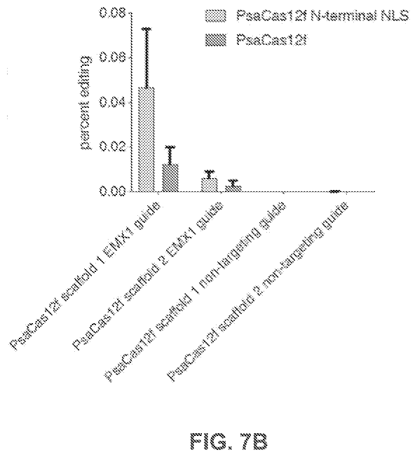

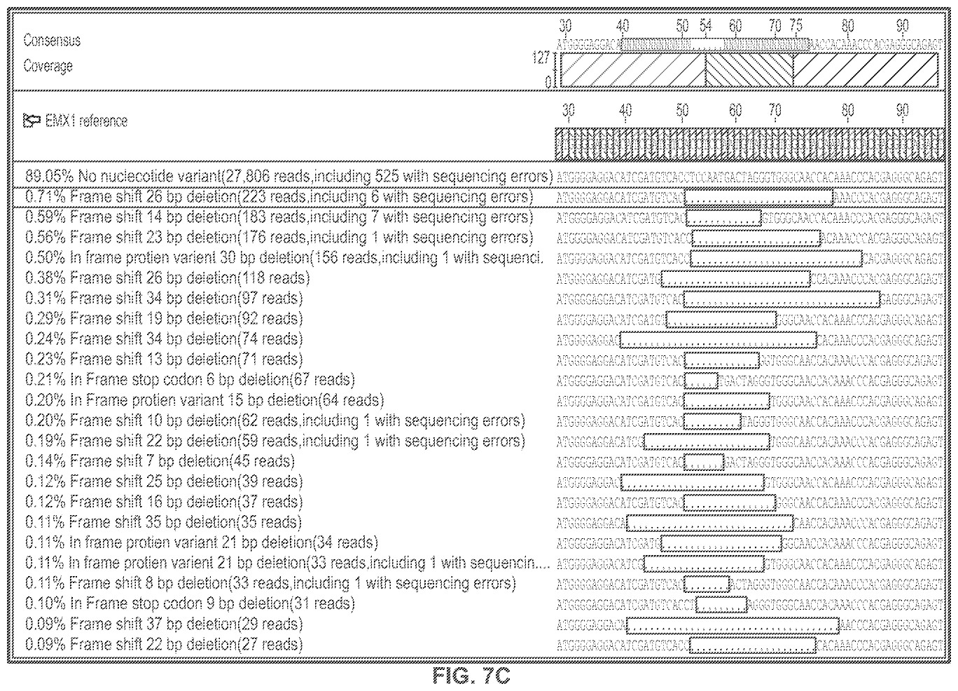

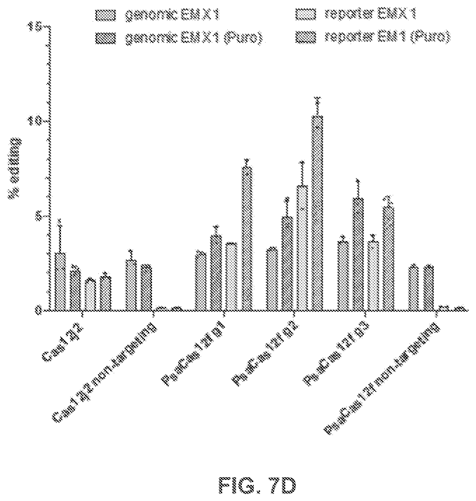

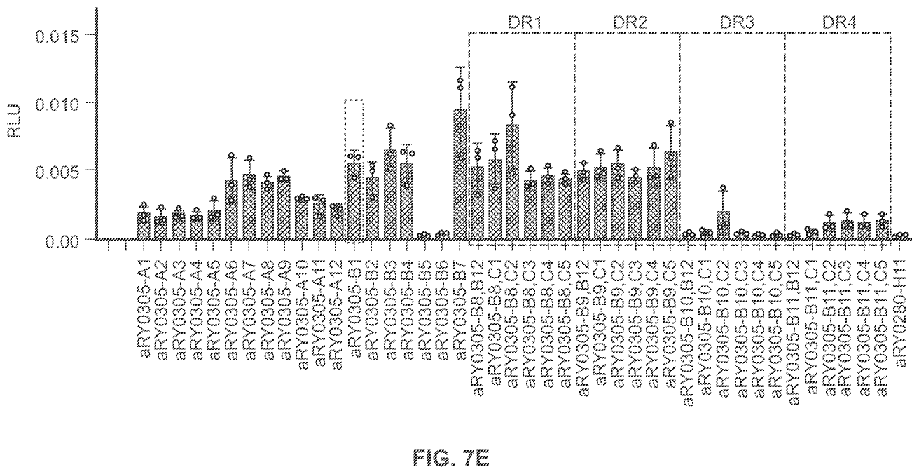

Initially, a set of miniature CRISPR nucleases can be engineered, drawn from both new nucleases and previously characterized Cas12 members, to enable genome editing. The novel nucleases can be human-codon optimized and cloned into mammalian expression constructs for genome editing on luciferase reporter constructs in HEK293FT cells. In this model, indels can inactivate the luciferase gene, allowing editing efficiency to be quantified by loss of luciferase signal (FIG. 7A). As localization of CRISPR enzymes can be a significant factor in their efficiency, top candidates can be selected and a panel of nuclear localization signals (NLS) can be fused on either the N-terminus, the C-terminus, or both to determine the effects on editing efficiency. Localization can be further verified by tagging of constructs with small HA epitope tags, which can then be interrogated using immunofluorescence microscopy. Beyond demonstrating evidence of localization, the accessibility of these tags can provide insights into the accessibility of the N- and C-termini of the protein, which can inform the engineering of activators.

Furthermore, as sgRNA expression and localization can be different in mammalian contexts than in vitro, the top sgRNA designs can be compared to further tune the efficiency of editing. Flexible insertions into the sgRNA can also be engineered, and the effects on cleavage efficiency can be tested to determine potential areas where binding loops can be inserted. Constructs with high cleavage efficiency can be validated against the disease-relevant endogenous gene EMX1. For example, editing tests from PsaCas12f family members for indel generation at EMX1 were performed as disclosed in Example 5 and FIG. 7B. Optimization of PsaCas12f in terms of codon, optimization expression, stabilization, and localization can allow for further increases in mammalian activity.

It is essential that genome editing tools such as CRISPR nucleases are active in a variety of contexts. Once the optimized enzyme and sgRNA constructs for mammalian editing are determined, these constructs can be tested for robust editing over a panel of cell lines and additional endogenous genes TRAC, VEGF, and Pdx1. As the specificity of these enzymes is an important factor into their use, both as basic research tools as well as potential future therapies, unbiased methods for profiling genome-wide specificity can be used. The best performing candidate can be subjected to a GUIDE-Seq genome-wide profiling pipeline. After knowing that these enzymes are effective and specific, they can be further engineered for activation-based applications.

### Engineering of Miniature CRISPR Activators for Programmable Gene Activation and Inhibition

Conversion of miniature CRISPR nucleases to programmable binding platforms for applications such as editing requires catalytic inactivation. To this end, conserved catalytic residues can be mutated in the RuvC domains of these type V effectors and loss of cleavage can be tested. The maintenance of binding activity can be validated by fusing an HA tag to the effector and determining binding locations by CHIP-Seq. If binding is still maintained in these catalytically inactivated mutants, CHIP signal should correspond to locations targeted by the sgRNA. Upon validation of binding in mammalian cells, this minimal programmable binding platform can be used to develop programmable activators.

To reconstitute programmable activators from the minimal CRISPR nucleases in mammalian cells, two parallel and synergistic approaches to recruit transcriptional activators can be taken. First, sets of transcriptional activators can be fused to the effector protein at either the N- or C-terminus. These fusions can be drawn from known sets of effectors, including VP64, p65, HSF1, and RTA, and these effectors can be tested in isolation or in combination of up to three effectors. In parallel, the sgRNA can be engineered to contain MS2 hairpin loops, which can bind the MCP protein. MS2 loops can then be inserted into potential predetermined accessible areas. These loops can bind MCP-activator fusions, such as MCP-VP64 or p65. These constructs can then be tested in isolation or in combination with the fusion activators to optimize the potency of activation. In order to conserve the size of constructs and avoid the need for a second promoter, a P2A fusion linker can be used to express both the minimal CRISPR nuclease and MCP-activators from a single promoter.

Candidates for transcriptional activation can be tested on luciferase reporter constructs in HEK293FT cells with a secreted luciferase downstream of a minimal promoter. This assay can allow screening of different activator constructs in throughput over multiple rounds to determine the most active construct. Importantly, the result construct from these rounds of optimization can be selected to be small enough for packaging into AAV. The activity of these constructs can be validated on endogenous genes through RT-qPCR. As recruitment of transcriptional activators and the resulting transcriptional machinery can be dependent on cell state, the optimal construct can be tested in a variety of cell types to guarantee robust activation in vivo. Lastly, the specificity of this activation system can be profiled by targeting the HBG gene in HEK293FT cells and measuring transcriptome-wide gene expression. If the activator is specific, the activation of HBG and no off-target activation should be observed. If the activator construct is specific, it can be prepared for in vivo delivery.

Transcriptional activators of the present disclosure may be targeted to specific target nucleic acids to induce activation/expression of the target nucleic acid. In some embodiments, the transcriptional activator polypeptide is targeted to the target nucleic acid via a heterologous DNA-binding domain. In this sense, a target nucleic acid of the present disclosure is targeted based on the particular nucleotide sequence in the target nucleic acid that is recognized by the targeting portion of the DNA-binding domain. In some embodiments, transcriptional activators activate expression of a target nucleic acid by being targeted to the nucleic acid with the assistance of a guide RNA (via CRISPR-based targeting). With CRISPR-based targeting, a target nucleic acid of the present disclosure can be targeted based on the particular nucleotide sequence in the target nucleic acid that is recognized by the targeting portion of the crRNA or guide RNA that is used according to the methods of the present disclosure.

Various types of nucleic acids may be targeted for activation of expression. The target nucleic acid may be located within the coding region of a target gene or upstream or downstream thereof. Moreover, the target nucleic acid may reside endogenously in a target gene or may be inserted into the gene, e.g., heterologous, for example, using techniques such as homologous recombination. For example, a target gene of the present disclosure can be operably linked to a control region, such as a promoter, which contains a sequence that can be recognized by e.g., a crRNA/tracrRNA and/or a guide RNA of the present disclosure such that a transcriptional activator of the present disclosure may be targeted to that sequence. In some embodiments, the target nucleic acid is not a target of and/or does not naturally associate with the naturally-occurring transcriptional activator polypeptide.

The target specific nucleases disclosed herein can be used with various CRISPR gene activation methods (see e.g., Konermann S, Brigham M D, Trevino A E, Joung J, Abudayyeh O O, Barcena C, Hsu P D, Habib N, Gootenberg J S, Nishimasu H, Nureki O, Zhang F. Genome-scale transcriptional activation by an engineered CRISPR-Cas9 complex. Nature. 2015 Jan. 29; 517(7536):583-8. doi: 10.1038/nature14136. Epub 2014 Dec 10. PMID: 25494202; PMCID: PMC4420636; David Bikard, Wenyan Jiang, Poulami Samai, Ann Hochschild, Feng Zhang, Luciano A. Marraffini, Programmable repression and activation of bacterial gene expression using an engineered CRISPR-Cas system, Nucleic Acids Research, Volume 41, Issue 15, 1 Aug. 2013, Pages 7429-7437, doi.org/10.1093/nar/gkt520; Perez-Pinera, P., Kocak, D., Vockley, C. et al. RNA-guided gene activation by CRISPR-Cas9-based transcription factors. Nat Methods 10, 973-976 (2013). doi.org/10.1038/nmeth.2600; Marvin E. Tanenbaum, Luke A. Gilbert, Lei S. Qi, Jonathan S. Weissman, Ronald D. Vale, “A Protein-Tagging System for Signal Amplification in Gene Expression and Fluorescence Imaging,” RESOURCE|VOLUME 159, ISSUE 3, P635-646, Oct. 23, 2014, DOI: doi.org/10.1016/j.cell.2014.09.039; Konermann S, Brigham M D, Trevino A E, Joung J, Abudayyeh O O, Barcena C, Hsu P D, Habib N, Gootenberg J S, Nishimasu H, Nureki O, Zhang F. Genome-scale transcriptional activation by an engineered CRISPR-Cas9 complex. Nature. 2015 Jan. 29; 517(7536):583-8. doi: 10.1038/nature14136. Epub 2014 Dec 10. PMID: 25494202; PMCID: PMC4420636; Chavez, A., Scheiman, J., Vora, S. et al. Highly efficient Cas9-mediated transcriptional programming. Nat. Methods 12, 326-328 (2015). doi.org/10.1038/nmeth.3312; Chavez, A., Tuttle, M., Pruitt, B. et al. Comparison of Cas9 activators in multiple species. Nat Methods 13, 563-567 (2016). doi.org/10.1038/nmeth.3871; and Sajwan, S., Mannervik, M. Gene activation by dCas9-CBP and the SAM system differ in target preference. Sci Rep 9, 18104 (2019). doi.org/10.1038/s41598-019-54179-x, which are incorporated herein by reference in their entirety).

Examples of CRISPR gene activation methods include, without limitation, dCas9-CBP CRISPR gene activation method, SPH CRISPR gene activation method, Synergistic Activation Mediator (SAM) CRISPR gene activation method, Sun Tag CRISPR gene activation method, VPR CRISPR gene activation method, and any alternative CRISPR gene activation methods therein. The dCas9-VP64 CRISPR gene activation method uses a nuclease lacking endonuclease ability and fused with VP64, a strong transcriptional activation domain. Guided by the nuclease, VP64 recruits transcriptional machinery to specific sequences, causing targeted gene regulation. This can be used to activate transcription during either initiation or elongation, depending on which sequence is targeted. The SAM CRISPR gene activation method uses engineered sgRNAs to increase transcription, which is done through creating a nuclease/VP64 fusion protein engineered with aptamers that bind to MS2 proteins. These MS2 proteins then recruit additional activation domains (HS1 and p65) to then activate genes. The Sun Tag CRISPR gene activation method uses, instead of a single copy of VP64 per each nuclease, a repeating peptide array to fused with multiple copies of VP64. By having multiple copies of VP64 at each loci of interest, this allows more transcriptional machinery to be recruited per targeted gene. The VPR CRISPR gene activation method uses a fused tripartite complex with a nuclease to activate transcription. This complex consists of the VP64 activator used in other CRISPR activation methods, as well as two other potent transcriptional activators (p65 and Rta). These transcriptional activators work in tandem to recruit transcription factors.

The target specific nucleases disclosed herein can be used as base editors for base editing (see e.g., Anzalone, A. V., Koblan, L. W. & Liu, D. R. Genome editing with CRISPR-Cas nucleases, base editors, transposases and prime editors. Nat Biotechnol 38, 824-844 (2020), which is incorporated herein by reference in its entirety). There are generally three classes of base editors: cytosine base editors (CBEs), adenine base editors (ABEs), and dual-deaminase editor (also called SPACE, synchronous programmable adenine and cytosine editor). Base editing requires a nickase or nuclease fused or coupled to a deaminase that makes the edit, a gRNA targeting the nuclease to a specific locus, and a target base for editing within the editing window specified by the nuclease.

Cytosine base editors (CBEs) uses a cytidine deaminase coupled with an inactive nuclease. These fusions convert cytosine to uracil without cutting DNA. Uracil is then subsequently converted to thymine through DNA replication or repair. Fusing an inhibitor of uracil DNA glycosylase (UGI) to a nuclease prevents base excision repair which changes the U back to a C mutation. To increase base editing efficiency, the cell can be forced to use the deaminated DNA strand as a template by using a nuclease nickase, instead of a nuclease. The resulting editor can nick the unmodified DNA strand so that it appears “newly synthesized” to the cell. Thus, the cell repairs the DNA using the U-containing strand as a template, copying the base edit.

Adenine base editors (ABEs) can convert adenine to inosine, resulting in an A to G change. Creating an adenine base editor requires an additional step because there are no known DNA adenine deaminases. Directed evolution can be used to create one from the RNA adenine deaminase TadA. While cytosine base editors often produce a mixed population of edits, some ABEs do not display significant A to non-G conversion at target loci. The removal of inosine from DNA is likely infrequent, thus preventing the induction of base excision repair. In terms of off-target effects, ABEs also generally compare favorably to other methods.

Suitable target nucleic acids will be readily apparent to one of skill in the art depending on the particular need or outcome. The target nucleic acid may be in a region of euchromatin (e.g., highly expressed gene), or the target nucleic acid may be in a region of heterochromatin (e.g., centromere DNA). Use of transcriptional activators according to the methods described herein to induce transcriptional activation in a region of heterochromatin or other highly methylated region of a plant genome may be especially useful in certain embodiments. A target nucleic acid of the present disclosure may be methylated, or it may be unmethylated.

The target gene can be any target gene used and/or known in the art. Exemplary target genes include, without limitation, Pdx1 and any variants thereof.

### Delivery of Miniature CRISPR Nucleases

In some embodiments, the target specific nuclease and/or peptide sequence are introduced into a cell as a nucleic acid encoding each protein. The nucleic acid introduced into the eukaryotic cell is a plasmid DNA or viral vector. In some embodiments, the target specific nuclease and/or peptide sequence are introduced into a cell via a ribonucleoprotein (RNP).

Delivery is in the form of a vector which may be a viral vector, such as a lenti- or baculo- or adeno-viral/adeno-associated viral vectors, but other means of delivery are known (such as yeast systems, microvesicles, gene guns/means of attaching vectors to gold nanoparticles) and are provided. The viral vector may be selected from a variety of families/genera of viruses, including, but not limited to Myoviridae, Siphoviridae, Podoviridae, Corticoviridae, Lipothrixviridae, Poxviridae, Iridoviridae, Adenoviridae, Polyomaviridae, Papillomaviridae, Mimiviridae, Pandoravirusa, Salterprovirusa, Inoviridae, Microviridae, Parvoviridae, Circoviridae, Hepadnaviridae, Caulimoviridae, Retroviridae, Cystoviridae, Reoviridae, Birnaviridae, Totiviridae, Partitiviridae, Filoviridae, Orthomyxoviridae, Deltavirusa, Leviviridae, Picornaviridae, Marnaviridae, Secoviridae, Potyviridae, Caliciviridae, Hepeviridae, Astroviridae, Nodaviridae, Tetraviridae, Luteoviridae, Tombusviridae, Coronaviridae, Arteriviridae, Flaviviridae, Togaviridae, Virgaviridae, Bromoviridae, Tymoviridae, Alphaflexiviridae, Sobemovirusa, or Idaeovirusa.

A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell. For example, baculoviruses may be used for expression in insect cells. These insect cells may, in turn be useful for producing large quantities of further vectors, such as AAV or lentivirus adapted for delivery of the present invention. Also envisaged is a method of delivering the target specific nuclease and/or peptide sequence comprising delivering to a cell mRNAs encoding each.

One of the values of miniature transcriptional activators is their capacity to be packaged in AAV. To this end, the optimal activators that are discovered can be cloned into AAV packaging vectors, and AAV2 containing the minimal activator can be purified. The activity of these AAV can be confirmed by delivery to HepG2 cells to confirm both liver targeting and activity. If titering or expression is found to be low, various liver-specific promoters can be tested, including the albumin and TBG promoters, to find minimal promoters with high expression to optimize delivery.

After confirming the delivery of the minimal construct in cell culture, expression in mice by hydrodynamic injection of promoter-less luciferase constructs can be assessed and followed by the tail-vein injection of minimal activator-AAV targeting the upstream region of these luciferase constructs. Luciferase expression can only be induced in the liver in the presence of successful activation, which can be measured by bioluminescence imaging.

To test the activation in a less perturbative model, Pdx1 can be activated. Pdx1 is a target of in vivo activation that had been performed with Cas9 activators in a Cas9-mouse model (see PMC5732045). Pdx1 overexpression in the liver can transdifferentiate hepatic cells in vivo to generate insulin-secreting cells. Pdx1 activation can be tested in cell culture using Hepa1-6 cells and expression can be measured by RT-qPCR to determine the optimal guide. These optimal Pdx1-targeting guides can be injected into mice via tail vein injection. These mice can be harvested 2 weeks post-injection to determine changes in Pdx1 expression as well as genes downstream from Pdx1 such as for example, without limitation, insulin and Pcsk1. To validate the phenotypic effects of Pdx1 targeting, mice can be treated with streptozotocin to produce hyperglycemia. The introduction of the Pdx1 activators can be tested to determine it can reduce blood glucose levels and increase serum insulin, as it has been found for Cas9 activators in a Cas9-mouse model.

Combinations of transcriptional activators can lead to successful activation. However, these combinations can be too large. If this is the case, activators can be truncated to find essential domains that allow for activation but have reduced size. Truncation of the guide RNA to modulate binding of novel Cas effectors and to quantitatively tune gene activation can be also assessed.

In some embodiments, expression of a nucleic acid sequence encoding the target specific nuclease and/or peptide sequence may be driven by a promoter. In some embodiments, the target specific nuclease is a Cas. In some embodiments, a single promoter drives expression of a nucleic acid sequence encoding a Cas and one or more of the guide sequences. In some embodiments, the Cas and guide sequence(s) are operably linked to and expressed from the same promoter. In some embodiments, the CRISPR enzyme and guide sequence(s) are expressed from different promoters. For example, the promoter(s) can be, but are not limited to, a UBC promoter, a PGK promoter, an EF1A promoter, a CMV promoter, an EFS promoter, a SV40 promoter, and a TRE promoter. The promoter may be a weak or a strong promoter. The promoter may be a constitutive promoter or an inducible promoter. In some embodiments, the promoter can also be an AAV ITR, and can be advantageous for eliminating the need for an additional promoter element, which can take up space in the vector. The additional space freed up by use of an AAV ITR can be used to drive the expression of additional elements, such as guide sequences. In some embodiments, the promoter may be a tissue specific promoter.

In some embodiments, an enzyme coding sequence encoding a target specific nuclease and/or peptide sequence is codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas protein correspond to the most frequently used codon for a particular amino acid.

In some embodiments, a vector encodes a target specific nuclease and/or peptide sequence comprising one or more nuclear localization sequences (NLSs), such as about or more than about 1, 2, 3, 4, 5, 6, 7, 8, 9, 10, or more NLSs. In some embodiments, the Cas protein comprises about or more than 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, bur other types of NLS are known. In some embodiments, the NLS is between two domains, for example between the Cas12 protein and the viral protein. The NLS may also be between two functional domains separated or flanked by a glycine-serine linker.

In general, the one or more NLSs are of sufficient strength to drive accumulation of the target specific nuclease and/or peptide sequence in a detectable amount in the nucleus of a eukaryotic cell. In general, strength of nuclear localization activity may derive from the number of NLSs in the target specific nuclease and/or other peptide sequences, the particular NLS used, or a combination of these factors. Detection of accumulation in the nucleus may be performed by any suitable technique. For example, a detectable marker may be fused to the target specific nuclease and/or peptide sequence, such that location within a cell may be visualized, such as in combination with a means for detecting the location of the nucleus (e.g., a stain specific for the nucleus such as DAPI). Examples of detectable markers include fluorescent proteins (such as green fluorescent proteins, or GFP; RFP; CFP), and epitope tags (HA tag, FLAG tag, SNAP tag). Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some respects, the invention provides methods comprising delivering one or more polynucleotides, such as one or more vectors as described herein, one or more transcripts thereof, and/or one or proteins transcribed therefrom, to a host cell. In some respects, the invention further provides cells produced by such methods, and organisms (such as animals, plants, or fungi) comprising or produced from such cells. In some embodiments, a Cas protein in combination with (and optionally complexed) with a guide sequence is delivered to a cell. Conventional viral and non-viral based gene transfer methods can be used to introduce nucleic acids in mammalian cells or target tissues. Such methods can be used to administer nucleic acids encoding a target specific nuclease and/or a blunting enzyme to cells in culture, or in a host organism. Non-viral vector delivery systems include DNA plasmids, RNA (e.g., a transcript of a vector described herein), naked nucleic acid, nucleic acid complexed with a delivery vehicle, such as a liposome, and ribonucleoprotein. Viral vector delivery systems include DNA and RNA viruses, which have either episomal or integrated genomes after delivery to the cell. For a review of gene therapy procedures, see Anderson, Science 256:808-8313 (1992); Navel and Felgner, TIBTECH 11:211-217 (1993); Mitani and Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer and Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

The target specific nuclease and/or peptide sequence can be delivered using adeno-associated virus (AAV), lentivirus, adenovirus, or other viral vector types, or combinations thereof. In some embodiments, Cas protein(s) and one or more guide RNAs can be packaged into one or more viral vectors. In some embodiments, the targeted trans-splicing system is delivered via AAV as a split intein system, similar to Levy et al. (Nature Biomedical Engineering, 2020, DOI: doi.org/10.1038/s41551-019-0501-5). In other embodiments, the target specific nuclease and/or peptide sequence can be delivered via AAV as a trans-splicing system, similar to Lai et al. (Nature Biotechnology, 2005, DOI: 10.1038/nbt1153). In some embodiments, the viral vector is delivered to the tissue of interest by, for example, an intramuscular injection, while other times the viral delivery is via intravenous, transdermal, intranasal, oral, mucosal, intrathecal, intracranial or other delivery methods. Such delivery may be either via a single dose, or multiple doses. One skilled in the art understands that the actual dosage to be delivered herein may vary greatly depending upon a variety of factors, such as the vector chosen, the target cell, organism, or tissue, the general condition of the subject to be treated, the degree of transformation/modification sought, the administration route, the administration mode, the type of transformation/modification sought, etc.

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo), or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues. Viral-mediated in vivo delivery of Cas13 and guide RNA provides a rapid and powerful technology for achieving precise mRNA perturbations within cells, especially in post-mitotic cells and tissues.

In certain embodiments, delivery of the target specific nuclease and/or peptide sequence to a cell is non-viral. In certain embodiments, the non-viral delivery system is selected from a ribonucleoprotein, cationic lipid vehicle, electroporation, nucleofection, calcium phosphate transfection, transfection through membrane disruption using mechanical shear forces, mechanical transfection, and nanoparticle delivery.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. Cell lines are available from a variety of sources known to those with skill in the art (see, e.g., the American Type Culture Collection (ATCC) (Manassas, VA). In some embodiments, a cell transfected with one or more vectors described herein is used to establish a new cell line comprising one or more vector-derived sequences.

### Diagnostics

The present disclosures provide target specific nucleases for diagnostic applications. The diagnostic applications include for example and without limitation molecular, amino acid, nucleic acid, and derivatives thereof diagnostics (see e.g., Harrington L B, Burstein D, Chen J S, Paez-Espino D, Ma E, Witte I P, Cofsky J C, Kyrpides N C, Banfield J F, Doudna J A. Programmed DNA destruction by miniature CRISPR-Cas14 enzymes. Science. 2018 Nov. 16; 362(6416):839-842. doi: 10.1126/science.aav4294. Epub 2018 Oct 18. PMID: 30337455; PMCID: PMC6659742; and Xiang X, Qian K, Zhang Z, Lin F, Xie Y, Liu Y, Yang Z. CRISPR-cas systems based molecular diagnostic tool for infectious diseases and emerging 2019 novel coronavirus (COVID-19) pneumonia. J Drug Target. 2020 August-September; 28(7-8):727-731. doi: 10.1080/1061186X.2020.1769637. Epub 2020 May 26. PMID: 32401064; PMCID: PMC7265108, which are incorporated herein by reference in their entirety). In one example, the target specific nuclease can be used with DETECTR, a DNA endonuclease-targeted CRISPR trans reporter technology for molecular diagnostics. This technique achieves high sensitivity for DNA detection by combining the activation of non-specific single-stranded deoxyribonuclease of Cas12 ssDNase with isothermal amplification that enables fast and specific detection of biologicals such as viruses. In this assay, a crRNA-Cas12a complex binds to a target DNA and induces an indiscriminate cleavage of ssDNA that is coupled to a fluorescent reporter. In another example, the target specific nuclease can be combined with a fluorescence-based point-of-care (POC) device. In this example, Cas12a/crRNA detects and binds to a targeting DNA, the Cas12a/crRNA/DNA complex then becomes activated and degrades a fluorescent ssDNA reporter to generate a signal.

### Kits

The present disclosure provides kits for carrying out a method. The present disclosure provides the invention provides kits containing any one or more of the elements disclosed in the above methods and compositions. In some embodiments, the kit comprises a vector system and instructions for using the kit. In some embodiments, the kit comprises a vector system comprising regulatory elements and polynucleotides encoding the target specific nuclease and/or peptide sequence. In some embodiments, the kit comprises a viral delivery system of the target specific nuclease and/or peptide sequence. In some embodiments, the kit comprises a non-viral delivery system of the target specific nuclease and/or peptide sequence. Elements may be provided individually or in combinations, and may be provided in any suitable container, such as a vial, a bottle, or a tube. In some embodiments, the kit includes instruction in one or more languages, for examples, in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element.

### Sequences

Sequences of target specific nucleases, guides, and nuclear localization signal (NLS) can be found in Table 1 below.

### TABLES

TABLE 1

| SEQ ID NO/ | |
|---|---|
| DESCRIPTION/SOURCE | SEQUENCE |
| SEQ ID NO: 1 | MPSETYITKTLSLKLIPSDEEKQALENYFITFQRAVNFAIDRIVDIRSSFRYLN |
| PsaCas12f | KNEQFPAVCDCCGKKEKIMYVNISNKTFKFKPSRNQKDRYTKDIYTIKPNA |
| (Artificial sequence) | HICKTCYSGVAGNMFIRKQMYPNDKEGWKVSRSYNIKVNAPGLTGTEYA |
| | MAIRKAISILRSFEKRRRNAERRIIEYEKSKKEYLELIDDVEKGKTNKIVVLE |
| | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDK |
| SEQ ID NO: 2 | MEEENFDNAEVTTGIKFKLKLNSETREKLNNYFNEYGKAINFAVRIIQKQL |
| Cas12f ortholog | ADDRFAGKAKLDENKKQLLDEDGKKIWDFPSESCSCGKQVVRYVNGKPF |
| 160429_1003 | CQECYRNKFSENGIRKRMYSAKGRKAEYDINIKNSTNRISKTHENYAIREAF |
| (Artificial sequence) | ILDKSIKKQRKERFRRLNDMMRKLQEFIDIREGKRLVCPKIERQKVERYIHP |
| | AWINKEKKIEEFRGYSLSVVNSKIKALDRNIKREEKSLKEKGQINFKARRLM |
| | LDKSVKFTDTNKVSFTISKSLPKEYELDLPKKEKRLNWLKEKIEIIKNQKPK |
| | YAYLLRRGDDFYLQYTLQTKPEIKTTHSGAVGIDRGISHIAVYTFVSNDGK |
| | NERPLFLSSSEILRLKNLQKERDKFLRRKHNKIRKKSNMRNIEDKIQLILHNY |
| | SKQIVDFAKEKNAFIVFEKLEKPKKSRSKMSKKEQYKLSLFTFKKLSDLVD |
| | YKAKREGIKVIYIEPAYTSKECSHCGEKVNTQRPFNGNYSLFKCNKCGIILN |
| | SDYNASINIAKKGLNIFNI |
| SEQ ID NO: 3 | MAKGEKNNDVLYRAVKFEIRPTLNQETILQRISSNLRLIWNEAWKERQDRY |
| Cas12f ortholog | EIFFKPIYERIYNAKKKALEKGFTDLWEKEVAKFSQQVLVKRGFPLQLVLE |
| 176283_308 | QKSLFAELKKAFEEHGITLYDQINALTAKRSLNTEFGLIPRNWQEETLDALD |
| (Artificial sequence) | GSFKSFFALRKRGDKDAKPPSERTNEDSFYKIPGRSGFKVTDDGKVIVSFGK |
| | LSETLVGRIPEYQQEKLSHAKNLKKFEIVRDERDMAKSGCFWISIAYEIPKPP |
| | ELPFNPSKAVFLAIGASWIGIISPRGEFCWRMPRPDFHWKPKINAVDERLKR |
| | VSKGSIKWKRLIFARSKMFAIMARQQKQHGQYEVIKRLLELGVCFVVTDL |
| | KVRSKEGSLADSSKAERGGSPFGANWSAQNTGNIANLVAKLTDHVSALGG |
| | MVIKRKSPELLVEEKRLPQEKRKILLAQKLKDEFLSLN |
| SEQ ID NO: 4 | MKNTKEEKWMQTYCFDLTDEEFGAENIRLATHISDSLVPLFNEVLLQVLKG |
| Cas12f ortholog | DETIKELKQEVKLRGRALKQKAKEAQMEDLWDRENNEIDDEEWLERGYD |
| 176287_13 | QEVIKEHRDYVDEIAKLYSENKVTAFDHNYHYAQENLEAIGCTAPYVNISA |
| (Artificial sequence) | GLRRGAIKNCHGAVDSWRKHLATGDYKSKPPGQQEVGKFYMLRCEPGCA |
| | VTKDRKNVRISLGDRKSSPVFELPGLDNSKNKPLHMLLRSDAKVKSFTLSR |
| | RSARNPDKKESDLQKPGVWRISINFELPLPEKKPATEYNTVALVIGSNYLGV |
| | ALHDSERNFPLNLPLPHKHWFPIIGDIEGRANVPWRKKGSKKWRRKMFGV |
| | QKKHSGGRQACYRYMARQQKQGEYETIADHLIGCGVHFVVSKPSINHPKG |
| | LADASAPDRGGDTGPNRIISSTGVNSLVLKLKQKVKEFGGSVTEMEAPPLP |
| | ERFRFWDSGPKKVIVAQLLRNQYLAQKK |
| SEQ ID NO: 5 | MVKQTTFFCKECNKNINIPRNIIKKLESNHISQDQAIKKAKERHNKKKHSLIL |
| Cas12f ortholog | GIKFKLYVKNKEDKEKLNSYFEEYAKAVTFAAQIIDKIKSGYLPQWKKDKK |
| 209659_1510 | LKRIIFPKGKCDFCGTKTEIGWISKRGKKICKNCYSKEYGENGIRKKLYATR |
| (Artificial sequence) | GRKVNPSYNIFNATKKLAATHYNYAIREAFQLLEANRKQRQERIRRLLRDK |
| | KRLREFEDLIEKPDRRIELPMKTRQREKRYIHISQKDKINELRGYTLHKIKEK |
| | IRILRRNTEREERALRKKTPIIFKGNRIMLFPQGIKFDKENNKVKITIAKNLPK |
| | EFIFSGTNVANKHGRRFFKEKLNLISQQKPKYAYLIRKQTKNSKKITDYDYY |
| | LQYTIETVYKIRKNYDGIIGIDRGINNLACLVLLEKNQEKPCGVKFYKGKEI |
| | NALKIKRRKQLYFLRRKHNRKQKQKRIRRIEPKINQILHIISKEIVELAKEKN |
| | FAIGLEQLEKPKKSRFRQRRKERYFLSLFNFKTLSTFIEYKAKKEGIRVIYIPP |
| | ERTSQICSHCAIKGDVHTNTIRPYRKPNAKKSSSSLFKCKKCGVELNADYN |
| | AAFNIAQKSLKILST |
| SEQ ID NO: 6 | MKIKEQSEVRELLKAYKYRIYPNKEQRLYLAKTFGCTRFIYNKMLSDRIKV |
| Cas12f ortholog | YEENKDLDIKKVKYPTPAQYKKEFTWLKEVDSLALANAQMNLDKAYKNF |
| 213082_2246 | FRDKSMGFPKFKSKKVNYYSYTTNNQKGTVYIEDGYIKLPKLKTMIKIKQH |
| (Artificial sequence) | RKFNGLIKSCTISKTPSNKYYISILVYTENKQLPKVDKKVGIDVGLKEFAITS |
| | NGEFFSNPKWLRKSEKRLRKLQKDLSRKQKGSNNRCKARLKVAKLHEKIT |
| | NQRKNFLHKLSIKLIRENQSIVIEDLKVKNMLQNHKLAKAISEVSWYEFRT |
| | MLEYKADWYGRELIIAPSNYASSQICSNCGYKNKEVKNLELREWVCPKCGI |
| | HHHRDINASKNLLKLAI |
| SEQ ID NO: 7 | MLVFEAKLRGTKEQYERLDEAIRTARFVRNSCLRYWMDNKGEKVGRYEL |
| Cas12f ortholog | SAYCAVLAKEFPWAKKLNSMARQASAERAWTAIARFYDNCKKKVSGKKG |
| 238436_2949 | FPKFKKYKTRDSVEYKTSGWKLSEDRRTITFTDGFKAGSFKTWGTRDLHFY |
| (Artificial sequence) | QLKQIKRVRVVRRADGYYVQFCIDQDRVEKREPTGTAIGLDVGLNHFYTD |
| | SDGQTVENPRHLRKSEKALKRLQRRLAKTQKGSKNRQKARNRLGRKHLK |
| | VSRQRKDFAVKTALCVVQSNDLVAYEDLKVRNMVKNHNLAKSISDAAWS |
| | TFRQWMEYFGKVFGVATVAVPPQYTSQNCSNCGEKIQKSLSTRTHRCPHC |
| | GFVADRDHNAAINILELGLSTVGHTETHASGDIDLCLGGETPQSKSSRRKRK |
| | PHQ |
| SEQ ID NO: 8 | MDQIIKGVKLRLYPNRGQKDKLWQMFGNDRFVWNQMLSMAKTRYQNNP |
| Cas12f ortholog | RASFINGYGMDTLLKVLKNEYPFLKESDSTSLQVVNHKLNQSFQMLFKHR |
| 265253_1259 | GGYPRFKSRKATKQAYTGKSKVSVVAKRCLKLPKIGYIKTSKTNQLVDTKI |
| (Artificial sequence) | KRYTVSYDATGRYYLSLQVEVPAPELLPKTGKVVGLDVGLADLAISSDGV |
| | KYGTFNAKWLDKQVNKWQSAYAKRKYRATIAVRQWNHNHKTVKEELN |
| | DYQNWQRARRYKARYQAKVANKRQDNLQKLTTELVKQYDVIVIEDLKTK |
| | NLQKNHHLAKSIANASWYQLRTMLEYKCAWYGRQLIIVKPNYTSQICSSC |
| | GYHNGPKPLKIREWTCSKCGVHHDRDINAAINILHKGLKANG |
| SEQ ID NO: 9 | MTSNKCAEEGQKKVSVTPITFNFWLTKVKDRIFELEDQTTVLLKDVSVDLS |
| Cas12f ortholog | RQVLKMLAGAWQSYFELRKRGDTEARPPSPKKEGWFQTMAWSNFTVRQG |
| 325997_390 | SIFVPGYQKNRIEIKLGDYLKRMVEDKEVAYVTLYRDRFSGEFNLSVVVKN |
| (Artificial sequence) | PAPKHIEHPKVIRAIDLGAGDIAVSDSSGAEYLIPARRPDKHWMPLIAQVEH |
| | RAERCIKGSRAYKRRMKARRVMHEKSGNQKDSYQRKLARALFSGEVEAIV |
| | IGKGKTRLGLAQSESGTPDQHYGAQNTGYLFRQLLYIKEKAKERGIPVVEF |
| | PDPQRKGELEDSQKKFFASRELLSLGCKKFKIEVPNSFVQGEFIFNQGKGGK |
| | PKVA |
| SEQ ID NO: 10 | MAITVHTAGVHYRWTDNPPEQLMRQLRLAHDLREDLVTLQLDYETAKAG |
| Cas12m ortholog | IWSSYPAVAAAETELADAESAAEQAAAAVSEERTKLRTKRITGPLAQKLTA |
| 58610_1188_protein_ | ARKRVREARSTRRAAISEVHEEAKGRLVDASDALKAQQKALYKTYCQDG |
| locus_of_contig_ | DLFWATFNDVLDHHKAAVKRIGQMRAAGQPAQLRHHRFDGTGSIAVQLQ |
| LFOD01000003_- | RQAGQPQRTPELIADVDGKYGRVLSVPWVQPDRWERIPRRERRMIGRVTV |
| Query_protein_ | RMRAGQLSGEPQWLDIPVQQHRMLPLDADITGARLTVTRTAGTLRAQISVT |
| (58610_1188)_ | AKIPDPEPVTDGPDVAVHLGWRNTDTGVRVARWRSTEPIEVPFDFRDTLTV |
| translation_(5) | DPGGRSGEIFVPEAVPRRVERAHLIASHRADRMNELRARLVDYLAETGPRP |
| Protein locus genbank | HPSREGEELGAGNVRMWKSPNRFAWLARVWADDESVSTDIREALAQWRH |
| annotated by | QDWISWHHQEGGRRRSAAQRLDVYRQVAAVLVSQAGRLVLDDTSYADIA |
| CrisprCasFinder for | QRSATTKTEELPNETAARINRRRAHAAPGELRQTLVAAADRDAVPVDTVS |
| protein 58610_1188 | HTGVSVVHAKCGHENPSDGRFMSVVVACDGCGEKYDQDESALTHMLTRA |
| from file 58610 | VQSAA |
| (Artificial sequence) | |
| SEQ ID NO: 11 | MTTMTVHTMGVHYKWQIPEVLRQQLWLAHNLREDLVSLQLAYDDDLKAI |
| Cas12m ortholog | WSSYPDVAQAEDTMAAAEADAVALSERVKQARIEARSKKISTELTQQLRD |
| 63461_4106_protein_ | AKKRLKDARQARRDAIAVVKDDAAERRKARSDQLAADQKALYGQYCRD |
| locus_of_contig_ | GDLYWASFNTVLDHHKTAVKRIAAQRASGKPATLRHHRFDGSGTIAVQLQ |
| LSK01000323- | RQAGAPPRTPMVLADEAGKYRNVLHIPGWTDPDVWEQMTRSQCRQSGRV |
| Query_protein_ | TVRMRCGSTDGQPQWIDLPVQVHRWLPADADITGAELVVTRVAGIYRAKL |
| (63461_4106)_ | CVTARIGDTEPVTSGPTVALHLGWRSTEEGTAVATWRSDAPLDIPFGLRTV |
| translation_(4) | MRVDAAGTSGIIVVPATIERRLTRTENIASSRSLALDALRDKVVGWLSDND |
| Protein locus genbank | APTYRDAPLEAATVKQWKSPQRFASLAHAWKDNGTEISDILWAWFSLDRK |
| annotated by | QWAQQENGRRKALGHRDDLYRQIAAVISDQAGHVLVDDTSVAELSARAM |
| CrisprCasFinder for | ERTELPTEVQQKIDRRRDHAAPGGLRASVVAAMTRDGVPVTIVAAADFTR |
| protein 63461_4106 | THSRCGHVNPADDRYLSNPVRCDGCGAMYDQDRSFVTLMLRAATAPSNP |
| from file 63461 | |
| (Artificial sequence) | |
| SEQ ID NO: 12 | MPDQLTQQLRLAHDLREDLVTLEYEYEDAVKAVWSSYPAVAALEAQVAE |
| Cas12m ortholog | LDERASELASTVKEEKSRQRTKRPSHPAVAQLAETRAQLKAAKASRREAIA |
| 21566_3969_protein_ | SVRDEATERLRTISDERYAAQKQLYRDYCTDGLLYWATFNAVLDHHKTAV |
| locus_of_contig_ | KRIAAHRKQGRAAQLRHHRWDGTGTISVQLQRQATDPARTPAIIADADTG |
| BAFB01000202_- | KWRSSLIVPWVNPDVWDTMDRASRRKAGRVVIRMRCGSSRNPDGTKTSE |
| Query_protein_ | WIDVPVQQHRMLPADADITAAQLTVRREGADLRATIGITAKIPDQGEVDEG |
| (21566_3969)_ | PTIAVHLGWRSSDHGTVVATWRSTEPLDIPETLRGVITTQSAERTVGSIVVP |
| translation_(4) | HRIEQRVHHHATVASHRDLAVDSIRDTLVAWLTEHGPQPHPYDGDPITAAS |
| Protein locus genbank | VQRWKAPRRFAWLALQWRDTPPPEGADIAETLEAWRRADKKLWLESEHG |
| annotated by | RGRALRHRTDLHRQVAAYFAGVAGRIVVDDSDIAQIAGTAKHSELLTDVD |
| CrisprCasFinder for | RQIARRRAIAAPGMLRAAIVAAATRDEVPTTTVSHTGLSRVHAACGHENPA |
| protein 21566_3969 | DDRYLMQPVLCDGCGRTYDTDLSATILMLQRASAATSN |
| from file 21566 | |
| (Artificial sequence) | |
| SEQ ID NO: 13 | MLRAYKYRIYPTDEQKVLFAKTFGCCRFVYNWALNLKITAYKERKETLGN |
| Cas12m ortholog | VYLTNLMKSELKVEHEWLSEVNSQSLQSSLRNLDTAYTNFFRNTKAVGFP |
| 633299_527_protein_ | RFKSRKDKQSFLCPQHCRVDFEKGTITIPKAKDIPAVLHRRFKGTVKTVTIS |
| locus_of_contig_ | MTPSGRYFASVLVDTSMQEMKPSEPMRDTTVGIDLGIKSLAVCSDGRTFAN |
| Scfld15_- | PKNLQRSLDRLKLLQKRLSRKQKGSANRNKARIRVARLQEHIANSRKDSLH |
| Query_protein_ | KITHALTHDSQVRTICMEDLNVKGMQRNHHLAQAVGDASFGMFLTLLEYK |
| (633299_527)_(4) | CSWYGVNLIKIDRFAPSSKTCGKCGHVYKGLNLSERSWTCPECGTHHDRDF |
| Protein | NAACNIKEFGLKALPTERGKVKPVDCPLVDDRPRVLKSNGRKKQEKRGGI |
| locus genbank | GISEAAKSLV |
| annotated by | |
| CrisprCasFinder for | |
| protein 633299_527 | |
| from file 633299 | |
| (Artificial sequence) | |
| SEQ ID NO: 14 | PQGIKFDKENNKVKITIAKNLPKEFIFSGTNVANKHGRRFFKEKLNLISQQKP |
| Cas12m ortholog | KYAYLIRKQTKNSKKITDYDYYLQYTIETVYKIRKNYDGIIGIDRGINNLAC |
| 209658_13971_protein_ | LVLLEKNQEKPCGVKFYKGKEINALKIKRRKQLYFLRRKHNRKQKQKRIRR |
| locus_of_contig_ | IEPKINQILHIISKEIVELAKEKNFAIGLEQLEKPKKSRFRQRRKERYFLSLFNF |
| Ga0190333_1001561_- | KTLSTFIEYKAKKEGIRVIYIPPERTSQICSHCAIKGDVHTNTIRPYRKPNAKK |
| Query_protein_ | SSSSLFKCKKCGVELNADYNAAFNIAQKSLKILST |
| (209658_13971)_(2) | |
| Protein locus genbank | |
| annotated by | |
| CrisprCasFinder for | |
| protein 20965_13971 | |
| from file 209658 | |
| (Artificial sequence) | |
| SEQ ID NO: 15 | DRGINNLACLVLLEKNQEKPCGVKFYKGKEINALKIKRRKQLYFLRRKHNR |
| Cas12m ortholog | KQKQKRIRRIEPKINQILHIISKEIVELAKEKNFAIGLEQLEKPKKSRFRQRRK |
| 209657_57738_protein_ | ERYFLSLFNFKTLSTFIEYKAKKEGIRVIYIPPERTSQICSHCAIKGDVHTNTIR |
| locus_of_contig_ | PYRKPNAKKSSSSLFKCKKCGVELNADYNAAFNIAQKSLKILST |
| Ga0190332_1015597_- | |
| Query_protein_ | |
| (209657_57738)_(2) | |
| Protein | |
| locus genbank | |
| annotated by | |
| CrisprCasFinder for | |
| protein 209657_57738 | |
| from file 209657 | |
| (Artificial sequence) | |
| SEQ ID NO: 16 | LLEKNQEKPCGVKFYKGKEINALKIKRRKQLYFLRRKHNRKQKQKRIRRIE |
| Cas12m ortholog | PKINQILHIISKEIVELAKEKNFAIGLEQLEKPKKSRFRQRRKERYFLSLFNFK |
| 209660_51257_protein_ | TLSTFIEYKAKKEGIRVIYIPPERTSQICSHCAIKGDVHTNTIRPYRKPNAKKS |
| locus_of_contig_ | SSSLFKCKKCGVELNADYNAAFNIAQKSLKILST |
| Ga0190335_1015156_- | |
| Query_protein_ | |
| (209660_51257)_(2) | |
| Protein | |
| locus genbank | |
| annotated by | |
| CrisprCasFinder for | |
| protein 209660_51257 | |
| from file 209660 | |
| (Artificial sequence) | |
| SEQ ID NO: 17 | MEYSYKFRVYPTAAQAEQIQRTFGCCRFVWNHYLALRKDLYEQDGKTMN |
| Cas12m ortholog | YNACSGDMTQLKKTLLWLREVDATALQSSLRDLDTAYQNFFRRVKKGEK |
| 466065_250_protein_ | PGYPKFKSKHHSKKSYKSKCVGTNIKVLDKAVQLPKLGLVKCRISKEVKGR |
| locus_of_contig_ | ILSATISQNPSGKYFVAICCTDVELEPLTSTGAVAGIDMGLKAFAITSDGVEY |
| SFKR01000004.1_- | PNHKYLTKSQKKLAKLQRQLSRKSKGSKRREKARIQVARLHEHVANQRQD |
| Query_protein_ | MLHKLSTDLVRNYDLIAIEDLAPSNMVKNHMLAKAISDASWGEFPRQLKY |
| (466065_250) | KAEWHGKKVVTVGRFFPSSQLCSNCGAQWSGTKDLSVRQWTCPVCGAIH |
| Protein locus | DRDMNAARNILNEGLRLMA |
| genbank annotated by | |
| CrisprCasFinder for | |
| protein 466065_250 | |
| from file 466065 | |
| (Artificial sequence) | |
| SEQ ID NO: 18 | VYNYFLSQRKEQYRLTGKSDNYYAQAKTLTALKKQEETAWLKEVNAQTL |
| Cas12m ortholog | QFAIKSLESAYTNFFKKSAKFPKFKSKHSKNSFTVPQSASVAGGRLFIPKFTE |
| 8971_2857_protein_ | GIKCSVHREIKGKIGKVTITKSPSGKYFVSVFTEEEYITQLEKTGKSIGLDMG |
| locus_of_contig_ | LKDLLITSEGEIFNNNRYTRRYECKLAKAQRHLSRKKKGSRGFENQRLKVA |
| OEJQ01000083.1_- | RLHEKIVNSRTDYLHKCSISLVRRYDIICIEDLNVKGMTKNHHLAKSITDAS |
| Query_protein_ | WGKFVSMLTYKAEWNNKKVVDVDRYFPSSQTCNVCGYVNKQIKDLSVRE |
| (8971_2857) | WECPHCHTHHDRDKNAAINILRIGLNNNISAGTVDYTGGEEVRTDLLESHS |
| Protein locus | SVKPEANEPLVHG |
| genbank annotated by | |
| CrisprCasFinder for | |
| protein 8971_2857 | |
| from file 8971 | |
| (Artificial sequence) | |
| SEQ ID NO: 19 | MLAKHFGCSRFVYNYFLSQRKEQYRLTGKSDNYYAQAKTLTALKKQEET |
| Cas12m ortholog | AWLKEVNAQTLQFAIKSLESAYTNFFKKSAKFPKFKSKHSKNSFTVPQSAS |
| 9265_901_protein_ | VAGGRLFIPKFTEGIKCSVHREIKGKIGKVTITKSPSGKYFVSVFTEEEYITQL |
| locus_of_contig_ | EKTGKSIGLDMGLKDLLITSEGEIFNNNRYTRRYECKLAKAQRHLSRKKKG |
| OEFX01000005.1_- | SRGFENQRLKVARLHEKIVNSRTDYLHKCSISLVRRYDIICIEDLNVKGMTK |
| Query_protein_ | NHHLAKSITDASWGKFVSMLTYKAEWNNKKVVDVDRYFPSSQTCNVCGY |
| (9265_901) | VNKQIKDLSVREWECPHCHTHHDRDKNAAINILRIGLNNNISAGTVDYTGG |
| Protein locus | EEVRTDLLESHSSVKPEANEPLVHG |
| genbank annotated by | |
| CrisprCasFinder for | |
| protein 9265_901 from | |
| file 9265 | |
| (Artificial sequence) | |
| SEQ ID NO: 20 | GATTGTATTATGCTCCACTTTAATAAGTGGTGCCTTCCAAAGCTATATGC |
| sgRNA 1 | TGAGGGAGGATGGGCGCTGTTGCAGCGTCTGCCCACCTCAGAGTGGGTA |
| (Artificial sequence) | TCCTTACCTATTGAAAACCCAAAGTAATAGGTCAAGGAATGCAAC |
| SEQ ID NO: 21 | GATTGTATTATGCTCCACTTTAATAAGTGGTGCCTTCCAAAGCTATATGC |
| sgRNA 2 | TGAGGGAGGATGGGCGCTGTTGCAGCGTCTGCCCACCTCAGAGTGGGTA |
| (Artificial sequence) | TCCTTACCTATTGAAAAGTAATAGGTCAAGGAATGCAAC |
| SEQ ID NO: 22 | GATTGTATTATGCTCCACTTTAATAAGTGGTGCCTTCCAAAGCTATATGC |
| sgRNA 3 | TGAGGGAGGATGGGCGCTGTTGCAGCGTCTGCCCACCTCAGAGTGGGTA |
| (Artificial sequence) | TCCTTACCTATTGAAAAATAGGTCAAGGAATGCAAC |
| SEQ ID NO: 23 | GATTGTATTATGCTCCACTTTAATAAGTGGTGCCTTCCAAAGCTATATGC |
| sgRNA 4 | TGAGGGAGGATGGGCGCTGTTGCAGCGTCTGCCCACCTCAGAGTGGGTA |
| (Artificial sequence) | TCCTTACCTATTGAAATAATAGGTCAAGGAATGCAAC |
| SEQ ID NO: 24 | GCTTTAATAAGTGGTGCCTTCCAAAGCTATATGCTGAGGGAGGATGGGC |
| sgRNA 5 | GCTGTTGCAGCGTCTGCCCACCTCAGAGTGGGTATCCTTACCTATTGAA |
| (Artificial sequence) | AACCCAAAGTAATAGGTCAAGGAATGCAAC |
| SEQ ID NO: 25 | GCTTTAATAAGTGGTGCCTTCCAAAGCTATATGCTGAGGGAGGATGGGC |
| sgRNA 6 | GCTGTTGCAGCGTCTGCCCACCTCAGAGTGGGTATCCTTACCTATTGAA |
| (Artificial sequence) | AAGTAATAGGTCAAGGAATGCAAC |
| SEQ ID NO: 26 | GCTTTAATAAGTGGTGCCTTCCAAAGCTATATGCTGAGGGAGGATGGGC |
| sgRNA 7 | GCTGTTGCAGCGTCTGCCCACCTCAGAGTGGGTATCCTTACCTATTGAA |
| (Artificial sequence) | AAATAGGTCAAGGAATGCAAC |
| SEQ ID NO: 27 | GCTTTAATAAGTGGTGCCTTCCAAAGCTATATGCTGAGGGAGGATGGGC |
| sgRNA 8 | GCTGTTGCAGCGTCTGCCCACCTCAGAGTGGGTATCCTTACCTATTGAA |
| (Artificial sequence) | ATAATAGGTCAAGGAATGCAAC |
| SEQ ID NO: 28 | GGTGCCTTCCAAAGCTATATGCTGAGGGAGGATGGGCGCTGTTGCAGCG |
| sgRNA 9 | TCTGCCCACCTCAGAGTGGGTATCCTTACCTATTGAAAACCCAAAGTAA |
| (Artificial sequence) | TAGGTCAAGGAATGCAAC |
| SEQ ID NO: 29 | GGTGCCTTCCAAAGCTATATGCTGAGGGAGGATGGGCGCTGTTGCAGCG |
| sgRNA 10 | TCTGCCCACCTCAGAGTGGGTATCCTTACCTATTGAAAAGTAATAGGTC |
| (Artificial sequence) | AAGGAATGCAAC |
| SEQ ID NO: 30 | GGTGCCTTCCAAAGCTATATGCTGAGGGAGGATGGGCGCTGTTGCAGCG |
| sgRNA 11 | TCTGCCCACCTCAGAGTGGGTATCCTTACCTATTGAAAAATAGGTCAAG |
| (Artificial sequence) | GAATGCAAC |
| SEQ ID NO: 31 | GGTGCCTTCCAAAGCTATATGCTGAGGGAGGATGGGCGCTGTTGCAGCG |
| sgRNA 1 | TCTGCCCACCTCAGAGTGGGTATCCTTACCTATTGAAATAATAGGTCAA |
| (Artificial sequence) | GGAATGCAAC |
| SEQ ID NO: 32 | GCCTTCCAAAGCTATATGCTGAGGGAGGATGGGCGCTGTTGCAGCGTCT |
| sgRNA 13 | GCCCACCTCAGAGTGGGTATCCTTACCTATTGAAAACCCAAAGTAATAG |
| (Artificial sequence) | GTCAAGGAATGCAAC |
| SEQ ID NO: 33 | GCCTTCCAAAGCTATATGCTGAGGGAGGATGGGCGCTGTTGCAGCGTCT |
| sgRNA 14 | GCCCACCTCAGAGTGGGTATCCTTACCTATTGAAAAGTAATAGGTCAAG |
| (Artificial sequence) | GAATGCAAC |
| SEQ ID NO: 34 | GCCTTCCAAAGCTATATGCTGAGGGAGGATGGGCGCTGTTGCAGCGTCT |
| sgRNA 15 | TGCCCACCTCAGAGTGGGTATCCTTACCTATTGAAAAATAGGTCAAGGA |
| (Artificial sequence) | ATGCAAC |
| SEQ ID NO: 35 | GCCTTCCAAAGCTATATGCTGAGGGAGGATGGGCGCTGTTGCAGCGTCT |
| sgRNA 16 | GCCCCACCTCAGAGTGGGTATCCTTACCTATTGAAATAATAGGTCAAGG |
| (Artificial sequence) | AATTGCAAC |
| SEQ ID NO: 36 | GTGCCTTCCAAAGCTATATGCTGAGGGAGGATGGGCGCTGTTGCAGCGT |
| sgRNA 17 | CTGCCCACCTCAGAGTGGGTATCCTTACCTATTGAAAACCCAAAGTAAT |
| (Artificial sequence) | AGGTCAAGGAATGCAAC |
| SEQ ID NO: 37 | GTGCCTTCCAAAGCTATATGCTGAGGGAGGATGGGCGCTGTTGCAGCGT |
| sgRNA 18 | CTGCCCACCTCAGAGTGGGTATCCTTACCTATTGAAAAGTAATAGGTCA |
| (Artificial sequence) | AGGAATGCAAC |
| SEQ ID NO: 38 | GTGCCTTCCAAAGCTATATGCTGAGGGAGGATGGGCGCTGTTGCAGCGT |
| sgRNA 19 | CTGCCCACCTCAGAGTGGGTATCCTTACCTATTGAAAAATAGGTCAAGG |
| (Artificial sequence) | AATGCAAC |
| SEQ ID NO: 39 | GTGCCTTCCAAAGCTATATGCTGAGGGAGGATGGGCGCTGTTGCAGCGT |
| sgRNA 20 | CTGCCCACCTCAGAGTGGGTATCCTTACCTATTGAAATAATAGGTCAAG |
| (Artificial sequence) | GAATGCAAC |
| SEQ ID NO: 40 | GTCCACTTTAATAAGTGGTGCCTTCCAAAGCTATATGCTGAGGGAGGAT |
| sgRNA 21 | GGGCGCTGTTGCAGCGTCTGCCCACCTCAGAGTGGGTATCCTTACCTAT |
| (Artificial sequence) | TGAAAACCCAAAGTAATAGGTCAAGGAATGCAAC |
| SEQ ID NO: 41 | GTCCACTTTAATAAGTGGTGCCTTCCAAAGCTATATGCTGAGGGAGGAT |
| sgRNA 22 | GGGCGCTGTTGCAGCGTCTGCCCACCTCAGAGTGGGTATCCTTACCTAT |
| (Artificial sequence) | TGAAAAGTAATAGGTCAAGGAATGCAAC |
| SEQ ID NO: 42 | GTCCACTTTAATAAGTGGTGCCTTCCAAAGCTATATGCTGAGGGAGGAT |
| sgRNA 23 | GGGCGCTGTTGCAGCGTCTGCCCACCTCAGAGTGGGTATCCTTACCTAT |
| (Artificial sequence) | TGAAAAATAGGTCAAGGAATGCAAC |
| SEQ ID NO: 43 | GTCCACTTTAATAAGTGGTGCCTTCCAAAGCTATATGCTGAGGGAGGAT |
| sgRNA 24 | GGGCGCTGTTGCAGCGTCTGCCCACCTCAGAGTGGGTATCCTTACCTAT |
| (Artificial sequence) | TGAAATAATAGGTCAAGGAATGCAAC |
| SEQ ID NO: 44 | EGAPKKKRKVGGSMPSETYITKTLSLKLIPSDEEKQALENYFITFQRAVNFAI |
| n-terminal NLS SV40 | DRIVDIRSSFRYLNKNEQFPAVCDCCGKKEKIMYVNISNKTFKFKPSRNQKD |
| large T antigen (from | RYTKDIYTIKPNAHICKTCYSGVAGNMFIRKQMYPNDKEGWKVSRSYNIK |
| plasmid) | VNAPGLTGTEYAMAIRKAISILRSFEKRRRNAERRIIEYEKSKKEYLELIDDV |
| (Artificial sequence) | EKGKTNKIVVLEKEGHQRVKRYKHKNWPEKWQGISLNKAKSKVKDIEKRI |
| | KKLKEWKHPTLNRPYVELHKNNVRIVGYETVELKLGNKMYTIHFASISNLR |
| | KPFRKQKKKSIEYLKHLLTLALKRNLETYPSIIKRGKNFFLQYPVRVTVKVP |
| | KLTKNFKAFGIDRGVNRLAVGCIISKDGKLTNKNIFFFHGKEAWAKENRYK |
| | KIRDRLYAMAKKLRGDKTKKIRLYHEIRKKFRHKVKYFRRNYLHNISKQIV |
| | EIAKENTPTVIVLEDLRYLRERTYRGKGRSKKAKKTNYKLNTFTYRMLIDM |
| | IKYKAEEAGVPVMIIDPRNTSRKCSKCGYVDENNRKQASFKCLKCGYSLNA |
| | DLNAAVNIAKAFYECPTFRWEEKLHAYVCSEPDK |
| SEQ ID NO: 45 | PKKKRKVGGSMPSETYITKTLSLKLIPSDEEKQALENYFITFQRAVNFAIDRI |
| n-terminal NLS SV40 | VDIRSSFRYLNKNEQFPAVCDCCGKKEKIMYVNISNKTFKFKPSRNQKDRY |
| large T antigen | TKDIYTIKPNAHICKTCYSGVAGNMFIRKQMYPNDKEGWKVSRSYNIKVN |
| (Artificial sequence) | APGLTGTEYAMAIRKAISILRSFEKRRRNAERRIIEYEKSKKEYLELIDDVEK |
| | GKTNKIVVLEKEGHQRVKRYKHKNWPEKWQGISLNKAKSKVKDIEKRIKK |
| | LKEWKHPTLNRPYVELHKNNVRIVGYETVELKLGNKMYTIHFASISNLRKP |
| | FRKQKKKSIEYLKHLLTLALKRNLETYPSIIKRGKNFFLQYPVRVTVKVPKL |
| | TKNFKAFGIDRGVNRLAVGCIISKDGKLTNKNIFFFHGKEAWAKENRYKKI |
| | RDRLYAMAKKLRGDKTKKIRLYHEIRKKFRHKVKYFRRNYLHNISKQIVEI |
| | AKENTPTVIVLEDLRYLRERTYRGKGRSKKAKKTNYKLNTFTYRMLIDMIK |
| | YKAEEAGVPVMIIDPRNTSRKCSKCGYVDENNRKQASFKCLKCGYSLNAD |
| | LNAAVNIAKAFYECPTFRWEEKLHAYVCSEPDK |
| SEQ ID NO: 46 | PAAKRVKLDGGSMPSETYITKTLSLKLIPSDEEKQALENYFITFQRAVNFAI |
| n-terminal NLS c-myc | DRIVDIRSSFRYLNKNEQFPAVCDCCGKKEKIMYVNISNKTFKFKPSRNQK |
| (Artificial sequence) | DRYTKDIYTIKPNAHICKTCYSGVAGNMFIRKQMYPNDKEGWKVSRSYNI |
| | KVNAPGLTGTEYAMAIRKAISILRSFEKRRRNAERRIIEYEKSKKEYLELIDD |
| | VEKGKTNKIVVLEKEGHQRVKRYKHKNWPEKWQGISLNKAKSKVKDIEK |
| | RIKKLKEWKHPTLNRPYVELHKNNVRIVGYETVELKLGNKMYTIHFASISN |
| | LRKPFRKQKKKSIEYLKHLLTLALKRNLETYPSIIKRGKNFFLQYPVRVTVK |
| | VPKLTKNFKAFGIDRGVNRLAVGCIISKDGKLTNKNIFFFHGKEAWAKENR |
| | YKKIRDRLYAMAKKLRGDKTKKIRLYHEIRKKFRHKVKYFRRNYLHNISK |
| | QIVEIAKENTPTVIVLEDLRYLRERTYRGKGRSKKAKKTNYKLNTFTYRML |
| | IDMIKYKAEEAGVPVMIIDPRNTSRKCSKCGYVDENNRKQASFKCLKCGYS |
| | LNADLNAAVNIAKAFYECPTFRWEEKLHAYVCSEPDK |
| SEQ ID NO: 47 | KLKIKRPVKGGSMPSETYITKTLSLKLIPSDEEKQALENYFITFQRAVNFAID |
| n-terminal NLS TUS | RIVDIRSSFRYLNKNEQFPAVCDCCGKKEKIMYVNISNKTFKFKPSRNQKDR |
| (Artificial sequence) | YTKDIYTIKPNAHICKTCYSGVAGNMFIRKQMYPNDKEGWKVSRSYNIKV |
| | NAPGLTGTEYAMAIRKAISILRSFEKRRRNAERRIIEYEKSKKEYLELIDDVE |
| | KGKTNKIVVLEKEGHQRVKRYKHKNWPEKWQGISLNKAKSKVKDIEKRIK |
| | KLKEWKHPTLNRPYVELHKNNVRIVGYETVELKLGNKMYTIHFASISNLRK |
| | PFRKQKKKSIEYLKHLLTLALKRNLETYPSIIKRGKNFFLQYPVRVTVKVPK |
| | LTKNFKAFGIDRGVNRLAVGCIISKDGKLTNKNIFFFHGKEAWAKENRYKK |
| | IRDRLYAMAKKLRGDKTKKIRLYHEIRKKFRHKVKYFRRNYLHNISKQIVE |
| | IAKENTPTVIVLEDLRYLRERTYRGKGRSKKAKKTNYKLNTFTYRMLIDMI |
| | KYKAEEAGVPVMIIDPRNTSRKCSKCGYVDENNRKQASFKCLKCGYSLNA |
| | DLNAAVNIAKAFYECPTFRWEEKLHAYVCSEPDK |
| SEQ ID NO: 48 | AVKRPAATKKAGQAKKKKLDGGSMPSETYITKTLSLKLIPSDEEKQALENY |
| n-terminal NLS NLP | FITFQRAVNFAIDRIVDIRSSFRYLNKNEQFPAVCDCCGKKEKIMYVNISNK |
| (Artificial sequence) | TFKFKPSRNQKDRYTKDIYTIKPNAHICKTCYSGVAGNMFIRKQMYPNDKE |
| | GWKVSRSYNIKVNAPGLTGTEYAMAIRKAISILRSFEKRRRNAERRIIEYEK |
| | SKKEYLELIDDVEKGKTNKIVVLEKEGHQRVKRYKHKNWPEKWQGISLNK |
| | AKSKVKDIEKRIKKLKEWKHPTLNRPYVELHKNNVRIVGYETVELKLGNK |
| | MYTIHFASISNLRKPFRKQKKKSIEYLKHLLTLALKRNLETYPSIIKRGKNFF |
| | LQYPVRVTVKVPKLTKNFKAFGIDRGVNRLAVGCIISKDGKLTNKNIFFFH |
| | GKEAWAKENRYKKIRDRLYAMAKKLRGDKTKKIRLYHEIRKKFRHKVKY |
| | FRRNYLHNISKQIVEIAKENTPTVIVLEDLRYLRERTYRGKGRSKKAKKTNY |
| | KLNTFTYRMLIDMIKYKAEEAGVPVMIIDPRNTSRKCSKCGYVDENNRKQ |
| | ASFKCLKCGYSLNADLNAAVNIAKAFYECPTFRWEEKLHAYVCSEPDK |
| SEQ ID NO: 49 | MPSETYITKTLSLKLIPSDEEKQALENYFITFQRAVNFAIDRIVDIRSSFRYLN |
| c-terminal NLS SV40 | KNEQFPAVCDCCGKKEKIMYVNISNKTFKFKPSRNQKDRYTKDIYTIKPNA |
| large T antigen (from | HICKTCYSGVAGNMFIRKQMYPNDKEGWKVSRSYNIKVNAPGLTGTEYA |
| plasmid) | MAIRKAISILRSFEKRRRNAERRIIEYEKSKKEYLELIDDVEKGKTNKIVVLE |
| (Artificial sequence) | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDKGGSEGAPKKKRKV |
| SEQ ID NO: 50 | MPSETYITKTLSLKLIPSDEEKQALENYFITFQRAVNFAIDRIVDIRSSFRYLN |
| c-terminal NLS SV40 | KNEQFPAVCDCCGKKEKIMYVNISNKTFKFKPSRNQKDRYTKDIYTIKPNA |
| large T antigen | HICKTCYSGVAGNMFIRKQMYPNDKEGWKVSRSYNIKVNAPGLTGTEYA |
| (Artificial sequence) | MAIRKAISILRSFEKRRRNAERRIIEYEKSKKEYLELIDDVEKGKTNKIVVLE |
| | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDKGGSPKKKRKV |
| SEQ ID NO: 51 | MPSETYITKTLSLKLIPSDEEKQALENYFITFQRAVNFAIDRIVDIRSSFRYLN |
| c-terminal NLS c-myc | KNEQFPAVCDCCGKKEKIMYVNISNKTFKFKPSRNQKDRYTKDIYTIKPNA |
| (Artificial sequence) | HICKTCYSGVAGNMFIRKQMYPNDKEGWKVSRSYNIKVNAPGLTGTEYA |
| | MAIRKAISILRSFEKRRRNAERRIIEYEKSKKEYLELIDDVEKGKTNKIVVLE |
| | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDKGGSPAAKRVKLD |
| SEQ ID NO: 52 | MPSETYITKTLSLKLIPSDEEKQALENYFITFQRAVNFAIDRIVDIRSSFRYLN |
| c-terminal NLS TUS | KNEQFPAVCDCCGKKEKIMYVNISNKTFKFKPSRNQKDRYTKDIYTIKPNA |
| (Artificial sequence) | HICKTCYSGVAGNMFIRKQMYPNDKEGWKVSRSYNIKVNAPGLTGTEYA |
| | MAIRKAISILRSFEKRRRNAERRIIEYEKSKKEYLELIDDVEKGKTNKIVVLE |
| | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDKGGSKLKIKRPVK |
| SEQ ID NO: 53 | MPSETYITKTLSLKLIPSDEEKQALENYFITFQRAVNFAIDRIVDIRSSFRYLN |
| c-terminal NLS NLP | KNEQFPAVCDCCGKKEKIMYVNISNKTFKFKPSRNQKDRYTKDIYTIKPNA |
| (Artificial sequence) | HICKTCYSGVAGNMFIRKQMYPNDKEGWKVSRSYNIKVNAPGLTGTEYA |
| | MAIRKAISILRSFEKRRRNAERRIIEYEKSKKEYLELIDDVEKGKTNKIVVLE |
| | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDKGGSAVKRPAATKKAGQAKKKKLD |
| SEQ ID NO: 54 | EGAPKKKRKVGGSMPSETYITKTLSLKLIPSDEEKQALENYFITFQRAVNFA |
| n- and c-terminal NLS | IDRIVDIRSSFRYLNKNEQFPAVCDCCGKKEKIMYVNISNKTFKFKPSRNQK |
| SV40 large T antigen | DRYTKDIYTIKPNAHICKTCYSGVAGNMFIRKQMYPNDKEGWKVSRSYNI |
| (from plasmid) | KVNAPGLTGTEYAMAIRKAISILRSFEKRRRNAERRIIEYEKSKKEYLELIDD |
| (Artificial sequence) | VEKGKTNKIVVLEKEGHQRVKRYKHKNWPEKWQGISLNKAKSKVKDIEK |
| | RIKKLKEWKHPTLNRPYVELHKNNVRIVGYETVELKLGNKMYTIHFASISN |
| | LRKPFRKQKKKSIEYLKHLLTLALKRNLETYPSIIKRGKNFFLQYPVRVTVK |
| | VPKLTKNFKAFGIDRGVNRLAVGCIISKDGKLTNKNIFFFHGKEAWAKENR |
| | YKKIRDRLYAMAKKLRGDKTKKIRLYHEIRKKFRHKVKYFRRNYLHNISK |
| | QIVEIAKENTPTVIVLEDLRYLRERTYRGKGRSKKAKKTNYKLNTFTYRML |
| | IDMIKYKAEEAGVPVMIIDPRNTSRKCSKCGYVDENNRKQASFKCLKCGYS |
| | LNADLNAAVNIAKAFYECPTFRWEEKLHAYVCSEPDKGGSEGAPKKKRKV |
| SEQ ID NO: 55 | PKKKRKVGGSMPSETYITKTLSLKLIPSDEEKQALENYFITFQRAVNFAIDRI |
| n- and c-terminal NLS | VDIRSSFRYLNKNEQFPAVCDCCGKKEKIMYVNISNKTFKFKPSRNQKDRY |
| SV40 large T antigen | TKDIYTIKPNAHICKTCYSGVAGNMFIRKQMYPNDKEGWKVSRSYNIKVN |
| (Artificial sequence) | APGLTGTEYAMAIRKAISILRSFEKRRRNAERRIIEYEKSKKEYLELIDDVEK |
| | GKTNKIVVLEKEGHQRVKRYKHKNWPEKWQGISLNKAKSKVKDIEKRIKK |
| | LKEWKHPTLNRPYVELHKNNVRIVGYETVELKLGNKMYTIHFASISNLRKP |
| | FRKQKKKSIEYLKHLLTLALKRNLETYPSIIKRGKNFFLQYPVRVTVKVPKL |
| | TKNFKAFGIDRGVNRLAVGCIISKDGKLTNKNIFFFHGKEAWAKENRYKKI |
| | RDRLYAMAKKLRGDKTKKIRLYHEIRKKFRHKVKYFRRNYLHNISKQIVEI |
| | AKENTPTVIVLEDLRYLRERTYRGKGRSKKAKKTNYKLNTFTYRMLIDMIK |
| | YKAEEAGVPVMIIDPRNTSRKCSKCGYVDENNRKQASFKCLKCGYSLNAD |
| | LNAAVNIAKAFYECPTFRWEEKLHAYVCSEPDKGGSPKKKRK |
| SEQ ID NO: 56 | PAAKRVKLDGGSMPSETYITKTLSLKLIPSDEEKQALENYFITFQRAVNFAI |
| n- and c-terminal NLS | DRIVDIRSSFRYLNKNEQFPAVCDCCGKKEKIMYVNISNKTFKFKPSRNQK |
| c-myc | DRYTKDIYTIKPNAHICKTCYSGVAGNMFIRKQMYPNDKEGWKVSRSYNI |
| (Artificial sequence) | KVNAPGLTGTEYAMAIRKAISILRSFEKRRRNAERRIIEYEKSKKEYLELIDD |
| | VEKGKTNKIVVLEKEGHQRVKRYKHKNWPEKWQGISLNKAKSKVKDIEK |
| | RIKKLKEWKHPTLNRPYVELHKNNVRIVGYETVELKLGNKMYTIHFASISN |
| | LRKPFRKQKKKSIEYLKHLLTLALKRNLETYPSIIKRGKNFFLQYPVRVTVK |
| | VPKLTKNFKAFGIDRGVNRLAVGCIISKDGKLTNKNIFFFHGKEAWAKENR |
| | YKKIRDRLYAMAKKLRGDKTKKIRLYHEIRKKFRHKVKYFRRNYLHNISK |
| | QIVEIAKENTPTVIVLEDLRYLRERTYRGKGRSKKAKKTNYKLNTFTYRML |
| | IDMIKYKAEEAGVPVMIIDPRNTSRKCSKCGYVDENNRKQASFKCLKCGYS |
| | LNADLNAAVNIAKAFYECPTFRWEEKLHAYVCSEPDKGGSPAAKRVKLD |
| SEQ ID NO: 57 | KLKIKRPVKGGSMPSETYITKTLSLKLIPSDEEKQALENYFITFQRAVNFAID |
| n- and c-terminal NLS | RIVDIRSSFRYLNKNEQFPAVCDCCGKKEKIMYVNISNKTFKFKPSRNQKDR |
| TUS | YTKDIYTIKPNAHICKTCYSGVAGNMFIRKQMYPNDKEGWKVSRSYNIKV |
| (Artificial sequence) | NAPGLTGTEYAMAIRKAISILRSFEKRRRNAERRIIEYEKSKKEYLELIDDVE |
| | KGKTNKIVVLEKEGHQRVKRYKHKNWPEKWQGISLNKAKSKVKDIEKRIK |
| | KLKEWKHPTLNRPYVELHKNNVRIVGYETVELKLGNKMYTIHFASISNLRK |
| | PFRKQKKKSIEYLKHLLTLALKRNLETYPSIIKRGKNFFLQYPVRVTVKVPK |
| | LTKNFKAFGIDRGVNRLAVGCIISKDGKLTNKNIFFFHGKEAWAKENRYKK |
| | IRDRLYAMAKKLRGDKTKKIRLYHEIRKKFRHKVKYFRRNYLHNISKQIVE |
| | IAKENTPTVIVLEDLRYLRERTYRGKGRSKKAKKTNYKLNTFTYRMLIDMI |
| | KYKAEEAGVPVMIIDPRNTSRKCSKCGYVDENNRKQASFKCLKCGYSLNA |
| | DLNAAVNIAKAFYECPTFRWEEKLHAYVCSEPDKGGSKLKIKRPVK |
| SEQ ID NO: 58 | AVKRPAATKKAGQAKKKKLDGGSMPSETYITKTLSLKLIPSDEEKQALENY |
| n- and c-terminal NLS | FITFQRAVNFAIDRIVDIRSSFRYLNKNEQFPAVCDCCGKKEKIMYVNISNK |
| NLP | TFKFKPSRNQKDRYTKDIYTIKPNAHICKTCYSGVAGNMFIRKQMYPNDKE |
| (Artificial sequence) | GWKVSRSYNIKVNAPGLTGTEYAMAIRKAISILRSFEKRRRNAERRIIEYEK |
| | SKKEYLELIDDVEKGKTNKIVVLEKEGHQRVKRYKHKNWPEKWQGISLNK |
| | AKSKVKDIEKRIKKLKEWKHPTLNRPYVELHKNNVRIVGYETVELKLGNK |
| | MYTIHFASISNLRKPFRKQKKKSIEYLKHLLTLALKRNLETYPSIIKRGKNFF |
| | LQYPVRVTVKVPKLTKNFKAFGIDRGVNRLAVGCIISKDGKLTNKNIFFFH |
| | GKEAWAKENRYKKIRDRLYAMAKKLRGDKTKKIRLYHEIRKKFRHKVKY |
| | FRRNYLHNISKQIVEIAKENTPTVIVLEDLRYLRERTYRGKGRSKKAKKTNY |
| | KLNTFTYRMLIDMIKYKAEEAGVPVMIIDPRNTSRKCSKCGYVDENNRKQ |
| | ASFKCLKCGYSLNADLNAAVNIAKAFYECPTFRWEEKLHAYVCSEPDKGG |
| | SAVKRPAATKKAGQAKKKKLD |
| SEQ ID NO: 59 | MPSETYITKTLSLKLIPSDEEKQALENYFITFQRAVNFAIDRIVDIRSSFRYLN |
| pCMV- | KNEQFPAVCDCCGKKEKIMYVNISNKTFKFKPSRNQKDRYTKDIYTIKPNA |
| hu191034_6034 Cas14 | HICKTCYSGVAGNMFIRKQMYPNDKEGWKVSRSYNIKVNAPGLTGTEYA |
| C (term msfGFP) | MAIRKAISILRSFEKRRRNAERRIIEYEKSKKEYLELIDDVEKGKTNKIVVLE |
| (Artificial sequence) | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDKGGSVSKGEELFTGVVPILVELDGDVN |
| | GHKFSVRGEGEGDATNGKLTLKFICTTGKLPVPWPTLVTTLTYGVQCFSRY |
| | PDHMKQHDFFKSAMPEGYVQERTISFKDDGTYKTRAEVKFEGDTLVNRIE |
| | LKGIDFKEDGNILGHKLEYNFNSHNVYITADKQKNGIKANFKIRHNVEDGS |
| | VQLADHYQQNTPIGDGPVLLPDNHYLSTQSKLSKDPNEKRDHMVLLEFVT |
| | AAGITLGMDELYK |
| SEQ ID NO: 60 | MPSETYITKTLSLKLIPSDEEKQALENYFITFQRAVNFAIDRIVDIRSSFRYLN |
| pCMV- | KNEQFPAVCDCCGKKEKIMYVNISNKTFKFKPSRNQKDRYTKDIYTIKPNA |
| hu191034_6034 Cas14 | HICKTCYSGVAGNMFIRKQMYPNDKEGWKVSRSYNIKVNAPGLTGTEYA |
| C (no NLS) | MAIRKAISILRSFEKRRRNAERRIIEYEKSKKEYLELIDDVEKGKTNKIVVLE |
| (Artificial sequence) | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGKLTNKNIFFFHGKEAWAKENRYKKIRDRLYAMA |
| | KKLRGDKTKKIRLYHEIRKKFRHKVKYFRRNYLHNISKQIVEIAKENTPTVI |
| | VLEDLRYLRERTYRGKGRSKKAKKTNYKLNTFTYRMLIDMIKYKAEEAGV |
| | PVMIIDPRNTSRKCSKCGYVDENNRKQASFKCLKCGYSLNADLNAAVNIA |
| | KAFYECPTFRWEEKLHAYVCSEPDK |
| SEQ ID NO: 61 | GCTTTAATAAGTGGTGCCTTCCAAAGCTATATGCTGAGGGAGGATGGGC |
| EMX1 5′ G guides | GCTGTTGCAGCGTCTGCCCACCTCAGAGTGGGTATCCTTACCTATTGAA |
| sgRNA 1 | AACCCAAAGTAATAGGTCAAGGAATGCAAC |
| (Artificial sequence) | |
| SEQ ID NO: 62 | GATTGTATTATGCTCCACTTTAATAAGTGGTGCCTTCCAAAGCTATATGC |
| EMX1 5′ G guides | TGAGGGAGGATGGGCGCTGTTGCAGCGTCTGCCCACCTCAGAGTGGGT |
| sgRNA 2 | ATCCTTACCTATTGAAAAATAGGTCAAGGAATGCAAC |
| (Artificial sequence) | |
| SEQ ID NO: 63 | GCTTTAATAAGTGGTGCCTTCCAAAGCTATATGCTGAGGGAGGATGGGC |
| EMX1 5′ G guides | GCTGTTGCAGCGTCTGCCCACCTCAGAGTGGGTATCCTTACCTATTGAA |
| sgRNA 3 | AAGTAATAGGTCAAGGAATGCAAC |
| (Artificial sequence) | |
| SEQ ID NO: 64 | GCTTTAATAAGTGGTGCCTTCCAAAGCTATATGCTGAGGGAGGATGGGC |
| EMX1 5′ G guides | GCTGTTGCAGCGTCTGCCCACCTCAGAGTGGGTATCCTTACCTATTGAA |
| sgRNA 4 | AAATAGGTCAAGGAATGCAAC |
| (Artificial sequence) | |
| SEQ ID NO: 65 | GCTTTAATAAGTGGTGCCTTCCAAAGCTATATGCTGAGGGAGGATGGGC |
| EMX1 5′ G guides | GCTGTTGCAGCGTCTGCCCACCTCAGAGTGGGTATCCTTACCTATTGAA |
| sgRNA 5 | ATAATAGGTCAAGGAATGCAAC |
| (Artificial sequence) | |
| SEQ ID NO: 66 | GGTGCCTTCCAAAGCTATATGCTGAGGGAGGATGGGCGCTGTTGCAGCG |
| EMX1 5′ G guides | TCTGCCCACCTCAGAGTGGGTATCCTTACCTATTGAAAACCCAAAGTAA |
| sgRNA 6 | TAGGTCAAGGAATGCAAC |
| (Artificial sequence) | |
| SEQ ID NO: 67 | GGTGCCTTCCAAAGCTATATGCTGAGGGAGGATGGGCGCTGTTGCAGCG |
| EMX1 5′ G guides | TCTGCCCACCTCAGAGTGGGTATCCTTACCTATTGAAAAGTAATAGGTC |
| sgRNA 7 | AAGGAATGCAAC |
| (Artificial sequence) | |
| SEQ ID NO: 68 | GGTGCCTTCCAAAGCTATATGCTGAGGGAGGATGGGCGCTGTTGCAGCG |
| EMX1 5′ G guides | TCTGCCCACCTCAGAGTGGGTATCCTTACCTATTGAAAAATAGGTCAAG |
| sgRNA 8 | GAATGCAAC |
| (Artificial sequence) | |
| SEQ ID NO: 69 | GGTGCCTTCCAAAGCTATATGCTGAGGGAGGATGGGCGCTGTTGCAGCG |
| EMX1 5′ G guides | TCTGCCCACCTCAGAGTGGGTATCCTTACCTATTGAAATAATAGGTCAA |
| sgRNA 9 | GGAATGCAAC |
| (Artificial sequence) | |
| SEQ ID NO: 70 | GATTGTATTATGCTCCACTTTAATAAGTGGTGCCTTCCAAAGCTATATGC |
| EMX1 5′ G guides | TGAGGGAGGATGGGCGCTGTTGCAGCGTCTGCCCACCTCAGAGTGGGT |
| sgRNA 10 | ATCCTTACCTATTGAAAACCCAAAGTAATAGGTCAAGGAATGCAAC |
| (Artificial sequence) | |
| SEQ ID NO: 71 | GATTGTATTATGCTCCACTTTAATAAGTGGTGCCTTCCAAAGCTATATGC |
| EMX1 5′ G guides | TGAGGGAGGATGGGCGCTGTTGCAGCGTCTGCCCACCTCAGATGGGTAT |
| sgRNA 11 | CCTTACCTATTGAAAAGTAATAGGTCAAGGAATGCAAC |
| (Artificial sequence) | |
| SEQ ID NO: 72 | GATTGTATTATGCTCCACTTTAATAAGTGGTGCCTTCCAAAGCTATATGC |
| EMX1 5′ G guides | TGAGGGAGGATGGGCGCTGTTGCAGCGTCTGCCCACCTCAGAGTGGGT |
| sgRNA 12 | ATCCTTACCTATTGAAATAATAGGTCAAGGAATGCAAC |
| (Artificial sequence) | |
| SEQ ID NO: 73 | GCCTTCCAAAGCTATATGCTGAGGGAGGATGGGCGCTGTTGCAGCGTCT |
| EMX1 5′ G guides | GCCCACCTCAGAGTGGGTATCCTTACCTATTGAAAACCCAAAGTAATAG |
| sgRNA 13 | GTCAAGGAATGCAAC |
| (Artificial sequence) | |
| SEQ ID NO: 74 | GCCTTCCAAAGCTATATGCTGAGGGAGGATGGGCGCTGTTGCAGCGTCT |
| EMX1 5′ G guides | GCCCACCTCAGAGTGGGTATCCTTACCTATTGAAAACCCAAAGTAATAG |
| sgRNA 14 | GTCAAGGAATGCAAC |
| (Artificial sequence) | |
| SEQ ID NO: 75 | GCCTTCCAAAGCTATATGCTGAGGGAGGATGGGCGCTGTTGCAGCGTCT |
| EMX1 5′ G guides | TGCCCACCTCAGAGTGGGTATCCTTACCTATTGAAAAATAGGTCAAGGA |
| sgRNA 15 | ATGCAAC |
| (Artificial sequence) | |
| SEQ ID NO: 76 | GTGCCTTCCAAAGCTATATGCTGAGGGAGGATGGGCGCTGTTGCAGCGT |
| EMX1 5′ G guides | CTGCCCACCTCAGAGTGGGTATCCTTACCTATTGAAAAATAGGTCAAGG |
| sgRNA 16 | AATGCAAC |
| (Artificial sequence) | |
| SEQ ID NO: 77 | GTGCCTTCCAAAGCTATATGCTGAGGGAGGATGGGCGCTGTTGCAGCGT |
| EMX1 5′ G guides | CTGCCCACCTCAGAGTGGGTATCCTTACCTATTGAAATAATAGGTCAAG |
| sgRNA 17 | GAATGCAAC |
| (Artificial sequence) | |
| SEQ ID NO: 78 | GTCCACTTTAATAAGTGGTGCCTTCCAAAGCTATATGCTGAGGGAGGAT |
| EMX1 5′ G guides | GGGCGCTGTTGCAGCGTCTGCCCACCTCAGAGTGGGTATCCTTACCTAT |
| sgRNA 18 | TGAAAAACCCAAAGTAATAGGTCAAGGAATGCAAC |
| SEQ ID NO: 79 | GTCCACTTTAATAAGTGGTGCCTTCCAAAGCTATATGCTGAGGGAGGAT |
| EMX1 5′ G guides | GGGCGCTGTTGCAGCGTCTGCCCACCTCAGAGTGGGTATCCTTACCTAT |
| sgRNA 19 | TGAAAA |
| (Artificial sequence) | |
| SEQ ID NO: 80 | GACCCAAAGTAATAGGTCAAGGAATGCAACTGGTTGCCCACCCTAGTC |
| DR only 1 | ATTG |
| (Artificial sequence) | |
| SEQ ID NO: 81 | GAGTAATAGGTCAAGGAATGCAACTGGTTGCCCACCCTAGTCATTG |
| DR only 2 | |
| (Artificial sequence) | |
| SEQ ID NO: 82 | GAATAGGTCAAGGAATGCAACTGGTTGCCCACCCTAGTCATTG |
| DR only 3 | |
| (Artificial sequence) | |
| SEQ ID NO: 83 | GTAATAGGTCAAGGAATGCAACTGGTTGCCCACCCTAGTCATTG |
| DR only 4 | |
| (Artificial sequence) | |
| SEQ ID NO: 84 | GATTGTATTATGCTCCACTTTAATAAGTGGTGCCTTCCAAAGCTATATGC |
| Tracr only 1 | TGAGGGAGGATGGGCGCTGTTGCAGCGTCTGCCCACCTCAGAGTGGGT |
| (Artificial sequence) | ATCCTTACCTA |
| SEQ ID NO: 85 | GCTTTAATAAGTGGTGCCTTCCAAAGCTATATGCTGAGGGAGGATGGGC |
| Tracr only 2 | GCTGTTGCAGCGTCTGCCCACCTCAGAGTGGGTATCCTTACCTA |
| (Artificial sequence) | |
| SEQ ID NO: 86 | GGTGCCTTCCAAAGCTATATGCTGAGGGAGGATGGGCGCTGTTGCAGCG |
| Tracr only 3 | TCTGCCCACCTCAGAGTGGGTATCCTTACCTA |
| (Artificial sequence) | |
| SEQ ID NO: 87 | GCCTTCCAAAGCTATATGCTGAGGGAGGATGGGCGCTGTTGCAGCGTCT |
| Tracr only 4 | GCCCACCTCAGAGTGGGTATCCTTACCTA |
| (Artificial sequence) | |
| SEQ ID NO: 88 | GATTGTATTATGCTCCACTTTAATAAGTGGTGCCTTCCAAAGCTATATGC |
| Tracr only 5 | TGAGGGAGGATGGGCGCTGTTGCAGCGTCTGCCCACCTCAGAGTGGGT |
| (Artificial sequence) | ATCCTTACCTA |
| SEQ ID NO: 89 | GTCCACTTTAATAAGTGGTGCCTTCCAAAGCTATATGCTGAGGGAGGAT |
| Tracr only 6 | GGGCGCTGTTGCAGCGTCTGCCCACCTCAGAGTGGGTATCCTTACCTA |
| (Artificial sequence) | |
| SEQ ID NO: 90 | G----------------- |
| Tracr only 6 | TGCCTTCCAAAGCTATATGCTGAGGGAGGATGGGCGCTGTTGCAGCGTC |
| (Artificial sequence) | TGCCCACCTCAGAGTGGGTATCCTTACCTA |
| SEQ ID NO: 91 | GTTATGCTCCACTTTAATAAGTGGTGCCTTCCAAAGCTATATGCTGAGG |
| 5pr_trunc_4 | GAGGATGGGCGCTGTTGCAGCGTCTGCCCACCTCAGAGTGGGTATCCTT |
| (Artificial sequence) | ACCTATTGAAAAGTAATAGGTCAAGGAATGCAAC |
| SEQ ID NO: 92 | GTATGCTCCACTTTAATAAGTGGTGCCTTCCAAAGCTATATGCTGAGGG |
| 5pr_trunc 5 | AGGATGGGCGCTGTTGCAGCGTCTGCCCACCTCAGAGTGGGTATCCTTA |
| (Artificial sequence) | CCTATTGAAAAGTAATAGGTCAAGGAATGCAAC |
| SEQ ID NO: 93 | GATGCTCCACTTTAATAAGTGGTGCCTTCCAAAGCTATATGCTGAGGGA |
| 5pr_trunc_6 | GGATGGGCGCTGTTGCAGCGTCTGCCCACCTCAGAGTGGGTATCCTTAC |
| (Artificial sequence) | CTATTGAAAAGTAATAGGTCAAGGAATGCAAC |
| SEQ ID NO: 94 | GTCCACTTTAATAAGTGGTGCCTTCCAAAGCTATATGCTGAGGGAGGAT |
| 5pr_trunc_7 | GGGCGCTGTTGCAGCGTCTGCCCACCTCAGAGTGGGTATCCTTACCTAT |
| (Artificial sequence) | TGAAAAGTAATAGGTCAAGGAATGCAAC |
| SEQ ID NO: 95 | GCTCCGCTTTAATAAGCGGTGCCTTCCAAAGCTATATGCTGAGGGAGGA |
| SL1_modification_1 | TGGGCGCTGTTGCAGCGTCTGCCCACCTCAGAGTGGGTATCCTTACCTA |
| (Artificial sequence) | TTGAAAAGTAATAGGTCAAGGAATGCAAC |
| SEQ ID NO: 96 | GCTCCACTTTACTAAGTGGTGCCTTCCAAAGCTATATGCTGAGGGAGGA |
| SL1_modification_2 | TGGGCGCTGTTGCAGCGTCTGCCCACCTCAGAGTGGGTATCCTTACCTA |
| (Artificial sequence) | TTGAAAAGTAATAGGTCAAGGAATGCAAC |
| SEQ ID NO: 97 | GCACCACTTTAATAAGTGGTGCCTTCCAAAGCTATATGCTGAGGGAGGA |
| SL1_modification_3 | TGGGCGCTGTTGCAGCGTCTGCCCACCTCAGAGTGGGTATCCTTACCTA |
| (Artificial sequence) | TTGAAAAGTAATAGGTCAAGGAATGCAAC |
| SEQ ID NO: 98 | GCTCCACTTTAATAAGTGGAGCCTTCCAAAGCTATATGCTGAGGGAGGA |
| SL1_modification_4 | TGGGCGCTGTTGCAGCGTCTGCCCACCTCAGAGTGGGTATCCTTACCTA |
| (Artificial sequence) | TTGAAAAGTAATAGGTCAAGGAATGCAAC |
| SEQ ID NO: 99 | GCTCCACTGTAATCAGTGGTGCCTTCCAAAGCTATATGCTGAGGGAGGA |
| SL1_modification_5 | TGGGCGCTGTTGCAGCGTCTGCCCACCTCAGAGTGGGTATCCTTACCTA |
| (Artificial sequence) | TTGAAAAGTAATAGGTCAAGGAATGCAAC |
| SEQ ID NO: 100 | GTGCTCCACTTTAATAAGTGGTGCATTCCAAAGCTATATGCTGAGGGAG |
| SL1_modification_6 | GATGGGCGCTGTTGCAGCGTCTGCCCACCTCAGAGTGGGTATCCTTACC |
| (Artificial sequence) | TATTGAAAAGTAATAGGTCAAGGAATGCAAC |
| SEQ ID NO: 101 | GCTCCACTTGTAATCAAGTGGTGCCTTCCAAAGCTATATGCTGAGGGAG |
| SL1_modification_7 | GATGGGCGCTGTTGCAGCGTCTGCCCACCTCAGAGTGGGTATCCTTACC |
| (Artificial sequence) | TATTGAAAAGTAATAGGTCAAGGAATGCAAC |
| SEQ ID NO: 102 | GCTCCACTTGCTAATGCAAGTGGTGCCTTCCAAAGCTATATGCTGAGGG |
| SL1_modification_8 | AGGATGGGCGCTGTTGCAGCGTCTGCCCACCTCAGAGTGGGTATCCTTA |
| (Artificial sequence) | CCTATTGAAAAGTAATAGGTCAAGGAATGCAAC |
| SEQ ID NO: 103 | GCTCCACTTGGCTAATGCCAAGTGGTGCCTTCCAAAGCTATATGCTGAG |
| SL1_modification_9 | GGAGGATGGGCGCTGTTGCAGCGTCTGCCCACCTCAGAGTGGGTATCCT |
| (Artificial sequence) | TACCTATTGAAAAGTAATAGGTCAAGGAATGCAAC |
| SEQ ID NO: 104 | GCTCCACTTGGCATAATTGCCAAGTGGTGCCTTCCAAAGCTATATGCTG |
| SL1_modification_1 | AGGGAGGATGGGCGCTGTTGCAGCGTCTGCCCACCTCAGAGTGGGTATC |
| (Artificial sequence) | CTTACCTATTGAAAAGTAATAGGTCAAGGAATGCAAC |
| SEQ ID NO: 105 | GCTCCACTTACATGAGGATCACCCATGTAAGTGGTGCCTTCCAAAGCTA |
| SL1_MS2_hp | TATGCTGAGGGAGGATGGGCGCTGTTGCAGCGTCTGCCCACCTCAGAGT |
| (Artificial sequence) | GGGTATCCTTACCTATTGAAAAGTAATAGGTCAAGGAATGCAAC |
| SEQ ID NO: 106 | GCTCCACTTTAATAAGTGGTGCCTTCCAAAGCTAATGCTGAGGGAGGAT |
| SL2_modification_1 | GGGCGCTGTTGCAGCGTCTGCCCACCTCAGAGTGGGTATCCTTACCTAT |
| (Artificial sequence) | TGAAAAGTAATAGGTCAAGGAATGCAAC |
| SEQ ID NO: 107 | GCTCCACTTTAATAAGTGGTGCCTTCCAAAGCTAAATGCTGAGGGAGGA |
| SL2_modification_2 | TGGGCGCTGTTGCAGCGTCTGCCCACCTCAGAGTGGGTATCCTTACCTA |
| (Artificial sequence) | TTGAAAAGTAATAGGTCAAGGAATGCAAC |
| SEQ ID NO: 108 | GCTCCACTTTAATAAGTGGTGCCTTCCAAAGCCTATATGGCTGAGGGAG |
| SL2_modification_3 | GATGGGCGCTGTTGCAGCGTCTGCCCACCTCAGAGTGGGTATCCTTACC |
| (Artificial sequence) | TATTGAAAAGTAATAGGTCAAGGAATGCAAC |
| SEQ ID NO: 109 | GCTCCACTTTAATAAGTGGTGCCTTCCAAAGCTGTATATCAGCTGAGGG |
| SL2_modification_4 | AGGATGGGCGCTGTTGCAGCGTCTGCCCACCTCAGAGTGGGTATCCTTA |
| (Artificial sequence) | CCTATTGAAAAGTAATAGGTCAAGGAATGCAAC |
| SEQ ID NO: 110 | GCTCCACTTTAATAAGTGGTGCCTTCCAAAGCTGCTATATGCAGCTGAG |
| SL2_modification_5 | GGAGGATGGGCGCTGTTGCAGCGTCTGCCCACCTCAGAGTGGGTATCCT |
| (Artificial sequence) | TACCTATTGAAAAGTAATAGGTCAAGGAATGCAAC |
| SEQ ID NO: 111 | GCTCCACTTTAATAAGTGGTGCCTTCCAAAGCTGCTTATATAGCAGCTG |
| SL2_modification_6 | AGGGAGGATGGGCGCTGTTGCAGCGTCTGCCCACCTCAGAGTGGGTATC |
| (Artificial sequence) | CTTACCTATTGAAAAGTAATAGGTCAAGGAATGCAAC |
| SEQ ID NO: 112 | GCTCCACTTTAATAAGTGGTGCCTTCCAAAGCTGCTGTATATCAGCAGC |
| SL2_modification_7 | TGAGGGAGGATGGGCGCTGTTGCAGCGTCTGCCCACCTCAGAGTGGGT |
| (Artificial sequence) | ATCCTTACCTATTGAAAAGTAATAGGTCAAGGAATGCAAC |
| SEQ ID NO: 113 | GCTCCACTTTAATAAGTGGTGCCTTCCAAAGCACATGAGGATCACCCAT |
| SL2_MS2_hp | GTGCTGAGGGAGGATGGGCGCTGTTGCAGCGTCTGCCCACCTCAGAGTG |
| (Artificial sequence) | GGTATCCTTACCTATTGAAAAGTAATAGGTCAAGGAATGCAAC |
| SEQ ID NO: 114 | GTCCACTTTAATAAGTGGTGCCTTCCAAAGCTATATGCTGAGGGAGGAT |
| increase_interaction_w_ | GGGCGCTGTTGCAAGCGTCTGCCCACCTCAGAGTGGGTATCCTTACCTA |
| crRNA_13 | TTGAAAAGTAATAGGTCAAGGATTGCAAC |
| (Artificial sequence) | |
| SEQ ID NO: 115 | GTCCACTTTAATAAGTGGTGCCTTCCAAAGCTATATGCTGAGGGAGGAT |
| increase_interaction_w_ | GGGCGCTGTTGCACGCGTCTGCCCACCTCAGAGTGGGTATCCTTACCTA |
| crRNA_14 | TTGAAAAGTAATAGGTCAAGGAGTGCAAC |
| (Artificial sequence) | |
| SEQ ID NO: 116 | GTCCACTTTAATAAGTGGTGCCTTCCAAAGCTATATGCTGAGGGAGGAT |
| increase_interaction_w_ | GGGCGCTGTTGCAGGCGTCTGCCCACCTCAGAGTGGGTATCCTTACCTA |
| crRNA_15 | TTGAAAAGTAATAGGTCAAGGACTGCAAC |
| (Artificial sequence) | |
| SEQ ID NO: 117 | GTCCACTTTAATAAGTGGTGCCTTCCAAAGCTATATGCTGAGGGAGGAT |
| increase_interaction_w_ | GGGCGCTGTTGCATGCGTCTGCCCACCTCAGAGTGGGTATCCTTACCTA |
| crRNA_16 | TTGAAAAGTAATAGGTCAAGGAATGCAAC |
| (Artificial sequence) | |
| SEQ ID NO: 118 | GTCCACTTTAATAAGTGGTGCCTTCCAAAGCTATATGCTGAGGGAGGAT |
| increase_interaction_w_ | GGGCGCTGTTCGATGCGTCTGCCCACCTCAGAGTGGGTATCCTTACCTA |
| crRNA_17 | TTGAAAAGTAATAGGTCAAGGAATCGAAC |
| (Artificial sequence) | |
| SEQ ID NO: 119 | GTCCACTTTAATAAGTGGTGCCTTCCAAAGCTATATGCTGAGGGAGGAT |
| increase_interaction_w_ | GGGCGCTGTTGAGTGCGTCTGCCCACCTCAGAGTGGGTATCCTTACCTA |
| crRNA_18 | TTGAAAAGTAATAGGTCAAGGAACTCAAC |
| (Artificial sequence) | |
| SEQ ID NO: 120 | GTCCACTTTAATAAGTGGTGCCTTCCAAAGCTATATGCTGAGGGAGGAT |
| increase_interaction_w_ | GGGCGCTGTTGCGTGCGTCTGCCCACCTCAGAGTGGGTATCCTTACCTA |
| crRNA_19 | TTGAAAAGTAATAGGTCAAGGAACGCAAC |
| (Artificial sequence) | |
| SEQ ID NO: 121 | GTCCACTTTAATAAGTGGTGCCTTCCAAAGCTATATGCTGAGGGAGGAT |
| increase_interaction_w_ | GGGCGCTGTTGTATGCGTCTGCCCACCTCAGAGTGGGTATCCTTACCTA |
| crRNA_20 | TTGAAAAGTAATAGGTCAAGGAATACAAC |
| (Artificial sequence) | |
| SEQ ID NO: 122 | GTCCACTTTAATAAGTGGTGCCTTCCAAAGCTATATGCTGAGGGAGGAT |
| increase_interaction_w_ | GGGCGCTGCCGCATGCGTCTGCCCACCTCAGAGTGGGTATCCTTACCTA |
| crRNA_21 | TTGAAAAGTAATAGGTCAAGGAATGCGGC |
| (Artificial sequence) | |
| SEQ ID NO: 123 | GTCCACTTTAATAAGTGGTGCCTTCCAAAGCTATATGCTGAGGGAGGAT |
| increase_interaction_w_ | GGGCGCTGCGGCATGCGTCTGCCCACCTCAGAGTGGGTATCCTTACCTA |
| crRNA_22 | TTGAAAAGTAATAGGTCAAGGAATGCCGC |
| (Artificial sequence) | |
| SEQ ID NO: 124 | GTCCACTTTAATAAGTGGTGCCTTCCAAAGCTATATGCTGAGGGAGGAT |
| increase_interaction_w_ | GGGCGCTGTTGCGGCGTCTGCCCACCTCAGAGTGGGTATCCTTACCTAT |
| crRNA_23 | TGAAAAGTAATAGGTCAAGGAACGCAAC |
| (Artificial sequence) | |
| SEQ ID NO: 125 | GTCCACTTTAATAAGTGGTGCCTTCCAAAGCTATATGCTGAGGGAGGAT |
| increase_interaction_w_ | GGGCGCTGTTGTAGCGTCTGCCCACCTCAGAGTGGGTATCCTTACCTAT |
| crRNA_24 | |
| (Artificial sequence) | TGAAAAGTAATAGGTCAAGGAATACAAC |
| SEQ ID NO: 126 | GTCCACTTTAATAAGTGGTGCCTTCCAAAGCTATATGCTGAGGGAGGAT |
| increase_interaction_w_ | GGGCGCTGCCGCAGCGTCTGCCCACCTCAGAGTGGGTATCCTTACCTAT |
| crRNA_25 | |
| (Artificial sequence) | TGAAAAGTAATAGGTCAAGGAATGCGGC |
| SEQ ID NO: 127 | GTCCACTTTAATAAGTGGTGCCTTCCAAAGCTATATGCTGAGGGAGGAT |
| increase_interaction_w_ | GGGCGCTGCGGCAGCGTCTGCCCACCTCAGAGTGGGTATCCTTACCTAT |
| crRNA_26 | TGAAAAGTAATAGGTCAAGGAATGCCGC |
| (Artificial sequence) | |
| SEQ ID NO: 128 | GCTCCACTTTAATAAGTGGTGCCTTCCAAAGCTATATGCTGAGGGAGGA |
| increase_interaction_of_ | TGGGCGCTGTTGCAGCGTCTGCCCACGCTAGACGTGGGTATCCTTACCT |
| SL4_3 | ATTGAAAAGTAATAGGTCAAGGAATGCAAC |
| (Artificial sequence) | |
| SEQ ID NO: 129 | GCTCCACTTTAATAAGTGGTGCCTTCCAAAGCTATATGCTGAGGGAGGA |
| increase_interaction_of_ | TGGGCGCTGTTGCAGCGTCTGCCCACTGCTAGACAGTGGGTATCCTTAC |
| SL4_4 | CTATTGAAAAGTAATAGGTCAAGGAATGCAAC |
| (Artificial sequence) | |
| SEQ ID NO: 130 | GCTCCACTTTAATAAGTGGTGCCTTCCAAAGCTATATGCTGAGGGAGGA |
| increase_interaction_of_ | TGGGCGCTGTTGCAGCGTCTGCCCACCTGCTAGACAGGTGGGTATCCTT |
| SL4_5 | ACCTATTGAAAAGTAATAGGTCAAGGAATGCAAC |
| (Artificial sequence) | |
| SEQ ID NO: 131 | GCTCCACTTTAATAAGTGGTGCCTTCCAAAGCTATATGCTGAGGGAGGA |
| increase_interaction_of_ | TGGGCGCTGTTGCAGCGTCTGCCCACGCTCAGACGTGGGTATCCTTACC |
| SL4_6 | TATTGAAAAGTAATAGGTCAAGGAATGCAAC |
| (Artificial sequence) | |
| SEQ ID NO: 132 | GCTCCACTTTAATAAGTGGTGCCTTCCAAAGCTATATGCTGAGGGAGGA |
| increase_interaction_of_ | TGGGCGCTGTTGCAGCGTCTGCCCACTGCTCAGACAGTGGGTATCCTTA |
| SL4_7 | CCTATTGAAAAGTAATAGGTCAAGGAATGCAAC |
| (Artificial sequence) | |
| SEQ ID NO: 133 | GCTCCACTTTAATAAGTGGTGCCTTCCAAAGCTATATGCTGAGGGAGGA |
| increase_interaction_of_ | TGGGCGCTGTTGCAGCGTCTGCCCACCTGCTCAGACAGGTGGGTATCCT |
| SL4_8 | TACCTATTGAAAAGTAATAGGTCAAGGAATGCAAC |
| (Artificial sequence) | |
| SEQ ID NO: 134 | GCTCCACTTTAATAAGTGGTGCCTTCCAAAGCTATATGCTGAGGGAGGA |
| increase_interaction_of_ | TGGGCGCTGTTGCAGCGTCTGCCCACGCTGCTCAGACAGCGTGGGTATC |
| SL4_9 | CTTACCTATTGAAAAGTAATAGGTCAAGGAATGCAAC |
| (Artificial sequence) | |
| SEQ ID NO: 135 | GCTCCACTTTAATAAGTGGTGCCTTCCAAAGCTATATGCTGAGGGAGGA |
| increase_interaction_of_ | TGGGCGCTGTTGCAGCGTCTGCCCACTGCTGCTCAGACAGCAGTGGGTA |
| SL4_10 | TCCTTACCTATTGAAAAGTAATAGGTCAAGGAATGCAAC |
| (Artificial sequence) | |
| SEQ ID NO: 136 | GCTCCACTTTAATAAGTGGTGCCTTCCAAAGCTATATGCTGAGGGAGGA |
| SL3_MS2_hp | TGGGCGCTGTTGCAGCGTCTGCCCACACATGAGGATCACCCATGTGTGG |
| (Artificial sequence) | GTATCCTTACCTATTGAAAAGTAATAGGTCAAGGAATGCAAC |
| SEQ ID NO: 137 | GTCCACTTTAATAAGTGGTGCCTTCCAAAGCTATATGCTGAGGGAGGAT |
| increase_interaction_of_ | GGGCGCTGTTGCAGCGTCTGCCCACCTCAGAGTGGGTATCCTTACCTAT |
| SL5_4 | TAAAAGTAATAGGTCAAGGAATGCAAC |
| (Artificial sequence) | |
| SEQ ID NO: 138 | GTCCACTTTAATAAGTGGTGCCTTCCAAAGCTATATGCTGAGGGAGGAT |
| increase_interaction_of_ | GGGCGCTGTTGCAGCGTCTGCCCACCTCAGAGTGGGTATCCTTACCTAT |
| SL5_5 | TGGAAAAGCTAATAGGTCAAGGAATGCAAC |
| (Artificial sequence) | |
| SEQ ID NO: 139 | GTCCACTTTAATAAGTGGTGCCTTCCAAAGCTATATGCTGAGGGAGGAT |
| increase_interaction_of_ | GGGCGCTGTTGCAGCGTCTGCCCACCTCAGAGTGGGTATCCTTACCTAT |
| SL5_6 | TGCTAAAAGAGTAATAGGTCAAGGAATGCAAC |
| (Artificial sequence) | |
| SEQ ID NO: 140 | GTCCACTTTAATAAGTGGTGCCTTCCAAAGCTATATGCTGAGGGAGGAT |
| increase_interaction_of_ | GGGCGCTGTTGCAGCGTCTGCCCACCTCAGAGTGGGTATCCTTACCTAT |
| SL5_7 | TGTGAAAAGCATAATAGGTCAAGGAATGCAAC |
| (Artificial sequence) | |
| SEQ ID NO: 141 | GTCCACTTTAATAAGTGGTGCCTTCCAAAGCTATATGCTGAGGGAGGAT |
| increase_interaction_of_ | GGGCGCTGTTGCAGCGTCTGCCCACCTCAGAGTGGGTATCCTTACCTAT |
| SL5_8 | TGCTGAAAAGCAGTAATAGGTCAAGGAATGCAAC |
| (Artificial sequence) | |
| SEQ ID NO: 142 | GTCCACTTTAATAAGTGGTGCCTTCCAAAGCTATATGCTGAGGGAGGAT |
| increase_interaction_of_ | GGGCGCTGTTGCAGCGTCTGCCCACCTCAGAGTGGGTATCCTTACCTAT |
| SL5_9 | TGGCTGAAAAGCAGCTAATAGGTCAAGGAATGCAAC |
| (Artificial sequence) | |
| SEQ ID NO: 143 | GTCCACTTTAATAAGTGGTGCCTTCCAAAGCTATATGCTGAGGGAGGAT |
| increase_interaction_of_ | GGGCGCTGTTGCAGCGTCTGCCCACCTCAGAGTGGGTATCCTTACCTAT |
| SL5_10 | TGTGCTGAAAAGCAGCATAATAGGTCAAGGAATGCAAC |
| (Artificial sequence) | |
| SEQ ID NO: 144 | GTCCACTTTAATAAGTGGTGCCTTCCAAAGCTATATGCTGAGGGAGGAT |
| SL4_MS2_hp | GGGCGCTGTTGCAGCGTCTGCCCACCTCAGAGTGGGTATCCTTACCTAT |
| (Artificial sequence) | TACATGAGGATCACCCATGTAATAGGTCAAGGAATGCAAC |

The percent identity of Cas12 ms to other Cas12 orthologs can be found in Tables 2-13 below.

TABLE 2

| | | | | | | Cas14d.3\| | Cas14d.1\| | |
|---|---|---|---|---|---|---|---|---|
| | | | | | | RIFCSPLOWO2_{—} | RIFCSPHIGHO2_{—} | |
| | | | | | | 01_FULL_{—} | 01_FULL_{—} | |
| | Cas14g.1\| | | | | | OD1_45_34b_{—} | CPR_46_36_{—} | |
| | RBG_13_{—} | Cas14g.2\| | | | | rifcsplowo2_{—} | rifcsphigho2_{—} | |
| | scaffold_{—} | 3300009652.a\| | | | | 01_scaffold_{—} | 01_scaffold_{—} | |
| | 1401_{—} | Ga0123330_{—} | | | | 3495_curated\| | 646_curated\| | |
| | curated\| | 1010394\| | | | | 25656 . . . 27605\| | 49808 . . . 51616\| | |
| | 15949 . . . 18180 | 2814 . . . 5123 | Cas12i2 | Cas12i1 | Cas12g1 | revcom | revcom | CasY5 |
| Cas14g.1\|RBG_{—} | | 18.819 | 5.239 | 5.689 | 10.024 | 7.355 | 6.225 | 4.971 |
| 13_scaffold_1401_{—} | | | | | | | | |
| curated\| | | | | | | | | |
| 15949 . . . 18180 | | | | | | | | |
| Cas14g.2\| | 18.819 | | 5.027 | 4.978 | 8.197 | 6.75 | 6.78 | 4.996 |
| 3300009652.a\| | | | | | | | | |
| Ga0123330_1010394\| | | | | | | | | |
| 2814 . . . 5123 | | | | | | | | |
| Cas12i2 | 5.239 | 5.027 | | 4.944 | 5.939 | 5.899 | 4.155 | 4.478 |
| Cas12i1 | 5.689 | 4.978 | 4.944 | | 4.46 | 5.688 | 4.461 | 6.058 |
| Cas12g1 | 10.024 | 8.197 | 5.939 | 4.46 | | 7.375 | 7.576 | 5.483 |
| Cas14d.3\| | 7.355 | 6.75 | 5.899 | 5.688 | 7.375 | | 10.271 | 4.31 |
| RIFCSPLOWO2_{—} | | | | | | | | |
| 01_FULL_OD1_45_{—} | | | | | | | | |
| 34b_rifcsplowo2_{—} | | | | | | | | |
| 01_scaffold_{—} | | | | | | | | |
| 3495_curated\| | | | | | | | | |
| 25656 . . . 27605\| | | | | | | | | |
| revcom | | | | | | | | |
| Cas14d.1\| | 6.225 | 6.78 | 4.155 | 4.461 | 7.576 | 10.271 | | 3.457 |
| RIFCSPHIGHO2_01_{—} | | | | | | | | |
| FULL_CPR_46_{—} | | | | | | | | |
| 36_rifcsphigho2_{—} | | | | | | | | |
| 01_scaffold_{—} | | | | | | | | |
| 646_curated\| | | | | | | | | |
| 49808 . . . 51616\| | | | | | | | | |
| revcom | | | | | | | | |
| CasY5 | 4.971 | 4.996 | 4.478 | 6.058 | 5.483 | 4.31 | 3.457 | |
| Cas14a.4\| | 8.029 | 7.91 | 3.986 | 4.859 | 6.178 | 6.734 | 6.186 | 3.336 |
| CG10big_fil_rev_8_{—} | | | | | | | | |
| 21_14_0.10_{—} | | | | | | | | |
| scaffold_20906_{—} | | | | | | | | |
| curated\| | | | | | | | | |
| 649 . . . 2829 | | | | | | | | |
| CasY6 | 5.089 | 5.319 | 4.61 | 6.114 | 4.878 | 4.6 | 4.351 | 6.205 |
| Cas14f.1\| | 5.415 | 7.185 | 4.476 | 4.6 | 6.072 | 7.925 | 6.364 | 6.332 |
| rifcsp13_1_sub10_{—} | | | | | | | | |
| scaffold_3_curated\| | | | | | | | | |
| 38906 . . . 41041 | | | | | | | | |
| Cas14f.2\| | 6.218 | 7.407 | 3.864 | 3.727 | 5.315 | 7.65 | 6.347 | 3.843 |
| 3300009991.a\| | | | | | | | | |
| Ga0105042_100140\| | | | | | | | | |
| 1624 . . . 3348 | | | | | | | | |
| Cas14a.6\| | 6.371 | 5.585 | 3.575 | 3.022 | 5.478 | 7.386 | 6.088 | 3.274 |
| 3300012359.a\| | | | | | | | | |
| Ga0137385_10000156\| | | | | | | | | |
| 41289 . . . 42734 | | | | | | | | |
| Cas12a_{—} | 3.643 | 3.157 | 5.548 | 4.833 | 4.397 | 3.972 | 4.869 | 5.552 |
| UPI00094EEDB4 | | | | | | | | |
| Cas12a_{—} | 4.519 | 3.519 | 6.326 | 5.434 | 4.604 | 5.118 | 4.828 | 5.773 |
| UPI000B4235CE | | | | | | | | |
| Cas12a_{—} | 4.525 | 3.451 | 6.335 | 5.512 | 4.535 | 5.126 | 4.758 | 5.71 |
| UPI000818CC52 | | | | | | | | |
| Cas12a_UPI000 | 4.519 | 3.519 | 6.326 | 5.505 | 4.604 | 5.118 | 4.828 | 5.773 |
| 7B78B7F | | | | | | | | |
| Cas12a_{—} | 4.519 | 3.519 | 6.326 | 5.501 | 4.604 | 5.118 | 4.828 | 5.773 |
| UPI000B4235F9 | | | | | | | | |
| Cas14e.2\| | 5.204 | 5.391 | 3.425 | 3.51 | 4.439 | 5.663 | 5.627 | 3.501 |
| rifcsplowo2_01_{—} | | | | | | | | |
| scaffold_81231_{—} | | | | | | | | |
| curated\| | | | | | | | | |
| 976 . . . 2217 | | | | | | | | |
| Cas14e.1\| | 6.039 | 6.595 | 4.207 | 3.321 | 6.144 | 4.903 | 6.19 | 3.298 |
| rifcsphigho2_01_{—} | | | | | | | | |
| scaffold_566_{—} | | | | | | | | |
| curated\| | | | | | | | | |
| 113069 . . . 114313 | | | | | | | | |
| Cas14e.3\| | 3.808 | 5.292 | 4.429 | 3.337 | 4.581 | 6.917 | 5.538 | 2.681 |
| rifcsphigho2_01_{—} | | | | | | | | |
| scaffold_4702_{—} | | | | | | | | |
| curated\| | | | | | | | | |
| 82881 . . . 84230\| | | | | | | | | |
| revcom | | | | | | | | |
| CasY4 | 6.058 | 4.651 | 5.598 | 3.922 | 6.556 | 4.348 | 3.766 | 6.522 |
| Cas14h.3\| | 7.333 | 5.063 | 3.626 | 3.053 | 5.27 | 6.97 | 5.952 | 3.469 |
| 3300009698.a\| | | | | | | | | |
| Ga0116216_10000905\| | | | | | | | | |
| 8005 . . . 9504 | | | | | | | | |
| Cas14h.1\| | 5.767 | 7.752 | 4.511 | 4.255 | 6.195 | 6.031 | 5.381 | 4.825 |
| 3300005602.a\| | | | | | | | | |
| Ga0070762_10001740\| | | | | | | | | |
| 7377 . . . 9071\| | | | | | | | | |
| revcom | | | | | | | | |
| Cas14h.2\| | 6.307 | 8.258 | 4.444 | 4.089 | 5.457 | 7.386 | 5.706 | 4.474 |
| 3300005921.a\| | | | | | | | | |
| Ga0070766_10011912\| | | | | | | | | |
| 384 . . . 2081 | | | | | | | | |
| Cas14c.1\| | 5.696 | 6.349 | 4.178 | 3.815 | 5.402 | 6.036 | 4.654 | 3.616 |
| CG10_big_fil_rev_8_{—} | | | | | | | | |
| 21_14_0.10_{—} | | | | | | | | |
| scaffold_4477_{—} | | | | | | | | |
| curated\| | | | | | | | | |
| 19327 . . . 20880\| | | | | | | | | |
| revcom | | | | | | | | |
| Cas12h1 | 6.801 | 6.015 | 5.403 | 5.47 | 6.919 | 6.586 | 4.432 | 5.237 |
| CasX1 | 7.116 | 5.52 | 6.421 | 6.225 | 6.724 | 6.571 | 5.714 | 5.849 |
| CasX2 | 7.033 | 5.592 | 5.867 | 5.341 | 6.796 | 6.522 | 5.28 | 6.061 |
| CasY1 | 6.31 | 4.979 | 7.038 | 4.286 | 6.423 | 4.376 | 4.513 | 6.407 |
| Cas14u.3\| | 7.628 | 7.483 | 4.688 | 4.377 | 6.883 | 9.741 | 9.105 | 2.842 |
| 19ft_2_nophage_{—} | | | | | | | | |
| noknown_scaffold_0_{—} | | | | | | | | |
| curated\| | | | | | | | | |
| 508188 . . . 509648 | | | | | | | | |
| Cas14u.7\| | 8.531 | 7.733 | 2.921 | 3.03 | 5.952 | 5.855 | 4 | 2.743 |
| 3300001256.a\| | | | | | | | | |
| JGI12210J13797_{—} | | | | | | | | |
| 10004690\| | | | | | | | | |
| 5792 . . . 7006 | | | | | | | | |
| Cas14u.8\| | 7.341 | 5.992 | 3.891 | 3.39 | 5.812 | 6.317 | 4.341 | 2.741 |
| 3300005660.a\| | | | | | | | | |
| Ga0073904_10021651\| | | | | | | | | |
| 765 . . . 1943 | | | | | | | | |
| Cas14u.4\| | 6.137 | 5.615 | 3.783 | 3.491 | 5.797 | 8.841 | 3.797 | 3.527 |
| rifcsp2_19_4_full_{—} | | | | | | | | |
| scaffold_168_curated\| | | | | | | | | |
| 84455 . . . 85657 | | | | | | | | |
| Cas14d.2\| | 7.444 | 5.898 | 4.051 | 3.707 | 6.045 | 11.318 | 9.486 | 3.495 |
| rifcsphigho2_01_{—} | | | | | | | | |
| scaffold_10981_curated\| | | | | | | | | |
| 5762 . . . 7246\| | | | | | | | | |
| revcom | | | | | | | | |
| Cas14c.2\| | 7.459 | 7.246 | 3.961 | 4.864 | 6.021 | 6.156 | 4.859 | 3.163 |
| 3300001245.a\| | | | | | | | | |
| JGI12048J13642_{—} | | | | | | | | |
| 10201286\| | | | | | | | | |
| 4257 . . . 5489\| | | | | | | | | |
| revcom | | | | | | | | |
| CasY3 | 5.921 | 4.781 | 6.715 | 4.958 | 5.753 | 4.456 | 3.918 | 6.795 |
| 633299_527_protein_{—} | 6.853 | 7.057 | 4.203 | 3.491 | 6.109 | 5.819 | 5.28 | 3.815 |
| locus_of_contig_{—} | | | | | | | | |
| Scfld15 - | | | | | | | | |
| Query protein | | | | | | | | |
| (633299_527) | | | | | | | | |
| (4) | | | | | | | | |
| 8971_2857_protein_{—} | 6.677 | 6.14 | 5.263 | 2.944 | 5.579 | 4.866 | 4.53 | 3.704 |
| locus_of_contig_{—} | | | | | | | | |
| OEJQ01000083.1 - | | | | | | | | |
| Query protein | | | | | | | | |
| (8971_2857) | | | | | | | | |
| 9265_901_protein_{—} | 6.567 | 6.043 | 5.203 | 3.012 | 5.493 | 4.942 | 4.444 | 3.759 |
| locus_of_contig_{—} | | | | | | | | |
| OEFX01000005.1 - | | | | | | | | |
| Query protein | | | | | | | | |
| (9265_901) | | | | | | | | |
| Cas14u.6\| | 7.317 | 8.101 | 4.094 | 2.993 | 6.806 | 6.484 | 5.663 | 3.206 |
| 3300006028.a\| | | | | | | | | |
| Ga0070717_10000077\| | | | | | | | | |
| 54519 . . . 56201\| | | | | | | | | |
| revcom | | | | | | | | |
| 466065_250_protein_{—} | 7.007 | 6.564 | 4.187 | 3.868 | 6.729 | 5.271 | 6.688 | 3.439 |
| locus_of_contig_{—} | | | | | | | | |
| SFKR01000004.1 - | | | | | | | | |
| Query protein | | | | | | | | |
| (466065_250) | | | | | | | | |
| Cas14a.5\| | 6.191 | 4.78 | 3.349 | 5.14 | 4.666 | 7.069 | 6.923 | 3.578 |
| rifcsplowo2_01_{—} | | | | | | | | |
| scaffold_34461_{—} | | | | | | | | |
| curated\| | | | | | | | | |
| 4968 . . . 6521 | | | | | | | | |
| CasY2 | 5.34 | 5.364 | 5.168 | 6.993 | 5.294 | 5.448 | 4.297 | 5.865 |
| Cas14a.3\|gwa1_{—} | 9.517 | 7.923 | 5.44 | 4.995 | 7.417 | 7.339 | 5.346 | 3.767 |
| scaffold_1795_{—} | | | | | | | | |
| curated\| | | | | | | | | |
| 25635 . . . 27224\| | | | | | | | | |
| revcom | | | | | | | | |
| Cas14a.1\| | 7.921 | 7.629 | 5.186 | 4.857 | 8.052 | 7.891 | 8.1 | 3.733 |
| rifcsphigho2_02_{—} | | | | | | | | |
| scaffold_2167_{—} | | | | | | | | |
| curated\| | | | | | | | | |
| 30296 . . . 31798\| | | | | | | | | |
| revcom | | | | | | | | |
| Cas14a.2\|gwa2_{—} | 7.983 | 7.422 | 5.442 | 4.447 | 7.403 | 6.98 | 7.944 | 3.534 |
| scaffold_18027_{—} | | | | | | | | |
| curated\| | | | | | | | | |
| 7105 . . . 8628 | | | | | | | | |
| Cas14b.4\|cg1_0.2_{—} | 9.986 | 9.823 | 4.608 | 4.135 | 8.105 | 8.739 | 5.295 | 3.826 |
| scaffold_785_c_{—} | | | | | | | | |
| curated\| | | | | | | | | |
| 32521 . . . 34155 | | | | | | | | |
| Cas14b.7\| | 9.655 | 8.243 | 5.366 | 4.846 | 6.839 | 8.204 | 6.818 | 4.074 |
| 3300013125.a\| | | | | | | | | |
| Ga0172369_10000737\| | | | | | | | | |
| 994 . . . 2652\| | | | | | | | | |
| revcom | | | | | | | | |
| Cas14u.2\| | 6.828 | 7.084 | 4.02 | 3.425 | 7.723 | 5.91 | 5.854 | 3.209 |
| 3300002172.a\| | | | | | | | | |
| JGI24730J26740_{—} | | | | | | | | |
| 1002785\| | | | | | | | | |
| 496 . . . 1605\| | | | | | | | | |
| revcom | | | | | | | | |
| Cas14b.3\| | 9.904 | 9.511 | 4.701 | 5.446 | 7.245 | 6.619 | 7.362 | 4.093 |
| rifcsphigho2_01_{—} | | | | | | | | |
| scaffold_36781_{—} | | | | | | | | |
| curated\| | | | | | | | | |
| 2592 . . . 4217 | | | | | | | | |
| Cas14b.2\| | 9.218 | 9.078 | 5.352 | 4.843 | 7.324 | 7.122 | 7.355 | 4.227 |
| rifcsplowo2_01_{—} | | | | | | | | |
| scaffold_282_{—} | | | | | | | | |
| curated\| | | | | | | | | |
| 77370 . . . 78983 | | | | | | | | |
| Cas14b.1\| | 9.986 | 8.071 | 4.931 | 5.104 | 7.029 | 7.069 | 7.199 | 4.029 |
| rifcsplowo2_01_{—} | | | | | | | | |
| scaffold_239_{—} | | | | | | | | |
| curated\| | | | | | | | | |
| 54653 . . . 56257 | | | | | | | | |
| Cas14b.8\| | 10.125 | 9.029 | 4.931 | 4.915 | 7.427 | 7.806 | 8.764 | 3.491 |
| 3300013125.a\| | | | | | | | | |
| Ga0172369_10010464\| | | | | | | | | |
| 885 . . . 2489\| | | | | | | | | |
| revcom | | | | | | | | |
| Cas14b.5\| | 10.028 | 8.038 | 4.322 | 5.239 | 8.216 | 7.932 | 7.207 | 5.446 |
| rifcsphigho2_02_{—} | | | | | | | | |
| scaffold_55589_{—} | | | | | | | | |
| curated\| | | | | | | | | |
| 1904 . . . 3598 | | | | | | | | |
| Cas14b.6\| | 10.633 | 8.311 | 5.604 | 5.365 | 7.97 | 7.402 | 6.149 | 5.013 |
| CG03_land_8_20_{—} | | | | | | | | |
| 14_0.80_scaffold_{—} | | | | | | | | |
| 2214_curated\| | | | | | | | | |
| 6634 . . . 8466\| | | | | | | | | |
| revcom | | | | | | | | |
| Cas14b.9\| | 10.852 | 9.041 | 5.408 | 5.07 | 8.503 | 8.146 | 6.147 | 4.732 |
| 3300013127.a\| | | | | | | | | |
| Ga0172365_10004421\| | | | | | | | | |
| 633 . . . 2366\| | | | | | | | | |
| revcom | | | | | | | | |
| 209658_13971_{—} | 11.434 | 8.289 | 5.032 | 4.11 | 5.732 | 8.818 | 6.2 | 3.591 |
| protein_locus_{—} | | | | | | | | |
| of_contig_{—} | | | | | | | | |
| Ga0190333_1001561 - | | | | | | | | |
| Query protein | | | | | | | | |
| (209658_13971) | | | | | | | | |
| (2) | | | | | | | | |
| 209657_57738 | 21.344 | 13.074 | 9.571 | 5.621 | 12.261 | 16.216 | 10.046 | 6.757 |
| protein_locus_{—} | | | | | | | | |
| of_contig_{—} | | | | | | | | |
| Ga0190332_1015597 - | | | | | | | | |
| Query protein | | | | | | | | |
| (209657_57738) | | | | | | | | |
| (2) | | | | | | | | |
| 209660_51257 | 20.661 | 13.91 | 9.31 | 5.288 | 12.295 | 16.588 | 10.096 | 6.516 |
| protein_locus_{—} | | | | | | | | |
| of_contig_{—} | | | | | | | | |
| Ga0190335_1015156 - | | | | | | | | |
| Query protein | | | | | | | | |
| (209660_51257) | | | | | | | | |
| (2) | | | | | | | | |
| Cas14b.14\| | 8.04 | 7.412 | 4.074 | 4.384 | 7.067 | 6.771 | 5.842 | 3.704 |
| gwc1_scaffold_{—} | | | | | | | | |
| 8732_curated\| | | | | | | | | |
| 2705 . . . 4537 | | | | | | | | |
| Cas14b.15\| | 8.09 | 8.85 | 4.356 | 4.093 | 8.864 | 7.084 | 6.723 | 3.951 |
| 3300010293.a\| | | | | | | | | |
| Ga0116204_1008574\| | | | | | | | | |
| 2134 . . . 4032 | | | | | | | | |
| Cas14b.12\|CG22_{—} | 8.391 | 7.859 | 4.906 | 6.029 | 7.915 | 6.409 | 6.349 | 4.228 |
| combo_CG10 - | | | | | | | | |
| 13_8_21_14_all_{—} | | | | | | | | |
| scaffold_2003_{—} | | | | | | | | |
| curated\| | | | | | | | | |
| 553 . . . 2880\| | | | | | | | | |
| revcom | | | | | | | | |
| Cas14b.13\| | 8.545 | 9.06 | 4.72 | 5.326 | 7.65 | 7.711 | 6.46 | 3.887 |
| rifcsphigho2_01_{—} | | | | | | | | |
| scaffold_82367_{—} | | | | | | | | |
| curated\| | | | | | | | | |
| 1523 . . . 3856\| | | | | | | | | |
| revcom | | | | | | | | |
| Cas14b.16\| | 8.607 | 6.86 | 5.529 | 5.009 | 9.554 | 8.604 | 8.247 | 3.53 |
| 3300005573.a\| | | | | | | | | |
| Ga0078972_1001015a\| | | | | | | | | |
| 33750 . . . 35627 | | | | | | | | |
| Cas14b.10\| | 8.969 | 9.031 | 4.981 | 6.187 | 8.217 | 7.255 | 6.647 | 4.974 |
| CG08_land_8_20_1 | | | | | | | | |
| 4_0.20_scaffold_{—} | | | | | | | | |
| 1609_curated\| | | | | | | | | |
| 6134 . . . 7975 | | | | | | | | |
| Cas14b.11\| | 9.151 | 7.513 | 4.803 | 5.097 | 8.805 | 7.714 | 7.829 | 4.01 |
| CG4_10_14_0.8_{—} | | | | | | | | |
| um_filter_{—} | | | | | | | | |
| scaffold_20762_{—} | | | | | | | | |
| curated\| | | | | | | | | |
| 1372 . . . 3219 | | | | | | | | |
| Cas14u.1\| | 7.801 | 6.658 | 2.761 | 3.636 | 6.992 | 6.535 | 7.085 | 2.599 |
| 3300009029.a\| | | | | | | | | |
| Ga0066793_10010091\| | | | | | | | | |
| 37 . . . 1113\| | | | | | | | | |
| revcom | | | | | | | | |
| Cas12c1 | 3.749 | 5.389 | 5.444 | 5.339 | 5.582 | 4.362 | 3.803 | 5.334 |
| Cas12c2 | 5.609 | 5.178 | 5.988 | 4.403 | 5.954 | 4.676 | 4.073 | 5.778 |
| Cas12a_{—} | 4.949 | 5.412 | 7.131 | 5.547 | 5.649 | 5.709 | 5.372 | 7.105 |
| UPI001113398F | | | | | | | | |
| Cas12b_{—} | 4.949 | 5.412 | 7.131 | 5.547 | 5.649 | 5.709 | 5.372 | 7.105 |
| UPI001113398F | | | | | | | | |
| Cas12b_tr\| | 4.818 | 5.541 | 7.248 | 5.708 | 5.434 | 5.585 | 5.461 | 7.186 |
| A0A1I7F1U9\| | | | | | | | | |
| A0A1I7F1U9_9BACL | | | | | | | | |
| Cas12a_{—} | 5.013 | 5.917 | 5.824 | 5.837 | 5.986 | 5.254 | 5.085 | 6.941 |
| UPI00083514A7 | | | | | | | | |
| Cas12b_{—} | 5.013 | 5.917 | 5.824 | 5.837 | 5.986 | 5.254 | 5.085 | 6.941 |
| UPI00083514A7 | | | | | | | | |
| Cas12a_{—} | 4.865 | 6.396 | 6.03 | 5.934 | 5.845 | 5.1 | 5.743 | 6.921 |
| UPI00097159F1 | | | | | | | | |
| Cas12b_{—} | 4.865 | 6.396 | 6.03 | 5.934 | 5.845 | 5.1 | 5.743 | 6.921 |
| UPI00097159F1 | | | | | | | | |
| Cas12b_sp\| | 4.865 | 6.396 | 6.03 | 5.934 | 5.845 | 5.1 | 5.743 | 6.921 |
| T0D7A2\| | | | | | | | | |
| CS12B_ALIAG | | | | | | | | |
| Cas12a_{—} | 4.865 | 6.396 | 6.03 | 5.934 | 5.935 | 5.1 | 5.743 | 6.838 |
| UPI0009715A14 | | | | | | | | |
| Cas12b_{—} | 4.865 | 6.396 | 6.03 | 5.934 | 5.935 | 5.1 | 5.743 | 6.838 |
| UPI0009715A14 | | | | | | | | |
| Cas12a_{—} | 4.865 | 6.396 | 6.03 | 5.934 | 5.935 | 5.1 | 5.743 | 6.915 |
| UPI00097159CF | | | | | | | | |
| Cas12b_{—} | 4.865 | 6.396 | 6.03 | 5.934 | 5.935 | 5.1 | 5.743 | 6.915 |
| UPI00097159CF | | | | | | | | |
| Cas12a_{—} | 4.861 | 6.218 | 6.114 | 6.008 | 5.75 | 5.369 | 6.011 | 6.843 |
| UPI000832F6D2 | | | | | | | | |
| Cas12b_{—} | 4.861 | 6.218 | 6.114 | 6.008 | 5.75 | 5.369 | 6.011 | 6.843 |
| UPI000832F6D2 | | | | | | | | |
| Cas12b_tr\| | 5.122 | 5.959 | 5.946 | 5.692 | 5.93 | 5.096 | 6.011 | 7.076 |
| A0A512CSX2\| | | | | | | | | |
| A0A512CSX2_9BACL | | | | | | | | |
| OspCas12c | 5.082 | 6.075 | 5.914 | 5.588 | 5.657 | 5.251 | 3.54 | 4.853 |
| Cas14u.5\| | 6.658 | 8.752 | 4.39 | 4.128 | 9.103 | 8.21 | 7.283 | 5.804 |
| 3300012532.a\| | | | | | | | | |
| Ga0137373_10000316\| | | | | | | | | |
| 3286 . . . 5286 | | | | | | | | |
| 63461_4106_{—} | 5.931 | 7.333 | 3.933 | 2.982 | 6.91 | 7.211 | 6.686 | 4.204 |
| protein_locus_{—} | | | | | | | | |
| of_contig_LSKL01 | | | | | | | | |
| 000323 - | | | | | | | | |
| Query protein | | | | | | | | |
| (63461_4106) | | | | | | | | |
| translation (4) | | | | | | | | |
| 58610_1188_{—} | 6.989 | 8.614 | 3.599 | 3.458 | 6.914 | 7.487 | 7.55 | 4.856 |
| protein_locus_{—} | | | | | | | | |
| of_contig_LFOD0 | | | | | | | | |
| 1000003 - | | | | | | | | |
| Query protein | | | | | | | | |
| (58610_1188) | | | | | | | | |
| translation (5) | | | | | | | | |
| 21566_3969_{—} | 6.465 | 7.995 | 3.937 | 3.451 | 8.56 | 6.098 | 6.676 | 4.668 |
| protein_locus_{—} | | | | | | | | |
| of_contig_{—} | | | | | | | | |
| BAFB01000202 - | | | | | | | | |
| Query protein | | | | | | | | |
| (21566_3969) | | | | | | | | |
| translation (4) | | | | | | | | |

TABLE 3

| | Cas14a.4\| | | | | |
|---|---|---|---|---|---|
| | CG10_big_{—} | | | | |
| | fil_rev_{—} | | | | |
| | 8_21_14_{—} | | Cas14f.1\| | | |
| | 0.10_{—} | | rifcsp13_{—} | Cas14f.2\| | Cas14a.6\| |
| | scaffold_{—} | | 1_sub10_{—} | 3300009991.a\| | 3300012359.a\| |
| | 20906_{—} | | scaffold_{—} | Ga0105042_{—} | Ga0137385_{—} |
| | curated\| | | 3_curated\| | 100140\| | 10000156\| |
| | 649 . . . 2829 | CasY6 | 38906 . . . 41041 | 1624 . . . 3348 | 41289 . . . 42734 |
| Cas14g.1\| | 8.029 | 5.089 | 5.415 | 6.218 | 6.371 |
| RBG_13_scaffold_{—} | | | | | |
| 1401_curated\| | | | | | |
| 15949 . . . 18180 | | | | | |
| Cas14g.2\| | 7.91 | 5.319 | 7.185 | 7.407 | 5.585 |
| 3300009652.a\| | | | | | |
| Ga012330_1010394\| | | | | | |
| 2814 . . . 5123 | | | | | |
| Cas12i2 | 3.986 | 4.61 | 4.476 | 3.864 | 3.575 |
| Cas12i1 | 4.859 | 6.114 | 4.6 | 3.727 | 3.022 |
| Cas12g1 | 6.178 | 4.878 | 6.072 | 5.315 | 5.478 |
| Cas14d.3\| | 6.734 | 4.6 | 7.925 | 7.65 | 7.386 |
| RIFCSPLOWO2_{—} | | | | | |
| 01_FULL_OD1_{—} | | | | | |
| 45_34b_{—} | | | | | |
| rifcsplowo2_{—} | | | | | |
| 01_scaffold_{—} | | | | | |
| 3495_curated | | | | | |
| \|25656 . . . 27605\| | | | | | |
| revcom | | | | | |
| Cas14d.1\| | 6.186 | 4.351 | 6.364 | 6.347 | 6.088 |
| RIFCSPHIGHO2_01 | | | | | |
| FULL_CPR_46_36_{—} | | | | | |
| rifcsphigho2_01_{—} | | | | | |
| scaffold_646_{—} | | | | | |
| curated\| | | | | | |
| 49808 . . . 51616\| | | | | | |
| revcom | | | | | |
| CasY5 | 3.336 | 6.205 | 6.332 | 3.843 | 3.274 |
| Cas14a.4\|CG10_{—} | | 4.691 | 5.862 | 5.07 | 9.029 |
| big_fil_rev_8_{—} | | | | | |
| 21_14_0.10_{—} | | | | | |
| scaffold_20906_{—} | | | | | |
| curated\| | | | | | |
| 649 . . . 2829 | | | | | |
| CasY6 | 4.691 | | 6.434 | 3.704 | 3.819 |
| Cas14f.1\| | 5.862 | 6.434 | | 23.19 | 6.846 |
| rifcsp13_1_sub10_{—} | | | | | |
| scaffold_3_curated | | | | | |
| \|38906 . . . 41041 | | | | | |
| Cas14f.2\| | 5.07 | 3.704 | 23.19 | | 6.352 |
| 3300009991.a\| | | | | | |
| Ga0105042_100140\| | | | | | |
| 1624 . . . 3348 | | | | | |
| Cas14a.6\| | 9.029 | 3.819 | 6.846 | 6.352 | |
| 3300012359.a\| | | | | | |
| Ga0137385_10000156\| | | | | | |
| 41289 . . . 42734 | | | | | |
| Cas12a_{—} | 4.555 | 6.452 | 3.92 | 2.595 | 2.313 |
| UPI00094EEDB4 | | | | | |
| Cas12a_{—} | 4.758 | 6.443 | 4.278 | 2.961 | 3.241 |
| UPI000B4235CE | | | | | |
| Cas12a_{—} | 4.758 | 6.452 | 4.278 | 2.966 | 3.241 |
| UPI000818CC52 | | | | | |
| Cas12a_{—} | 4.758 | 6.443 | 4.278 | 2.961 | 3.241 |
| UPI0007B78B7F | | | | | |
| Cas12a_{—} | 4.758 | 6.443 | 4.278 | 2.961 | 3.241 |
| UPI000B4235F9 | | | | | |
| Cas14e.2\| | 6.259 | 3.609 | 6.964 | 8.233 | 6.705 |
| rifcsplowo2_01_{—} | | | | | |
| scaffokd_81231_{—} | | | | | |
| curated\| | | | | | |
| 976 . . . 2217 | | | | | |
| Cas14e.1\| | 5.817 | 3.6 | 5.93 | 6.777 | 7.529 |
| rifcsphigho2_01_{—} | | | | | |
| scaffold_566_{—} | | | | | |
| curated\| | | | | | |
| 113069 . . . 114313 | | | | | |
| Cas14e.3\| | 7.083 | 3.852 | 6.868 | 6.623 | 6.936 |
| rifcsphigho2_01_{—} | | | | | |
| scaffold_4702_{—} | | | | | |
| curated\| | | | | | |
| 82881 . . . 84230\| | | | | | |
| revcom | | | | | |
| CasY4 | 4.635 | 9.225 | 6.672 | 4.25 | 3.466 |
| Cas14h.3\| | 7.077 | 3.424 | 8.026 | 8.847 | 8.672 |
| 3300009698.a\| | | | | | |
| Ga0116216_10000905\| | | | | | |
| 8005 . . . 9504 | | | | | |
| Cas14h.1\| | 5.875 | 4.481 | 7.652 | 7.413 | 7.333 |
| 3300005602.a\| | | | | | |
| Ga0070762_10001740\| | | | | | |
| 7377 . . . 9071\| | | | | | |
| revcom | | | | | |
| Cas14h.2\| | 5.643 | 3.633 | 7.477 | 7.362 | 6.588 |
| 3300005921.a\| | | | | | |
| Ga0070766_10011912\| | | | | | |
| 384 . . . 2081 | | | | | |
| Cas14c.1\|CG10_{—} | 6.472 | 2.96 | 6.95 | 8.05 | 8.818 |
| big_fil_rev_8_{—} | | | | | |
| 21_14_0.10_{—} | | | | | |
| scaffold_{—} | | | | | |
| 4477_curated\| | | | | | |
| 19327 . . . 20880\| | | | | | |
| revcom | | | | | |
| Cas12h1 | 5.527 | 6.121 | 6.416 | 5.131 | 4.61 |
| CasX1 | 5.443 | 6 | 5.825 | 3.887 | 5.123 |
| CasX2 | 6.279 | 7.645 | 5.859 | 3.854 | 6.515 |
| CasY1 | 5.178 | 6.381 | 6.047 | 3.874 | 4.736 |
| Cas14u.3\|19ft_{—} | 7.945 | 4.077 | 7.343 | 6.518 | 9.524 |
| 2_nophage_noknown_{—} | | | | | |
| scaffold_{—} | | | | | |
| 0_curated\| | | | | | |
| 508188 . . . 509648 | | | | | |
| Cas14u.7\| | 7.448 | 2.927 | 7.542 | 9.769 | 8.554 |
| 3300001256.a\| | | | | | |
| JGI12210J13797_{—} | | | | | |
| 10004690\| | | | | | |
| 5792 . . . 7006 | | | | | |
| Cas14u.8\| | 7.26 | 3.712 | 7.972 | 8.099 | 8.704 |
| 3300005660.a\| | | | | | |
| Ga0073904_10021651\| | | | | | |
| 765 . . . 1943 | | | | | |
| Cas14u.4\| | 5.761 | 2.776 | 4.33 | 7.317 | 10.2 |
| rifcsp2_19_4_full_{—} | | | | | |
| scaffold_{—} | | | | | |
| 168_curated\| | | | | | |
| 84455 . . . 85657 | | | | | |
| Cas14d.2\| | 6.389 | 3.772 | 7.412 | 7.026 | 11.132 |
| rifcsphigho2_01_{—} | | | | | |
| scaffold_{—} | | | | | |
| 10981_curated\| | | | | | |
| 5762 . . . 7246\| | | | | | |
| revcom | | | | | |
| Cas14c.2\| | 7.191 | 2.675 | 6.658 | 5.415 | 9.312 |
| 3300001245.a\| | | | | | |
| JGI12048J13642_{—} | | | | | |
| 10201286\| | | | | | |
| 4257 . . . 5489\| | | | | | |
| revcom | | | | | |
| CasY3 | 5.481 | 8.333 | 5.316 | 3.772 | 3.416 |
| 633299_527_{—} | 6.474 | 3.323 | 7.832 | 7.679 | 9.298 |
| protein locus_of_{—} | | | | | |
| contig_Scfld15 - | | | | | |
| Query protein | | | | | |
| (633299_527) | | | | | |
| (4) | | | | | |
| 8971_2857_{—} | 6.922 | 3.078 | 7.059 | 8.098 | 10.478 |
| protein_locus_of_{—} | | | | | |
| contig_OEJQ01000083.1 - | | | | | |
| Query protein | | | | | |
| (8971_2857) | | | | | |
| 9265_901_protein_{—} | 6.812 | 3.133 | 6.946 | 7.934 | 10.222 |
| locus_of_contig_{—} | | | | | |
| OEFX01000005.1 - | | | | | |
| Query protein | | | | | |
| (9265_901) | | | | | |
| Cas14u.6\| | 6.292 | 3.917 | 9.655 | 10.224 | 6.623 |
| 3300006028.a\| | | | | | |
| Ga0070717_10000077\| | | | | | |
| 54519 . . . 56201\| | | | | | |
| revcom | | | | | |
| 466065_250_{—} | 6.936 | 2.76 | 9.272 | 9.324 | 10.23 |
| protein_locus_of_{—} | | | | | |
| contig_SFKR01000004.1 - | | | | | |
| Query protein | | | | | |
| (466065_250) | | | | | |
| Cas14a.5\| | 5.658 | 2.441 | 5.27 | 4.647 | 6.549 |
| rifcsplowo2_{—} | | | | | |
| 01_scaffold_{—} | | | | | |
| 34461_curated\| | | | | | |
| 4968 . . . 6521 | | | | | |
| CasY2 | 4.878 | 6.471 | 4.818 | 2.85 | 4.903 |
| Cas14a.3\|gwa1_{—} | 12.273 | 4.194 | 7.65 | 7.267 | 17.056 |
| scaffold_1795_{—} | | | | | |
| curated\| | | | | | |
| 25635 . . . 27224\| | | | | | |
| revcom | | | | | |
| Cas14a.1\| | 12.188 | 5.436 | 6.827 | 7.401 | 19.342 |
| rifcsphigho2_02_{—} | | | | | |
| scaffold_2167_{—} | | | | | |
| curated\| | | | | | |
| 30296 . . . 31798\| | | | | | |
| revcom | | | | | |
| Cas14a.2\|gwa2_{—} | 11.523 | 5.485 | 6.426 | 7.049 | 19.923 |
| scaffold_18027_{—} | | | | | |
| curated\| | | | | | |
| 7105 . . . 8628 | | | | | |
| Cas14b.4\| | 7.367 | 3.512 | 6.711 | 7.764 | 8.305 |
| cg1_0.2_scaffold_785_{—} | | | | | |
| c_curated\| | | | | | |
| 32521 . . . 34155 | | | | | |
| Cas14b.7\| | 8.713 | 3.816 | 7.662 | 8.75 | 8.819 |
| 3300013125.a\| | | | | | |
| Ga0172369_10000737\| | | | | | |
| 994 . . . 2652\| | | | | | |
| revcom | | | | | |
| Cas14u.2\| | 7.022 | 2.718 | 5.618 | 5.965 | 8 |
| 3300002172.a\| | | | | | |
| JGI24730J26740_1002785\| | | | | | |
| 496 . . . 1605\| | | | | | |
| revcom | | | | | |
| Cas14b.3\| | 8.647 | 3.987 | 8.422 | 7.75 | 10.616 |
| rifcsphigho2_01_{—} | | | | | |
| scaffold_36781_{—} | | | | | |
| curated\| | | | | | |
| 2592 . . . 4217 | | | | | |
| Cas14b.2\| | 10.57 | 4.19 | 8.56 | 6.615 | 8.848 |
| rifcsplowo2_01_{—} | | | | | |
| scaffold_282_curated\| | | | | | |
| 77370 . . . 78983 | | | | | |
| Cas14b.1\| | 10.497 | 4.093 | 8.548 | 7.373 | 10.067 |
| rifcsplowo2_01_{—} | | | | | |
| scaffold_239_curated\| | | | | | |
| 54653 . . . 56257 | | | | | |
| Cas14b.8\| | 10.083 | 3.692 | 7.87 | 7.988 | 9.564 |
| 3300013125.a\| | | | | | |
| Ga0172369_10010464\| | | | | | |
| 885 . . . 2489 \| | | | | | |
| revcom | | | | | |
| Cas14b.5\| | 8.482 | 3.92 | 6.937 | 6.202 | 10.282 |
| rifcsphigho2_02_{—} | | | | | |
| scaffold_55589_{—} | | | | | |
| curated\| | | | | | |
| 1904 . . . 3598 | | | | | |
| Cas14b.6\|CG03_{—} | 9.707 | 4.124 | 7.412 | 6.724 | 9.35 |
| land_8_20_14_{—} | | | | | |
| 0.80_scaffold_{—} | | | | | |
| 2214_curated\| | | | | | |
| 6634 . . . 8466\| | | | | | |
| revcom | | | | | |
| Cas14b.9\| | 10.174 | 5.044 | 8.524 | 7.364 | 9.076 |
| 3300013127.a\| | | | | | |
| Ga0172365_10004421\| | | | | | |
| 633 . . . 2366\| | | | | | |
| revcom | | | | | |
| 209658_13971_{—} | 8.733 | 3.531 | 6.709 | 7.4 | 11.616 |
| protein_locus_{—} | | | | | |
| of_contig_{—} | | | | | |
| Ga0190333_1001561 - | | | | | |
| Query protein | | | | | |
| (209658_13971) | | | | | |
| (2) | | | | | |
| 209657_57738 | 13.531 | 5.979 | 12.057 | 10.37 | 16.667 |
| protein_locus_{—} | | | | | |
| of_contig_Ga019 | | | | | |
| 0332 1015597 - | | | | | |
| Query protein | | | | | |
| (209657_57738) | | | | | |
| (2) | | | | | |
| 209660_51257_{—} | 12.329 | 5.696 | 12.546 | 10.811 | 16.129 |
| protein_locus_{—} | | | | | |
| of_contig_{—} | | | | | |
| Ga0190335_1015156 - | | | | | |
| Query protein | | | | | |
| (209660_51257) | | | | | |
| (2) | | | | | |
| Cas14b.14\|gwc1_{—} | 7.393 | 3.543 | 5.728 | 5.503 | 8.423 |
| scaffold_8732_{—} | | | | | |
| curated\| | | | | | |
| 2705 . . . 4537 | | | | | |
| Cas14b.15\| | 7.345 | 4.282 | 6.633 | 4.809 | 9.56 |
| 3300010293.a\| | | | | | |
| Ga0116204_1008574\| | | | | | |
| 2134 . . . 4032 | | | | | |
| Cas14b.12\| | 7.078 | 3.909 | 5.122 | 4.492 | 6.076 |
| CG22_combo_CG10 - | | | | | |
| 13_8_21_14_all_{—} | | | | | |
| scaffold_2003_{—} | | | | | |
| curated\| | | | | | |
| 553 . . . 2880\| | | | | | |
| revcom | | | | | |
| Cas14b.13\| | 7.441 | 3.876 | 6.034 | 5.232 | 6.378 |
| rifcsphigho2_01_{—} | | | | | |
| scaffold_82367_{—} | | | | | |
| curated\| | | | | | |
| 1523 . . . 3856\| | | | | | |
| revcom | | | | | |
| Cas14b.16\| | 7.294 | 4.444 | 8.161 | 7.123 | 9.385 |
| 3300005573.a\| | | | | | |
| Ga0078972_1001015a\| | | | | | |
| 33750 . . . 35627 | | | | | |
| Cas14b.10\| | 8.621 | 4.167 | 7.412 | 7.613 | 8.661 |
| CG08_land_8_20_{—} | | | | | |
| 14_0.20_scaffold_{—} | | | | | |
| 1609_curated\| | | | | | |
| 6134 . . . 7975 | | | | | |
| Cas14b.11\| | 6.974 | 4.567 | 7.263 | 6.589 | 9.291 |
| CG_4_10_14_0.8_{—} | | | | | |
| um_filter_scaffold_{—} | | | | | |
| 20762_curated\| | | | | | |
| 1372 . . . 3219 | | | | | |
| Cas14u.1\| | 7.865 | 2.972 | 6.276 | 7.279 | 8.884 |
| 3300009029.a\| | | | | | |
| Ga0066793_10010091\| | | | | | |
| 37 . . . 1113\| | | | | | |
| revcom | | | | | |
| Cas12c1 | 3.943 | 7.076 | 5.155 | 3.681 | 3.421 |
| Cas12c2 | 4.396 | 6.856 | 4.448 | 3.598 | 4.153 |
| Cas12a_{—} | 3.91 | 7.015 | 6.356 | 4.2 | 2.899 |
| UPI001113398F | | | | | |
| Cas12b_{—} | 3.91 | 7.015 | 6.356 | 4.2 | 2.899 |
| UPI001113398F | | | | | |
| Cas12b_tr\| | 3.747 | 6.942 | 6.394 | 4.259 | 2.893 |
| A0A1I7F1U9\| | | | | | |
| A0A1I7F1U9_9BACL | | | | | |
| Cas12a_{—} | 4.391 | 6.428 | 6.014 | 4.541 | 4.159 |
| UPI00083514A7 | | | | | |
| Cas12b_{—} | 4.391 | 6.428 | 6.014 | 4.541 | 4.159 |
| UPI00083514A7 | | | | | |
| Cas12a_{—} | 5.165 | 6.133 | 6.324 | 4.558 | 2.69 |
| UPI00097159F1 | | | | | |
| Cas12b_{—} | 5.165 | 6.133 | 6.324 | 4.558 | 2.69 |
| UPI00097159F1 | | | | | |
| Cas12b_sp\| | 5.165 | 6.133 | 6.324 | 4.558 | 2.69 |
| T0D7A2\|CS12B_{—} | | | | | |
| ALIAG | | | | | |
| Cas12a_{—} | 5.165 | 6.058 | 6.324 | 4.649 | 2.69 |
| UPI0009715A14 | | | | | |
| Cas12b_{—} | 5.165 | 6.058 | 6.324 | 4.649 | 2.69 |
| UPI0009715A14 | | | | | |
| Cas12a_{—} | 5.165 | 6.133 | 6.324 | 4.558 | 2.69 |
| UPI00097159CF | | | | | |
| Cas12b_{—} | 5.165 | 6.133 | 6.324 | 4.558 | 2.69 |
| UPI00097159CF | | | | | |
| Cas12a_{—} | 5.33 | 6.502 | 6.416 | 4.831 | 2.966 |
| UPI000832F6D2 | | | | | |
| Cas12b_{—} | 5.33 | 6.502 | 6.416 | 4.831 | 2.966 |
| UPI000832F6D2 | | | | | |
| Cas12b_tr\| | 5.161 | 6.353 | 6.416 | 4.649 | 2.966 |
| A0A512CSX2\| | | | | | |
| A0A512CSX2_9BACL | | | | | |
| OspCas12c | 4.021 | 7.595 | 5.314 | 4.073 | 3.471 |
| Cas14u.5\| | 6.591 | 5.418 | 6.436 | 5.503 | 6.078 |
| 3300012532.a\| | | | | | |
| Ga0137373_10000316\| | | | | | |
| 3286 . . . 5286 | | | | | |
| 63461_4106_{—} | 5.284 | 3.692 | 7.015 | 7.794 | 5.063 |
| protein_locus_of | | | | | |
| contig_LSKL01000323 - | | | | | |
| Query protein | | | | | |
| (63461_4106) | | | | | |
| translation (4) | | | | | |
| 58610_1188_{—} | 7.097 | 3.668 | 6.435 | 6.984 | 5.91 |
| protein_locus_of | | | | | |
| contig_LFOD01000003 - | | | | | |
| Query protein | | | | | |
| (58610_1188) | | | | | |
| translation (5) | | | | | |
| 21566_3969_{—} | 6.684 | 3.462 | 5.92 | 6.726 | 6.171 |
| protein_locus_of | | | | | |
| contig_BAFB01000202 - | | | | | |
| Query protein | | | | | |
| (21566_3969) | | | | | |
| translation (4) | | | | | |

| | Cas12a_{—} | Cas12a_{—} | Cas12a_{—} |
|---|---|---|---|
| | UPI00094EEDB4 | UPI000B4235CE | UPI000818CC52 |
| Cas14g.1\| | 3.643 | 4.519 | 4.525 |
| RBG_13_scaffold_{—} | | | |
| 1401_curated\| | | | |
| 15949 . . . 18180 | | | |
| Cas14g.2\| | 3.157 | 3.519 | 3.451 |
| 3300009652.a\| | | | |
| Ga012330_1010394\| | | | |
| 2814 . . . 5123 | | | |
| Cas12i2 | 5.548 | 6.326 | 6.335 |
| Cas12i1 | 4.833 | 5.434 | 5.512 |
| Cas12g1 | 4.397 | 4.604 | 4.535 |
| Cas14d.3\| | 3.972 | 5.118 | 5.126 |
| RIFCSPLOWO2_{—} | | | |
| 01_FULL_OD1_{—} | | | |
| 45_34b_{—} | | | |
| rifcsplowo2_{—} | | | |
| 01_scaffold_{—} | | | |
| 3495_curated | | | |
| \|25656 . . . 27605\| | | | |
| revcom | | | |
| Cas14d.1\| | 4.869 | 4.828 | 4.758 |
| RIFCSPHIGHO2_01 | | | |
| FULL_CPR_46_36_{—} | | | |
| rifcsphigho2_01_{—} | | | |
| scaffold_646_{—} | | | |
| curated\| | | | |
| 49808 . . . 51616\| | | | |
| revcom | | | |
| CasY5 | 5.552 | 5.773 | 5.71 |
| Cas14a.4\|CG10_{—} | 4.555 | 4.758 | 4.758 |
| big_fil_rev_8_{—} | | | |
| 21_14_0.10_{—} | | | |
| scaffold_20906_{—} | | | |
| curated\| | | | |
| 649 . . . 2829 | | | |
| CasY6 | 6.452 | 6.443 | 6.452 |
| Cas14f.1\| | 3.92 | 4.278 | 4.278 |
| rifcsp13_1_sub10_{—} | | | |
| scaffold_3_curated | | | |
| \|38906 . . . 41041 | | | |
| Cas14f.2\| | 2.595 | 2.961 | 2.966 |
| 3300009991.a\| | | | |
| Ga0105042_100140\| | | | |
| 1624 . . . 3348 | | | |
| Cas14a.6\| | 2.313 | 3.241 | 3.241 |
| 3300012359.a\| | | | |
| Ga0137385_10000156\| | | | |
| 41289 . . . 42734 | | | |
| Cas12a_{—} | | 41.921 | 41.996 |
| UPI00094EEDB4 | | | |
| Cas12a_{—} | 41.921 | | 99.618 |
| UPI000B4235CE | | | |
| Cas12a_{—} | 41.996 | 99.618 | |
| UPI000818CC52 | | | |
| Cas12a_{—} | 42.07 | 99.771 | 99.847 |
| UPI0007B78B7F | | | |
| Cas12a_{—} | 42.039 | 99.466 | 99.389 |
| UPI000B4235F9 | | | |
| Cas14e.2\| | 2.73 | 3.191 | 3.191 |
| rifcsplowo2_01_{—} | | | |
| scaffokd_81231_{—} | | | |
| curated\| | | | |
| 976 . . . 2217 | | | |
| Cas14e.1\| | 2.886 | 3.183 | 3.183 |
| rifcsphigho2_01_{—} | | | |
| scaffold_566_{—} | | | |
| curated\| | | | |
| 113069 . . . 114313 | | | |
| Cas14e.3\| | 3.196 | 3.658 | 3.658 |
| rifcsphigho2_01_{—} | | | |
| scaffold_4702_{—} | | | |
| curated\| | | | |
| 82881 . . . 84230\| | | | |
| revcom | | | |
| CasY4 | 5.765 | 6.089 | 6.098 |
| Cas14h.3\| | 3.248 | 2.877 | 2.877 |
| 3300009698.a\| | | | |
| Ga0116216_10000905\| | | | |
| 8005 . . . 9504 | | | |
| Cas14h.1\| | 3.752 | 3.979 | 3.979 |
| 3300005602.a\| | | | |
| Ga0070762_10001740\| | | | |
| 7377 . . . 9071\| | | | |
| revcom | | | |
| Cas14h.2\| | 3.379 | 3.991 | 3.991 |
| 3300005921.a\| | | | |
| Ga0070766_10011912\| | | | |
| 384 . . . 2081 | | | |
| Cas14c.1\|CG10_{—} | 3.414 | 3.104 | 3.104 |
| big_fil_rev_8_{—} | | | |
| 21_14_0.10_{—} | | | |
| scaffold_{—} | | | |
| 4477_curated\| | | | |
| 19327 . . . 20880\| | | | |
| revcom | | | |
| Cas12h1 | 3.627 | 5.205 | 5.066 |
| CasX1 | 5.151 | 6.204 | 6.213 |
| CasX2 | 4.716 | 5.564 | 5.572 |
| CasY1 | 5.234 | 5.688 | 5.626 |
| Cas14u.3\|19ft_{—} | 3.73 | 3.026 | 3.026 |
| 2_nophage_noknown_{—} | | | |
| scaffold_{—} | | | |
| 0_curated\| | | | |
| 508188 . . . 509648 | | | |
| Cas14u.7\| | 2.846 | 3.007 | 3.007 |
| 3300001256.a\| | | | |
| JGI12210J13797_{—} | | | |
| 10004690\| | | | |
| 5792 . . . 7006 | | | |
| Cas14u.8\| | 2.771 | 3.075 | 3.075 |
| 3300005660.a\| | | | |
| Ga0073904_10021651\| | | | |
| 765 . . . 1943 | | | |
| Cas14u.4\| | 3.082 | 3.077 | 3.077 |
| rifcsp2_19_4_full_{—} | | | |
| scaffold_{—} | | | |
| 168_curated\| | | | |
| 84455 . . . 85657 | | | |
| Cas14d.2\| | 3.991 | 4.372 | 4.372 |
| rifcsphigho2_01_{—} | | | |
| scaffold_{—} | | | |
| 10981_curated\| | | | |
| 5762 . . . 7246\| | | | |
| revcom | | | |
| Cas14c.2\| | 2.822 | 3.351 | 3.351 |
| 3300001245.a\| | | | |
| JGI12048J13642_{—} | | | |
| 10201286\| | | | |
| 4257 . . . 5489\| | | | |
| revcom | | | |
| CasY3 | 5.999 | 6.877 | 6.887 |
| 633299_527_{—} | 3.009 | 3.236 | 3.236 |
| protein locus_of_{—} | | | |
| contig_Scfld15 - | | | |
| Query protein | | | |
| (633299_527) | | | |
| (4) | | | |
| 8971_2857_{—} | 2.659 | 3.223 | 3.223 |
| protein_locus_of_{—} | | | |
| contig_OEJQ01000083.1 - | | | |
| Query protein | | | |
| (8971_2857) | | | |
| 9265_901_protein_{—} | 2.716 | 3.195 | 3.195 |
| locus_of_contig_{—} | | | |
| OEFX01000005.1 - | | | |
| Query protein | | | |
| (9265_901) | | | |
| Cas14u.6\| | 2.868 | 4.189 | 4.189 |
| 3300006028.a\| | | | |
| Ga0070717_10000077\| | | | |
| 54519 . . . 56201\| | | | |
| revcom | | | |
| 466065_250_{—} | 2.679 | 2.518 | 2.518 |
| protein_locus_of_{—} | | | |
| contig_SFKR01000004.1 - | | | |
| Query protein | | | |
| (466065_250) | | | |
| Cas14a.5\| | 3.966 | 5.004 | 5.008 |
| rifcsplowo2_{—} | | | |
| 01_scaffold_{—} | | | |
| 34461_curated\| | | | |
| 4968 . . . 6521 | | | |
| CasY2 | 6.557 | 6.424 | 6.362 |
| Cas14a.3\|gwa1_{—} | 4.855 | 3.909 | 3.909 |
| scaffold_1795_{—} | | | |
| curated\| | | | |
| 25635 . . . 27224\| | | | |
| revcom | | | |
| Cas14a.1\| | 3.801 | 4.425 | 4.425 |
| rifcsphigho2_02_{—} | | | |
| scaffold_2167_{—} | | | |
| curated\| | | | |
| 30296 . . . 31798\| | | | |
| revcom | | | |
| Cas14a.2\|gwa2_{—} | 3.395 | 4.17 | 4.17 |
| scaffold_18027_{—} | | | |
| curated\| | | | |
| 7105 . . . 8628 | | | |
| Cas14b.4\| | 3.807 | 3.106 | 3.106 |
| cg1_0.2_scaffold_785_{—} | | | |
| c_curated\| | | | |
| 32521 . . . 34155 | | | |
| Cas14b.7\| | 4.338 | 3.464 | 3.464 |
| 3300013125.a\| | | | |
| Ga0172369_10000737\| | | | |
| 994 . . . 2652\| | | | |
| revcom | | | |
| Cas14u.2\| | 2.644 | 2.638 | 2.638 |
| 3300002172.a\| | | | |
| JGI24730J26740_1002785\| | | | |
| 496 . . . 1605\| | | | |
| revcom | | | |
| Cas14b.3\| | 4.439 | 4.5 | 4.507 |
| rifcsphigho2_01_{—} | | | |
| scaffold_36781_{—} | | | |
| curated\| | | | |
| 2592 . . . 4217 | | | |
| Cas14b.2\| | 4.471 | 4.15 | 4.15 |
| rifcsplowo2_01_{—} | | | |
| scaffold_282_curated\| | | | |
| 77370 . . . 78983 | | | |
| Cas14b.1\| | 4.766 | 4.29 | 4.29 |
| rifcsplowo2_01_{—} | | | |
| scaffold_239_curated\| | | | |
| 54653 . . . 56257 | | | |
| Cas14b.8\| | 4.375 | 4.29 | 4.29 |
| 3300013125.a\| | | | |
| Ga0172369_10010464\| | | | |
| 885 . . . 2489 \| | | | |
| revcom | | | |
| Cas14b.5\| | 3.724 | 4.267 | 4.267 |
| rifcsphigho2_02_{—} | | | |
| scaffold_55589_{—} | | | |
| curated\| | | | |
| 1904 . . . 3598 | | | |
| Cas14b.6\|CG03_{—} | 4.08 | 3.92 | 3.926 |
| land_8_20_14_{—} | | | |
| 0.80_scaffold_{—} | | | |
| 2214_curated\| | | | |
| 6634 . . . 8466\| | | | |
| revcom | | | |
| Cas14b.9\| | 4.405 | 4.099 | 4.099 |
| 3300013127.a\| | | | |
| Ga0172365_10004421\| | | | |
| 633 . . . 2366\| | | | |
| revcom | | | |
| 209658_13971_{—} | 2.914 | 3.265 | 3.265 |
| protein_locus_{—} | | | |
| of_contig_{—} | | | |
| Ga0190333_1001561 - | | | |
| Query protein | | | |
| (209658_13971) | | | |
| (2) | | | |
| 209657_57738 | 5.092 | 6.061 | 6.061 |
| protein_locus_{—} | | | |
| of_contig_Ga019 | | | |
| 0332 1015597 - | | | |
| Query protein | | | |
| (209657_57738) | | | |
| (2) | | | |
| 209660_51257_{—} | 4.792 | 5.992 | 5.992 |
| protein_locus_{—} | | | |
| of_contig_{—} | | | |
| Ga0190335_1015156 - | | | |
| Query protein | | | |
| (209660_51257) | | | |
| (2) | | | |
| Cas14b.14\|gwc1_{—} | 3.917 | 3.514 | 3.514 |
| scaffold_8732_{—} | | | |
| curated\| | | | |
| 2705 . . . 4537 | | | |
| Cas14b.15\| | 4.012 | 5.174 | 5.174 |
| 3300010293.a\| | | | |
| Ga0116204_1008574\| | | | |
| 2134 . . . 4032 | | | |
| Cas14b.12\| | 3.474 | 4.502 | 4.508 |
| CG22_combo_CG10 - | | | |
| 13_8_21_14_all_{—} | | | |
| scaffold_2003_{—} | | | |
| curated\| | | | |
| 553 . . . 2880\| | | | |
| revcom | | | |
| Cas14b.13\| | 3.479 | 5.469 | 5.477 |
| rifcsphigho2_01_{—} | | | |
| scaffold_82367_{—} | | | |
| curated\| | | | |
| 1523 . . . 3856\| | | | |
| revcom | | | |
| Cas14b.16\| | 5.104 | 5.097 | 5.104 |
| 3300005573.a\| | | | |
| Ga0078972_1001015a\| | | | |
| 33750 . . . 35627 | | | |
| Cas14b.10\| | 4.224 | 4.587 | 4.671 |
| CG08_land_8_20_{—} | | | |
| 14_0.20_scaffold_{—} | | | |
| 1609_curated\| | | | |
| 6134 . . . 7975 | | | |
| Cas14b.11\| | 4.228 | 4.82 | 4.904 |
| CG_4_10_14_0.8_{—} | | | |
| um_filter_scaffold_{—} | | | |
| 20762_curated\| | | | |
| 1372 . . . 3219 | | | |
| Cas14u.1\| | 2.422 | 3.04 | 3.04 |
| 3300009029.a\| | | | |
| Ga0066793_10010091\| | | | |
| 37 . . . 1113\| | | | |
| revcom | | | |
| Cas12c1 | 7.387 | 7.064 | 7.074 |
| Cas12c2 | 5.411 | 6.555 | 6.564 |
| Cas12a_{—} | 5.679 | 5.297 | 5.233 |
| UPI001113398F | | | |
| Cas12b_{—} | 5.679 | 5.297 | 5.233 |
| UPI001113398F | | | |
| Cas12b_tr\| | 5.575 | 5.323 | 5.259 |
| A0A1I7F1U9\| | | | |
| A0A1I7F1U9_9BACL | | | |
| Cas12a_{—} | 6.026 | 5.583 | 5.448 |
| UPI00083514A7 | | | |
| Cas12b_{—} | 6.026 | 5.583 | 5.448 |
| UPI00083514A7 | | | |
| Cas12a_{—} | 6.82 | 6.017 | 5.882 |
| UPI00097159F1 | | | |
| Cas12b_{—} | 6.82 | 6.017 | 5.882 |
| UPI00097159F1 | | | |
| Cas12b_sp\| | 6.82 | 6.017 | 5.882 |
| T0D7A2\|CS12B_{—} | | | |
| ALIAG | | | |
| Cas12a_{—} | 6.82 | 6.017 | 5.882 |
| UPI0009715A14 | | | |
| Cas12b_{—} | 6.82 | 6.017 | 5.882 |
| UPI0009715A14 | | | |
| Cas12a_{—} | 6.82 | 6.017 | 5.882 |
| UPI00097159CF | | | |
| Cas12b_{—} | 6.82 | 6.017 | 5.882 |
| UPI00097159CF | | | |
| Cas12a_{—} | 6.671 | 5.87 | 5.735 |
| UPI000832F6D2 | | | |
| Cas12b_{—} | 6.671 | 5.87 | 5.735 |
| UPI000832F6D2 | | | |
| Cas12b_tr\| | 6.671 | 5.941 | 5.806 |
| A0A512CSX2\| | | | |
| A0A512CSX2_9BACL | | | |
| OspCas12c | 6.104 | 7.567 | 7.436 |
| Cas14u.5\| | 3.74 | 4.064 | 4.064 |
| 3300012532.a\| | | | |
| Ga0137373_10000316\| | | | |
| 3286 . . . 5286 | | | |
| 63461_4106_{—} | 2.937 | 3.303 | 3.303 |
| protein_locus_of | | | |
| contig_LSKL01000323 - | | | |
| Query protein | | | |
| (63461_4106) | | | |
| translation (4) | | | |
| 58610_1188_{—} | 4.321 | 3.988 | 3.988 |
| protein_locus_of | | | |
| contig_LFOD01000003 - | | | |
| Query protein | | | |
| (58610_1188) | | | |
| translation (5) | | | |
| 21566_3969_{—} | 3.181 | 3.627 | 3.627 |
| protein_locus_of | | | |
| contig_BAFB01000202 - | | | |
| Query protein | | | |
| (21566_3969) | | | |
| translation (4) | | | |

TABLE 4

| | | | Cas14e.2\| | Cas14e.1\| | Cas14e.3\| | | | |
|---|---|---|---|---|---|---|---|---|
| | | | rifcsplowo2_{—} | rifcsphigho2_{—} | rifcsphigho2_{—} | | Cas14h.3\| | Cas14h.1\| |
| | | | 01_scaffold_{—} | 01_scaffold_{—} | 01_scaffold_{—} | | 3300009698.a\| | 3300005602.a\| |
| | Cas12a_{—} | Cas12a_{—} | 81231_curated\| | 566_curated\| | 4702_curated\| | | Ga0116216_10000905\| | Ga0070762_10001740\| |
| | UPI0007B78B7F | UPI000B4235F9 | 976 . . . 2217 | 113069 . . . 114313 | 82881 . . . 84230\|revcom | CasY4 | 8005 . . . 9504 | 7377 . . . 9071\|revcom |
| Cas14g.1\|RBG_13_{—} | 4.519 | 4.519 | 5.204 | 6.039 | 3.808 | 6.058 | 7.333 | 5.767 |
| scaffold_1401_{—} | | | | | | | | |
| curated\|15949 . . . 18180 | | | | | | | | |
| Cas14g.2\|3300009652.a\| | 3.519 | 3.519 | 5.391 | 6.595 | 5.292 | 4.651 | 5.063 | 7.752 |
| Ga0123330_1010394\| | | | | | | | | |
| 2814 . . . 5123 | | | | | | | | |
| Cas12i2 | 6.326 | 6.326 | 3.425 | 4.207 | 4.429 | 5.598 | 3.626 | 4.511 |
| Cas12i1 | 5.505 | 5.501 | 3.51 | 3.321 | 3.337 | 3.922 | 3.053 | 4.255 |
| Cas12g1 | 4.604 | 4.604 | 4.439 | 6.144 | 4.581 | 6.556 | 5.27 | 6.195 |
| Cas14d.3\|RIFCSPLOWO2_{—} | 5.118 | 5.118 | 5.663 | 4.903 | 6.917 | 4.348 | 6.97 | 6.031 |
| 01_FULL_OD1_45_34b_{—} | | | | | | | | |
| rifcsplowo2_01_{—} | | | | | | | | |
| scaffold_3495_curated\| | | | | | | | | |
| 25656 . . . 27605\|revcom | | | | | | | | |
| Cas14d.1\|RIFCSPHIGHO2_{—} | 4.828 | 4.828 | 5.627 | 6.19 | 5.538 | 3.766 | 5.952 | 5.381 |
| 01_FULL_CPR_46_36_{—} | | | | | | | | |
| rifcsphigho2_01_{—} | | | | | | | | |
| scaffold_646_curated\| | | | | | | | | |
| 49808 . . . 51616\|revcom | | | | | | | | |
| CasY5 | 5.773 | 5.773 | 3.501 | 3.298 | 2.681 | 6.522 | 3.469 | 4.825 |
| Cas14a.4\|CG10_big_fil_rev_{—} | 4.758 | 4.758 | 6.259 | 5.817 | 7.083 | 4.635 | 7.077 | 5.875 |
| 8_21_14_0.10_scaffold_{—} | | | | | | | | |
| 20906_curated\| | | | | | | | | |
| 1649 . . . 2829 | | | | | | | | |
| CasY6 | 6.443 | 6.443 | 3.609 | 3.6 | 3.852 | 9.225 | 3.424 | 4.481 |
| Cas14f.1\| rifcsp13_1_{—} | 4.278 | 4.278 | 6.964 | 5.93 | 6.868 | 6.672 | 8.026 | 7.652 |
| sub10_scaffold_3_curated\| | | | | | | | | |
| 38906 . . . 41041 | | | | | | | | |
| Cas14f.2\|3300009991.a\| | 2.961 | 2.961 | 8.233 | 6.777 | 6.623 | 4.25 | 8.847 | 7.413 |
| Ga0105042_100140\| | | | | | | | | |
| 1624 . . . 3348 | | | | | | | | |
| Cas14a.6\|3300012359.a\| | 3.241 | 3.241 | 6.705 | 7.529 | 6.936 | 3.466 | 8.672 | 7.333 |
| Ga0137385_10000156\| | | | | | | | | |
| 41289 . . . 42734 | | | | | | | | |
| Cas12a_UPI00094EEDB4 | 42.07 | 42.039 | 2.73 | 2.886 | 3.196 | 5.765 | 3.248 | 3.752 |
| Cas12a_UPI000B4235CE | 99.771 | 99.466 | 3.191 | 3.183 | 3.658 | 6.089 | 2.877 | 3.979 |
| Cas12a_UPI000818CC52 | 99.847 | 99.389 | 3.191 | 3.183 | 3.658 | 6.098 | 2.877 | 3.979 |
| Cas12a_UPI0007B78B7F | | 99.542 | 3.191 | 3.183 | 3.658 | 6.089 | 2.877 | 3.979 |
| Cas12a_UPI000B4235F9 | 99.542 | | 3.191 | 3.183 | 3.658 | 6.089 | 2.877 | 3.979 |
| Cas14e.2\|rifcsplowo2_01_{—} | 3.191 | 3.191 | | 22.222 | 23.108 | 2.723 | 6.346 | 5.354 |
| scaffold_81231_curated\| | | | | | | | | |
| 976 . . . 2217 | | | | | | | | |
| Cas14e.1\|rifcsphigho2_01_{—} | 3.183 | 3.183 | 22.222 | | 20.816 | 2.553 | 7.57 | 6.879 |
| scaffold_566_curated\| | | | | | | | | |
| 113069 . . . 114313 | | | | | | | | |
| Cas14e.3\|rifcsphigho2_01_{—} | 3.658 | 3.658 | 23.108 | 20.816 | | 2.726 | 6.168 | 6.146 |
| scaffold_4702_curated\| | | | | | | | | |
| 82881 . . . 84230\|revcom | | | | | | | | |
| CasY4 | 6.089 | 6.089 | 2.723 | 2.553 | 2.726 | | 3.48 | 3.361 |
| Cas14h.3\|3300009698.a\| | 2.877 | 2.877 | 6.346 | 7.57 | 6.168 | 3.48 | | 13.942 |
| Ga0116216_10000905\| | | | | | | | | |
| 8005 . . . 9504 | | | | | | | | |
| Cas14h.1\|3300005602.a\| | 3.979 | 3.979 | 5.354 | 6.879 | 6.146 | 3.361 | 13.942 | |
| Ga0070762_10001740\| | | | | | | | | |
| 7377 . . . 9071\|revcom | | | | | | | | |
| Cas14h.2\|3300005921.a\| | 3.991 | 3.991 | 5.448 | 6.154 | 7.179 | 2.773 | 14.56 | 65.12 |
| Ga0070766_10011912\| | | | | | | | | |
| 384 . . . 2081 | | | | | | | | |
| Cas14c.1\|CG10_big_fil_rev_{—} | 3.104 | 3.104 | 8.63 | 8.443 | 6.964 | 2.927 | 9.589 | 8.889 |
| 8_21_14_0.10_scaffold_{—} | | | | | | | | |
| 4477_curated\|19327 . . . | | | | | | | | |
| 20880\|revcom | | | | | | | | |
| Cas12h1 | 5.205 | 5.205 | 5.396 | 5.383 | 4.556 | 3.965 | 5.166 | 4.577 |
| CasX1 | 6.13 | 6.13 | 4.041 | 3.316 | 4.063 | 7.065 | 5.217 | 4.709 |
| CasX2 | 5.564 | 5.49 | 4.603 | 3.556 | 4.316 | 7.422 | 5.489 | 4.044 |
| CasY1 | 5.688 | 5.688 | 3.306 | 4.033 | 4.5 | 6.984 | 3.908 | 3.953 |
| Cas14u.3\|19ft_2_nophage_{—} | 3.026 | 3.026 | 7.579 | 8.598 | 7.895 | 3.495 | 7.679 | 6.408 |
| noknown_scaffold_0_{—} | | | | | | | | |
| curated\|508188 . . . 509648 | | | | | | | | |
| Cas14u.7\|3300001256.a\| | 3.007 | 3.007 | 8.463 | 8.609 | 9.298 | 4.114 | 13.546 | 10.764 |
| JGI12210J13797_{—} | | | | | | | | |
| 10004690\|5792 . . . 7006 | | | | | | | | |
| Cas14u.8\|3300005660.a\| | 3.075 | 3.075 | 8.036 | 8.869 | 7.438 | 3.28 | 12.749 | 9.457 |
| Ga0073904_10021651\| | | | | | | | | |
| 765 . . . 1943 | | | | | | | | |
| Cas14u.4\|rifcsp2_19_4_{—} | 3.077 | 3.077 | 8.15 | 6.813 | 5.809 | 2.521 | 8.984 | 7.863 |
| full_scaffold_168_curated\| | | | | | | | | |
| 84455 . . . 85657 | | | | | | | | |
| Cas14d.2\|rifcsphigho2_01_{—} | 4.372 | 4.372 | 6.191 | 7.836 | 7.076 | 3.757 | 7.218 | 7.445 |
| scaffold_10981_curated\| | | | | | | | | |
| 5762 . . . 7246\|revcom | | | | | | | | |
| Cas14c.2\|3300001245.a\| | 3.351 | 3.351 | 7.463 | 6.438 | 7.6 | 3.763 | 13.112 | 8.263 |
| JGI12048J13642_{—} | | | | | | | | |
| 10201286\|4257 . . . | | | | | | | | |
| 5489 \|revcom | | | | | | | | |
| CasY3 | 6.877 | 6.877 | 3.198 | 2.936 | 3.128 | 7.777 | 3.926 | 3.568 |
| 633299_527_protein_locus_{—} | 3.236 | 3.236 | 9.888 | 10.811 | 10.669 | 3.788 | 10.097 | 9.091 |
| of_contig_Scfld15 - | | | | | | | | |
| Query protein | | | | | | | | |
| (633299_527) (4) | | | | | | | | |
| 8971_2857_protein_locus_{—} | 3.223 | 3.223 | 9.832 | 8.794 | 7.586 | 4.111 | 12.281 | 9.594 |
| of_contig_OEJQ01000083.1 - | | | | | | | | |
| Query protein | | | | | | | | |
| (8971_2857) | | | | | | | | |
| 9265_901_protein_locus_{—} | 3.195 | 3.195 | 9.579 | 8.557 | 7.399 | 4.248 | 12.42 | 9.946 |
| of_contig_OEFX01000005.1 - | | | | | | | | |
| Query protein | | | | | | | | |
| (9265_901) | | | | | | | | |
| Cas14u.6\|3300006028.a\| | 4.189 | 4.189 | 7.611 | 5.146 | 5.651 | 4.23 | 11.058 | 12.342 |
| Ga0070717_10000077\| | | | | | | | | |
| 54519 . . . 56201\|revcom | | | | | | | | |
| 466065_250_protein_locus_{—} | 2.518 | 2.518 | 10.909 | 10.633 | 8.457 | 3.972 | 12.527 | 10.584 |
| of_contig_SFKR01000004.1 - | | | | | | | | |
| Query protein | | | | | | | | |
| (466065_250) | | | | | | | | |
| Cas14a.5\|rifcsplowo2_01_{—} | 5.004 | 5.004 | 6.285 | 6.667 | 6.947 | 3.333 | 5.308 | 4.944 |
| scaffold_34461_curated\| | | | | | | | | |
| 4968 . . . 6521 | | | | | | | | |
| CasY2 | 6.424 | 6.424 | 3.072 | 2.728 | 2.647 | 8.408 | 3.686 | 3.431 |
| Cas14a.3\|gwa1_scaffold_{—} | 3.909 | 3.909 | 7.679 | 7.527 | 7.482 | 5.06 | 8.6 | 8.531 |
| 1795_curated\| | | | | | | | | |
| 25635 . . . 27224\|revcom | | | | | | | | |
| Cas14a.1\|rifcsphigho2_02_{—} | 4.425 | 4.425 | 7.076 | 9.441 | 8.253 | 3.98 | 8.734 | 7.667 |
| scaffold_2167_curated\| | | | | | | | | |
| 30296 . . . 31798\|revcom | | | | | | | | |
| Cas14a.2\|gwa2_scaffold_18027_{—} | 4.17 | 4.17 | 5.959 | 8.285 | 7.678 | 3.62 | 8.099 | 7.258 |
| curated\|7105 . . . 8628 | | | | | | | | |
| Cas14b.4\|cg1_0.2_scaffold_{—} | 3.106 | 3.103 | 7.356 | 7.638 | 6.667 | 4.488 | 8.829 | 7.571 |
| 785_c_curated\|32521 . . . | | | | | | | | |
| 34155 | | | | | | | | |
| Cas14b.7\|3300013125.a\| | 3.464 | 3.462 | 6.713 | 6.768 | 6.04 | 4.73 | 8.795 | 7.166 |
| Ga0172369_10000737\| | | | | | | | | |
| 994 . . . 2652\|revcom | | | | | | | | |
| Cas14u.2\|3300002172.a\| | 2.638 | 2.638 | 8.844 | 8.924 | 9.013 | 2.981 | 10.581 | 8.289 |
| JGI24730J26740_{—} | | | | | | | | |
| 1002785\|496 . . . | | | | | | | | |
| 1605\|revcom | | | | | | | | |
| Cas14b.3\|rifcsphigho2_01_{—} | 4.5 | 4.5 | 7.5 | 8.007 | 6.885 | 5.344 | 8.543 | 8.458 |
| scaffold_36781_curated\| | | | | | | | | |
| 2592 . . . 4217 | | | | | | | | |
| Cas14b.2\|rifcsplowo2_01_{—} | 4.15 | 4.15 | 8.185 | 7.143 | 7.317 | 4.713 | 9.318 | 8.143 |
| scaffold_282_curated\| | | | | | | | | |
| 77370 . . . 78983 | | | | | | | | |
| Cas14b.1\|rifcsplowo2_01_{—} | 4.29 | 4.29 | 7.871 | 8.174 | 7.813 | 4.778 | 9.03 | 8.224 |
| scaffold_239_curated\| | | | | | | | | |
| 54653 . . . 56257 | | | | | | | | |
| Cas14b.8\|3300013125.a\| | 4.29 | 4.29 | 7.168 | 7.292 | 6.424 | 4.863 | 8.543 | 8.581 |
| Ga0172369_10010464\| | | | | | | | | |
| 885 . . . 2489\|revcom | | | | | | | | |
| Cas14b.5\|rifcsphigho2_02_{—} | 4.267 | 4.267 | 6.914 | 7.155 | 6.096 | 5.518 | 8.401 | 7.827 |
| scaffold_55589_curated\| | | | | | | | | |
| 1904 . . . 3598 | | | | | | | | |
| Cas14b.6\|CG03_land_8_20_14_{—} | 3.92 | 3.92 | 7.12 | 6.421 | 5.696 | 5.887 | 8.372 | 8.359 |
| 0.80_scaffold_2214_curated\| | | | | | | | | |
| 6634 . . . 8466\|revcom | | | | | | | | |
| Cas14b.9\|3300013127.a\| | 4.099 | 4.099 | 8.483 | 6.874 | 5.769 | 5.442 | 8.703 | 8.399 |
| Ga0172365_10004421\| | | | | | | | | |
| 633 . . . 2366\|revcom | | | | | | | | |
| 209658_13971_protein_locus_{—} | 3.265 | 3.265 | 7.305 | 7.532 | 7.071 | 4.388 | 9.176 | 8.515 |
| of_contig_Ga0190333_1001561 - | | | | | | | | |
| Query protein | | | | | | | | |
| (209658_13971) | | | | | | | | |
| (2) | | | | | | | | |
| 209657_57738_protein_locus_{—} | 6.061 | 6.061 | 9.417 | 10.909 | 10.502 | 8.592 | 14 | 13.061 |
| of_contig_Ga0190332_1015597 - | | | | | | | | |
| Query protein | | | | | | | | |
| (209657_57738) | | | | | | | | |
| (2) | | | | | | | | |
| 209660_51257_protein_locus_{—} | 5.992 | 5.992 | 9.434 | 11.005 | 10.096 | 8.416 | 13.808 | 12.719 |
| of_contig_Ga0190335_1015156 - | | | | | | | | |
| Query protein | | | | | | | | |
| (209660_51257) | | | | | | | | |
| (2) | | | | | | | | |
| Cas14b.14\|gwc1_scaffold_8732_{—} | 3.514 | 3.511 | 6.636 | 7.302 | 5.521 | 4.519 | 7.209 | 5.968 |
| curated\|2705 . . . 4537 | | | | | | | | |
| Cas14b.15\|3300010293.a\| | 5.174 | 5.174 | 6.467 | 7.165 | 7.87 | 5.303 | 6.957 | 8.859 |
| Ga0116204_1008574\| | | | | | | | | |
| 2134 . . . 4032 | | | | | | | | |
| Cas14b.12\|CG22_combo_CG10- | 4.502 | 4.502 | 6.049 | 5.122 | 5.398 | 5.229 | 5.289 | 5.577 |
| 13_8_21_14_all_scaffold_2003_{—} | | | | | | | | |
| curated\|553 . . . | | | | | | | | |
| 2880\|revcom | | | | | | | | |
| Cas14b.13\|rifcsphigho2_01_{—} | 5.469 | 5.469 | 6.12 | 5.837 | 4.967 | 5.048 | 6.304 | 6.361 |
| scaffold_82367_curated\| | | | | | | | | |
| 1523 . . . 3856\|revcom | | | | | | | | |
| Cas14b.16\|3300005573.a\| | 5.097 | 5.015 | 8.544 | 6.552 | 7.899 | 5.401 | 7.553 | 5.655 |
| Ga0078972_1001015a\| | | | | | | | | |
| 33750 . . . 35627 | | | | | | | | |
| Cas14b.10\|CG08_land_8_20_14_{—} | 4.587 | 4.431 | 8.416 | 5.366 | 7.084 | 5.755 | 7.951 | 6.212 |
| 0.20_scaffold_1609_curated\| | | | | | | | | |
| 6134 . . . 7975 | | | | | | | | |
| Cas14b.11\|CG_4_10_14_0.8_{—} | 4.82 | 4.82 | 9.36 | 7.553 | 8.94 | 5.356 | 7.034 | 7.251 |
| um_filter_scaffold_20762_{—} | | | | | | | | |
| curated\|1372 . . . 3219 | | | | | | | | |
| Cas14u.1\|3300009029.a\| | 3.04 | 3.04 | 8.12 | 9.013 | 8.678 | 3.168 | 11.469 | 7.005 |
| Ga0066793_10010091\| | | | | | | | | |
| 37 . . . 1113\|revcom | | | | | | | | |
| Cas12c1 | 7.064 | 7.059 | 2.875 | 4.003 | 3.68 | 6.734 | 3.969 | 3.965 |
| Cas12c2 | 6.555 | 6.485 | 2.421 | 3.003 | 2.836 | 5.498 | 3.997 | 3.846 |
| Cas12a_UPI001113398F | 5.225 | 5.225 | 3.768 | 3.483 | 5.239 | 6.737 | 4.758 | 5.206 |
| Cas12b_UPI001113398F | 5.225 | 5.225 | 3.768 | 3.483 | 5.239 | 6.737 | 4.758 | 5.206 |
| Cas12b_tr\|A0A1I7F1U9\| | 5.252 | 5.252 | 3.772 | 3.388 | 5.133 | 6.546 | 4.633 | 5.306 |
| A0A1I7F1U9_9BACL | | | | | | | | |
| Cas12a_UPI00083514A7 | 5.44 | 5.512 | 3.846 | 3.822 | 4.388 | 5.998 | 4.112 | 4.749 |
| Cas12b_UPI00083514A7 | 5.44 | 5.512 | 3.846 | 3.822 | 4.388 | 5.998 | 4.112 | 4.749 |
| Cas12a_UPI00097159F1 | 5.874 | 5.946 | 4.03 | 3.825 | 5.717 | 5.998 | 4.093 | 5.225 |
| Cas12b_UPI00097159F1 | 5.874 | 5.946 | 4.03 | 3.825 | 5.717 | 5.998 | 4.093 | 5.225 |
| Cas12b_sp\|T0D7A2\| | 5.874 | 5.946 | 4.03 | 3.825 | 5.717 | 5.998 | 4.122 | 5.225 |
| CS12B_ALIAG | | | | | | | | |
| Cas12a_UPI0009715A14 | 5.874 | 5.946 | 4.03 | 3.825 | 5.717 | 6.074 | 4.122 | 5.225 |
| Cas12b_UPI0009715A14 | 5.874 | 5.946 | 4.03 | 3.825 | 5.717 | 6.074 | 4.122 | 5.225 |
| Cas12a_UPI00097159CF | 5.874 | 5.946 | 4.03 | 3.825 | 5.717 | 6.074 | 4.122 | 5.225 |
| Cas12b_UPI00097159CF | 5.874 | 5.946 | 4.03 | 3.825 | 5.717 | 6.074 | 4.122 | 5.225 |
| Cas12a_UPI000832F6D2 | 5.727 | 5.798 | 4.213 | 3.918 | 5.524 | 6.226 | 3.939 | 5.316 |
| Cas12b_UPI000832F6D2 | 5.727 | 5.798 | 4.213 | 3.918 | 5.524 | 6.226 | 3.939 | 5.316 |
| Cas12b_tr\|A0A512CSX2\| | 5.798 | 5.87 | 4.213 | 3.731 | 5.337 | 6.302 | 4.029 | 5.316 |
| A0A512CSX2_9BACL | | | | | | | | |
| OspCas12c | 7.426 | 7.567 | 2.922 | 3.084 | 3.328 | 5.58 | 3.325 | 4.133 |
| Cas14u.5\|3300012532.a\| | 4.064 | 4.064 | 4.154 | 6.37 | 6.038 | 5.068 | 6.96 | 9.531 |
| Ga0137373_10000316\| | | | | | | | | |
| 3286 . . . 5286 | | | | | | | | |
| 63461_4106_protein_locus_{—} | 3.303 | 3.303 | 5.096 | 5.949 | 5.512 | 4.017 | 6.192 | 7.657 |
| of_contig_LSKL01000323 - | | | | | | | | |
| Query protein | | | | | | | | |
| (63461_4106) | | | | | | | | |
| translation (4) | | | | | | | | |
| 58610_1188_protein_locus_{—} | 3.988 | 3.988 | 4.416 | 6.19 | 4.212 | 4.693 | 7.099 | 8.769 |
| of_contig_LFOD01000003 - | | | | | | | | |
| Query protein | | | | | | | | |
| (58610_1188) | | | | | | | | |
| translation (5) | | | | | | | | |
| 21566_3969_protein_locus_{—} | 3.627 | 3.627 | 5.76 | 6.924 | 4.944 | 4.014 | 8.791 | 7.351 |
| of_contig_BAFB01000202 - | | | | | | | | |
| Query protein | | | | | | | | |
| (21566_3969) | | | | | | | | |
| translation (4) | | | | | | | | |

TABLE 5

| | | Cas14c.1\| | | | | | Cas14u.3\| | |
|---|---|---|---|---|---|---|---|---|
| | | CG10_big_{—} | | | | | 19ft_2_{—} | |
| | | fil_rev_8_21_{—} | | | | | nophage_{—} | |
| | Cas14h.2\| | 14_0.10_{—} | | | | | noknown_{—} | Cas14u.7\| |
| | 3300005921.a\| | scaffold_4477_{—} | | | | | scaffold_{—} | 3300001256.a\| |
| | Ga0070766_{—} | curated\| | | | | | 0_curated\| | JGI12210J13797_{—} |
| | 10011912\| | 19327 . . . | | | | | 508188 . . . | 10004690\| |
| | 384 . . . 2081 | 20880\|revcom | Cas12h1 | CasX1 | CasX2 | CasY1 | 509648 | 5792 . . . 7006 |
| Cas14g.1\|RBG_13_{—} | 6.307 | 5.696 | 6.801 | 7.116 | 7.033 | 6.31 | 7.628 | 8.531 |
| scaffold_1401_{—} | | | | | | | | |
| curated\|15949 . . . 18180 | | | | | | | | |
| Cas14g.2\|3300009652.a\| | 8.258 | 6.349 | 6.015 | 5.52 | 5.592 | 4.979 | 7.483 | 7.733 |
| Ga0123330_1010394\| | | | | | | | | |
| 2814 . . . 5123 | | | | | | | | |
| Cas12i2 | 4.444 | 4.178 | 5.403 | 6.421 | 5.867 | 7.038 | 4.688 | 2.921 |
| Cas12i1 | 4.089 | 3.815 | 5.47 | 6.225 | 5.341 | 4.286 | 4.377 | 3.03 |
| Cas12g1 | 5.457 | 5.402 | 6.919 | 6.724 | 6.796 | 6.423 | 6.883 | 5.952 |
| Cas14d.3\|RIFCSPLOWO2_{—} | 7.386 | 6.036 | 6.586 | 6.571 | 6.522 | 4.376 | 9.741 | 5.855 |
| 01_FULL_OD1_45_34b_{—} | | | | | | | | |
| rifcsplowo2_01_{—} | | | | | | | | |
| scaffold_3495_curated\| | | | | | | | | |
| 25656 . . . 27605\|revcom | | | | | | | | |
| Cas14d.1\|RIFCSPHIGHO2_{—} | 5.706 | 4.654 | 4.432 | 5.714 | 5.28 | 4.513 | 9.105 | 4 |
| 01_FULL_CPR_46_36_{—} | | | | | | | | |
| rifcsphigho2_01_{—} | | | | | | | | |
| scaffold_646_curated\| | | | | | | | | |
| 49808 . . . 51616\|revcom | | | | | | | | |
| CasY5 | 4.474 | 3.616 | 5.237 | 5.849 | 6.061 | 6.407 | 2.842 | 2.743 |
| Cas14a.4\|CG10_big_fil_rev_{—} | 5.643 | 6.472 | 5.527 | 5.443 | 6.279 | 5.178 | 7.945 | 7.448 |
| 8_21_14_0.10_scaffold_{—} | | | | | | | | |
| 20906_curated\| | | | | | | | | |
| 649 . . . 2829 | | | | | | | | |
| CasY6 | 3.633 | 2.96 | 6.121 | 6 | 7.645 | 6.381 | 4.077 | 2.927 |
| Cas14f.1\|rifcsp13_1_{—} | 7.477 | 6.95 | 6.416 | 5.825 | 5.859 | 6.047 | 7.343 | 7.542 |
| sub10_scaffold_3_curated\| | | | | | | | | |
| 38906 . . . 41041 | | | | | | | | |
| Cas14f.2\|3300009991.a\| | 7.362 | 8.05 | 5.131 | 3.887 | 3.854 | 3.874 | 6.518 | 9.769 |
| Ga0105042_100140\| | | | | | | | | |
| 1624 . . . 3348 | | | | | | | | |
| Cas14a.6\|3300012359.a\| | 6.588 | 8.818 | 4.61 | 5.123 | 6.515 | 4.736 | 9.524 | 8.554 |
| Ga0137385_10000156\| | | | | | | | | |
| 41289 . . . 42734 | | | | | | | | |
| Cas12a_UPI00094EEDB4 | 3.379 | 3.414 | 3.627 | 5.151 | 4.716 | 5.234 | 3.73 | 2.846 |
| Cas12a_UPI000B4235CE | 3.991 | 3.104 | 5.205 | 6.204 | 5.564 | 5.688 | 3.026 | 3.007 |
| Cas12a_UPI000818CC52 | 3.991 | 3.104 | 5.066 | 6.213 | 5.572 | 5.626 | 3.026 | 3.007 |
| Cas12a_UPI0007B78B7F | 3.991 | 3.104 | 5.205 | 6.13 | 5.564 | 5.688 | 3.026 | 3.007 |
| Cas12a_UPI000B4235F9 | 3.991 | 3.104 | 5.205 | 6.13 | 5.49 | 5.688 | 3.026 | 3.007 |
| Cas14e.2\|rifcsplowo2_01_{—} | 5.448 | 8.63 | 5.396 | 4.041 | 4.603 | 3.306 | 7.579 | 8.463 |
| scaffold_81231_curated\| | | | | | | | | |
| 976 . . . 2217 | | | | | | | | |
| Cas14e.1\|rifcsphigho2_01_{—} | 6.154 | 8.443 | 5.383 | 3.316 | 3.556 | 4.033 | 8.598 | 8.609 |
| scaffold_566_curated\| | | | | | | | | |
| 113069 . . . 114313 | | | | | | | | |
| Cas14e.3\|rifcsphigho2_01_{—} | 7.179 | 6.964 | 4.556 | 4.063 | 4.316 | 4.5 | 7.895 | 9.298 |
| scaffold_4702_curated\| | | | | | | | | |
| 82881 . . . 84230\|revcom | | | | | | | | |
| CasY4 | 2.773 | 2.927 | 3.965 | 7.065 | 7.422 | 6.984 | 3.495 | 4.114 |
| Cas14h.3\|3300009698.a\| | 14.56 | 9.589 | 5.166 | 5.217 | 5.489 | 3.908 | 7.679 | 13.546 |
| Ga0116216_10000905\| | | | | | | | | |
| 8005 . . . 9504 | | | | | | | | |
| Cas14h.1\|3300005602.a\| | 65.12 | 8.889 | 4.577 | 4.709 | 4.044 | 3.953 | 6.408 | 10.764 |
| Ga0070762_10001740\| | | | | | | | | |
| 7377 . . . 9071\|revcom | | | | | | | | |
| Cas14h.2\|3300005921.a\| | | 8.293 | 4.93 | 4.5 | 4.541 | 4.324 | 6.229 | 10.175 |
| Ga0070766_10011912\| | | | | | | | | |
| 384 . . . 2081 | | | | | | | | |
| Cas14c.1\|CG10_big_fil_rev_{—} | 8.293 | | 4.881 | 3.969 | 4.382 | 4.758 | 7.705 | 14.801 |
| 8_21_14_0.10_scaffold_{—} | | | | | | | | |
| 4477_curated\| | | | | | | | | |
| 19327 . . . 20880\|revcom | | | | | | | | |
| Cas12h1 | 4.93 | 4.881 | | 5.945 | 6.267 | 4.718 | 4.875 | 4.745 |
| CasX1 | 4.5 | 3.969 | 5.945 | | 51.406 | 7.309 | 5.864 | 5.664 |
| CasX2 | 4.541 | 4.382 | 6.267 | 51.406 | | 7.535 | 5.497 | 5.411 |
| CasY1 | 4.324 | 4.758 | 4.718 | 7.309 | 7.535 | | 5.474 | 5.249 |
| Cas14u.3\|19ft_2_nophage_{—} | 6.229 | 7.705 | 4.875 | 5.864 | 5.497 | 5.474 | | 9.145 |
| noknown_scaffold_0_{—} | | | | | | | | |
| curated\|508188 . . . 509648 | | | | | | | | |
| Cas14u.7\|3300001256.a\| | 10.175 | 14.801 | 4.745 | 5.664 | 5.411 | 5.249 | 9.145 | |
| JGI12210J13797_10004690\| | | | | | | | | |
| 5792 . . . 7006 | | | | | | | | |
| Cas14u.8\|3300005660.a\| | 9.507 | 12.5 | 4.255 | 6.192 | 5.521 | 3.87 | 10.6 | 28.261 |
| Ga0073904_10021651\| | | | | | | | | |
| 765 . . . 1943 | | | | | | | | |
| Cas14u.4\|rifcsp2_19_4_full_{—} | 7.958 | 9.228 | 4.014 | 3.905 | 5.083 | 3.436 | 7.171 | 12.156 |
| scaffold_168_curated\| | | | | | | | | |
| 84455 . . . 85657 | | | | | | | | |
| Cas14d.2\|rifcsphigho2_01_{—} | 9.029 | 7.009 | 5.156 | 5.079 | 5.769 | 4.424 | 13.996 | 7.828 |
| scaffold_10981_curated\| | | | | | | | | |
| 5762 . . . 7246\|revcom | | | | | | | | |
| Cas14c.2\|3300001245.a\| | 8.844 | 12.104 | 5.041 | 5.397 | 5.139 | 3.953 | 8.35 | 18.075 |
| JGI12048J13642_10201286\| | | | | | | | | |
| 4257 . . . 5489\|revcom | | | | | | | | |
| CasY3 | 3.574 | 4.225 | 5.462 | 9.297 | 8.394 | 7.062 | 3.962 | 4.17 |
| 633299_527_protein_locus_{—} | 9.705 | 15.356 | 5.226 | 5.041 | 4.673 | 4.344 | 9.486 | 25.935 |
| of_contig_Scfld15 - | | | | | | | | |
| Query protein | | | | | | | | |
| (633299_527) (4) | | | | | | | | |
| 8971_2857_protein_locus_{—} | 10.261 | 14.228 | 4.701 | 6.12 | 5.96 | 4.607 | 10 | 25.515 |
| of_contig_OEJQ01000083.1 - | | | | | | | | |
| Query protein | | | | | | | | |
| (8971_2857) | | | | | | | | |
| 9265_901_protein_locus_{—} | 10.42 | 14.712 | 4.762 | 6.156 | 5.889 | 4.558 | 9.978 | 26.316 |
| of_contig_OEFX01000005.1 - | | | | | | | | |
| Query protein | | | | | | | | |
| (9265_901) | | | | | | | | |
| Cas14u.6\|3300006028.a\| | 11.774 | 7.573 | 5.38 | 4.67 | 5.123 | 3.815 | 6.436 | 10.071 |
| Ga0070717_10000077\| | | | | | | | | |
| 54519 . . . 56201\|revcom | | | | | | | | |
| 466065_250_protein_locus_{—} | 12.222 | 15.464 | 4.423 | 5.65 | 5.92 | 5.019 | 9.776 | 29.563 |
| of_contig_SFKR01000004.1 - | | | | | | | | |
| Query protein | | | | | | | | |
| (466065_250) | | | | | | | | |
| Cas14a.5\|rifcsplowo2_01_{—} | 5.016 | 5.873 | 5.012 | 5.061 | 5.231 | 3.597 | 7.584 | 7.635 |
| scaffold_34461_curated\| | | | | | | | | |
| 4968 . . . 6521 | | | | | | | | |
| CasY2 | 3.529 | 2.977 | 5.167 | 7.529 | 8.089 | 6.977 | 4.255 | 3.442 |
| Cas14a.3\|gwa1_scaffold_1795_{—} | 8.065 | 9.431 | 6.36 | 7.611 | 7.257 | 5.355 | 9.206 | 9.108 |
| curated\|25635 . . . | | | | | | | | |
| 27224\|revcom | | | | | | | | |
| Cas14a.1\|rifcsphigho2_02_{—} | 7.155 | 8.919 | 6.683 | 7.21 | 7.278 | 5.119 | 8.379 | 10.6 |
| scaffold_2167_curated\| | | | | | | | | |
| 30296 . . . 31798\|revcom | | | | | | | | |
| Cas14a.2\|gwa2_scaffold_18027_{—} | 7.401 | 8.136 | 7.101 | 7.78 | 7.749 | 5.086 | 8.561 | 11.637 |
| curated\|7105 . . . 8628 | | | | | | | | |
| Cas14b.4\|cg1_0.2_scaffold_785_{—} | 8.833 | 8.108 | 5.945 | 7.07 | 7.446 | 5.839 | 9.508 | 9.141 |
| c_curated\|32521 . . . 34155 | | | | | | | | |
| Cas14b.7\|3300013125.a\| | 8.095 | 8.217 | 5.813 | 7.026 | 7.202 | 5.641 | 9.903 | 9.091 |
| Ga0172369_10000737\| | | | | | | | | |
| 994 . . . 2652\|revcom | | | | | | | | |
| Cas14u.2\|3300002172.a\| | 8.496 | 10.291 | 4.207 | 5.981 | 5.932 | 3.751 | 9.919 | 13.35 |
| JGI24730J26740_{—} | | | | | | | | |
| 1002785\|496 . . . | | | | | | | | |
| 1605\|revcom | | | | | | | | |
| Cas14b.3\|rifcsphigho2_01_{—} | 8.804 | 8.373 | 6.413 | 6.9 | 6.861 | 4.666 | 9.402 | 10.929 |
| scaffold_36781_curated\| | | | | | | | | |
| 2592 . . . 4217 | | | | | | | | |
| Cas14b.2\|rifcsplowo2_01_{—} | 8.76 | 7.813 | 6.475 | 6.191 | 6.78 | 4.625 | 10.517 | 10.83 |
| scaffold_282_curated\| | | | | | | | | |
| 77370 . . . 78983 | | | | | | | | |
| Cas14b.1\|rifcsplowo2_01_{—} | 9.349 | 7.559 | 6.325 | 6.263 | 6.533 | 4.972 | 9.879 | 10.969 |
| scaffold_239_curated\| | | | | | | | | |
| 54653 . . . 56257 | | | | | | | | |
| Cas14b.8\|3300013125.a\| | 8.878 | 6.951 | 6.205 | 5.741 | 6.639 | 5.249 | 11.092 | 10.275 |
| Ga0172369_10010464\| | | | | | | | | |
| 885 . . . 2489\|revcom | | | | | | | | |
| Cas14b.5\|rifcsphigho2_02_{—} | 8.333 | 7.562 | 5.917 | 6.076 | 6.757 | 6.141 | 10.611 | 10.247 |
| scaffold_55589_curated\| | | | | | | | | |
| 1904 . . . 3598 | | | | | | | | |
| Cas14b.6\|CG03_land_8_20_14_{—} | 8.217 | 7.852 | 6.936 | 5.906 | 8.016 | 7.182 | 9.365 | 10.351 |
| 0.80_scaffold_2214_curated\| | | | | | | | | |
| 6634 . . . 8466\|revcom | | | | | | | | |
| Cas14b.9\|3300013127.a\| | 8.517 | 7.519 | 6.746 | 6.475 | 8.091 | 6.9 | 9.532 | 11.379 |
| Ga0172365_10004421\| | | | | | | | | |
| 633 . . . 2366\|revcom | | | | | | | | |
| 209658_13971_protein_locus_{—} | 8.37 | 9.534 | 5.522 | 5.695 | 6.032 | 5.614 | 11.058 | 14.481 |
| of_contig_Ga0190333_1001561 - | | | | | | | | |
| Query protein | | | | | | | | |
| (209658_13971) | | | | | | | | |
| (2) | | | | | | | | |
| 209657_57738_protein_locus_{—} | 12.863 | 11.189 | 8.434 | 11.905 | 12.04 | 9.346 | 17.593 | 20.657 |
| of_contig_Ga0190332_1015597 - | | | | | | | | |
| Query protein | | | | | | | | |
| (209657_57738) | | | | | | | | |
| (2) | | | | | | | | |
| 209660_51257_protein_locus_{—} | 13.043 | 10.545 | 8.202 | 10.601 | 10.764 | 8.633 | 17.073 | 20.297 |
| of_contig_Ga0190335_1015156 - | | | | | | | | |
| Query protein | | | | | | | | |
| (209660_51257) | | | | | | | | |
| (2) | | | | | | | | |
| Cas14b.14\|gwc1_scaffold_{—} | 6.696 | 10.836 | 6.466 | 6.97 | 7.446 | 5.626 | 7.903 | 9.6 |
| 8732_curated\|2705 . . . 4537 | | | | | | | | |
| Cas14b.15\|3300010293.a\| | 9.531 | 7.349 | 3.913 | 7.419 | 7.369 | 6.806 | 8.788 | 7.741 |
| Ga0116204_1008574\| | | | | | | | | |
| 2134 . . . 4032 | | | | | | | | |
| Cas14b.12\|CG22_combo_CG10- | 6.21 | 6.835 | 5.509 | 7.486 | 6.907 | 6.643 | 7.226 | 7.642 |
| 13_8_21_14_all_scaffold_2003_{—} | | | | | | | | |
| curated\|553 . . . | | | | | | | | |
| 2880\|revcom | | | | | | | | |
| Cas14b.13\|rifcsphigho2_01_{—} | 6.555 | 8.087 | 5.943 | 6.167 | 6.997 | 5.948 | 8.042 | 6.762 |
| scaffold_82367_curated\| | | | | | | | | |
| 1523 . . . 3856\|revcom | | | | | | | | |
| Cas14b.16\|3300005573.a\| | 5.891 | 8.921 | 6.171 | 6.612 | 6.66 | 6.818 | 9.176 | 7.865 |
| Ga0078972_1001015a\| | | | | | | | | |
| 33750 . . . 35627 | | | | | | | | |
| Cas14b.10\|CG08_land_8_20_14_{—} | 7.187 | 8.837 | 5.977 | 6.984 | 7.464 | 6.828 | 10.098 | 10.231 |
| 0.20_scaffold_1609_curated\| | | | | | | | | |
| 6134 . . . 7975 | | | | | | | | |
| Cas14b.11\|CG_4_10_14_0.8_{—} | 8.346 | 7.965 | 5.963 | 7.419 | 7.906 | 5.951 | 9.82 | 9.091 |
| um_filter_scaffold_20762_{—} | | | | | | | | |
| curated\|1372 . . . 3219 | | | | | | | | |
| Cas14u.1\|3300009029.a\| | 7.951 | 7.129 | 3.865 | 5.456 | 5.191 | 4.048 | 10.331 | 12.528 |
| Ga0066793_10010091\| | | | | | | | | |
| 37 . . . 1113\|revcom | | | | | | | | |
| Cas12c1 | 4.196 | 3.75 | 5.352 | 7.083 | 7.192 | 7.049 | 3.92 | 3.259 |
| Cas12c2 | 4.01 | 3.207 | 5.016 | 6.63 | 5.915 | 5.659 | 3.172 | 3.185 |
| Cas12a_UPI001113398F | 4.668 | 3.856 | 5.598 | 6.371 | 6.209 | 5.166 | 4.269 | 3.249 |
| Cas12b_UPI001113398F | 4.668 | 3.856 | 5.598 | 6.371 | 6.209 | 5.166 | 4.269 | 3.249 |
| Cas12b_tr\|A0A1I7F1U9\| | 4.852 | 3.665 | 5.763 | 6.31 | 5.882 | 5.183 | 4.269 | 3.237 |
| A0A1I7F1U9_9BACL | | | | | | | | |
| Cas12a_UPI00083514A7 | 4.659 | 4.087 | 5.64 | 6.034 | 5.705 | 5.624 | 3.993 | 3.584 |
| Cas12b_UPI00083514A7 | 4.659 | 4.087 | 5.64 | 6.034 | 5.705 | 5.624 | 3.993 | 3.584 |
| Cas12a_UPI00097159F1 | 5.133 | 4.452 | 6.374 | 5.916 | 5.412 | 4.867 | 4.457 | 3.306 |
| Cas12b_UPI00097159F1 | 5.133 | 4.452 | 6.374 | 5.916 | 5.412 | 4.867 | 4.457 | 3.306 |
| Cas12b_sp\|T0D7A2\| | 5.133 | 4.452 | 6.374 | 5.916 | 5.412 | 4.867 | 4.457 | 3.306 |
| CS12B_ALIAG | | | | | | | | |
| Cas12a_UPI0009715A14 | 5.133 | 4.452 | 6.374 | 5.916 | 5.329 | 4.867 | 4.457 | 3.214 |
| Cas12b_UPI0009715A14 | 5.133 | 4.452 | 6.374 | 5.916 | 5.329 | 4.867 | 4.457 | 3.214 |
| Cas12a_UPI00097159CF | 5.133 | 4.452 | 6.374 | 5.916 | 5.412 | 4.867 | 4.457 | 3.306 |
| Cas12b_UPI00097159CF | 5.133 | 4.452 | 6.374 | 5.916 | 5.412 | 4.867 | 4.457 | 3.306 |
| Cas12a_UPI000832F6D2 | 5.225 | 4.27 | 5.938 | 6.076 | 5.74 | 5.102 | 4.731 | 3.394 |
| Cas12b_UPI000832F6D2 | 5.225 | 4.27 | 5.938 | 6.076 | 5.74 | 5.102 | 4.731 | 3.394 |
| Cas12b_tr\|A0A512CSX2\| | 5.133 | 4.27 | 5.766 | 5.993 | 5.657 | 5.102 | 4.453 | 3.394 |
| A0A512CSX2_9BACL | | | | | | | | |
| OspCas12c | 4.708 | 3.503 | 5.263 | 5.792 | 6.386 | 6.691 | 4.214 | 3.339 |
| Cas14u.5\|3300012532.a\| | 8.417 | 4.032 | 6.749 | 6.016 | 5.731 | 5.818 | 6.287 | 5.589 |
| Ga0137373_10000316\| | | | | | | | | |
| 3286 . . . 5286 | | | | | | | | |
| 63461_4106_protein_locus_{—} | 7.055 | 4.928 | 6.082 | 4.187 | 5.348 | 3.931 | 7.981 | 4.754 |
| of_contig_LSKL01000323 - | | | | | | | | |
| Query protein | | | | | | | | |
| (63461_4106) | | | | | | | | |
| translation (4) | | | | | | | | |
| 58610_1188_protein_locus_{—} | 7.154 | 5.24 | 6.176 | 5.123 | 5.184 | 4.182 | 6.955 | 6.139 |
| of_contig_LFOD01000003 - | | | | | | | | |
| Query protein | | | | | | | | |
| (58610_1188) | | | | | | | | |
| translation (5) | | | | | | | | |
| 21566_3969_protein_locus_{—} | 7.87 | 5.294 | 6.007 | 4.266 | 4.418 | 4.771 | 7.442 | 5.785 |
| of_contig_BAFB01000202 - | | | | | | | | |
| Query protein | | | | | | | | |
| (21566_3969) | | | | | | | | |
| translation (4) | | | | | | | | |

TABLE 6

| | Cas14u.8\| | Cas14u.4\| | Cas14d.2\| | Cas14c.2\| | | 633299_527_{—} | 8971_2857_protein_{—} | 9265_901_protein_{—} |
|---|---|---|---|---|---|---|---|---|
| | 3300005660.a\| | rifcsp2_19_4_{—} | rifcsphigho2_{—} | 3300001245.a\| | | protein_locus_of_{—} | locus_of_contig_{—} | locus_of_contig_{—} |
| | Ga0073904_{—} | full_scaffold_{—} | 01_scaffold_{—} | JGI12048J13642_{—} | | contig_Scfld15 - | OEJQ01000083.1 - | OEFX01000005.1 - |
| | 10021651\| | 168_curated\| | 10981_curated\| | 10201286\| | | Query protein | Query protein | Query protein |
| | 765 . . . 1943 | 84455 . . . 85657 | 5762 . . . 7246\|revcom | 4257 . . . 5489\|revcom | CasY3 | (633299_527) (4) | (8971_2857) | (9265_901) |
| Cas14g.1\|RBG_13_{—} | 7.341 | 6.137 | 7.444 | 7.459 | 5.921 | 6.853 | 6.677 | 6.567 |
| scaffold_1401_{—} | | | | | | | | |
| curated\|15949 . . . 18180 | | | | | | | | |
| Cas14g.2\|3300009652.a\| | 5.992 | 5.615 | 5.898 | 7.246 | 4.781 | 7.057 | 6.14 | 6.043 |
| Ga0123330_1010394\| | | | | | | | | |
| 2814 . . . 5123 | | | | | | | | |
| Cas12i2 | 3.891 | 3.783 | 4.051 | 3.961 | 6.715 | 4.203 | 5.263 | 5.203 |
| Cas12i1 | 3.39 | 3.491 | 3.707 | 4.864 | 4.958 | 3.491 | 2.944 | 3.012 |
| Cas12g1 | 5.812 | 5.797 | 6.045 | 6.021 | 5.753 | 6.109 | 5.579 | 5.493 |
| Cas14d.3\|RIFCSPLOWO2_{—} | 6.317 | 8.841 | 11.318 | 6.156 | 4.456 | 5.819 | 4.866 | 4.942 |
| 01_FULL_OD1_45_34b_{—} | | | | | | | | |
| rifcsplowo2_01_{—} | | | | | | | | |
| scaffold_3495_{—} | | | | | | | | |
| curated\|25656 . . . | | | | | | | | |
| 27605\|revcom | | | | | | | | |
| Cas14d.1\|RIFCSPHIGHO2_{—} | 4.341 | 3.797 | 9.486 | 4.859 | 3.918 | 5.28 | 4.53 | 4.444 |
| 01_FULL_CPR_46_36_{—} | | | | | | | | |
| rifcsphigho2_01_{—} | | | | | | | | |
| scaffold_646_curated\| | | | | | | | | |
| 49808 . . . 51616\|revcom | | | | | | | | |
| Cas Y5 | 2.741 | 3.527 | 3.495 | 3.163 | 6.795 | 3.815 | 3.704 | 3.759 |
| Cas14a.4\|CG10_big_fil_{—} | 7.26 | 5.761 | 6.389 | 7.191 | 5.481 | 6.474 | 6.922 | 6.812 |
| rev_8_21_14_0.10_{—} | | | | | | | | |
| scaffold_20906_{—} | | | | | | | | |
| curated\|649 . . . 2829 | | | | | | | | |
| CasY6 | 3.712 | 2.776 | 3.772 | 2.675 | 8.333 | 3.323 | 3.078 | 3.133 |
| Cas14f.1\|rifcsp13_1_{—} | 7.972 | 4.33 | 7.412 | 6.658 | 5.316 | 7.832 | 7.059 | 6.946 |
| sub10_scaffold_3_{—} | | | | | | | | |
| curated\|38906 . . . 41041 | | | | | | | | |
| Cas14f.2\|3300009991.a\| | 8.099 | 7.317 | 7.026 | 5.415 | 3.772 | 7.679 | 8.098 | 7.934 |
| Ga0105042_100140\| | | | | | | | | |
| 1624 . . . 3348 | | | | | | | | |
| Cas14a.6\|3300012359.a\| | 8.704 | 10.2 | 11.132 | 9.312 | 3.416 | 9.298 | 10.478 | 10.222 |
| Ga0137385_10000156\| | | | | | | | | |
| 41289 . . . 42734 | | | | | | | | |
| Cas12a_UPI00094EEDB4 | 2.771 | 3.082 | 3.991 | 2.822 | 5.999 | 3.009 | 2.659 | 2.716 |
| Cas12a_UPI000B4235CE | 3.075 | 3.077 | 4.372 | 3.351 | 6.877 | 3.236 | 3.223 | 3.195 |
| Cas12a_UPI000818CC52 | 3.075 | 3.077 | 4.372 | 3.351 | 6.887 | 3.236 | 3.223 | 3.195 |
| Cas12a_UPI0007B78B7F | 3.075 | 3.077 | 4.372 | 3.351 | 6.877 | 3.236 | 3.223 | 3.195 |
| Cas12a_UPI000B4235F9 | 3.075 | 3.077 | 4.372 | 3.351 | 6.877 | 3.236 | 3.223 | 3.195 |
| Cas14e.2\|rifcsplowo2_01_{—} | 8.036 | 8.15 | 6.191 | 7.463 | 3.198 | 9.888 | 9.832 | 9.579 |
| scaffold_81231_curated\| | | | | | | | | |
| 976 . . . 2217 | | | | | | | | |
| Cas14e.1\|rifcsphigho2_01_{—} | 8.869 | 6.813 | 7.836 | 6.438 | 2.936 | 10.811 | 8.794 | 8.557 |
| scaffold_566_curated\| | | | | | | | | |
| 113069 . . . 114313 | | | | | | | | |
| Cas14e.3\|rifcsphigho2_01_{—} | 7.438 | 5.809 | 7.076 | 7.6 | 3.128 | 10.669 | 7.586 | 7.399 |
| scaffold_4702_curated\| | | | | | | | | |
| 82881 . . . 84230\|revcom | | | | | | | | |
| CasY4 | 3.28 | 2.521 | 3.757 | 3.763 | 7.777 | 3.788 | 4.111 | 4.248 |
| Cas14h.3\|3300009698.a\| | 12.749 | 8.984 | 7.218 | 13.112 | 3.926 | 10.097 | 12.281 | 12.42 |
| Ga0116216_10000905\| | | | | | | | | |
| 8005 . . . 9504 | | | | | | | | |
| Cas14h.1\|3300005602.a\| | 9.457 | 7.863 | 7.445 | 8.263 | 3.568 | 9.091 | 9.594 | 9.946 |
| Ga0070762_10001740\| | | | | | | | | |
| 7377 . . . 9071\|revcom | | | | | | | | |
| Cas14h.2\|3300005921.a\| | 9.507 | 7.958 | 9.029 | 8.844 | 3.574 | 9.705 | 10.261 | 10.42 |
| Ga0070766_10011912\| | | | | | | | | |
| 384 . . . 2081 | | | | | | | | |
| Cas14c.1\|CG10_big_fil_{—} | 12.5 | 9.228 | 7.009 | 12.104 | 4.225 | 15.356 | 14.228 | 14.712 |
| rev_8_21_14_0.10_{—} | | | | | | | | |
| scaffold_4477_curated\| | | | | | | | | |
| 19327 . . . 20880\|revcom | | | | | | | | |
| Cas12h1 | 4.255 | 4.014 | 5.156 | 5.041 | 5.462 | 5.226 | 4.701 | 4.762 |
| CasX1 | 6.192 | 3.905 | 5.079 | 5.397 | 9.297 | 5.041 | 6.12 | 6.156 |
| CasX2 | 5.521 | 5.083 | 5.769 | 5.139 | 8.394 | 4.673 | 5.96 | 5.889 |
| CasY1 | 3.87 | 3.436 | 4.424 | 3.953 | 7.062 | 4.344 | 4.607 | 4.558 |
| Cas14u.3\|19ft_2_nophage_{—} | 10.6 | 7.171 | 13.996 | 8.35 | 3.962 | 9.486 | 10 | 9.978 |
| noknown_scaffold_{—} | | | | | | | | |
| 0_curated\|508188 . . . 509648 | | | | | | | | |
| Cas14u.7\|3300001256.a\| | 28.261 | 12.156 | 7.828 | 18.075 | 4.17 | 25.935 | 25.515 | 26.316 |
| JGI12210J13797_{—} | | | | | | | | |
| 10004690\|5792 . . . 7006 | | | | | | | | |
| Cas14u.8\|3300005660.a\| | | 12.121 | 9.742 | 15.529 | 4.174 | 30.288 | 33.6 | 34.456 |
| Ga0073904_10021651\| | | | | | | | | |
| 765 . . . 1943 | | | | | | | | |
| Cas14u.4\|rifcsp2_19_4_{—} | 12.121 | | 8.35 | 11.83 | 3.416 | 11.364 | 14.604 | 14.217 |
| full_scaffold_168_{—} | | | | | | | | |
| curated\|84455 . . . 85657 | | | | | | | | |
| Cas14d.2\|rifcsphigho2_01_{—} | 9.742 | 8.35 | | 6.526 | 4.352 | 8.876 | 8.096 | 8.12 |
| scaffold_10981_curated\| | | | | | | | | |
| 5762 . . . 7246\|revcom | | | | | | | | |
| Cas14c.2\|3300001245.a\| | 15.529 | 11.83 | 6.526 | | 5.089 | 17.29 | 22.572 | 21.939 |
| JGI12048J13642_{—} | | | | | | | | |
| 10201286\|4257 . . . 5489\| | | | | | | | | |
| revcom | | | | | | | | |
| CasY3 | 4.174 | 3.416 | 4.352 | 5.089 | | 4.437 | 4.277 | 4.414 |
| 633299_527_protein_locus_{—} | 30.288 | 11.364 | 8.876 | 17.29 | 4.437 | | 32.987 | 33.838 |
| of_contig_Scfld15 - | | | | | | | | |
| Query protein | | | | | | | | |
| (633299_527) (4) | | | | | | | | |
| 8971_2857_protein_locus_{—} | 33.6 | 14.604 | 8.096 | 22.572 | 4.277 | 32.987 | | 100 |
| of_contig_OEJQ01000083.1 - | | | | | | | | |
| Query protein | | | | | | | | |
| (8971_2857) | | | | | | | | |
| 9265_901_protein_locus_{—} | 34.456 | 14.217 | 8.12 | 21.939 | 4.414 | 33.838 | 100 | |
| of_contig_OEFX01000005.1 - | | | | | | | | |
| Query protein | | | | | | | | |
| (9265_901) | | | | | | | | |
| Cas14u.6\|3300006028.a\| | 9.769 | 7.193 | 7.143 | 8.448 | 4.663 | 8.772 | 9.851 | 9.836 |
| Ga0070717_10000077\| | | | | | | | | |
| 54519 . . . 56201\|revcom | | | | | | | | |
| 466065_250_protein_locus_{—} | 31.759 | 13.022 | 9.562 | 19.851 | 4.474 | 37.047 | 44.092 | 44.134 |
| of_contig_SFKR01000004.1 - | | | | | | | | |
| Query protein | | | | | | | | |
| (466065_250) | | | | | | | | |
| Cas14a.5\|rifcsplowo2_01_{—} | 5.056 | 5.311 | 7.04 | 5.263 | 2.703 | 6.642 | 5.394 | 5.882 |
| scaffold_34461_curated\| | | | | | | | | |
| 4968 . . . 6521 | | | | | | | | |
| CasY2 | 3.61 | 4.373 | 4.195 | 3.833 | 8.24 | 3.987 | 3.467 | 3.433 |
| Cas14a.3\|gwa1_scaffold_{—} | 9.125 | 8.939 | 8.711 | 11.481 | 4.613 | 12.008 | 8.264 | 8.283 |
| 1795_curated\|25635 . . . | | | | | | | | |
| 27224\|revcom | | | | | | | | |
| Cas14a.1\|rifcsphigho2_02_{—} | 8.73 | 9.703 | 9.444 | 11.637 | 4.483 | 12.176 | 10.067 | 10.044 |
| scaffold_2167_curated\| | | | | | | | | |
| 30296 . . . 31798\|revcom | | | | | | | | |
| Cas14a.2\|gwa2_scaffold_{—} | 9.393 | 10.352 | 9.444 | 12.84 | 4.713 | 13.189 | 9.692 | 9.677 |
| 18027_curated\|7105 . . . 8628 | | | | | | | | |
| Cas14b.4\|cg1_0.2_scaffold_{—} | 10.127 | 8.288 | 8.562 | 9.369 | 5.077 | 9.672 | 10.569 | 10.537 |
| 785_c_curated\|32521 . . . 34155 | | | | | | | | |
| Cas14b.7\|3300013125.a\| | 8.913 | 9.964 | 9.864 | 9.414 | 4.889 | 10.536 | 9.827 | 9.811 |
| Ga0172369_10000737\| | | | | | | | | |
| 994 . . . 2652\|revcom | | | | | | | | |
| Cas14u.2\|3300002172.a\| | 14.356 | 13.115 | 8.048 | 12.319 | 3.279 | 16.708 | 14.286 | 13.874 |
| JGI24730J26740_1002785\| | | | | | | | | |
| 496 . . . 1605\|revcom | | | | | | | | |
| Cas14b.3\|rifcsphigho2_01_{—} | 12.044 | 11.636 | 9.898 | 9.222 | 6.024 | 9.926 | 11.858 | 11.799 |
| scaffold_36781_curated\| | | | | | | | | |
| 2592 . . . 4217 | | | | | | | | |
| Cas14b.2\|rifcsplowo2_01_{—} | 11.615 | 11.232 | 10.881 | 9.369 | 6.463 | 10.766 | 10.02 | 10 |
| scaffold_282_curated\| | | | | | | | | |
| 77370 . . . 78983 | | | | | | | | |
| Cas14b.1\|rifcsplowo2_01_{—} | 10.806 | 10.929 | 10.745 | 9.42 | 6.261 | 9.963 | 8.946 | 8.949 |
| scaffold_239_curated\| | | | | | | | | |
| 54653 . . . 56257 | | | | | | | | |
| Cas14b.8\|3300013125.a\| | 11.029 | 11.7 | 10.727 | 8.696 | 5.739 | 10.37 | 9.381 | 9.375 |
| Ga0172369_10010464\| | | | | | | | | |
| 885 . . . 2489\|revcom | | | | | | | | |
| Cas14b.5\|rifcsphigho2_02_{—} | 9.397 | 9.894 | 8.081 | 10.783 | 5.786 | 9.22 | 10.667 | 10.634 |
| scaffold_55589_curated\| | | | | | | | | |
| 1904 . . . 3598 | | | | | | | | |
| Cas14b.6\|CG03_land_8_20_{—} | 9.901 | 8.731 | 8.618 | 8.483 | 5.214 | 9.5 | 8.955 | 8.958 |
| 14_0.80_scaffold_2214_{—} | | | | | | | | |
| curated\|6634 . . . 8466\|revcom | | | | | | | | |
| Cas14b.9\|3300013127.a\| | 9.54 | 10.374 | 8.483 | 9.966 | 7.087 | 10 | 8.511 | 8.523 |
| Ga0172365_10004421\| | | | | | | | | |
| 633 . . . 2366\|revcom | | | | | | | | |
| 209658_13971_protein_{—} | 13.812 | 12.963 | 10.448 | 13.202 | 4.834 | 13.536 | 13.165 | 13.165 |
| locus_of_contig_Ga0190333_1001561 - | | | | | | | | |
| Query protein | | | | | | | | |
| (209658_13971) | | | | | | | | |
| (2) | | | | | | | | |
| 209657 57738_protein_{—} | 18.224 | 19.725 | 15.962 | 17.371 | 9.487 | 19.048 | 17.143 | 17.143 |
| locus_of_contig_Ga0190332_1015597 - | | | | | | | | |
| Query protein | | | | | | | | |
| (209657_57738) | | | | | | | | |
| (2) | | | | | | | | |
| 209660_51257_protein_{—} | 17.241 | 19.807 | 14.851 | 17.327 | 9.019 | 18.593 | 16.08 | 16.08 |
| locus_of_contig_Ga0190335_1015156 - | | | | | | | | |
| Query protein | | | | | | | | |
| (209660_51257) | | | | | | | | |
| (2) | | | | | | | | |
| Cas14b.14\|gwc1_scaffold_{—} | 8.682 | 8.786 | 6.38 | 7.455 | 7.18 | 9.179 | 10.14 | 9.949 |
| 8732_curated\|2705 . . . 4537 | | | | | | | | |
| Cas14b.15\|3300010293.a\| | 8.019 | 8.805 | 8.116 | 8.025 | 5.766 | 9.365 | 7.731 | 7.921 |
| Ga0116204_1008574\| | | | | | | | | |
| 2134 . . . 4032 | | | | | | | | |
| Cas14b.12\|CG22_combo_CG10- | 6.162 | 5.905 | 7.031 | 6.282 | 6.567 | 6.865 | 7.173 | 7.202 |
| 13_8_21_14_all_scaffold_{—} | | | | | | | | |
| 2003_curated\|553 . . . 2880\|revcom | | | | | | | | |
| Cas14b.13\|rifcsphigho2_01_{—} | 7.004 | 6.986 | 7.833 | 6.914 | 6.833 | 7.672 | 7.714 | 7.736 |
| scaffold_82367_curated\| | | | | | | | | |
| 1523 . . . 3856\|revcom | | | | | | | | |
| Cas14b.16\|3300005573.a\| | 8.64 | 8.28 | 8.1 | 7.547 | 5.056 | 8.9 | 9.424 | 9.589 |
| Ga0078972_1001015a\| | | | | | | | | |
| 33750 . . . 35627 | | | | | | | | |
| Cas14b.10\|CG08_land_8_20_{—} | 8.553 | 10.164 | 7.98 | 8.347 | 5.702 | 8.099 | 9.386 | 9.381 |
| 14_0.20_scaffold_1609_{—} | | | | | | | | |
| curated\|6134 . . . 7975 | | | | | | | | |
| Cas14b.11\|CG_4_10_14_0.8_{—} | 8.224 | 8.867 | 7.516 | 8.039 | 5.541 | 8.609 | 9.567 | 9.381 |
| um_filter_scaffold_20762_{—} | | | | | | | | |
| curated\|1372 . . . 3219 | | | | | | | | |
| Cas14u.1\|3300009029.a\| | 14.151 | 13.122 | 8.876 | 10.502 | 3.643 | 13.318 | 13.384 | 13.022 |
| Ga0066793_10010091\| | | | | | | | | |
| 37 . . . 1113\|revcom | | | | | | | | |
| Cas12c1 | 3.016 | 3.41 | 4.177 | 3.085 | 6.218 | 3.819 | 3.541 | 3.509 |
| Cas12c2 | 3.598 | 3.434 | 4.362 | 3.156 | 7.863 | 3.275 | 3.226 | 3.283 |
| Cas12a_UPI001113398F | 3.96 | 3.156 | 4.779 | 3.142 | 5.779 | 3.258 | 2.486 | 2.554 |
| Cas12b_UPI001113398F | 3.96 | 3.156 | 4.779 | 3.142 | 5.779 | 3.258 | 2.486 | 2.554 |
| Cas12b_tr\|A0A1I7F1U9\| | 3.957 | 3.055 | 4.867 | 3.139 | 5.807 | 3.348 | 2.481 | 2.55 |
| A0A1I7F1U9_{—} | | | | | | | | |
| 9BACL | | | | | | | | |
| Cas12a_UPI00083514A7 | 3.136 | 3.232 | 4.487 | 2.594 | 6.591 | 2.599 | 2.657 | 2.723 |
| Cas12b_UPI00083514A7 | 3.136 | 3.232 | 4.487 | 2.594 | 6.591 | 2.599 | 2.657 | 2.723 |
| Cas12a_UPI00097159F1 | 2.661 | 2.663 | 4.503 | 3.294 | 6.298 | 3.643 | 2.242 | 2.314 |
| Cas12b_UPI00097159F1 | 2.661 | 2.663 | 4.503 | 3.294 | 6.298 | 3.643 | 2.242 | 2.314 |
| Cas12b_sp\|T0D7A2\| | 2.661 | 2.663 | 4.503 | 3.294 | 6.298 | 3.578 | 2.242 | 2.314 |
| CS12B_ALIAG | | | | | | | | |
| Cas12a_UPI0009715A14 | 2.661 | 2.663 | 4.503 | 3.294 | 6.298 | 3.578 | 2.242 | 2.314 |
| Cas12b_UPI0009715A14 | 2.661 | 2.663 | 4.503 | 3.294 | 6.298 | 3.578 | 2.242 | 2.314 |
| Cas12a_UPI00097159CF | 2.661 | 2.663 | 4.503 | 3.294 | 6.298 | 3.578 | 2.242 | 2.314 |
| Cas12b_UPI00097159CF | 2.661 | 2.663 | 4.503 | 3.294 | 6.298 | 3.578 | 2.242 | 2.314 |
| Cas12a_UPI000832F6D2 | 2.75 | 2.849 | 4.592 | 3.294 | 6.523 | 3.483 | 2.045 | 2.119 |
| Cas12b_UPI000832F6D2 | 2.75 | 2.849 | 4.592 | 3.294 | 6.523 | 3.483 | 2.045 | 2.119 |
| Cas12b_tr\|A0A512CSX2\| | 2.841 | 2.755 | 4.592 | 3.294 | 6.37 | 3.391 | 2.142 | 2.216 |
| A0A512CSX2_9BACL | | | | | | | | |
| OspCas12c | 3.496 | 2.685 | 3.504 | 3.89 | 7.179 | 2.941 | 3.38 | 3.519 |
| Cas14u.5\|3300012532.a\| | 6.938 | 5.556 | 5.588 | 6.577 | 4.038 | 5.918 | 6.988 | 7.026 |
| Ga0137373_10000316\| | | | | | | | | |
| 3286 . . . 5286 | | | | | | | | |
| 63461_4106_protein_{—} | 7.084 | 5.307 | 6.907 | 6.743 | 3.362 | 6.988 | 5.302 | 5.197 |
| locus_of_contig_LSKL01000323 - | | | | | | | | |
| Query protein | | | | | | | | |
| (63461_4106) | | | | | | | | |
| translation (4) | | | | | | | | |
| 58610_1188_protein_{—} | 7.792 | 4.693 | 7.121 | 7.27 | 3.531 | 7.143 | 6.329 | 6.206 |
| locus_of_contig_LFOD01000003 - | | | | | | | | |
| Query protein | | | | | | | | |
| (58610_1188) | | | | | | | | |
| translation (5) | | | | | | | | |
| 21566_3969_protein_{—} | 6.988 | 5.473 | 5.643 | 7.82 | 2.431 | 6.425 | 5.935 | 5.82 |
| locus_of_contig_BAFB01000202 - | | | | | | | | |
| Query protein | | | | | | | | |
| (21566_3969) | | | | | | | | |
| translation (4) | | | | | | | | |

TABLE 7

| | Cas14u.6\| | 466065_250_protein_{—} | | | | | | |
|---|---|---|---|---|---|---|---|---|
| | 3300006028.a\| | locus_of_contig_{—} | Cas14a.5\|rifcsplowo2_{—} | | Cas14a.3\|gwa1_{—} | Cas14a.1\|rifcsphigho2_{—} | Cas14a.2\|gwa2_{—} | Cas14b.4\|cg1_0.2_{—} |
| | Ga0070717_{—} | SFKR01000004.1 - | 01_scaffold_34461_{—} | | scaffold_1795_{—} | 02_scaffold_2167_{—} | scaffold_18027_{—} | scaffold_785_c_{—} |
| | 10000077\|54519 . . . | Query protein | curated\|4968 . . . | | curated\|25635 . . . | curated\|30296 . . . | curated\|7105 . . . | curated\|32521 . . . |
| | 56201\|revcom | (466065_250) | 6521 | CasY2 | 27224\|revcom | 31798\|revcom | 8628 | 34155 |
| Cas14g.1\|RBG_13_{—} | 7.317 | 7.007 | 6.191 | 5.34 | 9.517 | 7.921 | 7.983 | 9.986 |
| scaffold_1401_{—} | | | | | | | | |
| curated\|15949 . . . | | | | | | | | |
| 18180 | | | | | | | | |
| Cas14g.2\|3300009652.a\| | 8.101 | 6.564 | 4.78 | 5.364 | 7.923 | 7.629 | 7.422 | 9.823 |
| Ga0123330_1010394\|2814 . . . | | | | | | | | |
| 5123 | | | | | | | | |
| Cas12i2 | 4.094 | 4.187 | 3.349 | 5.168 | 5.44 | 5.186 | 5.442 | 4.608 |
| Cas12i1 | 2.993 | 3.868 | 5.14 | 6.993 | 4.995 | 4.857 | 4.447 | 4.135 |
| Cas12g1 | 6.806 | 6.729 | 4.666 | 5.294 | 7.417 | 8.052 | 7.403 | 8.105 |
| Cas14d.3\|RIFCSPLOWO2_{—} | 6.484 | 5.271 | 7.069 | 5.448 | 7.339 | 7.891 | 6.98 | 8.739 |
| 01_FULL_OD1_45_34b_{—} | | | | | | | | |
| rifcsplowo2_01_{—} | | | | | | | | |
| scaffold_3495_{—} | | | | | | | | |
| curated\|25656 . . . | | | | | | | | |
| 27605\|revcom | | | | | | | | |
| Cas14d.1\|RIFCSPHIGHO2_{—} | 5.663 | 6.688 | 6.923 | 4.297 | 5.346 | 8.1 | 7.944 | 5.295 |
| 01_FULL_CPR 46_36_{—} | | | | | | | | |
| rifcsphigho2_01_{—} | | | | | | | | |
| scaffold_646_{—} | | | | | | | | |
| curated\|49808 . . . | | | | | | | | |
| 51616\|revcom | | | | | | | | |
| CasY5 | 3.206 | 3.439 | 3.578 | 5.865 | 3.767 | 3.733 | 3.534 | 3.826 |
| Cas14a.4\|CG10_{—} | 6.292 | 6.936 | 5.658 | 4.878 | 12.273 | 12.188 | 11.523 | 7.367 |
| big_fil_rev_8_21_{—} | | | | | | | | |
| 14_0.10_scaffold_{—} | | | | | | | | |
| 20906_curated\|649 . . . | | | | | | | | |
| 2829 | | | | | | | | |
| CasY6 | 3.917 | 2.76 | 2.441 | 6.471 | 4.194 | 5.436 | 5.485 | 3.512 |
| Cas14f.1\|rifcsp13_1_{—} | 9.655 | 9.272 | 5.27 | 4.818 | 7.65 | 6.827 | 6.426 | 6.711 |
| sub10_scaffold_3_{—} | | | | | | | | |
| curated\|38906 . . . 41041 | | | | | | | | |
| Cas14f.2\|3300009991.a\| | 10.224 | 9.324 | 4.647 | 2.85 | 7.267 | 7.401 | 7.049 | 7.764 |
| Ga0105042_100140\|1624 . . . | | | | | | | | |
| 3348 | | | | | | | | |
| Cas14a.6\|3300012359.a\| | 6.623 | 10.23 | 6.549 | 4.903 | 17.056 | 19.342 | 19.923 | 8.305 |
| Ga0137385_10000156\|41289 . . . | | | | | | | | |
| 42734 | | | | | | | | |
| Cas12a_UPI00094EEDB4 | 2.868 | 2.679 | 3.966 | 6.557 | 4.855 | 3.801 | 3.395 | 3.807 |
| Cas12a_UPI000B4235CE | 4.189 | 2.518 | 5.004 | 6.424 | 3.909 | 4.425 | 4.17 | 3.106 |
| Cas12a_UPI000818CC52 | 4.189 | 2.518 | 5.008 | 6.362 | 3.909 | 4.425 | 4.17 | 3.106 |
| Cas12a_UPI0007B78B7F | 4.189 | 2.518 | 5.004 | 6.424 | 3.909 | 4.425 | 4.17 | 3.106 |
| Cas12a_UPI000B4235F9 | 4.189 | 2.518 | 5.004 | 6.424 | 3.909 | 4.425 | 4.17 | 3.103 |
| Cas14e.2\|rifcsplowo2_{—} | 7.611 | 10.909 | 6.285 | 3.072 | 7.679 | 7.076 | 5.959 | 7.356 |
| 01_scaffold_81231_{—} | | | | | | | | |
| curated\|976 . . . 2217 | | | | | | | | |
| Cas14e.1\|rifcsphigho2_{—} | 5.146 | 10.633 | 6.667 | 2.728 | 7.527 | 9.441 | 8.285 | 7.638 |
| 01_scaffold_566_{—} | | | | | | | | |
| curated\|113069 . . . 114313 | | | | | | | | |
| Cas14e.3\|rifcsphigho2_{—} | 5.651 | 8.457 | 6.947 | 2.647 | 7.482 | 8.253 | 7.678 | 6.667 |
| 01_scaffold_4702_{—} | | | | | | | | |
| curated\|82881 . . . | | | | | | | | |
| 84230\|revcom | | | | | | | | |
| CasY4 | 4.23 | 3.972 | 3.333 | 8.408 | 5.06 | 3.98 | 3.62 | 4.488 |
| Cas14h.3\|3300009698.a\| | 11.058 | 12.527 | 5.308 | 3.686 | 8.6 | 8.734 | 8.099 | 8.829 |
| Ga0116216_10000905\|8005 . . . | | | | | | | | |
| 9504 | | | | | | | | |
| Cas14h.1\|3300005602.a\| | 12.342 | 10.584 | 4.944 | 3.431 | 8.531 | 7.667 | 7.258 | 7.571 |
| Ga0070762_10001740\|7377 . . . | | | | | | | | |
| 9071\|revcom | | | | | | | | |
| Cas14h.2\|3300005921.a\| | 11.774 | 12.222 | 5.016 | 3.529 | 8.065 | 7.155 | 7.401 | 8.833 |
| Ga0070766_10011912\|384 . . . | | | | | | | | |
| 2081 | | | | | | | | |
| Cas14c.1\|CG10_big_fil_rev_{—} | 7.573 | 15.464 | 5.873 | 2.977 | 9.431 | 8.919 | 8.136 | 8.108 |
| 8_21_14_0.10_scaffold_4477_{—} | | | | | | | | |
| curated\|19327 . . . | | | | | | | | |
| 20880\|revcom | | | | | | | | |
| Cas12h1 | 5.38 | 4.423 | 5.012 | 5.167 | 6.36 | 6.683 | 7.101 | 5.945 |
| CasX1 | 4.67 | 5.65 | 5.061 | 7.529 | 7.611 | 7.21 | 7.78 | 7.07 |
| CasX2 | 5.123 | 5.92 | 5.231 | 8.089 | 7.257 | 7.278 | 7.749 | 7.446 |
| CasY1 | 3.815 | 5.019 | 3.597 | 6.977 | 5.355 | 5.119 | 5.086 | 5.839 |
| Cas14u.3\|19ft_2_{—} | 6.436 | 9.776 | 7.584 | 4.255 | 9.206 | 8.379 | 8.561 | 9.508 |
| nophage_noknown_{—} | | | | | | | | |
| scaffold_0_curated\| | | | | | | | | |
| 508188 . . . 509648 | | | | | | | | |
| Cas14u.7\|3300001256.a\| | 10.071 | 29.563 | 7.635 | 3.442 | 9.108 | 10.6 | 11.637 | 9.141 |
| JGI12210J13797_10004690\| | | | | | | | | |
| 5792 . . . 7006 | | | | | | | | |
| Cas14u.8\|3300005660.a\| | 9.769 | 31.759 | 5.056 | 3.61 | 9.125 | 8.73 | 9.393 | 10.127 |
| Ga0073904_10021651\|765 . . . | | | | | | | | |
| 1943 | | | | | | | | |
| Cas14u.4\|rifcsp2_19_4_full_{—} | 7.193 | 13.022 | 5.311 | 4.373 | 8.939 | 9.703 | 10.352 | 8.288 |
| scaffold_168_curated\| | | | | | | | | |
| 84455 . . . 85657 | | | | | | | | |
| Cas14d.2\|rifcsphigho2_{—} | 7.143 | 9.562 | 7.04 | 4.195 | 8.711 | 9.444 | 9.444 | 8.562 |
| 01_scaffold_10981_curated\| | | | | | | | | |
| 5762 . . . 7246\|revcom | | | | | | | | |
| Cas14c.2\|3300001245.a\| | 8.448 | 19.851 | 5.263 | 3.833 | 11.481 | 11.637 | 12.84 | 9.369 |
| JGI12048J13642_10201286\| | | | | | | | | |
| 4257 . . . 5489 \|revcom | | | | | | | | |
| CasY3 | 4.663 | 4.474 | 2.703 | 8.24 | 4.613 | 4.483 | 4.713 | 5.077 |
| 633299_527_protein_locus_{—} | 8.772 | 37.047 | 6.642 | 3.987 | 12.008 | 12.176 | 13.189 | 9.672 |
| of_contig_Scfld15 - | | | | | | | | |
| Query protein | | | | | | | | |
| (633299_527) (4) | | | | | | | | |
| 8971_2857_protein_locus_{—} | 9.851 | 44.092 | 5.394 | 3.467 | 8.264 | 10.067 | 9.692 | 10.569 |
| of_contig_OEJQ01000083.1 - | | | | | | | | |
| Query protein | | | | | | | | |
| (8971_2857) | | | | | | | | |
| 9265_901_protein_locus_{—} | 9.836 | 44.134 | 5.882 | 3.433 | 8.283 | 10.044 | 9.677 | 10.537 |
| of_contig_OEFX01000005.1 - | | | | | | | | |
| Query protein | | | | | | | | |
| (9265_901) | | | | | | | | |
| Cas14u.6\|3300006028.a\| | | 10.929 | 3.662 | 4 | 8.609 | 8.013 | 6.777 | 7.448 |
| Ga0070717_10000077\|54519 . . . | | | | | | | | |
| 56201\|revcom | | | | | | | | |
| 466065_250_protein_locus_{—} | 10.929 | | 5.469 | 3.976 | 8.571 | 10.883 | 11.294 | 9.515 |
| of_contig_SFKR01000004.1 - | | | | | | | | |
| Query protein | | | | | | | | |
| (466065_250) | | | | | | | | |
| Cas14a.5\|rifcsplowo2_01_{—} | 3.662 | 5.469 | | 3.682 | 9.275 | 11.607 | 12.169 | 7.273 |
| scaffold_34461_curated\| | | | | | | | | |
| 4968 . . . 6521 | | | | | | | | |
| CasY2 | 4 | 3.976 | 3.682 | | 5.665 | 4.847 | 5.41 | 4.588 |
| Cas14a.3\|gwa1_scaffold_{—} | 8.609 | 8.571 | 9.275 | 5.665 | | 36.43 | 35.519 | 10.697 |
| 1795_curated\|25635 . . . | | | | | | | | |
| 27224\|revcom | | | | | | | | |
| Cas14a.1\|rifcsphigho2_02_{—} | 8.013 | 10.883 | 11.607 | 4.847 | 36.43 | | 81.6 | 10.788 |
| scaffold_2167_curated\| | | | | | | | | |
| 30296 . . . 31798\|revcom | | | | | | | | |
| Cas14a.2\|gwa2_scaffold_18027_{—} | 6.777 | 11.294 | 12.169 | 5.41 | 35.519 | 81.6 | | 10.103 |
| curated\|7105 . . . 8628 | | | | | | | | |
| Cas14b.4\|cg1_0.2_scaffold_785_{—} | 7.448 | 9.515 | 7.273 | 4.588 | 10.697 | 10.788 | 10.103 | |
| c_curated\|32521 . . . 34155 | | | | | | | | |
| Cas14b.7\|3300013125.a\| | 7.372 | 9.222 | 6.656 | 4.73 | 11.058 | 11.185 | 10.851 | 42.708 |
| Ga0172369_10000737\|994 | | | | | | | | |
| . . . 2652\|revcom | | | | | | | | |
| Cas14u.2\|3300002172.a\| | 7.881 | 15.99 | 6.818 | 4.34 | 11.364 | 10.664 | 10.913 | 10.681 |
| JGI24730J26740_1002785\| | | | | | | | | |
| 496 . . . 1605\|revcom | | | | | | | | |
| Cas14b.3\|rifcsphigho2_01_{—} | 6.602 | 10.478 | 7.967 | 5.187 | 11.519 | 11.356 | 12.034 | 16.723 |
| scaffold_36781_curated\| | | | | | | | | |
| 2592 . . . 4217 | | | | | | | | |
| Cas14b.2\|rifcsplowo2_01_{—} | 6.897 | 10.256 | 8.007 | 5.326 | 10.316 | 9.241 | 8.911 | 15.92 |
| scaffold_282_curated\| | | | | | | | | |
| 77370 . . . 78983 | | | | | | | | |
| Cas14b.1\|rifcsplowo2_01_{—} | 6.393 | 10.019 | 8.02 | 5.475 | 12.02 | 10.248 | 10.248 | 16.279 |
| scaffold_239_curated\| | | | | | | | | |
| 54653 . . . 56257 | | | | | | | | |
| Cas14b.8\|3300013125.a\| | 6.579 | 10.575 | 8.183 | 5.047 | 11.39 | 10.282 | 9.453 | 16.5 |
| Ga0172369_10010464\|885 . . . | | | | | | | | |
| 2489\|revcom | | | | | | | | |
| Cas14b.5\|rifcsphigho2_02_{—} | 8.401 | 10.48 | 8.293 | 5.963 | 12.841 | 11.675 | 11.675 | 19.224 |
| scaffold_55589_curated\| | | | | | | | | |
| 1904 . . . 3598 | | | | | | | | |
| Cas14b.6\|CG03_land 8_20_14_{—} | 7.176 | 8.968 | 9.37 | 5.56 | 11.42 | 11.22 | 11.87 | 19.677 |
| 0.80_scaffold_2214_curated\| | | | | | | | | |
| 6634 . . . 8466\|revcom | | | | | | | | |
| Cas14b.9\|3300013127.a\| | 8.58 | 9.343 | 6.75 | 5.812 | 12.324 | 10.561 | 10.891 | 19.569 |
| Ga0172365_10004421\|633 . . . | | | | | | | | |
| 2366\|revcom | | | | | | | | |
| 209658_13971_protein_locus_{—} | 9.015 | 12.707 | 8.294 | 5.468 | 13.024 | 13.3 | 13.547 | 19.861 |
| of_contig_Ga0190333_1001561 - | | | | | | | | |
| Query protein | | | | | | | | |
| (209658_13971) | | | | | | | | |
| (2) | | | | | | | | |
| 209657_57738_protein_locus_{—} | 15.164 | 17.788 | 11.814 | 8.836 | 22.326 | 20.183 | 19.725 | 30.374 |
| of_contig_Ga0190332_1015597 - | | | | | | | | |
| Query protein | | | | | | | | |
| (209657_57738) | | | | | | | | |
| (2) | | | | | | | | |
| 209660_51257_protein_locus_{—} | 14.592 | 17.259 | 11.062 | 8.168 | 22.549 | 20.29 | 19.807 | 29.557 |
| of_contig_Ga0190335_1015156 - | | | | | | | | |
| Query protein | | | | | | | | |
| (209660_51257) | | | | | | | | |
| (2) | | | | | | | | |
| Cas14b.14\|gwc1_scaffold_8732_{—} | 5.832 | 8.838 | 5.433 | 5.241 | 8.728 | 8.636 | 9.242 | 13.557 |
| curated\|2705 . . . 4537 | | | | | | | | |
| Cas14b.15\|3300010293.a\| | 7.447 | 10.841 | 6.871 | 5.626 | 9.954 | 11.145 | 10.502 | 11.458 |
| Ga0116204_1008574\|2134 . . . | | | | | | | | |
| 4032 | | | | | | | | |
| Cas14b.12\|CG22_combo_CG10- | 5.625 | 7.171 | 5.941 | 6.029 | 6.804 | 7.445 | 7.28 | 11.14 |
| 13_8_21_14_all_scaffold_2003_{—} | | | | | | | | |
| curated\|553 . . . 2880\| | | | | | | | | |
| revcom | | | | | | | | |
| Cas14b.13\|rifcsphigho2_01_{—} | 7.098 | 7.867 | 5.882 | 6.426 | 8.564 | 8.073 | 8.29 | 11.211 |
| scaffold_82367_curated\| | | | | | | | | |
| 1523 . . . 3856\|revcom | | | | | | | | |
| Cas14b.16\|3300005573.a\| | 7.264 | 9.493 | 8.722 | 5.719 | 11.502 | 9.969 | 9.502 | 13.509 |
| Ga0078972_1001015a\|33750 . . . | | | | | | | | |
| 35627 | | | | | | | | |
| Cas14b.10\|CG08_land_8_20_14_{—} | 10.502 | 10.94 | 6.891 | 5.491 | 10.129 | 8.654 | 9.206 | 13.744 |
| 0.20_scaffold_1609_curated\| | | | | | | | | |
| 6134 . . . 7975 | | | | | | | | |
| Cas14b.11\|CG_4_10_14_0.8_{—} | 8.976 | 10.427 | 7.573 | 5.008 | 10.129 | 9.807 | 8.931 | 11.765 |
| um_filter_scaffold_20762_{—} | | | | | | | | |
| curated\|1372 . . . 3219 | | | | | | | | |
| Cas14u.1\|3300009029.a\| | 7.584 | 13.318 | 7.707 | 3.336 | 9.982 | 13.069 | 12.871 | 8.834 |
| Ga0066793_10010091\|37 . . . | | | | | | | | |
| 1113\|revcom | | | | | | | | |
| Cas12c1 | 4.286 | 3.647 | 2.584 | 6.014 | 4.106 | 4.466 | 4.203 | 4.24 |
| Cas12c2 | 4.424 | 4.135 | 3.878 | 6.632 | 5.117 | 5.518 | 5.184 | 4.854 |
| Cas12a_UPI001113398F | 5.068 | 2.971 | 5.103 | 6.712 | 5.418 | 4.288 | 5.077 | 4.117 |
| Cas12b_UPI001113398F | 5.068 | 2.971 | 5.103 | 6.712 | 5.418 | 4.288 | 5.077 | 4.117 |
| Cas12b_tr\|A0A1I7F1U9\| | 5.158 | 3.058 | 5.169 | 6.642 | 5.142 | 4.189 | 4.977 | 4.026 |
| A0A1I7F1U9_9BACL | | | | | | | | |
| Cas12a_UPI00083514A7 | 4.599 | 2.308 | 4.728 | 5.927 | 4.487 | 4.455 | 4.517 | 4.45 |
| Cas12b_UPI00083514A7 | 4.599 | 2.308 | 4.728 | 5.927 | 4.487 | 4.455 | 4.517 | 4.45 |
| Cas12a_UPI00097159F1 | 4.428 | 2.844 | 5.302 | 6.616 | 4.69 | 4.944 | 5.097 | 3.911 |
| Cas12b_UPI00097159F1 | 4.428 | 2.844 | 5.302 | 6.616 | 4.69 | 4.944 | 5.097 | 3.911 |
| Cas12b_sp\|T0D7A2\|CS12B_{—} | 4.428 | 2.844 | 5.302 | 6.656 | 4.69 | 4.944 | 5.097 | 3.911 |
| ALIAG | | | | | | | | |
| Cas12a_UPI0009715A14 | 4.428 | 2.844 | 5.302 | 6.656 | 4.69 | 4.944 | 5.097 | 3.911 |
| Cas12b_UPI0009715A14 | 4.428 | 2.844 | 5.302 | 6.656 | 4.69 | 4.944 | 5.097 | 3.911 |
| Cas12a_UPI00097159CF | 4.428 | 2.844 | 5.302 | 6.656 | 4.69 | 4.944 | 5.097 | 3.911 |
| Cas12b_UPI00097159CF | 4.428 | 2.844 | 5.302 | 6.656 | 4.69 | 4.944 | 5.097 | 3.911 |
| Cas12a_UPI000832F6D2 | 4.7 | 2.746 | 5.297 | 6.886 | 4.592 | 4.846 | 4.907 | 3.814 |
| Cas12b_UPI000832F6D2 | 4.7 | 2.746 | 5.297 | 6.886 | 4.592 | 4.846 | 4.907 | 3.814 |
| Cas12b_tr\|A0A512CSX2\| | 4.885 | 2.841 | 5.205 | 6.58 | 4.686 | 4.939 | 5.093 | 3.907 |
| A0A512CSX2_9BACL | | | | | | | | |
| OspCas12c | 4.217 | 3.859 | 2.885 | 5.808 | 4.327 | 4.302 | 4.383 | 4.475 |
| Cas14u.5\|3300012532.a\| | 8.626 | 6.991 | 4.119 | 4.227 | 7.225 | 6.755 | 6.461 | 8.346 |
| Ga0137373_10000316\|3286 . . . | | | | | | | | |
| 5286 | | | | | | | | |
| 63461_4106_protein_locus_of_{—} | 8.15 | 5.351 | 5.14 | 4.503 | 9.451 | 6.656 | 6.815 | 7.309 |
| contig_LSKL01000323 - | | | | | | | | |
| Query protein | | | | | | | | |
| (63461_4106) | | | | | | | | |
| translation (4) | | | | | | | | |
| 58610_1188_protein_locus_of_{—} | 8.423 | 6.931 | 4.695 | 3.976 | 6.577 | 6.211 | 5.745 | 5.828 |
| contig_LFOD01000003 - | | | | | | | | |
| Query protein | | | | | | | | |
| (58610_1188) | | | | | | | | |
| translation (5) | | | | | | | | |
| 21566_3969_protein_locus_of_{—} | 7.402 | 6.187 | 4.409 | 4.174 | 7.553 | 6.667 | 7.302 | 6.202 |
| contig_BAFB01000202 - | | | | | | | | |
| Query protein | | | | | | | | |
| (21566_3969) | | | | | | | | |
| translation (4) | | | | | | | | |

TABLE 8

| | Cas14b.7\| | Cas14u.2\| | Cas14b.3\| | Cas14b.2 \| | Cas14b.1\| | Cas14b.8\| | | Cas14b.6\|CG03_{—} |
|---|---|---|---|---|---|---|---|---|
| | 3300013125.a\| | 3300002172.a\| | rifcsphigho2_01_{—} | rifcsplowo2_01_{—} | rifwo2csplo_01_{—} | 3300013125.a\| | Cas14b.5\| | land_8_20_{—} |
| | Ga0172369_{—} | JGI24730J26740_{—} | scaffold_36781_{—} | scaffold_282_{—} | scaffold_239_{—} | Ga0172369_{—} | rifcsphigho2_{—} | 14_0.80_scaffold_{—} |
| | 10000737\|994 . . . | 1002785\|496 . . . | curated\| | curated\| | curated\| | 10010464\|885 . . . | 02_scaffold_55589_{—} | 2214_curated\|6634 . . . |
| | 2652\|revcom | 1605\|revcom | 2592 . . . 4217 | 77370 . . . 78983 | 54653. . . 56257 | 2489\|revcom | curated\|1904 . . . 3598 | 8466\|revcom |
| Cas14g.1\|RBG_13_{—} | 9.655 | 6.828 | 9.904 | 9.218 | 9.986 | 10.125 | 10.028 | 10.633 |
| scaffold_1401_{—} | | | | | | | | |
| curated\|15949 . . . 18180 | | | | | | | | |
| Cas14g.2\|3300009652.a\| | 8.243 | 7.084 | 9.511 | 9.078 | 8.071 | 9.029 | 8.038 | 8.311 |
| Ga0123330_1010394\|2814 . . . | | | | | | | | |
| 5123 | | | | | | | | |
| Cas12i2 | 5.366 | 4.02 | 4.701 | 5.352 | 4.931 | 4.931 | 4.322 | 5.604 |
| Cas12i1 | 4.846 | 3.425 | 5.446 | 4.843 | 5.104 | 4.915 | 5.239 | 5.365 |
| Cas12g1 | 6.839 | 7.723 | 7.245 | 7.324 | 7.029 | 7.427 | 8.216 | 7.97 |
| Cas14d.3\|RIFCSPLOWO2_{—} | 8.204 | 5.91 | 6.619 | 7.122 | 7.069 | 7.806 | 7.932 | 7.402 |
| 01_FULL_OD1_45_{—} | | | | | | | | |
| 34b_rifcsplowo2_{—} | | | | | | | | |
| 01_scaffold_3495_{—} | | | | | | | | |
| curated\|25656 . . . | | | | | | | | |
| 27605\|revcom | | | | | | | | |
| Cas14d.1\|RIFCSPHIGHO2_{—} | 6.818 | 5.854 | 7.362 | 7.355 | 7.199 | 8.764 | 7.207 | 6.149 |
| 01_FULL_CPR_46_36_{—} | | | | | | | | |
| rifcsphigho2_01_{—} | | | | | | | | |
| scaffold_646_curated\| | | | | | | | | |
| 49808 . . . 51616\|revcom | | | | | | | | |
| CasY5 | 4.074 | 3.209 | 4.093 | 4.227 | 4.029 | 3.491 | 5.446 | 5.013 |
| Cas14a.4\|CG10_{—} | 8.713 | 7.022 | 8.647 | 10.57 | 10.497 | 10.083 | 8.482 | 9.707 |
| big_fil_rev_8_21_{—} | | | | | | | | |
| 14_0.10_scaffold_{—} | | | | | | | | |
| 20906_curated\|649 . . . | | | | | | | | |
| 2829 | | | | | | | | |
| CasY6 | 3.816 | 2.718 | 3.987 | 4.19 | 4.093 | 3.692 | 3.92 | 4.124 |
| Cas14f.1\|rifcsp13_{—} | 7.662 | 5.618 | 8.422 | 8.56 | 8.548 | 7.87 | 6.937 | 7.412 |
| 1_sub10_scaffold_{—} | | | | | | | | |
| 3_curated\|38906 . . . | | | | | | | | |
| 41041 | | | | | | | | |
| Cas14f.2\|3300009991.a\| | 8.75 | 5.965 | 7.75 | 6.615 | 7.373 | 7.988 | 6.202 | 6.724 |
| Ga0105042_100140\|1624 . . . | | | | | | | | |
| 3348 | | | | | | | | |
| Cas14a.6\|3300012359.a\| | 8.819 | 8 | 10.616 | 8.848 | 10.067 | 9.564 | 10.282 | 9.35 |
| Ga0137385_10000156\| | | | | | | | | |
| 41289 . . . 42734 | | | | | | | | |
| Cas12a_UPI00094EEDB4 | 4.338 | 2.644 | 4.439 | 4.471 | 4.766 | 4.375 | 3.724 | 4.08 |
| Cas12a_UPI000B4235CE | 3.464 | 2.638 | 4.5 | 4.15 | 4.29 | 4.29 | 4.267 | 3.92 |
| Cas12a_UPI000818CC52 | 3.464 | 2.638 | 4.507 | 4.15 | 4.29 | 4.29 | 4.267 | 3.926 |
| Cas12a_UPI0007B78B7F | 3.464 | 2.638 | 4.5 | 4.15 | 4.29 | 4.29 | 4.267 | 3.92 |
| Cas12a_UPI000B4235F9 | 3.462 | 2.638 | 4.5 | 4.15 | 4.29 | 4.29 | 4.267 | 3.92 |
| Cas14e.2\|rifcsplowo2_01_{—} | 6.713 | 8.844 | 7.5 | 8.185 | 7.871 | 7.168 | 6.914 | 7.12 |
| scaffold_81231_curated\| | | | | | | | | |
| 976 . . . 2217 | | | | | | | | |
| Cas14e.1\|rifcsphigho2_01_{—} | 6.768 | 8.924 | 8.007 | 7.143 | 8.174 | 7.292 | 7.155 | 6.421 |
| scaffold_566_curated\| | | | | | | | | |
| 113069 . . . 114313 | | | | | | | | |
| Cas14e.3\|rifcsphigho2_01_{—} | 6.04 | 9.013 | 6.885 | 7.317 | 7.813 | 6.424 | 6.096 | 5.696 |
| scaffold_4702_curated\| | | | | | | | | |
| 82881 . . . 84230\|revcom | | | | | | | | |
| CasY4 | 4.73 | 2.981 | 5.344 | 4.713 | 4.778 | 4.863 | 5.518 | 5.887 |
| Cas14h.3\|3300009698.a\| | 8.795 | 10.581 | 8.543 | 9.318 | 9.03 | 8.543 | 8.401 | 8.372 |
| Ga0116216_10000905\| | | | | | | | | |
| 8005 . . . 9504 | | | | | | | | |
| Cas14h.1\|3300005602.a\| | 7.166 | 8.289 | 8.458 | 8.143 | 8.224 | 8.581 | 7.827 | 8.359 |
| Ga0070762_10001740\| | | | | | | | | |
| 7377 . . . 9071\|revcom | | | | | | | | |
| Cas14h.2\|3300005921.a\| | 8.095 | 8.496 | 8.804 | 8.76 | 9.349 | 8.878 | 8.333 | 8.217 |
| Ga0070766_10011912\|384 . . . 2081 | | | | | | | | |
| Cas14c.1\|CG10_big_fil_rev_8_21_{—} | 8.217 | 10.291 | 8.373 | 7.813 | 7.559 | 6.951 | 7.562 | 7.852 |
| 14_0.10_scaffold_4477_{—} | | | | | | | | |
| curated\|19327 . . . 20880\| | | | | | | | | |
| revcom | | | | | | | | |
| Cas12h1 | 5.813 | 4.207 | 6.413 | 6.475 | 6.325 | 6.205 | 5.917 | 6.936 |
| CasX1 | 7.026 | 5.981 | 6.9 | 6.191 | 6.263 | 5.741 | 6.076 | 5.906 |
| CasX2 | 7.202 | 5.932 | 6.861 | 6.78 | 6.533 | 6.639 | 6.757 | 8.016 |
| CasY1 | 5.641 | 3.751 | 4.666 | 4.625 | 4.972 | 5.249 | 6.141 | 7.182 |
| Cas14u.3\|19ft_2_nophage_{—} | 9.903 | 9.919 | 9.402 | 10.517 | 9.879 | 11.092 | 10.611 | 9.365 |
| noknown_scaffold_0_{—} | | | | | | | | |
| curated\|508188 . . . 509648 | | | | | | | | |
| Cas14u.7\|3300001256.a\| | 9.091 | 13.35 | 10.929 | 10.83 | 10.969 | 10.275 | 10.247 | 10.351 |
| JGI12210J13797_10004690\| | | | | | | | | |
| 5792 . . . 7006 | | | | | | | | |
| Cas14u.8\|3300005660.a\| | 8.913 | 14.356 | 12.044 | 11.615 | 10.806 | 11.029 | 9.397 | 9.901 |
| Ga0073904_10021651\|765 . . . 1943 | | | | | | | | |
| Cas14u.4\|rifcsp2_19_4_full_{—} | 9.964 | 13.115 | 11.636 | 11.232 | 10.929 | 11.7 | 9.894 | 8.731 |
| scaffold_168_curated\| | | | | | | | | |
| 84455 . . . 85657 | | | | | | | | |
| Cas14d.2\|rifcsphigho2_01_{—} | 9.864 | 8.048 | 9.898 | 10.881 | 10.745 | 10.727 | 8.081 | 8.618 |
| scaffold_10981_curated\| | | | | | | | | |
| 5762 . . . 7246\|revcom | | | | | | | | |
| Cas14c.2\|3300001245.a\| | 9.414 | 12.319 | 9.222 | 9.369 | 9.42 | 8.696 | 10.783 | 8.483 |
| JGI12048J13642_10201286\| | | | | | | | | |
| 4257 . . . 5489\|revcom | | | | | | | | |
| CasY3 | 4.889 | 3.279 | 6.024 | 6.463 | 6.261 | 5.739 | 5.786 | 5.214 |
| 633299_527_protein_locus_{—} | 10.536 | 16.708 | 9.926 | 10.766 | 9.963 | 10.37 | 9.22 | 9.5 |
| of_contig_Scfld15 - | | | | | | | | |
| Query protein | | | | | | | | |
| (633299_527) (4) | | | | | | | | |
| 8971_2857_protein_locus_{—} | 9.827 | 14.286 | 11.858 | 10.02 | 8.946 | 9.381 | 10.667 | 8.955 |
| of_contig_OEJQ01000083.1 - | | | | | | | | |
| Query protein | | | | | | | | |
| (8971_2857) | | | | | | | | |
| 9265_901_protein_locus_{—} | 9.811 | 13.874 | 11.799 | 10 | 8.949 | 9.375 | 10.634 | 8.958 |
| of_contig_OEFX01000005.1 - | | | | | | | | |
| Query protein | | | | | | | | |
| (9265_901) | | | | | | | | |
| Cas14u.6\|3300006028.a\| | 7.372 | 7.881 | 6.602 | 6.897 | 6.393 | 6.579 | 8.401 | 7.176 |
| Ga0070717_10000077\| | | | | | | | | |
| 54519 . . . 56201\|revcom | | | | | | | | |
| 466065_250_protein_locus_{—} | 9.222 | 15.99 | 10.478 | 10.256 | 10.019 | 10.575 | 10.48 | 8.968 |
| of_contig_SFKR01000004.1 - | | | | | | | | |
| Query protein | | | | | | | | |
| (466065_250) | | | | | | | | |
| Cas14a.5\|rifcsplowo2_01_{—} | 6.656 | 6.818 | 7.967 | 8.007 | 8.02 | 8.183 | 8.293 | 9.37 |
| scaffold_34461_curated\| | | | | | | | | |
| 4968 . . . 6521 | | | | | | | | |
| CasY2 | 4.73 | 4.34 | 5.187 | 5.326 | 5.475 | 5.047 | 5.963 | 5.56 |
| Cas14a.3\|gwa1_scaffold_1795_{—} | 11.058 | 11.364 | 11.519 | 10.316 | 12.02 | 11.39 | 12.841 | 11.42 |
| curated\|25635 . . . 27224\|revcom | | | | | | | | |
| Cas14a.1\|rifcsphigho2_02_{—} | 11.185 | 10.664 | 11.356 | 9.241 | 10.248 | 10.282 | 11.675 | 11.22 |
| scaffold_2167_curated\| | | | | | | | | |
| 30296 . . . 31798\|revcom | | | | | | | | |
| Cas14a.2\|gwa2_scaffold_18027_{—} | 10.851 | 10.913 | 12.034 | 8.911 | 10.248 | 9.453 | 11.675 | 11.87 |
| curated\|7105 . . . 8628 | | | | | | | | |
| Cas14b.4\|cg1_0.2_scaffold_{—} | 42.708 | 10.681 | 16.723 | 15.92 | 16.279 | 16.5 | 19.224 | 19.677 |
| 785_ccurated\|32521 . . . 34155 | | | | | | | | |
| Cas14b.7\|3300013125.a\|Ga0172369_{—} | | 10.669 | 20.27 | 19.595 | 21.922 | 20.405 | 21.124 | 20.537 |
| 10000737\|994 . . . 2652\|revcom | | | | | | | | |
| Cas14u.2\|3300002172.a\| | 10.669 | | 12.897 | 13.704 | 13.133 | 12.994 | 12.029 | 11.933 |
| JGI24730J26740_1002785\| | | | | | | | | |
| 496 . . . 1605\|revcom | | | | | | | | |
| Cas14b.3\|rifcsphigho2_01_{—} | 20.27 | 12.897 | | 54.336 | 56.15 | 55.95 | 23.913 | 26.108 |
| scaffold_36781_curated\| | | | | | | | | |
| 2592 . . . 4217 | | | | | | | | |
| Cas14b.2\|rifcsplowo2_01_{—} | 19.595 | 13.704 | 54.336 | | 73.743 | 70.896 | 23.777 | 24.165 |
| scaffold_282_curated\| | | | | | | | | |
| 77370 . . . 78983 | | | | | | | | |
| Cas14b.1\|rifcsplowo2_01_{—} | 21.922 | 13.133 | 56.15 | 73.743 | | 77.632 | 24.456 | 24.921 |
| scaffold_239_curated\| | | | | | | | | |
| 54653 . . . 56257 | | | | | | | | |
| Cas14b.8\|3300013125.a\| | 20.405 | 12.994 | 55.95 | 70.896 | 77.632 | | 23.873 | 24.132 |
| Ga0172369_10010464\| | | | | | | | | |
| 885 . . . 2489\|revcom | | | | | | | | |
| Cas14b.5\|rifcsphigho2_02_{—} | 21.124 | 12.029 | 23.913 | 23.777 | 24.456 | 23.873 | | 31.111 |
| scaffold_55589_curated\| | | | | | | | | |
| 1904 . . . 3598 | | | | | | | | |
| Cas14b.6\|CG03_land_8_20_14_{—} | 20.537 | 11.933 | 26.108 | 24.165 | 24.921 | 24.132 | 31.111 | |
| 0.80_scaffold_2214_curated\| | | | | | | | | |
| 6634 . . .8466\|revcom | | | | | | | | |
| Cas14b.9\|3300013127.a\| | 21.626 | 10.764 | 24.463 | 23.453 | 25.081 | 24.032 | 31.759 | 42.479 |
| Ga0172365_10004421\| | | | | | | | | |
| 633 . . . 2366\|revcom | | | | | | | | |
| 209658_13971_protein_locus_{—} | 19.495 | 16.427 | 27.602 | 26.637 | 27.765 | 26.411 | 32.118 | 38.636 |
| of_contig_Ga0190333_1001561 - | | | | | | | | |
| Query protein | | | | | | | | |
| (209658_13971) | | | | | | | | |
| (2) | | | | | | | | |
| 209657_57738_protein_locus_{—} | 30.841 | 22.488 | 45.146 | 41.063 | 44.444 | 42.995 | 53.241 | 70.588 |
| of_contig_Ga0190332_1015597 - | | | | | | | | |
| Query protein | | | | | | | | |
| (209657_57738) | | | | | | | | |
| (2) | | | | | | | | |
| 209660_51257_protein_locus_{—} | 30.049 | 22.222 | 45.128 | 40.306 | 44.898 | 43.367 | 52.683 | 69.43 |
| of_contig_Ga0190335_1015156 - | | | | | | | | |
| Query protein | | | | | | | | |
| (209660_51257) | | | | | | | | |
| (2) | | | | | | | | |
| Cas14b.14\|gwc1_scaffold_8732_{—} | 13.324 | 7.792 | 13.108 | 13.15 | 14.574 | 12.735 | 11.864 | 12.624 |
| curated\|2705 . . . 4537 | | | | | | | | |
| Cas14b.15\|3300010293.a\| | 11.51 | 10.4 | 13.546 | 14.353 | 13.777 | 13.622 | 15.152 | 13.025 |
| Ga0116204_1008574\|2134 . . . 4032 | | | | | | | | |
| Cas14b.12\|CG22_combo_CG10- | 12.891 | 6.649 | 12.125 | 13.816 | 13.203 | 12.941 | 12.211 | 10.553 |
| 13_8_21_14_all_scaffold_2003_{—} | | | | | | | | |
| curated\|553 . . . 2880\|revcom | | | | | | | | |
| Cas14b.13\|rifcsphigho2_01_{—} | 11.494 | 7.208 | 11.765 | 12.844 | 12.37 | 11.979 | 11.795 | 11.139 |
| scaffold_82367_curated\|1523 . . . | | | | | | | | |
| 3856\|revcom | | | | | | | | |
| Cas14b.16\|3300005573.a\| | 13.077 | 9.431 | 15.147 | 15.335 | 15.848 | 14.263 | 15.822 | 14.074 |
| Ga0078972_1001015a\| | | | | | | | | |
| 33750 . . . 35627 | | | | | | | | |
| Cas14b.10\|CG08_land_8_20_14_{—} | 14.6 | 10.483 | 15.397 | 16.066 | 15.285 | 15.122 | 14.33 | 12.254 |
| 0.20_scaffold_1609_curated\| | | | | | | | | |
| 6134 . . . 7975 | | | | | | | | |
| Cas14b.11\|CG_4_10_14_0.8_um_{—} | 14.396 | 10.333 | 12.711 | 15.798 | 15.994 | 15.024 | 15.373 | 12.236 |
| filter_scaffold_20762_curated\| | | | | | | | | |
| 1372 . . . 3219 | | | | | | | | |
| Cas14u.1\|3300009029.a\| | 9.414 | 17.115 | 11.314 | 11.151 | 11.84 | 11.883 | 10.106 | 10.114 |
| Ga0066793_10010091\| | | | | | | | | |
| 37 . . . 1113\|revcom | | | | | | | | |
| Cas12c1 | 4.629 | 4.157 | 5.671 | 4.919 | 5.221 | 5.783 | 4.48 | 5.242 |
| Cas12c2 | 4 | 3.12 | 3.827 | 3.782 | 4.603 | 4.603 | 4.841 | 5.12 |
| Cas12a_UPI001113398F | 4.662 | 2.74 | 3.653 | 3.509 | 4.136 | 3.86 | 4.209 | 4.039 |
| Cas12b_UPI001113398F | 4.662 | 2.74 | 3.653 | 3.509 | 4.136 | 3.86 | 4.209 | 4.039 |
| Cas12b_tr\|A0A1I7F1U9\| | 4.662 | 2.742 | 3.653 | 3.506 | 4.132 | 3.857 | 4.209 | 4.032 |
| A0A1I7F1U9_9BACL | | | | | | | | |
| Cas12a_UPI00083514A7 | 3.993 | 3.279 | 3.822 | 3.036 | 3.388 | 3.663 | 4.011 | 4.383 |
| Cas12b_UPI00083514A7 | 3.993 | 3.279 | 3.822 | 3.036 | 3.388 | 3.663 | 4.011 | 4.383 |
| Cas12a_UPI00097159F1 | 4.735 | 2.796 | 4.186 | 3.857 | 4.026 | 4.588 | 4.007 | 3.85 |
| Cas12b_UPI00097159F1 | 4.735 | 2.796 | 4.186 | 3.857 | 4.026 | 4.588 | 4.007 | 3.85 |
| Cas12b_sp\|T0D7A2\|CS12B_{—} | 4.735 | 2.796 | 4.186 | 3.857 | 4.026 | 4.588 | 4.007 | 3.85 |
| ALIAG | | | | | | | | |
| Cas12a_UPI0009715A14 | 4.735 | 2.796 | 4.186 | 3.857 | 4.026 | 4.588 | 4.007 | 3.85 |
| Cas12b_UPI0009715A14 | 4.735 | 2.796 | 4.186 | 3.857 | 4.026 | 4.588 | 4.007 | 3.85 |
| Cas12a_UPI00097159CF | 4.735 | 2.796 | 4.186 | 3.857 | 4.026 | 4.588 | 4.007 | 3.85 |
| Cas12b_UPI00097159CF | 4.735 | 2.796 | 4.186 | 3.857 | 4.026 | 4.588 | 4.007 | 3.85 |
| Cas12a_UPI000832F6D2 | 4.36 | 2.889 | 4.089 | 3.665 | 3.835 | 4.303 | 4.19 | 4.029 |
| Cas12b_UPI000832F6D2 | 4.36 | 2.889 | 4.089 | 3.665 | 3.835 | 4.303 | 4.19 | 4.029 |
| Cas12b_tr\|A0A512CSX2\| | 4.267 | 2.889 | 4.182 | 3.665 | 3.742 | 4.21 | 4.19 | 4.304 |
| A0A512CSX2_9BACL | | | | | | | | |
| OspCas12c | 4.302 | 3.358 | 5.348 | 4.583 | 5.134 | 4.971 | 5.195 | 6.667 |
| Cas14u.5\|3300012532.a\| | 8.453 | 6.697 | 6.314 | 7.544 | 6.618 | 7.038 | 6.877 | 5.698 |
| Ga0137373_10000316\| | | | | | | | | |
| 3286 . . . 5286 | | | | | | | | |
| 63461_4106_protein_locus_{—} | 7.883 | 7.5 | 7.74 | 7.834 | 7.963 | 8.129 | 7.198 | 7.38 |
| of_contig_LSKL01000323 - | | | | | | | | |
| Query protein | | | | | | | | |
| (63461_4106) | | | | | | | | |
| translation (4) | | | | | | | | |
| 58610_1188_protein_locus_{—} | 7.023 | 8.007 | 7.317 | 6.787 | 7.681 | 6.949 | 6.949 | 7.887 |
| of_contig_LFOD01000003 - | | | | | | | | |
| Query protein | | | | | | | | |
| (58610_1188) | | | | | | | | |
| translation (5) | | | | | | | | |
| 21566_3969_protein_locus_{—} | 6.583 | 8.789 | 5.376 | 7.492 | 7.187 | 6.585 | 7.309 | 6.994 |
| of_contig_BAFB01000202 - | | | | | | | | |
| Query protein | | | | | | | | |
| (21566_3969) | | | | | | | | |
| translation (4) | | | | | | | | |

TABLE 9

| | | 209658_13971_{—} | 209657_57738_{—} | 209660_51257_{—} | | | | |
|---|---|---|---|---|---|---|---|---|
| | | protein_locus_{—} | protein_locus_{—} | protein_locus_{—} | | | | |
| | | of_contig_{—} | of_contig_{—} | of_contig_{—} | | | Cas14b.12\|CG22_{—} | Cas14b.13\| |
| | Cas14b.9\| | Ga0190333_{—} | Ga0190332_{—} | Ga0190335_{—} | | Cas14b.15\| | combo_CG10- | rifcsphigho2_{—} |
| | 3300013127.a\| | 1001561 - | 1015597 - | 1015156 - | Cas14b.14\| | 3300010293.a\| | 13_8_21_14_{—} | 01_scaffold_823 |
| | Ga0172365_{—} | Query protein | Query protein | Query protein | gwc1_scaffold_8732_{—} | Ga0116204_{—} | all_scaffold_2003_{—} | 67_{—} |
| | 10004421\|633 . . . | (209658_13971) | (209657_57738) | (209660_51257) | curated\|2705 . . . | 1008574\|2134 . . . | curated\|553 . . . | curated\|1523 . . . |
| | 2366\|revcom | (2) | (2) | (2) | 4537 | 4032 | 2880\|revcom | 3856\|revcom |
| Cas14g.1\|RBG_13_{—} | 10.852 | 11.434 | 21.344 | 20.661 | 8.04 | 8.09 | 8.391 | 8.545 |
| scaffold_1401_{—} | | | | | | | | |
| curated\|15949 . . . 18180 | | | | | | | | |
| Cas14g.2\|3300009652.a\| | 9.041 | 8.289 | 13.074 | 13.91 | 7.412 | 8.85 | 7.859 | 9.06 |
| Ga0123330_1010394\| | | | | | | | | |
| 2814 . . . 5123 | | | | | | | | |
| Cas12i2 | 5.408 | 5.032 | 9.571 | 9.31 | 4.074 | 4.356 | 4.906 | 4.72 |
| Cas12i1 | 5.07 | 4.11 | 5.621 | 5.288 | 4.384 | 4.093 | 6.029 | 5.326 |
| Cas12g1 | 8.503 | 5.732 | 12.261 | 12.295 | 7.067 | 8.864 | 7.915 | 7.65 |
| Cas14d.3\|RIFCSPLOWO2_{—} | 8.146 | 8.818 | 16.216 | 16.588 | 6.771 | 7.084 | 6.409 | 7.711 |
| 01_FULL_OD1_45_34b_{—} | | | | | | | | |
| rifcsplowo2_{—} | | | | | | | | |
| 01_scaffold_3495_{—} | | | | | | | | |
| curated\|25656 . . . | | | | | | | | |
| 27605\|revcom | | | | | | | | |
| Cas14d.1\|RIFCSPHIGHO2_{—} | 6.147 | 6.2 | 10.046 | 10.096 | 5.842 | 6.723 | 6.349 | 6.46 |
| 01_FULL_CPR_46_36_{—} | | | | | | | | |
| rifcsphigho2_{—} | | | | | | | | |
| 01_scaffold_646_{—} | | | | | | | | |
| curated\|49808 . . . | | | | | | | | |
| 51616\|revcom | | | | | | | | |
| CasY5 | 4.732 | 3.591 | 6.757 | 6.516 | 3.704 | 3.951 | 4.228 | 3.887 |
| Cas14a.4\|CG10_{—} | 10.174 | 8.733 | 13.531 | 12.329 | 7.393 | 7.345 | 7.078 | 7.441 |
| big_fil_rev_8_21_{—} | | | | | | | | |
| 14_0.10_scaffold_{—} | | | | | | | | |
| 20906_curated\| | | | | | | | | |
| 649 . . . 2829 | | | | | | | | |
| CasY6 | 5.044 | 3.531 | 5.979 | 5.696 | 3.543 | 4.282 | 3.909 | 3.876 |
| Cas14f.1\|rifcsp13_{—} | 8.524 | 6.709 | 12.057 | 12.546 | 5.728 | 6.633 | 5.122 | 6.034 |
| 1_sub10_scaffold_{—} | | | | | | | | |
| 3_curated\| | | | | | | | | |
| 38906 . . . 41041 | | | | | | | | |
| Cas14f.2\|3300009991.a\| | 7.364 | 7.4 | 10.37 | 10.811 | 5.503 | 4.809 | 4.492 | 5.232 |
| Ga0105042_100140\| | | | | | | | | |
| 1624 . . . 3348 | | | | | | | | |
| Cas14a.6\|3300012359.a\| | 9.076 | 11.616 | 16.667 | 16.129 | 8.423 | 9.56 | 6.076 | 6.378 |
| Ga0137385_10000156\| | | | | | | | | |
| 41289 . . . 42734 | | | | | | | | |
| Cas12a_UPI00094EEDB4 | 4.405 | 2.914 | 5.092 | 4.792 | 3.917 | 4.012 | 3.474 | 3.479 |
| Cas12a_UPI000B4235CE | 4.099 | 3.265 | 6.061 | 5.992 | 3.514 | 5.174 | 4.502 | 5.469 |
| Cas12a_UPI000818CC52 | 4.099 | 3.265 | 6.061 | 5.992 | 3.514 | 5.174 | 4.508 | 5.477 |
| Cas12a_UPI0007B78B7F | 4.099 | 3.265 | 6.061 | 5.992 | 3.514 | 5.174 | 4.502 | 5.469 |
| Cas12a_UPI000B4235F9 | 4.099 | 3.265 | 6.061 | 5.992 | 3.511 | 5.174 | 4.502 | 5.469 |
| Cas14e.2\|rifcsplowo2_01_{—} | 8.483 | 7.305 | 9.417 | 9.434 | 6.636 | 6.467 | 6.049 | 6.12 |
| scaffold_81231_curated\| | | | | | | | | |
| 976 . . . 2217 | | | | | | | | |
| Cas14e.1\|rifcsphigho2_01_{—} | 6.874 | 7.532 | 10.909 | 11.005 | 7.302 | 7.165 | 5.122 | 5.837 |
| scaffold_566_curated\| | | | | | | | | |
| 113069 . . . 114313 | | | | | | | | |
| Cas14e.3\|rifcsphigho2_01_{—} | 5.769 | 7.071 | 10.502 | 10.096 | 5.521 | 7.87 | 5.398 | 4.967 |
| scaffold_4702_curated\| | | | | | | | | |
| 82881 . . . 84230\|revcom | | | | | | | | |
| CasY4 | 5.442 | 4.388 | 8.592 | 8.416 | 4.519 | 5.303 | 5.229 | 5.048 |
| Cas14h.3\|3300009698.a\| | 8.703 | 9.176 | 14 | 13.808 | 7.209 | 6.957 | 5.289 | 6.304 |
| Ga0116216_10000905\| | | | | | | | | |
| 8005 . . . 9504 | | | | | | | | |
| Cas14h.1\|3300005602.a\| | 8.399 | 8.515 | 13.061 | 12.719 | 5.968 | 8.859 | 5.577 | 6.361 |
| Ga0070762_10001740\| | | | | | | | | |
| 7377 . . . 9071\|revcom | | | | | | | | |
| Cas14h.2\|3300005921.a\| | 8.517 | 8.37 | 12.863 | 13.043 | 6.696 | 9.531 | 6.21 | 6.555 |
| Ga0070766_10011912\| | | | | | | | | |
| 384 . . . 2081 | | | | | | | | |
| Cas14c.1\|CG10_big_fil_rev_{—} | 7.519 | 9.534 | 11.189 | 10.545 | 10.836 | 7.349 | 6.835 | 8.087 |
| 8_21_14_0.10_scaffold_4477_{—} | | | | | | | | |
| curated\| | | | | | | | | |
| 19327 . . . 20880\|revcom | | | | | | | | |
| Cas12h1 | 6.746 | 5.522 | 8.434 | 8.202 | 6.466 | 3.913 | 5.509 | 5.943 |
| CasX1 | 6.475 | 5.695 | 11.905 | 10.601 | 6.97 | 7.419 | 7.486 | 6.167 |
| CasX2 | 8.091 | 6.032 | 12.04 | 10.764 | 7.446 | 7.369 | 6.907 | 6.997 |
| CasY1 | 6.9 | 5.614 | 9.346 | 8.633 | 5.626 | 6.806 | 6.643 | 5.948 |
| Cas14u.3\|19ft_2_{—} | 9.532 | 11.058 | 17.593 | 17.073 | 7.903 | 8.788 | 7.226 | 8.042 |
| nophage_noknown_scaffold_0_{—} | | | | | | | | |
| curated\|508188 . . . 509648 | | | | | | | | |
| Cas14u.7\|3300001256.a\| | 11.379 | 14.481 | 20.657 | 20.297 | 9.6 | 7.741 | 7.642 | 6.762 |
| JGI12210J13797_10004690\| | | | | | | | | |
| 5792 . . . 7006 | | | | | | | | |
| Cas14u.8\|3300005660.a\| | 9.54 | 13.812 | 18.224 | 17.241 | 8.682 | 8.019 | 6.162 | 7.004 |
| Ga0073904_10021651\| | | | | | | | | |
| 765 . . . 1943 | | | | | | | | |
| Cas14u.4\|rifcsp2_19_4_full_{—} | 10.374 | 12.963 | 19.725 | 19.807 | 8.786 | 8.805 | 5.905 | 6.986 |
| scaffold_168_curated\| | | | | | | | | |
| 84455 . . . 85657 | | | | | | | | |
| Cas14d.2\|rifcsphigho2_01_{—} | 8.483 | 10.448 | 15.962 | 14.851 | 6.38 | 8.116 | 7.031 | 7.833 |
| scaffold_10981_curated\| | | | | | | | | |
| 5762 . . . 7246\|revcom | | | | | | | | |
| Cas14c.2\|3300001245.a\| | 9.966 | 13.202 | 17.371 | 17.327 | 7.455 | 8.025 | 6.282 | 6.914 |
| JGI12048J13642_10201286\| | | | | | | | | |
| 4257 . . . 5489\|revcom | | | | | | | | |
| CasY3 | 7.087 | 4.834 | 9.487 | 9.019 | 7.18 | 5.766 | 6.567 | 6.833 |
| 633299_527_protein_locus_{—} | 10 | 13.536 | 19.048 | 18.593 | 9.179 | 9.365 | 6.865 | 7.672 |
| of_contig_Scfld15 - | | | | | | | | |
| Query protein | | | | | | | | |
| (633299_527) (4) | | | | | | | | |
| 8971_2857_protein_locus_{—} | 8.511 | 13.165 | 17.143 | 16.08 | 10.14 | 7.731 | 7.173 | 7.714 |
| of_contig_OEJQ01000083.1 - | | | | | | | | |
| Query protein | | | | | | | | |
| (8971_2857) | | | | | | | | |
| 9265_901_protein_locus_{—} | 8.523 | 13.165 | 17.143 | 16.08 | 9.949 | 7.921 | 7.202 | 7.736 |
| of_contig_OEFX01000005.1 - | | | | | | | | |
| Query protein | | | | | | | | |
| (9265_901) | | | | | | | | |
| Cas14u.6\|3300006028.a\| | 8.58 | 9.015 | 15.164 | 14.592 | 5.832 | 7.447 | 5.625 | 7.098 |
| Ga0070717_10000077\| | | | | | | | | |
| 54519 . . . 56201\|revcom | | | | | | | | |
| 466065_250_protein_locus_{—} | 9.343 | 12.707 | 17.788 | 17.259 | 8.838 | 10.841 | 7.171 | 7.867 |
| of_contig_SFKR01000004.1 - | | | | | | | | |
| Query protein | | | | | | | | |
| (466065_250) | | | | | | | | |
| Cas14a.5\|rifcsplowo2_01_{—} | 6.75 | 8.294 | 11.814 | 11.062 | 5.433 | 6.871 | 5.941 | 5.882 |
| scaffold_34461_curated\| | | | | | | | | |
| 4968 . . . 6521 | | | | | | | | |
| CasY2 | 5.812 | 5.468 | 8.836 | 8.168 | 5.241 | 5.626 | 6.029 | 6.426 |
| Cas14a.3\|gwal_scaffold_1795_{—} | 12.324 | 13.024 | 22.326 | 22.549 | 8.728 | 9.954 | 6.804 | 8.564 |
| curated\|25635 . . . | | | | | | | | |
| 27224\|revcom | | | | | | | | |
| Cas14a.1\|rifcsphigho2_02_{—} | 10.561 | 13.3 | 20.183 | 20.29 | 8.636 | 11.145 | 7.445 | 8.073 |
| scaffold_2167_curated\| | | | | | | | | |
| 30296 . . . 31798\|revcom | | | | | | | | |
| Cas14a.2\|gwa2_scaffold_18027_{—} | 10.891 | 13.547 | 19.725 | 19.807 | 9.242 | 10.502 | 7.28 | 8.29 |
| curated\|7105 . . . 8628 | | | | | | | | |
| Cas14b.4\|cg1_0.2_scaffold_785_{—} | 19.569 | 19.861 | 30.374 | 29.557 | 13.557 | 11.458 | 11.14 | 11.211 |
| c_curated\|32521 . . . 34155 | | | | | | | | |
| Cas14b.7\|3300013125.a\| | 21.626 | 19.495 | 30.841 | 30.049 | 13.324 | 11.51 | 12.891 | 11.494 |
| Ga0172369_10000737\| | | | | | | | | |
| 994 . . . 2652\|revcom | | | | | | | | |
| Cas14u.2\|3300002172.a\| | 10.764 | 16.427 | 22.488 | 22.222 | 7.792 | 10.4 | 6.649 | 7.208 |
| JGI24730J26740_1002785\| | | | | | | | | |
| 496 . . . 1605\|revcom | | | | | | | | |
| Cas14b.3\|rifcsphigho2_01_{—} | 24.463 | 27.602 | 45.146 | 45.128 | 13.108 | 13.546 | 12.125 | 11.765 |
| scaffold_36781_curated\| | | | | | | | | |
| 2592 . . . 4217 | | | | | | | | |
| Cas14b.2\|rifcsplowo2_01_{—} | 23.453 | 26.637 | 41.063 | 40.306 | 13.15 | 14.353 | 13.816 | 12.844 |
| scaffold_282_curated\| | | | | | | | | |
| 77370 . . . 78983 | | | | | | | | |
| Cas14b.1\|rifcsplowo2_01_{—} | 25.081 | 27.765 | 44.444 | 44.898 | 14.574 | 13.777 | 13.203 | 12.37 |
| scaffold_239_curated\| | | | | | | | | |
| 54653 . . . 56257 | | | | | | | | |
| Cas14b.8\|3300013125.a\| | 24.032 | 26.411 | 42.995 | 43.367 | 12.735 | 13.622 | 12.941 | 11.979 |
| Ga0172369_10010464\| | | | | | | | | |
| 885 . . . 2489 \|revcom | | | | | | | | |
| Cas14b.5\|rifcsphigho2_02_{—} | 31.759 | 32.118 | 53.241 | 52.683 | 11.864 | 15.152 | 12.211 | 11.795 |
| scaffold_55589_curated\| | | | | | | | | |
| 1904 . . . 3598 | | | | | | | | |
| Cas14b.6\|CG03_land_8_20_14_{—} | 42.479 | 38.636 | 70.588 | 69.43 | 12.624 | 13.025 | 10.553 | 11.139 |
| 0.80_scaffold_2214_curated\| | | | | | | | | |
| 6634 . . . 8466\|revcom | | | | | | | | |
| Cas14b.9\|3300013127.a\| | | 40.941 | 67.317 | 66.495 | 13 | 13.343 | 12.272 | 11.454 |
| Ga0172365_10004421\| | | | | | | | | |
| 633 . . . 2366\|revcom | | | | | | | | |
| 209658_13971_protein_locus_{—} | 40.941 | | 100 | 100 | 13.993 | 14.286 | 12.871 | 13.531 |
| of_contig_Ga0190333_1001561 - | | | | | | | | |
| Query protein | | | | | | | | |
| (209658_13971) | | | | | | | | |
| (2) | | | | | | | | |
| 209657_57738_protein_locus_{—} | 67.317 | 100 | | 100 | 18.272 | 24.242 | 18.927 | 18.927 |
| of_contig_Ga0190332_1015597 - | | | | | | | | |
| Query protein | | | | | | | | |
| (209657_57738) | | | | | | | | |
| (2) | | | | | | | | |
| 209660_51257_protein_locus_{—} | 66.495 | 100 | 100 | | 17.931 | 22.831 | 18.301 | 18.301 |
| of_contig_Ga0190335_1015156 - | | | | | | | | |
| Query protein | | | | | | | | |
| (209660_51257) | | | | | | | | |
| (2) | | | | | | | | |
| Cas14b.14\|gwc1_scaffold_8732_{—} | 13 | 13.993 | 18.272 | 17.931 | | 16.712 | 27.394 | 23.047 |
| curated\|2705 . . . 4537 | | | | | | | | |
| Cas14b.15\|3300010293.a\| | 13.343 | 14.286 | 24.242 | 22.831 | 16.712 | | 14.951 | 18.385 |
| Ga0116204_1008574\| | | | | | | | | |
| 2134 . . . 4032 | | | | | | | | |
| Cas14b.12\|CG22_combo_CG10- | 12.272 | 12.871 | 18.927 | 18.301 | 27.394 | 14.951 | | 40.772 |
| 13_8_21_14_all_scaffold_2003_{—} | | | | | | | | |
| curated\|553 . . . | | | | | | | | |
| 2880\|revcom | | | | | | | | |
| Cas14b.13\|rifcsphigho2_01_{—} | 11.454 | 13.531 | 18.927 | 18.301 | 23.047 | 18.385 | 40.772 | |
| scaffold_82367_curated\| | | | | | | | | |
| 1523 . . . 3856\|revcom | | | | | | | | |
| Cas14b.16\|3300005573.a\| | 14.286 | 16.364 | 26.126 | 25.592 | 18.759 | 21.333 | 19.549 | 20.411 |
| Ga0078972_1001015a\| | | | | | | | | |
| 33750 . . . 35627 | | | | | | | | |
| Cas14b.10\|CG08_land_8_20_14_{—} | 15.123 | 15.565 | 24.554 | 23.944 | 18.091 | 23.263 | 19.798 | 19.898 |
| 0.20_scaffold_1609_{—} | | | | | | | | |
| curated\|6134 . . . 7975 | | | | | | | | |
| Cas14b.11\|CG_4_10_14_0.8_um_{—} | 14.701 | 14.468 | 24.554 | 23.944 | 17.236 | 22.87 | 19.75 | 21.673 |
| filter_scaffold_20762_{—} | | | | | | | | |
| curated\|1372 . . . 3219 | | | | | | | | |
| Cas14u.1\|3300009029.a\| | 10.152 | 12.983 | 19.005 | 19.048 | 7.932 | 8.73 | 6.727 | 6.43 |
| Ga0066793_10010091\| | | | | | | | | |
| 37 . . . 1113\|revcom | | | | | | | | |
| Cas12c1 | 5.293 | 4.287 | 8.495 | 8.608 | 5.141 | 5.008 | 5.988 | 5.478 |
| Cas12c2 | 4.519 | 4.063 | 8.753 | 8.611 | 3.878 | 3.897 | 5.064 | 5.263 |
| Cas12a_UPI001113398F | 4.479 | 3.345 | 6.516 | 5.605 | 5.328 | 5.481 | 4.476 | 5.171 |
| Cas12b_UPI001113398F | 4.479 | 3.345 | 6.516 | 5.605 | 5.328 | 5.481 | 4.476 | 5.171 |
| Cas12b_tr\|A0A1I7F1U9\| | 4.388 | 3.341 | 6.497 | 5.588 | 5.236 | 5.476 | 4.476 | 5.254 |
| A0A117F1U9_9BACL | | | | | | | | |
| Cas12a_UPI00083514A7 | 3.731 | 3.1 | 7.102 | 6.805 | 4.522 | 5.112 | 4.614 | 5.329 |
| Cas12b_UPI00083514A7 | 3.731 | 3.1 | 7.102 | 6.805 | 4.522 | 5.112 | 4.614 | 5.329 |
| Cas12a_UPI00097159F1 | 4.66 | 2.966 | 5.698 | 5.935 | 4.626 | 5.316 | 4.46 | 5.344 |
| Cas12b_UPI00097159F1 | 4.66 | 2.966 | 5.698 | 5.935 | 4.626 | 5.316 | 4.46 | 5.344 |
| Cas12b_sp\|T0D7A2\| | 4.66 | 2.966 | 5.698 | 5.935 | 4.626 | 5.316 | 4.46 | 5.344 |
| CS12B_ALIAG | | | | | | | | |
| Cas12a_UPI0009715A14 | 4.66 | 2.966 | 5.698 | 5.935 | 4.626 | 5.316 | 4.46 | 5.344 |
| Cas12b_UPI0009715A14 | 4.66 | 2.966 | 5.698 | 5.935 | 4.626 | 5.316 | 4.46 | 5.344 |
| Cas12a_UPI00097159CF | 4.66 | 2.966 | 5.698 | 5.935 | 4.626 | 5.316 | 4.46 | 5.344 |
| Cas12b_UPI00097159CF | 4.66 | 2.966 | 5.698 | 5.935 | 4.626 | 5.316 | 4.46 | 5.344 |
| Cas12a_UPI000832F6D2 | 5.028 | 2.962 | 5.966 | 6.213 | 4.8 | 5.128 | 4.799 | 5.254 |
| Cas12b_UPI000832F6D2 | 5.028 | 2.962 | 5.966 | 6.213 | 4.8 | 5.128 | 4.799 | 5.254 |
| Cas12b_tr\|A0A512CSX2\| | 5.307 | 2.962 | 5.966 | 6.213 | 4.711 | 4.945 | 4.713 | 5.508 |
| A0A512CSX2_9BACL | | | | | | | | |
| OspCas12c | 5.537 | 4.028 | 7.71 | 7.477 | 4.309 | 5.263 | 5.016 | 4.71 |
| Cas14u.5\|3300012532.a\| | 8.213 | 5.056 | 9.412 | 10.084 | 5.078 | 6.46 | 5.788 | 4.436 |
| Ga0137373_10000316\| | | | | | | | | |
| 3286 . . . 5286 | | | | | | | | |
| 63461_4106_protein_locus_{—} | 8.756 | 6.681 | 9.449 | 9.877 | 4.762 | 5.532 | 5.388 | 4.326 |
| of_contig_LSKL01000323 - | | | | | | | | |
| Query protein | | | | | | | | |
| (63461_4106) | | | | | | | | |
| translation (4) | | | | | | | | |
| 58610_1188_protein_locus_{—} | 5.615 | 5.749 | 8.365 | 8.13 | 5.321 | 6.601 | 4.316 | 5.179 |
| of_contig_LFOD01000003 - | | | | | | | | |
| Query protein | | | | | | | | |
| (58610_1188) | | | | | | | | |
| translation (5) | | | | | | | | |
| 21566_3969_protein_locus_{—} | 6.175 | 6.098 | 8.812 | 8.8 | 6.241 | 6.268 | 5.604 | 5.062 |
| of_contig_BAFB01000202 - | | | | | | | | |
| Query protein | | | | | | | | |
| (21566_3969) | | | | | | | | |
| translation (4) | | | | | | | | |

TABLE 10

| | | | | Cas14u.1\| | | | | |
|---|---|---|---|---|---|---|---|---|
| | Cas14b.16\| | Cas14b.10\|CG08_{—} | Cas14b.11\|CG_4_{—} | 3300009029.a\| | | | | |
| | 3300005573a\| | land_8_20_14_{—} | 10_14_0.8_um_{—} | Ga0066793_{—} | | | | |
| | Ga078972_{—} | 0.20_scaffold_{—} | filter_scaffold_{—} | 10010091\| | | | | |
| | 1001015a\| | 1609_curated\| | 20762_curated\| | 37 . . . 1113\| | | | Cas12a_{—} | Cas12b_{—} |
| | 33750 . . . 35627 | 6134 . . . 7975 | 1372 . . . 3219 | revcom | Cas12c1 | Cas12c2 | UPI1113398F | UPI001113398F |
| Cas14g.1\|RBG_13_{—} | 8.607 | 8.969 | 9.151 | 7.801 | 3.749 | 5.609 | 4.949 | 4.949 |
| scaffold_1401_curated\| | | | | | | | | |
| 15949 . . . 18180 | | | | | | | | |
| Cas14g.2\|3300009652.a\| | 6.86 | 9.031 | 7.513 | 6.658 | 5.389 | 5.178 | 5.412 | 5.412 |
| Ga0123330_1010394\| | | | | | | | | |
| 2814 . . . 5123 | | | | | | | | |
| Cas12i2 | 5.529 | 4.981 | 4.803 | 2.761 | 5.444 | 5.988 | 7.131 | 7.131 |
| Cas12i1 | 5.009 | 6.187 | 5.097 | 3.636 | 5.339 | 4.403 | 5.547 | 5.547 |
| Cas12g1 | 9.554 | 8.217 | 8.805 | 6.992 | 5.582 | 5.954 | 5.649 | 5.649 |
| Cas14d.3\|RIFCSPLOWO2_{—} | 8.604 | 7.255 | 7.714 | 6.535 | 4.362 | 4.676 | 5.709 | 5.709 |
| 01_FULL_OD1_45_34b_{—} | | | | | | | | |
| rifcsplowo2_01_{—} | | | | | | | | |
| scaffold_3495_curated\| | | | | | | | | |
| 25656 . . . 27605\|revcom | | | | | | | | |
| Cas14d.1\|RIFCSPHIGHO2_{—} | 8.247 | 6.647 | 7.829 | 7.085 | 3.803 | 4.073 | 5.372 | 5.372 |
| 01_FULL_CPR_46_36_{—} | | | | | | | | |
| rifcsphigho2_01_{—} | | | | | | | | |
| scaffold_646_curated\| | | | | | | | | |
| 49808 . . . 51616\|revcom | | | | | | | | |
| CasY5 | 3.53 | 4.974 | 4.01 | 2.599 | 5.334 | 5.778 | 7.105 | 7.105 |
| Cas14a.4\|CG10_big_fil_{—} | 7.294 | 8.621 | 6.974 | 7.865 | 3.943 | 4.396 | 3.91 | 3.91 |
| rev_8_21_14_0.10_{—} | | | | | | | | |
| scaffold_20906_curated\| | | | | | | | | |
| 649 . . . 2829 | | | | | | | | |
| CasY6 | 4.444 | 4.167 | 4.567 | 2.972 | 7.076 | 6.856 | 7.015 | 7.015 |
| Cas14f.1\|rifcsp13_1_sub10_{—} | 8.161 | 7.412 | 7.263 | 6.276 | 5.155 | 4.448 | 6.356 | 6.356 |
| scaffold_3_curated\| | | | | | | | | |
| 38906 . . . 41041 | | | | | | | | |
| Cas14f.2\|3300009991.a\| | 7.123 | 7.613 | 6.589 | 7.279 | 3.681 | 3.598 | 4.2 | 4.2 |
| Ga0105042_100140\| | | | | | | | | |
| 1624 . . . 3348 | | | | | | | | |
| Cas14a.6\|3300012359.a\| | 9.385 | 8.661 | 9.291 | 8.884 | 3.421 | 4.153 | 2.899 | 2.899 |
| Ga0137385_10000156\| | | | | | | | | |
| 41289 . . . 42734 | | | | | | | | |
| Cas12a_UPI00094EEDB4 | 5.104 | 4.224 | 4.228 | 2.422 | 7.387 | 5.411 | 5.679 | 5.679 |
| Cas12a_UPI000B4235CE | 5.097 | 4.587 | 4.82 | 3.04 | 7.064 | 6.555 | 5.297 | 5.297 |
| Cas12a_UPI000818CC52 | 5.104 | 4.671 | 4.904 | 3.04 | 7.074 | 6.564 | 5.233 | 5.233 |
| Cas12a_UPI0007B78B7F | 5.097 | 4.587 | 4.82 | 3.04 | 7.064 | 6.555 | 5.225 | 5.225 |
| Cas12a_UPI000B4235F9 | 5.015 | 4.431 | 4.82 | 3.04 | 7.059 | 6.485 | 5.225 | 5.225 |
| Cas14e.2\|rifcsplowo2_01_{—} | 8.544 | 8.416 | 9.36 | 8.12 | 2.875 | 2.421 | 3.768 | 3.768 |
| scaffold_81231_curated\| | | | | | | | | |
| 976 . . . 2217 | | | | | | | | |
| Cas14e.1\|rifcsphigho2_01_{—} | 6.552 | 5.366 | 7.553 | 9.013 | 4.003 | 3.003 | 3.483 | 3.483 |
| scaffold_566_curated\| | | | | | | | | |
| 113069 . . . 114313 | | | | | | | | |
| Cas14e.3\|rifcsphigho2_01_{—} | 7.899 | 7.084 | 8.94 | 8.678 | 3.68 | 2.836 | 5.239 | 5.239 |
| scaffold_4702_curated\| | | | | | | | | |
| 82881 . . . 84230\|revcom | | | | | | | | |
| CasY4 | 5.401 | 5.755 | 5.356 | 3.168 | 6.734 | 5.498 | 6.737 | 6.737 |
| Cas14h.3\|3300009698.a\| | 7.553 | 7.951 | 7.034 | 11.469 | 3.969 | 3.997 | 4.758 | 4.758 |
| Ga0116216_10000905\| | | | | | | | | |
| 8005 . . . 9504 | | | | | | | | |
| Cas14h.1\|3300005602.a\| | 5.655 | 6.212 | 7.251 | 7.005 | 3.965 | 3.846 | 5.206 | 5.206 |
| Ga0070762_10001740\| | | | | | | | | |
| 7377 . . . 9071\|revcom | | | | | | | | |
| Cas14h.2\|3300005921.a\| | 5.891 | 7.187 | 8.346 | 7.951 | 4.196 | 4.01 | 4.668 | 4.668 |
| Ga0070766_10011912\| | | | | | | | | |
| 384 . . . 2081 | | | | | | | | |
| Cas14c.1\|CG10_big_fil_{—} | 8.921 | 8.837 | 7.965 | 7.129 | 3.75 | 3.207 | 3.856 | 3.856 |
| rev_8_21_14_0.10_{—} | | | | | | | | |
| scaffold_4477_curated\| | | | | | | | | |
| 19327 . . . 20880\|revcom | | | | | | | | |
| Cas12h1 | 6.171 | 5.977 | 5.963 | 3.865 | 5.352 | 5.016 | 5.598 | 5.598 |
| CasX1 | 6.612 | 6.984 | 7.419 | 5.456 | 7.083 | 6.63 | 6.371 | 6.371 |
| CasX2 | 6.66 | 7.464 | 7.906 | 5.191 | 7.192 | 5.915 | 6.209 | 6.209 |
| CasY1 | 6.818 | 6.828 | 5.951 | 4.048 | 7.049 | 5.659 | 5.166 | 5.166 |
| Cas14u.3\|19ft_2_nophage_{—} | 9.176 | 10.098 | 9.82 | 10.331 | 3.92 | 3.172 | 4.269 | 4.269 |
| noknown_scaffold_0_curated\| | | | | | | | | |
| 508188 . . . 59648 | | | | | | | | |
| Cas14u.7\|3300001256.a\| | 7.865 | 10.231 | 9.091 | 12.528 | 3.259 | 3.185 | 3.249 | 3.249 |
| JGI12210J13797_10004690\| | | | | | | | | |
| 5792 . . . 7006 | | | | | | | | |
| Cas14u.8\|3300005660.a\| | 8.64 | 8.553 | 8.224 | 14.151 | 3.016 | 3.598 | 3.96 | 3.96 |
| Ga0073904_10021651\| | | | | | | | | |
| 765 . . . 1943 | | | | | | | | |
| Cas14u.4\|rifcsp2_19_4_full_{—} | 8.28 | 10.164 | 8.867 | 13.122 | 3.41 | 3.434 | 3.156 | 3.156 |
| scaffold_168_curated\| | | | | | | | | |
| 84455 . . . 85657 | | | | | | | | |
| Cas14d.2\|rifcsphigho2_01_{—} | 8.1 | 7.98 | 7.516 | 8.876 | 4.177 | 4.362 | 4.779 | 4.779 |
| scaffold_10981_curated\| | | | | | | | | |
| 5762 . . . 7246\|revcom | | | | | | | | |
| Cas14c.2\|3300001245.a\| | 7.547 | 8.347 | 8.039 | 10.502 | 3.085 | 3.156 | 3.142 | 3.142 |
| JGI12048J13642_10201286\| | | | | | | | | |
| 4257 . . . 5489\|revcom | | | | | | | | |
| CasY3 | 5.056 | 5.702 | 5.541 | 3.643 | 6.218 | 7.863 | 5.779 | 5.779 |
| 633299_527_protein_locus_{—} | 8.9 | 8.099 | 8.609 | 13.318 | 3.819 | 3.275 | 3.258 | 3.258 |
| of_contig_Scfld15 - Query | | | | | | | | |
| protein (633299_527) (4) | | | | | | | | |
| 8971_2857_protein_locus_{—} | 9.424 | 9.386 | 9.567 | 13.384 | 3.541 | 3.226 | 2.486 | 2.486 |
| of_contig_OEJQ01000083.1 - | | | | | | | | |
| Query protein (8971_2857) | | | | | | | | |
| 9265_901_protein_locus_{—} | 9.589 | 9.381 | 9.381 | 13.022 | 3.509 | 3.283 | 2.554 | 2.554 |
| of_contig_OEFX01000005.1 - | | | | | | | | |
| Query protein (9265_901) | | | | | | | | |
| Cas14u.6\|3300006028.a\| | 7.264 | 10.502 | 8.976 | 7.584 | 4.286 | 4.424 | 5.068 | 5.068 |
| Ga0070717_10000077\| | | | | | | | | |
| 54519 . . . 56201\|revcom | | | | | | | | |
| 466065_250_protein_locus_{—} | 9.493 | 10.94 | 10.427 | 13.318 | 3.647 | 4.135 | 2.971 | 2.971 |
| of_contig_SFKR01000004.1 - | | | | | | | | |
| Query protein (466065_250) | | | | | | | | |
| Cas14a.5\|rifcsplowo2_01_{—} | 8.722 | 6.891 | 7.573 | 7.707 | 2.584 | 3.878 | 5.103 | 5.103 |
| scaffold_34461_curated\| | | | | | | | | |
| 4968 . . . 6521 | | | | | | | | |
| CasY2 | 5.719 | 5.491 | 5.008 | 3.336 | 6.014 | 6.632 | 6.712 | 6.712 |
| Cas14a.3\|gwa1_{—} | 11.502 | 10.129 | 10.129 | 9.982 | 4.106 | 5.117 | 5.418 | 5.418 |
| scaffold_1795_curated\| | | | | | | | | |
| 25635 . . . 27224\|revcom | | | | | | | | |
| Cas14a.1\|rifcsphigho2_02_{—} | 9.969 | 8.654 | 9.807 | 13.069 | 4.466 | 5.518 | 4.288 | 4.288 |
| scaffold_2167_curated\| | | | | | | | | |
| 30296 . . . 31798\|revcom | | | | | | | | |
| Cas14a.2\|gwa2_{—} | 9.502 | 9.206 | 8.931 | 12.871 | 4.203 | 5.184 | 5.077 | 5.077 |
| scaffold_18027_curated\| | | | | | | | | |
| 7105 . . . 8628 | | | | | | | | |
| Cas14b.4\|cg1_0.2_{—} | 13.509 | 13.744 | 11.765 | 8.834 | 4.24 | 4.854 | 4.117 | 4.117 |
| scaffold_785_c_curated\| | | | | | | | | |
| 32521 . . . 34155 | | | | | | | | |
| Cas14b.7\|3300013125.a\| | 13.077 | 14.6 | 14.396 | 9.414 | 4.629 | 4 | 4.662 | 4.662 |
| Ga0172369_10000737\| | | | | | | | | |
| 994 . . . 2652\|revcom | | | | | | | | |
| Cas14u.2\|3300002172.a\| | 9.431 | 10.483 | 10.333 | 17.115 | 4.157 | 3.12 | 2.74 | 2.74 |
| JGI24730J26740_1002785\| | | | | | | | | |
| 496 . . . 1605\|revcom | | | | | | | | |
| Cas14b.3\|rifcsphigho2_01_{—} | 15.147 | 15.397 | 12.711 | 11.314 | 5.671 | 3.827 | 3.653 | 3.653 |
| scaffold_36781_curated\| | | | | | | | | |
| 2592 . . . 4217 | | | | | | | | |
| Cas14b.2\|rifcsplowo2_01_{—} | 15.335 | 16.066 | 15.798 | 11.151 | 4.919 | 3.782 | 3.509 | 3.509 |
| scaffold_282_curated\| | | | | | | | | |
| 77370 . . . 78983 | | | | | | | | |
| Cas14b.1\|rifcsplowo2_01_{—} | 15.848 | 15.285 | 15.994 | 11.84 | 5.221 | 4.603 | 4.136 | 4.136 |
| scaffold_239_curated\| | | | | | | | | |
| 54653 . . . 56257 | | | | | | | | |
| Cas14b.8\|3300013125.a\| | 14.263 | 15.122 | 15.024 | 11.883 | 5.783 | 4.603 | 3.86 | 3.86 |
| Ga0172369_10010464\| | | | | | | | | |
| 885 . . . 2489\|revcom | | | | | | | | |
| Cas14b.5\|rifcsphigho2_02_{—} | 15.822 | 14.33 | 15.373 | 10.106 | 4.48 | 4.841 | 4.209 | 4.209 |
| scaffold_55589_curated\| | | | | | | | | |
| 1904 . . . 3598 | | | | | | | | |
| Cas14b.6\|CG03_land_{—} | 14.074 | 12.254 | 12.236 | 10.114 | 5.242 | 5.12 | 4.039 | 4.039 |
| 8_20_14_0.80_{—} | | | | | | | | |
| scaffold_2214_curated\| | | | | | | | | |
| 6634 . . . 8466\|revcom | | | | | | | | |
| Cas14b.9\|3300013127.a\| | 14.286 | 15.123 | 14.701 | 10.152 | 5.293 | 4.519 | 4.479 | 4.479 |
| Ga0172365_10004421\| | | | | | | | | |
| 633 . . . 2366\|revcom | | | | | | | | |
| 209658_13971_protein_{—} | 16.364 | 15.565 | 14.468 | 12.983 | 4.287 | 4.063 | 3.345 | 3.345 |
| locus_of_contig_Ga0190333_{—} | | | | | | | | |
| 1001561 - Query protein | | | | | | | | |
| (209658_13971) (2) | | | | | | | | |
| 209657_57738_protein_{—} | 26.126 | 24.554 | 24.554 | 19.005 | 8.495 | 8.753 | 6.516 | 6.516 |
| locus_of_contig_Ga0190332_{—} | | | | | | | | |
| 1015597 - Query protein | | | | | | | | |
| (209657_57738) (2) | | | | | | | | |
| 209660_51257_protein_{—} | 25.592 | 23.944 | 23.944 | 19.048 | 8.608 | 8.611 | 5.605 | 5.605 |
| locus_of_contig_Ga0190335_{—} | | | | | | | | |
| 1015156 - Query protein | | | | | | | | |
| (209660_51257) (2) | | | | | | | | |
| Cas14b.14\|gwc1_{—} | 18.759 | 18.091 | 17.236 | 7.932 | 5.141 | 3.878 | 5.328 | 5.328 |
| scaffold_8732_curated\| | | | | | | | | |
| 2705 . . . 4537 | | | | | | | | |
| Cas14b.15\|3300010293.a\| | 21.333 | 23.263 | 22.87 | 8.73 | 5.008 | 3.897 | 5.481 | 5.481 |
| Ga0116204_1008574\| | | | | | | | | |
| 2134 . . . 4032 | | | | | | | | |
| Cas14b.12\|CG22_combo_{—} | 19.549 | 19.798 | 19.75 | 6.727 | 5.988 | 5.064 | 4.476 | 4.476 |
| CG10-13_8_21_14_all_{—} | | | | | | | | |
| scaffold_2003_curated\| | | | | | | | | |
| 553 . . . 2880\|revcom | | | | | | | | |
| Cas14b.13\|rifcsphigho2_01_{—} | 20.411 | 19.898 | 21.673 | 6.43 | 5.478 | 5.263 | 5.171 | 5.171 |
| scaffold_82367_curated\| | | | | | | | | |
| 1523 . . . 3856\|revcom | | | | | | | | |
| Cas14b.16\|3300005573.a\| | | 30.901 | 31.394 | 7.581 | 4.864 | 5.033 | 4.41 | 4.41 |
| Ga0078972_1001015a\| | | | | | | | | |
| 33750 . . . 35627 | | | | | | | | |
| Cas14b.10\|CG08_land_{—} | 30.901 | | 46.582 | 9 | 4.265 | 5.359 | 4.715 | 4.715 |
| 8_20_14_0.20_{—} | | | | | | | | |
| scaffold_1609_curated\| | | | | | | | | |
| 6134 . . . 7975 | | | | | | | | |
| Cas14b.11\|CG_4_10_{—} | 31.394 | 46.582 | | 7.667 | 4.657 | 4.455 | 4.267 | 4.267 |
| 14_0.8_um_filter_{—} | | | | | | | | |
| scaffold_20762_curated\| | | | | | | | | |
| 1372 . . . 3219 | | | | | | | | |
| Cas14u.1\|3300009029.a\| | 7.581 | 9 | 7.667 | | 3.05 | 3.193 | 3.768 | 3.768 |
| Ga0066793_10010091\| | | | | | | | | |
| 37 . . . 1113\|revcom | | | | | | | | |
| Cas12c1 | 4.864 | 4.265 | 4.657 | 3.05 | | 10.725 | 6.353 | 6.353 |
| Cas12c2 | 5.033 | 5.359 | 4.455 | 3.193 | 10.725 | | 6.867 | 6.867 |
| Cas12a_UPI001113398F | 4.41 | 4.715 | 4.267 | 3.768 | 6.353 | 6.867 | | 100 |
| Cas12b_UPI001113398F | 4.41 | 4.715 | 4.267 | 3.768 | 6.353 | 6.867 | 100 | |
| Cas12b_tr\|A0A1I7F1U9\| | 4.586 | 4.711 | 4.085 | 3.952 | 6.334 | 6.809 | 93.916 | 93.916 |
| A0A1I7F1U9_9BACL | | | | | | | | |
| Cas12a_UPI00083514A7 | 4.301 | 5.221 | 5.142 | 3.571 | 6.796 | 6.507 | 52.754 | 52.754 |
| Cas12b_UPI00083514A7 | 4.301 | 5.221 | 5.142 | 3.571 | 6.796 | 6.507 | 52.754 | 52.754 |
| Cas12a_UPI00097159F1 | 4.312 | 4.801 | 4.265 | 4.124 | 6.796 | 6.274 | 51.817 | 51.817 |
| Cas12b_UPI00097159F1 | 4.312 | 4.801 | 4.265 | 4.124 | 6.796 | 6.274 | 51.817 | 51.817 |
| Cas12b_sp\|T0D7A2\| | 4.312 | 4.801 | 4.265 | 4.124 | 6.796 | 6.274 | 51.817 | 51.817 |
| CS12B_ALIAG | | | | | | | | |
| Cas12a_UPI0009715A14 | 4.312 | 4.801 | 4.265 | 4.124 | 6.791 | 6.274 | 51.557 | 51.557 |
| Cas12b_UPI0009715A14 | 4.312 | 4.801 | 4.265 | 4.124 | 6.791 | 6.274 | 51.557 | 51.557 |
| Cas12a_UPI00097159CF | 4.312 | 4.801 | 4.265 | 4.124 | 6.791 | 6.274 | 51.73 | 51.73 |
| Cas12b_UPI00097159CF | 4.312 | 4.801 | 4.265 | 4.124 | 6.791 | 6.274 | 51.73 | 51.73 |
| Cas12a_UPI000832F6D2 | 4.216 | 4.887 | 4.533 | 4.221 | 6.572 | 6.042 | 51.513 | 51.513 |
| Cas12b_UPI000832F6D2 | 4.216 | 4.887 | 4.533 | 4.221 | 6.572 | 6.042 | 51.513 | 51.513 |
| Cas12b_tr\|A0A512CSX2\| | 4.216 | 4.615 | 4.352 | 4.311 | 6.497 | 5.887 | 51.685 | 51.685 |
| A0A512CSX2_9BACL | | | | | | | | |
| OspCas12c | 4.835 | 4.75 | 4.593 | 3.102 | 7.138 | 7.704 | 5.243 | 5.243 |
| Cas14u.5\|3300012532.a\| | 5.501 | 7.203 | 7.433 | 5.706 | 3.739 | 4.269 | 5.596 | 5.596 |
| Ga0137373_10000316\| | | | | | | | | |
| 3286 . . . 5286 | | | | | | | | |
| 63461_4106_protein_locus_{—} | 6.021 | 6.466 | 7.292 | 7.19 | 3.262 | 3.621 | 4.818 | 4.818 |
| of_contig_LSKL01000323 - | | | | | | | | |
| Query protein (63461_4106) | | | | | | | | |
| translation (4) | | | | | | | | |
| 58610_1188_protein_locus_{—} | 6.676 | 6.686 | 6.765 | 6.139 | 4.344 | 4.534 | 4.932 | 4.932 |
| of_contig_LFOD01000003 - | | | | | | | | |
| Query protein (58610_1188) | | | | | | | | |
| translation (5) | | | | | | | | |
| 21566_3969_protein_locus_{—} | 5.333 | 7.669 | 6.897 | 8.086 | 3.21 | 4.105 | 5.105 | 5.105 |
| of_contig_BAFB01000202 - | | | | | | | | |
| Query protein (21566_3969) | | | | | | | | |
| translation (4) | | | | | | | | |

TABLE 11

| | Cas12b_tr\| | | | | | | | |
|---|---|---|---|---|---|---|---|---|
| | A0A1I7F1U9\| | | | | | Cas12b_sp\| | | |
| | A0A1I7F1U9_{—} | Cas12a_{—} | Cas12b_{—} | Cas12a_{—} | Cas12b_{—} | T0D7A2\| | Cas12a_{—} | Cas12b_{—} |
| | 9BACL | UPI00083514A7 | UPI00083514A7 | UPI00097159F1 | UPI00097159F1 | CS12B_ALIAG | UPI0009715A14 | UPI0009715A14 |
| Cas14g.1\|RBG_13_{—} | 4.818 | 5.013 | 5.013 | 4.865 | 4.865 | 4.865 | 4.865 | 4.865 |
| scaffold_1401_curated\| | | | | | | | | |
| 15949 . . . 18180 | | | | | | | | |
| Cas14g.2\|3300009652.a\| | | | | | | | | |
| Ga0123330_1010394\| | | | | | | | | |
| 2814 . . . 5123 | | | | | | | | |
| Cas12i2 | 5.541 | 5.917 | 5.917 | 6.396 | 6.396 | 6.396 | 6.396 | 6.396 |
| Cas12i1 | 7.248 | 5.824 | 5.824 | 6.03 | 6.03 | 6.03 | 6.03 | 6.03 |
| Cas12g1 | 5.708 | 5.837 | 5.837 | 5.934 | 5.934 | 5.934 | 5.934 | 5.934 |
| Cas14d.3\|RIFCSPLOWO2_{—} | 5.434 | 5.986 | 5.986 | 5.845 | 5.845 | 5.845 | 5.935 | 5.935 |
| 01_FULL_OD1_45_34b_{—} | | | | | | | | |
| rifcsplowo2_01_{—} | | | | | | | | |
| scaffold_3495_curated\| | | | | | | | | |
| 25656 . . . 27605\|revcom | | | | | | | | |
| Cas14d.1\|RIFCSPHIGHO2_{—} | 5.585 | 5.254 | 5.254 | 5.1 | 5.1 | 5.1 | 5.1 | 5.1 |
| 01_FULL_CPR_46_36_{—} | | | | | | | | |
| rifcsphigho2_01_{—} | | | | | | | | |
| scaffold_646_curated\| | | | | | | | | |
| 49808 . . . 51616\|revcom | | | | | | | | |
| CasY5 | 5.461 | 5.085 | 5.085 | 5.743 | 5.743 | 5.743 | 5.743 | 5.743 |
| Cas14a.4\|CG10_big_fil_{—} | 7.186 | 6.941 | 6.941 | 6.921 | 6.921 | 6.921 | 6.838 | 6.838 |
| rev_8_21_14_0.10_{—} | | | | | | | | |
| scaffold_20906_curated\| | | | | | | | | |
| 649 . . . 2829 | | | | | | | | |
| CasY6 | 3.747 | 4.391 | 4.391 | 5.165 | 5.165 | 5.165 | 5.165 | 5.165 |
| Cas14f.1\|rifcsp13_1_sub10_{—} | 6.942 | 6.428 | 6.428 | 6.133 | 6.133 | 6.133 | 6.058 | 6.058 |
| scaffold_3_curated\| | | | | | | | | |
| 38906 . . . 41041 | | | | | | | | |
| Cas14f.2\|3300009991.a\| | 6.394 | 6.014 | 6.014 | 6.324 | 6.324 | 6.324 | 6.324 | 6.324 |
| Ga0105042_100140\| | | | | | | | | |
| 1624 . . . 3348 | | | | | | | | |
| Cas14a.6\|3300012359.a\| | 4.259 | 4.541 | 4.541 | 4.558 | 4.558 | 4.558 | 4.649 | 4.649 |
| Ga0137385_10000156\| | | | | | | | | |
| 41289 . . . 42734 | | | | | | | | |
| Cas12a_UPI00094EEDB4 | 2.893 | 4.159 | 4.159 | 2.69 | 2.69 | 2.69 | 2.69 | 2.69 |
| Cas12a_UPI000B4235CE | 5.575 | 6.026 | 6.026 | 6.82 | 6.82 | 6.82 | 6.82 | 6.82 |
| Cas12a_UPI000818CC52 | 5.323 | 5.583 | 5.583 | 6.017 | 6.017 | 6.017 | 6.017 | 6.017 |
| Cas12a_UPI0007B78B7F | 5.259 | 5.448 | 5.448 | 5.882 | 5.882 | 5.882 | 5.882 | 5.882 |
| Cas12a_UPI000B4235F9 | 5.252 | 5.44 | 5.44 | 5.874 | 5.874 | 5.874 | 5.874 | 5.874 |
| Cas14e.2\|rifcsplowo2_01_{—} | 5.252 | 5.512 | 5.512 | 5.946 | 5.946 | 5.946 | 5.946 | 5.946 |
| scaffold_81231_curated\| | | | | | | | | |
| 976 . . . 2217 | | | | | | | | |
| Cas14e.1\|rifcsphigho2_01_{—} | 3.772 | 3.846 | 3.846 | 4.03 | 4.03 | 4.03 | 4.03 | 4.03 |
| scaffold_566_curated\| | | | | | | | | |
| 113069 . . . 114313 | | | | | | | | |
| Cas14e.3\|rifcsphigho2_01_{—} | 3.388 | 3.822 | 3.822 | 3.825 | 3.825 | 3.825 | 3.825 | 3.825 |
| scaffold_4702_curated\| | | | | | | | | |
| 82881 . . . 84230\|revcom | | | | | | | | |
| CasY4 | 5.133 | 4.388 | 4.388 | 5.717 | 5.717 | 5.717 | 5.717 | 5.717 |
| Cas14h.3\|3300009698.a\| | 6.546 | 5.998 | 5.998 | 5.998 | 5.998 | 5.998 | 6.074 | 6.074 |
| Ga0116216_10000905\| | | | | | | | | |
| 8005 . . . 9504 | | | | | | | | |
| Cas14h.1\|3300005602.a\| | 4.633 | 4.112 | 4.112 | 4.093 | 4.093 | 4.122 | 4.122 | 4.122 |
| Ga0070762_10001740\| | | | | | | | | |
| 7377 . . . 9071\|revcom | | | | | | | | |
| Cas14h.2\|3300005921.a\| | 5.306 | 4.749 | 4.749 | 5.225 | 5.225 | 5.225 | 5.225 | 5.225 |
| Ga0070766_10011912\| | | | | | | | | |
| 384 . . . 2081 | | | | | | | | |
| Cas14c.1\|CG10_big_fil_{—} | 4.852 | 4.659 | 4.659 | 5.133 | 5.133 | 5.133 | 5.133 | 5.133 |
| rev_8_21_14_0.10_{—} | | | | | | | | |
| scaffold_4477_curated\| | | | | | | | | |
| 19327 . . . 20880\|revcom | | | | | | | | |
| Cas12h1 | 3.665 | 4.087 | 4.087 | 4.452 | 4.452 | 4.452 | 4.452 | 4.452 |
| CasX1 | 5.763 | 5.64 | 5.64 | 6.374 | 6.374 | 6.374 | 6.374 | 6.374 |
| CasX2 | 6.31 | 6.034 | 6.034 | 5.916 | 5.916 | 5.916 | 5.916 | 5.916 |
| CasY1 | 5.882 | 5.705 | 5.705 | 5.412 | 5.412 | 5.412 | 5.329 | 5.329 |
| Cas14u.3\|19ft_2_nophage_{—} | 5.183 | 5.624 | 5.624 | 4.867 | 4.867 | 4.867 | 4.867 | 4.867 |
| noknown_scaffold_0_curated\| | | | | | | | | |
| 508188 . . . 59648 | | | | | | | | |
| Cas14u.7\|3300001256.a\| | 4.269 | 3.993 | 3.993 | 4.457 | 4.457 | 4.457 | 4.457 | 4.457 |
| JGI12210J13797_10004690\| | | | | | | | | |
| 5792 . . . 7006 | | | | | | | | |
| Cas14u.8\|3300005660.a\| | 3.237 | 3.584 | 3.584 | 3.306 | 3.306 | 3.306 | 3.214 | 3.214 |
| Ga0073904_10021651\| | | | | | | | | |
| 765 . . . 1943 | | | | | | | | |
| Cas14u.4\|rifcsp2_19_4_full_{—} | 3.957 | 3.136 | 3.136 | 2.661 | 2.661 | 2.661 | 2.661 | 2.661 |
| scaffold_168_curated\| | | | | | | | | |
| 84455 . . . 85657 | | | | | | | | |
| Cas14d.2\|rifcsphigho2_01_{—} | 3.055 | 3.232 | 3.232 | 2.663 | 2.663 | 2.663 | 2.663 | 2.663 |
| scaffold_10981_curated\| | | | | | | | | |
| 5762 . . . 7246\|revcom | | | | | | | | |
| Cas14c.2\|3300001245.a\| | 4.867 | 4.487 | 4.487 | 4.503 | 4.503 | 4.503 | 4.503 | 4.503 |
| JGI12048J13642_10201286\| | | | | | | | | |
| 4257 . . . 5489\|revcom | | | | | | | | |
| CasY3 | 3.139 | 2.594 | 2.594 | 3.294 | 3.294 | 3.294 | 3.294 | 3.294 |
| 633299_527_protein_locus_{—} | 5.807 | 6.591 | 6.591 | 6.298 | 6.298 | 6.298 | 6.298 | 6.298 |
| of_contig_Scfld15 - Query | | | | | | | | |
| protein (633299_527) (4) | | | | | | | | |
| 8971_2857_protein_locus_{—} | 3.348 | 2.599 | 2.599 | 3.643 | 3.643 | 3.578 | 3.578 | 3.578 |
| of_contig_OEJQ01000083.1 - | | | | | | | | |
| Query protein (8971_2857) | | | | | | | | |
| 9265_901_protein_locus_{—} | 2.481 | 2.657 | 2.657 | 2.242 | 2.242 | 2.242 | 2.242 | 2.242 |
| of_contig_OEFX01000005.1 - | | | | | | | | |
| Query protein (9265_901) | | | | | | | | |
| Cas14u.6\|3300006028.a\| | 2.55 | 2.723 | 2.723 | 2.314 | 2.314 | 2.314 | 2.314 | 2.314 |
| Ga0070717_10000077\| | | | | | | | | |
| 54519 . . . 56201\|revcom | | | | | | | | |
| 466065_250_protein_locus_{—} | 5.158 | 4.599 | 4.599 | 4.428 | 4.428 | 4.428 | 4.428 | 4.428 |
| of_contig_SFKR01000004.1 - | | | | | | | | |
| Query protein (466065_250) | | | | | | | | |
| Cas14a.5\|rifcsplowo2_01_{—} | 3.058 | 2.308 | 2.308 | 2.844 | 2.844 | 2.844 | 2.844 | 2.844 |
| scaffold_34461_curated\| | | | | | | | | |
| 4968 . . . 6521 | | | | | | | | |
| CasY2 | 5.169 | 4.728 | 4.728 | 5.302 | 5.302 | 5.302 | 5.302 | 5.302 |
| Cas14a.3\|gwa1_{—} | 6.642 | 5.927 | 5.927 | 6.616 | 6.616 | 6.656 | 6.656 | 6.656 |
| scaffold_1795_curated\| | | | | | | | | |
| 25635 . . . 27224\|revcom | | | | | | | | |
| Cas14a.1\|rifcsphigho2_02_{—} | 5.142 | 4.487 | 4.487 | 4.69 | 4.69 | 4.69 | 4.69 | 4.69 |
| scaffold_2167_curated\| | | | | | | | | |
| 30296 . . . 31798\|revcom | | | | | | | | |
| Cas14a.2\|gwa2_{—} | 4.189 | 4.455 | 4.455 | 4.944 | 4.944 | 4.944 | 4.944 | 4.944 |
| scaffold_18027_curated\| | | | | | | | | |
| 7105 . . . 8628 | | | | | | | | |
| Cas14b.4\|cg1_0.2_{—} | 4.977 | 4.517 | 4.517 | 5.097 | 5.097 | 5.097 | 5.097 | 5.097 |
| scaffold_785_c_curated\| | | | | | | | | |
| 32521 . . . 34155 | | | | | | | | |
| Cas14b.7\|3300013125.a\| | 4.026 | 4.45 | 4.45 | 3.911 | 3.911 | 3.911 | 3.911 | 3.911 |
| Ga0172369_10000737\| | | | | | | | | |
| 994 . . . 2652\|revcom | | | | | | | | |
| Cas14u.2\|3300002172.a\| | 4.662 | 3.993 | 3.993 | 4.735 | 4.735 | 4.735 | 4.735 | 4.735 |
| JGI24730J26740_1002785\| | | | | | | | | |
| 496 . . . 1605\|revcom | | | | | | | | |
| Cas14b.3\|rifcsphigho2_01_{—} | 2.742 | 3.279 | 3.279 | 2.796 | 2.796 | 2.796 | 2.796 | 2.796 |
| scaffold_36781_curated\| | | | | | | | | |
| 2592 . . . 4217 | | | | | | | | |
| Cas14b.2\|rifcsplowo2_01_{—} | 3.653 | 3.822 | 3.822 | 4.186 | 4.186 | 4.186 | 4.186 | 4.186 |
| scaffold_282_curated\| | | | | | | | | |
| 77370 . . . 78983 | | | | | | | | |
| Cas14b.1\|rifcsplowo2_01_{—} | 3.506 | 3.036 | 3.036 | 3.857 | 3.857 | 3.857 | 3.857 | 3.857 |
| scaffold_239_curated\| | | | | | | | | |
| 54653 . . . 56257 | | | | | | | | |
| Cas14b.8\|3300013125.a\| | 4.132 | 3.388 | 3.388 | 4.026 | 4.026 | 4.026 | 4.026 | 4.026 |
| Ga0172369_10010464\| | | | | | | | | |
| 885 . . . 2489\|revcom | | | | | | | | |
| Cas14b.5\|rifcsphigho2_02_{—} | 3.857 | 3.663 | 3.663 | 4.588 | 4.588 | 4.588 | 4.588 | 4.588 |
| scaffold_55589_curated\| | | | | | | | | |
| 1904 . . . 3598 | | | | | | | | |
| Cas14b.6\|CG03_land_{—} | 4.209 | 4.011 | 4.011 | 4.007 | 4.007 | 4.007 | 4.007 | 4.007 |
| 8_20_14_0.80_{—} | | | | | | | | |
| scaffold_2214_curated\| | | | | | | | | |
| 6634 . . . 8466\|revcom | | | | | | | | |
| Cas14b.9\|3300013127.a\| | 4.032 | 4.383 | 4.383 | 3.85 | 3.85 | 3.85 | 3.85 | 3.85 |
| Ga0172365_10004421\| | | | | | | | | |
| 633 . . . 2366\|revcom | | | | | | | | |
| 209658_13971_protein_{—} | 4.388 | 3.731 | 3.731 | 4.66 | 4.66 | 4.66 | 4.66 | 4.66 |
| locus_of_contig_Ga0190333_{—} | | | | | | | | |
| 1001561 - Query protein | | | | | | | | |
| (209658_13971) (2) | | | | | | | | |
| 209657_57738_protein_{—} | 3.341 | 3.1 | 3.1 | 2.966 | 2.966 | 2.966 | 2.966 | 2.966 |
| locus_of_contig_Ga0190332_{—} | | | | | | | | |
| 1015597 - Query protein | | | | | | | | |
| (209657_57738) (2) | | | | | | | | |
| 209660_51257_protein_{—} | 6.497 | 7.102 | 7.102 | 5.698 | 5.698 | 5.698 | 5.698 | 5.698 |
| locus_of_contig_Ga0190335_{—} | | | | | | | | |
| 1015156 - Query protein | | | | | | | | |
| (209660_51257) (2) | | | | | | | | |
| Cas14b.14\|gwc1_{—} | 5.588 | 6.805 | 6.805 | 5.935 | 5.935 | 5.935 | 5.935 | 5.935 |
| scaffold_8732_curated\| | | | | | | | | |
| 2705 . . . 4537 | | | | | | | | |
| Cas14b.15\|3300010293.a\| | 5.236 | 4.522 | 4.522 | 4.626 | 4.626 | 4.626 | 4.626 | 4.626 |
| Ga0116204_1008574\| | | | | | | | | |
| 2134 . . . 4032 | | | | | | | | |
| Cas14b.12\|CG22_combo_{—} | 5.476 | 5.112 | 5.112 | 5.316 | 5.316 | 5.316 | 5.316 | 5.316 |
| CG10-13_8_21_14_all_{—} | | | | | | | | |
| scaffold_2003_curated\| | | | | | | | | |
| 553 . . . 2880\|revcom | | | | | | | | |
| Cas14b.13\|rifcsphigho2_01_{—} | 4.476 | 4.614 | 4.614 | 4.46 | 4.46 | 4.46 | 4.46 | 4.46 |
| scaffold_82367_curated\| | | | | | | | | |
| 1523 . . . 3856\|revcom | | | | | | | | |
| Cas14b.16\|3300005573.a\| | 5.254 | 5.329 | 5.329 | 5.344 | 5.344 | 5.344 | 5.344 | 5.344 |
| Ga0078972_1001015a\| | | | | | | | | |
| 33750 . . . 35627 | | | | | | | | |
| Cas14b.10\|CG08_land_{—} | 4.586 | 4.301 | 4.301 | 4.312 | 4.312 | 4.312 | 4.312 | 4.312 |
| 8_20_14_0.20_{—} | | | | | | | | |
| scaffold_1609_curated\| | | | | | | | | |
| 6134 . . . 7975 | | | | | | | | |
| Cas14b.11\|CG_4_10_{—} | 4.711 | 5.221 | 5.221 | 4.801 | 4.801 | 4.801 | 4.801 | 4.801 |
| 14_0.8_um_filter_{—} | | | | | | | | |
| scaffold_20762_curated\| | | | | | | | | |
| 1372 . . . 3219 | | | | | | | | |
| Cas14u.1\|3300009029.a\| | 4.085 | 5.142 | 5.142 | 4.265 | 4.265 | 4.265 | 4.265 | 4.265 |
| Ga0066793_10010091\| | | | | | | | | |
| 37 . . . 1113\|revcom | | | | | | | | |
| Cas12c1 | 3.952 | 3.571 | 3.571 | 4.124 | 4.124 | 4.124 | 4.124 | 4.124 |
| Cas12c2 | 6.334 | 6.796 | 6.796 | 6.796 | 6.796 | 6.796 | 6.791 | 6.791 |
| Cas12a_UPI001113398F | 6.809 | 6.507 | 6.507 | 6.274 | 6.274 | 6.274 | 6.274 | 6.274 |
| Cas12b_UPI001113398F | 93.916 | 52.754 | 52.754 | 51.817 | 51.817 | 51.817 | 51.557 | 51.557 |
| Cas12b_tr\|A0A1I7F1U9\| | 93.916 | 52.754 | 52.754 | 51.817 | 51.817 | 51.817 | 51.557 | 51.557 |
| A0A1I7F1U9_9BACL | | | | | | | | |
| Cas12a_UPI00083514A7 | | 50.676 | 50.676 | 49.661 | 49.661 | 49.661 | 49.407 | 49.407 |
| Cas12b_UPI00083514A7 | 50.676 | | 100 | 55.45 | 55.45 | 55.45 | 55.19 | 55.19 |
| Cas12a_UPI00097159F1 | 50.676 | 100 | | 55.45 | 55.45 | 55.45 | 55.19 | 55.19 |
| Cas12b_UPI00097159F1 | 49.661 | 55.45 | 55.45 | | 100 | 100 | 99.734 | 99.734 |
| Cas12b_sp\|T0D7A2\| | 49.661 | 55.45 | 55.45 | 100 | | 100 | 99.734 | 99.734 |
| CS12B_ALIAG | | | | | | | | |
| Cas12a_UPI0009715A14 | 49.661 | 55.45 | 55.45 | 100 | 100 | | 99.734 | 99.734 |
| Cas12b_UPI0009715A14 | 49.407 | 55.19 | 55.19 | 99.734 | 99.734 | 99.734 | | 100 |
| Cas12a_UPI00097159CF | 49.407 | 55.19 | 55.19 | 99.734 | 99.734 | 99.734 | 100 | |
| Cas12b_UPI00097159CF | 49.576 | 55.363 | 55.363 | 99.911 | 99.911 | 99.911 | 99.823 | 99.823 |
| Cas12a_UPI000832F6D2 | 49.576 | 55.363 | 55.363 | 99.911 | 99.911 | 99.911 | 99.823 | 99.823 |
| Cas12b_UPI000832F6D2 | 49.619 | 55.796 | 55.796 | 93.546 | 93.546 | 93.546 | 93.28 | 93.28 |
| Cas12b_tr\|A0A512CSX2\| | 49.619 | 55.796 | 55.796 | 93.546 | 93.546 | 93.546 | 93.28 | 93.28 |
| A0A512CSX2_9BACL | | | | | | | | |
| OspCas12c | 49.619 | 55.969 | 55.969 | 92.838 | 92.838 | 92.838 | 92.573 | 92.573 |
| Cas14u.5\|3300012532.a\| | 5.283 | 6.169 | 6.169 | 5.864 | 5.864 | 5.864 | 5.864 | 5.864 |
| Ga0137373_10000316\| | | | | | | | | |
| 3286 . . . 5286 | | | | | | | | |
| 63461_4106_protein_locus_{—} | 5.42 | 5.121 | 5.121 | 5.796 | 5.796 | 5.796 | 5.796 | 5.796 |
| of_contig_LSKL01000323 - | | | | | | | | |
| Query protein (63461_4106) | | | | | | | | |
| translation (4) | | | | | | | | |
| 58610_1188_protein_locus_{—} | 4.914 | 4.163 | 4.163 | 5.005 | 5.005 | 5.005 | 5.097 | 5.097 |
| of_contig_LFOD01000003 - | | | | | | | | |
| Query protein (58610_1188) | | | | | | | | |
| translation (5) | | | | | | | | |
| 21566_3969_protein_locus_{—} | 5.027 | 4.277 | 4.277 | 4.753 | 4.753 | 4.753 | 4.753 | 4.753 |
| of_contig_BAFB01000202 - | | | | | | | | |
| Query protein (21566_3969) | | | | | | | | |
| translation (4) | | | | | | | | |
| 21566_3969_protein_locus_{—} | 5.1 | 4.628 | 4.628 | 3.993 | 3.993 | 3.993 | 3.9 | 3.9 |
| of_contig_BAFB01000202_- | | | | | | | | |
| _Queryprotein(21566_3969) | | | | | | | | |
| translation_(4) | | | | | | | | |

TABLE 12

| | | | | | | | | 63461_4106_{—} |
|---|---|---|---|---|---|---|---|---|
| | | | | | | | | protein_locus_{—} |
| | | | | | | | Cas14u.5\| | of_contig_{—} |
| | | | | | Cas12b_tr\| | | 3312532.a\| | LSKL01323 - |
| | | | | | A0A512CSX2\| | | Ga0137373_{—} | Query_protein |
| | Cas12a_{—} | Cas12b_{—} | Cas12a_{—} | Cas12b_{—} | A0A512CSX2_{—} | | 10000316\| | (63461_4106) |
| | UPI00097159CF | UPI00097159CF | UPI000832F6D2 | UPI000832F6D2 | 9BACL | OspCas12c | 3286 . . . 5286 | translation (4) |
| Cas14g.1\|RBG_13_{—} | 4.865 | 4.865 | 4.861 | 4.861 | 5.122 | 5.082 | 6.658 | 5.931 |
| scaffold_1401_curated\| | | | | | | | | |
| 15949 . . . 18180 | | | | | | | | |
| Cas14g.2\|3300009652.a\| | 6.396 | 6.396 | 6.218 | 6.218 | 5.959 | 6.075 | 8.752 | 7.333 |
| Ga0123330_1010394\| | | | | | | | | |
| 2814 . . . 5123 | | | | | | | | |
| Cas12i2 | 6.03 | 6.03 | 6.114 | 6.114 | 5.946 | 5.914 | 4.39 | 3.933 |
| Cas12i1 | 5.934 | 5.934 | 6.008 | 6.008 | 5.692 | 5.588 | 4.128 | 2.982 |
| Cas12g1 | 5.935 | 5.935 | 5.75 | 5.75 | 5.93 | 5.657 | 9.103 | 6.91 |
| Cas14d.3\|RIFCSPLOWO2_{—} | 5.1 | 5.1 | 5.369 | 5.369 | 5.096 | 5.251 | 8.21 | 7.211 |
| 01_FULL_OD1_45_34b_{—} | | | | | | | | |
| rifcsplowo2_01_{—} | | | | | | | | |
| scaffold_3495_curated\| | | | | | | | | |
| 25656 . . . 27605\|revcom | | | | | | | | |
| Cas14d.1\|RIFCSPHIGHO2_{—} | 5.743 | 5.743 | 6.011 | 6.011 | 6.011 | 3.54 | 7.283 | 6.686 |
| 01_FULL_CPR_46_36_{—} | | | | | | | | |
| rifcsphigho2_01_{—} | | | | | | | | |
| scaffold_646_curated\| | | | | | | | | |
| 49808 . . . 51616\|revcom | | | | | | | | |
| CasY5 | 6.915 | 6.915 | 6.843 | 6.843 | 7.076 | 4.853 | 5.804 | 4.204 |
| Cas14a.4\|CG10_big_fil_{—} | 5.165 | 5.165 | 5.33 | 5.33 | 5.161 | 4.021 | 6.591 | 5.284 |
| rev_8_21_14_0.10_{—} | | | | | | | | |
| scaffold_20906_curated\| | | | | | | | | |
| 649 . . . 2829 | | | | | | | | |
| CasY6 | 6.133 | 6.133 | 6.502 | 6.502 | 6.353 | 7.595 | 5.418 | 3.692 |
| Cas14f.1\|rifcsp13_1_sub10_{—} | 6.324 | 6.324 | 6.416 | 6.416 | 6.416 | 5.314 | 6.436 | 7.015 |
| scaffold_3_curated\| | | | | | | | | |
| 38906 . . . 41041 | | | | | | | | |
| Cas14f.2\|3300009991.a\| | 4.558 | 4.558 | 4.831 | 4.831 | 4.649 | 4.073 | 5.503 | 7.794 |
| Ga0105042_100140\| | | | | | | | | |
| 1624 . . . 3348 | | | | | | | | |
| Cas14a.6\|3300012359.a\| | 2.69 | 2.69 | 2.966 | 2.966 | 2.966 | 3.471 | 6.078 | 5.063 |
| Ga0137385_10000156\| | | | | | | | | |
| 41289 . . . 42734 | | | | | | | | |
| Cas12a_UPI00094EEDB4 | 6.82 | 6.82 | 6.671 | 6.671 | 6.671 | 6.104 | 3.74 | 2.937 |
| Cas12a_UPI000B4235CE | 6.017 | 6.017 | 5.87 | 5.87 | 5.941 | 7.567 | 4.064 | 3.303 |
| Cas12a_UPI000818CC52 | 5.882 | 5.882 | 5.735 | 5.735 | 5.806 | 7.436 | 4.064 | 3.303 |
| Cas12a_UPI0007B78B7F | 5.874 | 5.874 | 5.727 | 5.727 | 5.798 | 7.426 | 4.064 | 3.303 |
| Cas12a_UPI000B4235F9 | 5.946 | 5.946 | 5.798 | 5.798 | 5.87 | 7.567 | 4.064 | 3.303 |
| Cas14e.2\|rifcsplowo2_01_{—} | 4.03 | 4.03 | 4.213 | 4.213 | 4.213 | 2.922 | 4.154 | 5.096 |
| scaffold_81231_curated\| | | | | | | | | |
| 976 . . . 2217 | | | | | | | | |
| Cas14e.1\|rifcsphigho2_01_{—} | 3.825 | 3.825 | 3.918 | 3.918 | 3.731 | 3.084 | 6.37 | 5.949 |
| scaffold_566_curated\| | | | | | | | | |
| 113069 . . . 114313 | | | | | | | | |
| Cas14e.3\|rifcsphigho2_01_{—} | 5.717 | 5.717 | 5.524 | 5.524 | 5.337 | 3.328 | 6.038 | 5.512 |
| scaffold_4702_curated\| | | | | | | | | |
| 82881 . . . 84230\|revcom | | | | | | | | |
| CasY4 | 6.074 | 6.074 | 6.226 | 6.226 | 6.302 | 5.58 | 5.068 | 4.017 |
| Cas14h.3\|3300009698.a\| | 4.122 | 4.122 | 3.939 | 3.939 | 4.029 | 3.325 | 6.96 | 6.192 |
| Ga0116216_10000905\| | | | | | | | | |
| 8005 . . . 9504 | | | | | | | | |
| Cas14h.1\|3300005602.a\| | 5.225 | 5.225 | 5.316 | 5.316 | 5.316 | 4.133 | 9.531 | 7.657 |
| Ga0070762_10001740\| | | | | | | | | |
| 7377 . . . 9071\|revcom | | | | | | | | |
| Cas14h.2\|3300005921.a\| | 5.133 | 5.133 | 5.225 | 5.225 | 5.133 | 4.708 | 8.417 | 7.055 |
| Ga0070766_10011912\| | | | | | | | | |
| 384 . . . 2081 | | | | | | | | |
| Cas14c.1\|CG10_big_fil_{—} | 4.452 | 4.452 | 4.27 | 4.27 | 4.27 | 3.503 | 4.032 | 4.928 |
| rev_8_21_14_0.10_{—} | | | | | | | | |
| scaffold_4477_curated\| | | | | | | | | |
| 19327 . . . 20880\|revcom | | | | | | | | |
| Cas12h1 | 6.374 | 6.374 | 5.938 | 5.938 | 5.766 | 5.263 | 6.749 | 6.082 |
| CasX1 | 5.916 | 5.916 | 6.076 | 6.076 | 5.993 | 5.792 | 6.016 | 4.187 |
| CasX2 | 5.412 | 5.412 | 5.74 | 5.74 | 5.657 | 6.386 | 5.731 | 5.348 |
| CasY1 | 4.867 | 4.867 | 5.102 | 5.102 | 5.102 | 6.691 | 5.818 | 3.931 |
| Cas14u.3\|19ft_2_nophage_{—} | 4.457 | 4.457 | 4.731 | 4.731 | 4.453 | 4.214 | 6.287 | 7.981 |
| noknown_scaffold_0_curated\| | | | | | | | | |
| 508188 . . . 59648 | | | | | | | | |
| Cas14u.7\|3300001256.a\| | 3.306 | 3.306 | 3.394 | 3.394 | 3.394 | 3.339 | 5.589 | 4.754 |
| JGI12210J13797_10004690\| | | | | | | | | |
| 5792 . . . 7006 | | | | | | | | |
| Cas14u.8\|3300005660.a\| | 2.661 | 2.661 | 2.75 | 2.75 | 2.841 | 3.496 | 6.938 | 7.084 |
| Ga0073904_10021651\| | | | | | | | | |
| 765 . . . 1943 | | | | | | | | |
| Cas14u.4\|rifcsp2_19_4_full_{—} | 2.663 | 2.663 | 2.849 | 2.849 | 2.755 | 2.685 | 5.556 | 5.307 |
| scaffold_168_curated\| | | | | | | | | |
| 84455 . . . 85657 | | | | | | | | |
| Cas14d.2\|rifcsphigho2_01_{—} | 4.503 | 4.503 | 4.592 | 4.592 | 4.592 | 3.504 | 5.588 | 6.907 |
| scaffold_10981_curated\| | | | | | | | | |
| 5762 . . . 7246\|revcom | | | | | | | | |
| Cas14c.2\|3300001245.a\| | 3.294 | 3.294 | 3.294 | 3.294 | 3.294 | 3.89 | 6.577 | 6.743 |
| JGI12048J13642_10201286\| | | | | | | | | |
| 4257 . . . 5489\|revcom | | | | | | | | |
| CasY3 | 6.298 | 6.298 | 6.523 | 6.523 | 6.37 | 7.179 | 4.038 | 3.362 |
| 633299_527_protein_locus_{—} | 3.578 | 3.578 | 3.483 | 3.483 | 3.391 | 2.941 | 5.918 | 6.988 |
| of_contig_Scfld15 - Query | | | | | | | | |
| protein (633299_527) (4) | | | | | | | | |
| 8971_2857_protein_locus_{—} | 2.242 | 2.242 | 2.045 | 2.045 | 2.142 | 3.38 | 6.988 | 5.302 |
| of_contig_OEJQ01000083.1 - | | | | | | | | |
| Query protein (8971_2857) | | | | | | | | |
| 9265_901_protein_locus_{—} | 2.314 | 2.314 | 2.119 | 2.119 | 2.216 | 3.519 | 7.026 | 5.197 |
| of_contig_OEFX01000005.1 - | | | | | | | | |
| Query protein (9265_901) | | | | | | | | |
| Cas14u.6\|3300006028.a\| | 4.428 | 4.428 | 4.7 | 4.7 | 4.885 | 4.217 | 8.626 | 8.15 |
| Ga0070717_10000077\| | | | | | | | | |
| 54519 . . . 56201\|revcom | | | | | | | | |
| 466065_250_protein_locus_{—} | 2.844 | 2.844 | 2.746 | 2.746 | 2.841 | 3.859 | 6.991 | 5.351 |
| of_contig_SFKR01000004.1 - | | | | | | | | |
| Query protein (466065_250) | | | | | | | | |
| Cas14a.5\|rifcsplowo2_01_{—} | 5.302 | 5.302 | 5.297 | 5.297 | 5.205 | 2.885 | 4.119 | 5.14 |
| scaffold_34461_curated\| | | | | | | | | |
| 4968 . . . 6521 | | | | | | | | |
| CasY2 | 6.656 | 6.656 | 6.886 | 6.886 | 6.58 | 5.808 | 4.227 | 4.503 |
| Cas14a.3\|gwa1_{—} | 4.69 | 4.69 | 4.592 | 4.592 | 4.686 | 4.327 | 7.225 | 9.451 |
| scaffold_1795_curated\| | | | | | | | | |
| 25635 . . . 27224\|revcom | | | | | | | | |
| Cas14a.1\|rifcsphigho2_02_{—} | 4.944 | 4.944 | 4.846 | 4.846 | 4.939 | 4.302 | 6.755 | 6.656 |
| scaffold_2167_curated\| | | | | | | | | |
| 30296 . . . 31798\|revcom | | | | | | | | |
| Cas14a.2\|gwa2_{—} | 5.097 | 5.097 | 4.907 | 4.907 | 5.093 | 4.383 | 6.461 | 6.815 |
| scaffold_18027_curated\| | | | | | | | | |
| 7105 . . . 8628 | | | | | | | | |
| Cas14b.4\|cg1_0.2_{—} | 3.911 | 3.911 | 3.814 | 3.814 | 3.907 | 4.475 | 8.346 | 7.309 |
| scaffold_785_c_curated\| | | | | | | | | |
| 32521 . . . 34155 | | | | | | | | |
| Cas14b.7\|3300013125.a\| | 4.735 | 4.735 | 4.36 | 4.36 | 4.267 | 4.302 | 8.453 | 7.883 |
| Ga0172369_10000737\| | | | | | | | | |
| 994 . . . 2652\|revcom | | | | | | | | |
| Cas14u.2\|3300002172.a\| | 2.796 | 2.796 | 2.889 | 2.889 | 2.889 | 3.358 | 6.697 | 7.5 |
| JGI24730J26740_1002785\| | | | | | | | | |
| 496 . . . 1605\|revcom | | | | | | | | |
| Cas14b.3\|rifcsphigho2_01_{—} | 4.186 | 4.186 | 4.089 | 4.089 | 4.182 | 5.348 | 6.314 | 7.74 |
| scaffold_36781_curated\| | | | | | | | | |
| 2592 . . . 4217 | | | | | | | | |
| Cas14b.2\|rifcsplowo2_01_{—} | 3.857 | 3.857 | 3.665 | 3.665 | 3.665 | 4.583 | 7.544 | 7.834 |
| scaffold_282_curated\| | | | | | | | | |
| 77370 . . . 78983 | | | | | | | | |
| Cas14b.1\|rifcsplowo2_01_{—} | 4.026 | 4.026 | 3.835 | 3.835 | 3.742 | 5.134 | 6.618 | 7.963 |
| scaffold_239_curated\| | | | | | | | | |
| 54653 . . . 56257 | | | | | | | | |
| Cas14b.8\|3300013125.a\| | 4.588 | 4.588 | 4.303 | 4.303 | 4.21 | 4.971 | 7.038 | 8.129 |
| Ga0172369_10010464\| | | | | | | | | |
| 885 . . . 2489\|revcom | | | | | | | | |
| Cas14b.5\|rifcsphigho2_02_{—} | 4.007 | 4.007 | 4.19 | 4.19 | 4.19 | 5.195 | 6.877 | 7.198 |
| scaffold_55589_curated\| | | | | | | | | |
| 1904 . . . 3598 | | | | | | | | |
| Cas14b.6\|CG03_land_{—} | 3.85 | 3.85 | 4.029 | 4.029 | 4.304 | 6.667 | 5.698 | 7.38 |
| 8_20_14_0.80_{—} | | | | | | | | |
| scaffold_2214_curated\| | | | | | | | | |
| 6634 . . . 8466\|revcom | | | | | | | | |
| Cas14b.9\|3300013127.a\| | 4.66 | 4.66 | 5.028 | 5.028 | 5.307 | 5.537 | 8.213 | 8.756 |
| Ga0172365_10004421\| | | | | | | | | |
| 633 . . . 2366\|revcom | | | | | | | | |
| 209658_13971_protein_{—} | 2.966 | 2.966 | 2.962 | 2.962 | 2.962 | 4.028 | 5.056 | 6.681 |
| locus_of_contig_Ga0190333_{—} | | | | | | | | |
| 1001561 - Query protein | | | | | | | | |
| (209658_13971) (2) | | | | | | | | |
| 209657_57738_protein_{—} | 5.698 | 5.698 | 5.966 | 5.966 | 5.966 | 7.71 | 9.412 | 9.449 |
| locus_of_contig_Ga0190332_{—} | | | | | | | | |
| 1015597 - Query protein | | | | | | | | |
| (209657_57738) (2) | | | | | | | | |
| 209660_51257_protein_{—} | 5.935 | 5.935 | 6.213 | 6.213 | 6.213 | 7.477 | 10.084 | 9.877 |
| locus_of_contig_Ga0190335_{—} | | | | | | | | |
| 1015156 - Query protein | | | | | | | | |
| (209660_51257) (2) | | | | | | | | |
| Cas14b.14\|gwc1_{—} | 4.626 | 4.626 | 4.8 | 4.8 | 4.711 | 4.309 | 5.078 | 4.762 |
| scaffold_8732_curated\| | | | | | | | | |
| 2705 . . . 4537 | | | | | | | | |
| Cas14b.15\|3300010293.a\| | 5.316 | 5.316 | 5.128 | 5.128 | 4.945 | 5.263 | 6.46 | 5.532 |
| Ga0116204_1008574\| | | | | | | | | |
| 2134 . . . 4032 | | | | | | | | |
| Cas14b.12\|CG22_combo_{—} | 4.46 | 4.46 | 4.799 | 4.799 | 4.713 | 5.016 | 5.788 | 5.388 |
| CG10-13_8_21_14_all_{—} | | | | | | | | |
| scaffold_2003_curated\| | | | | | | | | |
| 553 . . . 2880\|revcom | | | | | | | | |
| Cas14b.13\|rifcsphigho2_01_{—} | 5.344 | 5.344 | 5.254 | 5.254 | 5.508 | 4.71 | 4.436 | 4.326 |
| scaffold_82367_curated\| | | | | | | | | |
| 1523 . . . 3856\|revcom | | | | | | | | |
| Cas14b.16\|3300005573.a\| | 4.312 | 4.312 | 4.216 | 4.216 | 4.216 | 4.835 | 5.501 | 6.021 |
| Ga0078972_1001015a\| | | | | | | | | |
| 33750 . . . 35627 | | | | | | | | |
| Cas14b.10\|CG08_land_{—} | 4.801 | 4.801 | 4.887 | 4.887 | 4.615 | 4.75 | 7.203 | 6.466 |
| 8_20_14_0.20_{—} | | | | | | | | |
| scaffold_1609_curated\| | | | | | | | | |
| 6134 . . . 7975 | | | | | | | | |
| Cas14b.11\|CG_4_10_{—} | 4.265 | 4.265 | 4.533 | 4.533 | 4.352 | 4.593 | 7.433 | 7.292 |
| 14_0.8_um_filter_{—} | | | | | | | | |
| scaffold_20762_curated\| | | | | | | | | |
| 1372 . . . 3219 | | | | | | | | |
| Cas14u.1\|3300009029.a\| | 4.124 | 4.124 | 4.221 | 4.221 | 4.311 | 3.102 | 5.706 | 7.19 |
| Ga0066793_10010091\| | | | | | | | | |
| 37 . . . 1113\|revcom | | | | | | | | |
| Cas12c1 | 6.791 | 6.791 | 6.572 | 6.572 | 6.497 | 7.138 | 3.739 | 3.262 |
| Cas12c2 | 6.274 | 6.274 | 6.042 | 6.042 | 5.887 | 7.704 | 4.269 | 3.621 |
| Cas12a_UPI001113398F | 51.73 | 51.73 | 51.513 | 51.513 | 51.685 | 5.243 | 5.596 | 4.818 |
| Cas12b_UPI001113398F | 51.73 | 51.73 | 51.513 | 51.513 | 51.685 | 5.243 | 5.596 | 4.818 |
| Cas12b_tr\|A0A1I7F1U9\| | 49.576 | 49.576 | 49.619 | 49.619 | 49.619 | 5.283 | 5.42 | 4.914 |
| A0A1I7F1U9_9BACL | | | | | | | | |
| Cas12a_UPI00083514A7 | 55.363 | 55.363 | 55.796 | 55.796 | 55.969 | 6.169 | 5.121 | 4.163 |
| Cas12b_UPI00083514A7 | 55.363 | 55.363 | 55.796 | 55.796 | 55.969 | 6.169 | 5.121 | 4.163 |
| Cas12a_UPI00097159F1 | 99.911 | 99.911 | 93.546 | 93.546 | 92.838 | 5.864 | 5.796 | 5.005 |
| Cas12b_UPI00097159F1 | 99.911 | 99.911 | 93.546 | 93.546 | 92.838 | 5.864 | 5.796 | 5.005 |
| Cas12b_sp\|T0D7A2\| | 99.911 | 99.911 | 93.546 | 93.546 | 92.838 | 5.864 | 5.796 | 5.005 |
| CS12B_ALIAG | | | | | | | | |
| Cas12a_UPI0009715A14 | 99.823 | 99.823 | 93.28 | 93.28 | 92.573 | 5.864 | 5.796 | 5.097 |
| Cas12b_UPI0009715A14 | 99.823 | 99.823 | 93.28 | 93.28 | 92.573 | 5.864 | 5.796 | 5.097 |
| Cas12a_UPI00097159CF | | 100 | 93.457 | 93.457 | 92.75 | 5.864 | 5.796 | 5.097 |
| Cas12b_UPI00097159CF | 100 | | 93.457 | 93.457 | 92.75 | 5.864 | 5.796 | 5.097 |
| Cas12a_UPI000832F6D2 | 93.457 | 93.457 | | 100 | 95.664 | 5.941 | 5.974 | 4.727 |
| Cas12b_UPI000832F6D2 | 93.457 | 93.457 | 100 | | 95.664 | 5.941 | 5.974 | 4.727 |
| Cas12b_tr\|A0A512CSX2\| | 92.75 | 92.75 | 95.664 | 95.664 | | 5.788 | 5.79 | 4.912 |
| A0A512CSX2_9BACL | | | | | | | | |
| OspCas12c | 5.864 | 5.864 | 5.941 | 5.941 | 5.788 | | 3.769 | 3.395 |
| Cas14u.5\|3300012532.a\| | 5.796 | 5.796 | 5.974 | 5.974 | 5.79 | 3.769 | | 21.912 |
| Ga0137373_10000316\| | | | | | | | | |
| 3286 . . . 5286 | | | | | | | | |
| 63461_4106_protein_locus_{—} | 5.097 | 5.097 | 4.727 | 4.727 | 4.912 | 3.395 | 21.912 | |
| of_contig_LSKL01000323 - | | | | | | | | |
| Query protein (63461_4106) | | | | | | | | |
| translation (4) | | | | | | | | |
| 58610_1188_protein_locus_{—} | 4.753 | 4.753 | 4.66 | 4.66 | 4.753 | 3.325 | 21.358 | 38.208 |
| of_contig_LFOD01000003 - | | | | | | | | |
| Query protein (58610_1188) | | | | | | | | |
| translation (5) | | | | | | | | |
| 21566_3969_protein_locus_{—} | 3.9 | 3.9 | 3.993 | 3.993 | 4.085 | 4.065 | 23.547 | 36.783 |
| of_contig_BAFB01000202 - | | | | | | | | |
| Query protein (21566_3969) | | | | | | | | |
| translation (4) | | | | | | | | |

TABLE 13

| | 58610_1188_protein_{—} | 21566_3969_protein_{—} |
|---|---|---|
| | locus_of_contig_LFO | locus_of_contig_BAFB |
| | D01000003 - Query | 01000202 - Query |
| | protein (58610_1188) | protein (21566_3969) |
| | translation (5) | translation (4) |
| Cas14g.1\|RBG_13_scaffold_1401_curated\|15949 . . . | 6.989 | 6.465 |
| 18180 | | |
| Cas14g.2\|3300009652.a\|Ga0123330_{—} | 8.614 | 7.995 |
| 1010394\|2814 . . . 5123 | | |
| Cas12i2 | 3.599 | 3.937 |
| Cas12i1 | 3.458 | 3.451 |
| Cas12g1 | 6.914 | 8.56 |
| Cas14d.3\|RIFCSPLOWO2_01_FULL_OD1_45_34b_{—} | 7.487 | 6.098 |
| rifcsplowo2_01_scaffold_3495_curated\|25656 . . . | | |
| 27605\|revcom | | |
| Cas14d.1\|RIFCSPHIGHO2_01_FULL_CPR_46 | 7.55 | 6.676 |
| 36_rifcsphigho2_01_scaffold_646_curated\|49808 . . . | | |
| 51616\|revcom | | |
| CasY5 | 4.856 | 4.668 |
| Cas14a.4\|CG10_big_fil_rev_8_21_14_0.10_scaffold_{—} | 7.097 | 6.684 |
| 20906_curated\|649 . . . 2829 | | |
| CasY6 | 3.668 | 3.462 |
| Cas14f.1\|rifcsp13_1_sub10_scaffold_3_{—} | 6.435 | 5.92 |
| curated\|38906 . . . 41041 | | |
| Cas14f.2\|3300009991.a\|Ga0105042_{—} | 6.984 | 6.726 |
| 100140\|1624 . . . 3348 | | |
| Cas14a.6\|3300012359.a\|Ga0137385_{—} | 5.91 | 6.171 |
| 10000156\|41289 . . . 42734 | | |
| Cas12a_UPI00094EEDB4 | 4.321 | 3.181 |
| Cas12a_UPI000B4235CE | 3.988 | 3.627 |
| Cas12a_UPI000818CC52 | 3.988 | 3.627 |
| Cas12a_UPI0007B78B7F | 3.988 | 3.627 |
| Cas12a_UPI000B4235F9 | 3.988 | 3.627 |
| Cas14e.2\|rifcsplowo2_01_scaffold_81231_{—} | 4.416 | 5.76 |
| curated\|976 . . . 2217 | | |
| Cas14e.1\|rifcsphigho2_01_scaffold_566_{—} | 6.19 | 6.924 |
| curated\|113069 . . . 114313 | | |
| Cas14e.3\|rifcsphigho2_01_scaffold_4702_{—} | 4.212 | 4.944 |
| curated\|82881 . . . 84230\|revcom | | |
| CasY4 | 4.693 | 4.014 |
| Cas14h.3\|3300009698.a\|Ga0116216_{—} | 7.099 | 8.791 |
| 10000905\|8005 . . . 9504 | | |
| Cas14h.1\|3300005602.a\|Ga0070762_{—} | 8.769 | 7.351 |
| 10001740\|7377 . . . 9071\|revcom | | |
| Cas14h.2\|3300005921.a\|Ga0070766_{—} | 7.154 | 7.87 |
| 10011912\|384 . . . 2081 | | |
| Cas14c.1\|CG10_big_fil_rev_8_21_14_0.10_scaffold_{—} | 5.24 | 5.294 |
| 4477_curated\|19327 . . . 20880\|revcom | | |
| Cas12h1 | 6.176 | 6.007 |
| CasX1 | 5.123 | 4.266 |
| CasX2 | 5.184 | 4.418 |
| CasY1 | 4.182 | 4.771 |
| Cas14u.3\|19ft_2_nophage_noknown_scaffold_0_{—} | 6.955 | 7.442 |
| curated\|508188 . . . 509648 | | |
| Cas14u.7\|3300001256.a\|JGI12210J13797_{—} | 6.139 | 5.785 |
| 10004690\|5792 . . . 7006 | | |
| Cas14u.8\|3300005660.a\|Ga0073904_{—} | 7.792 | 6.988 |
| 10021651\|765 . . . 1943 | | |
| Cas14u.4\|rifcsp2_19_4_full_scaffold_168_{—} | 4.693 | 5.473 |
| curated\|84455 . . . 85657 | | |
| Cas14d.2\|rifcsphigho2_01_scaffold_10981_{—} | 7.121 | 5.643 |
| curated\|5762 . . . 7246\|revcom | | |
| Cas14c.2\|3300001245.a\|JGI12048J13642_{—} | 7.27 | 7.82 |
| 10201286\|4257 . . . 5489\|revcom | | |
| CasY3 | 3.531 | 2.431 |
| 633299_527_protein_locus_of_contig_Scfld15 - | 7.143 | 6.425 |
| Query protein (633299_527) (4) | | |
| 8971_2857_protein_locus_of_contig_OEJQ01000083.1 - | 6.329 | 5.935 |
| Query protein (8971_2857) | | |
| 9265_901_protein_locus_of_contig_OEFX01000005.1 - | 6.206 | 5.82 |
| Query protein (9265_901) | | |
| Cas14u.6\|3300006028.a\|Ga0070717_{—} | 8.423 | 7.402 |
| 10000077\|54519 . . . 56201\|revcom | | |
| 466065_250_protein_locus_of_contig_SFKR01000004.1 - | 6.931 | 6.187 |
| Query protein (466065_250) | | |
| Cas14a.5\|rifcsplowo2_01_scaffold_34461_{—} | 4.695 | 4.409 |
| curated\|4968 . . . 6521 | | |
| CasY2 | 3.976 | 4.174 |
| Cas14a.3\|gwa1_scaffold_1795_curated\|25635 . . . | 6.577 | 7.553 |
| 27224\|revcom | | |
| Cas14a.1\|rifcsphigho2_02_scaffold_2167_{—} | 6.211 | 6.667 |
| curated\|30296 . . . 31798\|revcom | | |
| Cas14a.2\|gwa2_scaffold_18027_curated\|7105 . . . | 5.745 | 7.302 |
| 8628 | | |
| Cas14b.4\|cg1_0.2_scaffold_785_c_{—} | 5.828 | 6.202 |
| curated\|32521 . . . 34155 | | |
| Cas14b.7\|3300013125.a\|Ga0172369_{—} | 7.023 | 6.583 |
| 10000737\|994 . . . 2652\|revcom | | |
| Cas14u.2\|3300002172.a\|JGI24730J26740_{—} | 8.007 | 8.789 |
| 1002785\|496 . . . 1605\|revcom | | |
| Cas14b.3\|rifcsphigho2_01_scaffold_36781_{—} | 7.317 | 5.376 |
| curated\|2592 . . . 4217 | | |
| Cas14b.2\|rifcsplowo2_01_scaffold_282_{—} | 6.787 | 7.492 |
| curated\|77370 . . . 78983 | | |
| Cas14b.1\|rifcsplowo2_01_scaffold_239_{—} | 7.681 | 7.187 |
| curated\|54653 . . . 56257 | | |
| Cas14b.8\|3300013125.a\|Ga0172369_{—} | 6.949 | 6.585 |
| 10010464\|885 . . . 2489\|revcom | | |
| Cas14b.5\|rifcsphigho2_02_scaffold_55589_{—} | 6.949 | 7.309 |
| curated\|1904 . . . 3598 | | |
| Cas14b.6\|CG03_land_8_20_14_0.80_scaffold_2214_{—} | 7.887 | 6.994 |
| curated\|6634 . . . 8466\|revcom | | |
| Cas14b.9\|3300013127.a\|Ga0172365_{—} | 5.615 | 6.175 |
| 10004421\|633 . . . 2366\|revcom | | |
| 209658_13971_protein_locus_of_contig_Ga0190333_{—} | 5.749 | 6.098 |
| 1001561 - Query protein (209658_13971) (2) | | |
| 209657_57738_protein_locus_of_contig_Ga0190332_{—} | 8.365 | 8.812 |
| 1015597 - Query protein (209657_57738) (2) | | |
| 209660_51257_protein_locus_of_contig_Ga0190335_{—} | 8.13 | 8.8 |
| 1015156 - Query protein (209660_51257) (2) | | |
| Cas14b.14\|gwc1_scaffold_8732_curated\|2705 . . . | 5.321 | 6.241 |
| 4537 | | |
| Cas14b.15\|3300010293.a\|Ga0116204_{—} | 6.601 | 6.268 |
| 1008574\|2134 . . . 4032 | | |
| Cas14b.12\|CG22_combo_CG10-13_8_21_14_all_scaffold_{—} | 4.316 | 5.604 |
| 2003_curated\|553 . . . 2880\|revcom | | |
| Cas14b.13\|rifcsphigho2_01_scaffold_82367_{—} | 5.179 | 5.062 |
| curated\|1523 . . . 3856\|revcom | | |
| Cas14b.16\|3300005573.a\|Ga0078972_{—} | 6.676 | 5.333 |
| 1001015a\|33750 . . . 35627 | | |
| Cas14b.10\|CG08_land_8_20_14_0.20_scaffold_1609_{—} | 6.686 | 7.669 |
| curated\|6134 . . . 7975 | | |
| Cas14b.11\|CG_4_10_14_0.8_um_filter_scaffold_20762_{—} | 6.765 | 6.897 |
| curated\|1372 . . . 3219 | | |
| Cas14u.1\|3300009029.a\|Ga0066793_{—} | 6.139 | 8.086 |
| 10010091\|37 . . . 1113\|revcom | | |
| Cas12c1 | 4.344 | 3.21 |
| Cas12c2 | 4.534 | 4.105 |
| Cas12a_UPI001113398F | 4.932 | 5.105 |
| Cas12b_UPI001113398F | 4.932 | 5.105 |
| Cas12b_tr\|A0A1I7F1U9\|A0A1I7F1U9_9BACL | 5.027 | 5.1 |
| Cas12a_UPI00083514A7 | 4.277 | 4.628 |
| Cas12b_UPI00083514A7 | 4.277 | 4.628 |
| Cas12a_UPI00097159F1 | 4.753 | 3.993 |
| Cas12b_UPI00097159F1 | 4.753 | 3.993 |
| Cas12b_sp\|T0D7A2\|CS12B_ALIAG | 4.753 | 3.993 |
| Cas12a_UPI0009715A14 | 4.753 | 3.9 |
| Cas12b_UPI0009715A14 | 4.753 | 3.9 |
| Cas12a_UPI00097159CF | 4.753 | 3.9 |
| Cas12b_UPI00097159CF | 4.753 | 3.9 |
| Cas12a_UPI000832F6D2 | 4.66 | 3.993 |
| Cas12b_UPI000832F6D2 | 4.66 | 3.993 |
| Cas12b_tr\|A0A512CSX2\|A0A512CSX2_9BACL | 4.753 | 4.085 |
| OspCas12c | 3.325 | 4.065 |
| Cas14u.5\|3300012532.a\|Ga0137373_{—} | 21.358 | 23.547 |
| 10000316\|3286 . . . 5286 | | |
| 63461_4106_protein_locus_of_contig_LSKL01000323 - | 38.208 | 36.783 |
| Query protein (63461_4106) translation (4) | | |
| 58610_1188_protein_locus_of_contig_LFOD01000003 - | | 31.115 |
| Query protein (58610_1188) translation (5) | | |
| 21566_3969_protein_locus_of_contig_BAFB01000202 - | 31.115 | |
| Query protein (21566_3969) translation (4) | | |

TABLE 14
5′ modification

| | |
|---|---|
| SEQ ID NO: 145 | GTTATGCTCCACTTTAATAAGTGGTGCCTTCCAAAGCTATATGCTGAGG |
| 5pr_trunc_4 | GAGGATGGGCGCTGTTGCAGCGTCTGCCCACCTCAGAGTGGGTATCCTT |
| | ACCTATTGAAAAGTAATAGGTCAAGGAATGCAACTGGTTGCCCACCCTA |
| | GTCATTG |
| SEQ ID NO: 146 | GTATGCTCCACTTTAATAAGTGGTGCCTTCCAAAGCTATATGCTGAGGG |
| 5pr_trunc_5 | AGGATGGGCGCTGTTGCAGCGTCTGCCCACCTCAGAGTGGGTATCCTTA |
| | CCTATTGAAAAGTAATAGGTCAAGGAATGCAACTGGTTGCCCACCCTAG |
| | TCATTG |
| SEQ ID NO: 147 | GATGCTCCACTTTAATAAGTGGTGCCTTCCAAAGCTATATGCTGAGGGA |
| 5pr_trunc_6 | GGATGGGCGCTGTTGCAGCGTCTGCCCACCTCAGAGTGGGTATCCTTAC |
| | CTATTGAAAAGTAATAGGTCAAGGAATGCAACTGGTTGCCCACCCTAGT |
| | CATTG |
| SEQ ID NO: 148 | GTCCACTTTAATAAGTGGTGCCTTCCAAAGCTATATGCTGAGGGAGGAT |
| 5pr_trunc_7 | GGGCGCTGTTGCAGCGTCTGCCCACCTCAGAGTGGGTATCCTTACCTAT |
| | TGAAAAGTAATAGGTCAAGGAATGCAACTGGTTGCCCACCCTAGTCATT |
| | G |

SL1_modification

| | |
|---|---|
| SEQ ID NO: 149 | GCTCCGCTTTAATAAGCGGTGCCTTCCAAAGCTATATGCTGAGGGAGGA |
| SL1_modification_1 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |
| SEQ ID NO: 150 | GCTCCACTTTACTAAGTGGTGCCTTCCAAAGCTATATGCTGAGGGAGGA |
| SL1_modification_2 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |
| SEQ ID NO: 151 | GCACCACTTTAATAAGTGGTGCCTTCCAAAGCTATATGCTGAGGGAGGA |
| SL1_modification_3 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |
| SEQ ID NO: 152 | GCTCCACTTTAATAAGTGGAGCCTTCCAAAGCTATATGCTGAGGGAGGA |
| SL1_modification_4 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |
| SEQ ID NO: 153 | GCTCCACTGTAATCAGTGGTGCCTTCCAAAGCTATATGCTGAGGGAGGA |
| SL1_modification_5 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |
| SEQ ID NO: 154 | GTGCTCCACTTTAATAAGTGGTGCATTCCAAAGCTATATGCTGAGGGAG |
| SL1_modification_6 | GATGGGCGCTGTTGCAGCGTCTGCCCACCTCAGAGTGGGTATCCTTACC |
| | TATTGAAAAGTAATAGGTCAAGGAATGCAACTGGTTGCCCACCCTAGTC |
| | ATTG |
| SEQ ID NO: 155 | GCTCCACTTGTAATCAAGTGGTGCCTTCCAAAGCTATATGCTGAGGGAG |
| SL1_modification_7 | GATGGGCGCTGTTGCAGCGTCTGCCCACCTCAGAGTGGGTATCCTTACC |
| | TATTGAAAAGTAATAGGTCAAGGAATGCAACTGGTTGCCCACCCTAGTC |
| | ATTG |
| SEQ ID NO: 156 | GCTCCACTTGCTAATGCAAGTGGTGCCTTCCAAAGCTATATGCTGAGGG |
| SL1_modification_8 | AGGATGGGCGCTGTTGCAGCGTCTGCCCACCTCAGAGTGGGTATCCTTA |
| | CCTATTGAAAAGTAATAGGTCAAGGAATGCAACTGGTTGCCCACCCTAG |
| | TCATTG |
| SEQ ID NO: 157 | GCTCCACTTGGCTAATGCCAAGTGGTGCCTTCCAAAGCTATATGCTGAG |
| SL1_modification_9 | GGAGGATGGGCGCTGTTGCAGCGTCTGCCCACCTCAGAGTGGGTATCCT |
| | TACCTATTGAAAAGTAATAGGTCAAGGAATGCAACTGGTTGCCCACCCT |
| | AGTCATTG |
| SEQ ID NO: 158 | GCTCCACTTGGCATAATTGCCAAGTGGTGCCTTCCAAAGCTATATGCTG |
| SL1_modification_10 | AGGGAGGATGGGCGCTGTTGCAGCGTCTGCCCACCTCAGAGTGGGTAT |
| | CCTTACCTATTGAAAAGTAATAGGTCAAGGAATGCAACTGGTTGCCCAC |
| | CCTAGTCATTG |
| SEQ ID NO: 159 | GCTCCACTTACATGAGGATCACCCATGTAAGTGGTGCCTTCCAAAGCTA |
| SL1_MS2_hp | TATGCTGAGGGAGGATGGGCGCTGTTGCAGCGTCTGCCCACCTCAGAGT |
| | GGGTATCCTTACCTATTGAAAAGTAATAGGTCAAGGAATGCAACTGGTT |
| | GCCCACCCTAGTCATTG |

SL2_modification

| | |
|---|---|
| SEQ ID NO: 160 | GCTCCACTTTAATAAGTGGTGCCTTCCAAAGCTAATGCTGAGGGAGGAT |
| SL2_modification_1 | GGGCGCTGTTGCAGCGTCTGCCCACCTCAGAGTGGGTATCCTTACCTAT |
| | TGAAAAGTAATAGGTCAAGGAATGCAACTGGTTGCCCACCCTAGTCATT |
| | G |
| SEQ ID NO: 161 | GCTCCACTTTAATAAGTGGTGCCTTCCAAAGCTAAATGCTGAGGGAGGA |
| SL2_modification_2 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |
| SEQ ID NO: 162 | GCTCCACTTTAATAAGTGGTGCCTTCCAAAGCCTATATGGCTGAGGGAG |
| SL2_modification_3 | GATGGGCGCTGTTGCAGCGTCTGCCCACCTCAGAGTGGGTATCCTTACC |
| | TATTGAAAAGTAATAGGTCAAGGAATGCAACTGGTTGCCCACCCTAGTC |
| | ATTG |
| SEQ ID NO: 163 | GCTCCACTTTAATAAGTGGTGCCTTCCAAAGCTGTATATCAGCTGAGGG |
| SL2_modification_4 | AGGATGGGCGCTGTTGCAGCGTCTGCCCACCTCAGAGTGGGTATCCTTA |
| | CCTATTGAAAAGTAATAGGTCAAGGAATGCAACTGGTTGCCCACCCTAG |
| | TCATTG |
| SEQ ID NO: 164 | GCTCCACTTTAATAAGTGGTGCCTTCCAAAGCTGCTATATGCAGCTGAG |
| SL2_modification_5 | GGAGGATGGGCGCTGTTGCAGCGTCTGCCCACCTCAGAGTGGGTATCCT |
| | TACCTATTGAAAAGTAATAGGTCAAGGAATGCAACTGGTTGCCCACCCT |
| | AGTCATTG |
| SEQ ID NO: 165 | GCTCCACTTTAATAAGTGGTGCCTTCCAAAGCTGCTTATATAGCAGCTG |
| SL2_modification_6 | AGGGAGGATGGGCGCTGTTGCAGCGTCTGCCCACCTCAGAGTGGGTAT |
| | CCTTACCTATTGAAAAGTAATAGGTCAAGGAATGCAACTGGTTGCCCAC |
| | CCTAGTCATTG |
| SEQ ID NO: 166 | GCTCCACTTTAATAAGTGGTGCCTTCCAAAGCTGCTGTATATCAGCAGC |
| SL2_modification_7 | TGAGGGAGGATGGGCGCTGTTGCAGCGTCTGCCCACCTCAGAGTGGGT |
| | ATCCTTACCTATTGAAAAGTAATAGGTCAAGGAATGCAACTGGTTGCCC |
| | ACCCTAGTCATTG |
| SEQ ID NO: 167 | GCTCCACTTTAATAAGTGGTGCCTTCCAAAGCACATGAGGATCACCCAT |
| SL2_MS2_hp | GTGCTGAGGGAGGATGGGCGCTGTTGCAGCGTCTGCCCACCTCAGAGT |
| | GGGTATCCTTACCTATTGAAAAGTAATAGGTCAAGGAATGCAACTGGTT |
| | GCCCACCCTAGTCATTG |

SL3 modification

| | |
|---|---|
| SEQ ID NO: 168 | GTCCACTTTAATAAGTGGTGCCTTCCAAAGCTATATGCTGAGGGAGGAT |
| increase_interaction_ | GGGCGCTGTTGCAAGCGTCTGCCCACCTCAGAGTGGGTATCCTTACCTA |
| w_crRNA_13 | TTGAAAAGTAATAGGTCAAGGATTGCAACTGGTTGCCCACCCTAGTCAT |
| | TG |
| SEQ ID NO: 169 | GTCCACTTTAATAAGTGGTGCCTTCCAAAGCTATATGCTGAGGGAGGAT |
| increase_interaction_ | GGGCGCTGTTGCACGCGTCTGCCCACCTCAGAGTGGGTATCCTTACCTA |
| w_crRNA_14 | TTGAAAAGTAATAGGTCAAGGAGTGCAACTGGTTGCCCACCCTAGTCAT |
| | TG |
| SEQ ID NO: 170 | GTCCACTTTAATAAGTGGTGCCTTCCAAAGCTATATGCTGAGGGAGGAT |
| increase_interaction_ | GGGCGCTGTTGCAGGCGTCTGCCCACCTCAGAGTGGGTATCCTTACCTA |
| w_crRNA_15 | TTGAAAAGTAATAGGTCAAGGACTGCAACTGGTTGCCCACCCTAGTCAT |
| | TG |
| SEQ ID NO: 171 | GTCCACTTTAATAAGTGGTGCCTTCCAAAGCTATATGCTGAGGGAGGAT |
| increase_interaction_ | GGGCGCTGTTGCATGCGTCTGCCCACCTCAGAGTGGGTATCCTTACCTA |
| w_crRNA_16 | TTGAAAAGTAATAGGTCAAGGAATGCAACTGGTTGCCCACCCTAGTCAT |
| | TG |
| SEQ ID NO: 172 | GTCCACTTTAATAAGTGGTGCCTTCCAAAGCTATATGCTGAGGGAGGAT |
| increase_interaction_ | GGGCGCTGTTCGATGCGTCTGCCCACCTCAGAGTGGGTATCCTTACCTA |
| w_crRNA_17 | TTGAAAAGTAATAGGTCAAGGAATCGAACTGGTTGCCCACCCTAGTCAT |
| | TG |
| SEQ ID NO: 173 | GTCCACTTTAATAAGTGGTGCCTTCCAAAGCTATATGCTGAGGGAGGAT |
| increase_interaction_ | GGGCGCTGTTGAGTGCGTCTGCCCACCTCAGAGTGGGTATCCTTACCTA |
| w_crRNA_18 | TTGAAAAGTAATAGGTCAAGGAACTCAACTGGTTGCCCACCCTAGTCAT |
| | TG |
| SEQ ID NO: 174 | GTCCACTTTAATAAGTGGTGCCTTCCAAAGCTATATGCTGAGGGAGGAT |
| increase_interaction_ | GGGCGCTGTTGCGTGCGTCTGCCCACCTCAGAGTGGGTATCCTTACCTA |
| w_crRNA_19 | TTGAAAAGTAATAGGTCAAGGAACGCAACTGGTTGCCCACCCTAGTCAT |
| | TG |
| SEQ ID NO: 175 | GTCCACTTTAATAAGTGGTGCCTTCCAAAGCTATATGCTGAGGGAGGAT |
| increase_interaction_ | GGGCGCTGTTGTATGCGTCTGCCCACCTCAGAGTGGGTATCCTTACCTA |
| w_crRNA_20 | TTGAAAAGTAATAGGTCAAGGAATACAACTGGTTGCCCACCCTAGTCAT |
| | TG |
| SEQ ID NO: 176 | GTCCACTTTAATAAGTGGTGCCTTCCAAAGCTATATGCTGAGGGAGGAT |
| increase_interaction_ | GGGCGCTGCCGCATGCGTCTGCCCACCTCAGAGTGGGTATCCTTACCTA |
| w_crRNA_21 | TTGAAAAGTAATAGGTCAAGGAATGCGGCTGGTTGCCCACCCTAGTCAT |
| | TG |
| SEQ ID NO: 177 | GTCCACTTTAATAAGTGGTGCCTTCCAAAGCTATATGCTGAGGGAGGAT |
| increase_interaction_ | GGGCGCTGCGGCATGCGTCTGCCCACCTCAGAGTGGGTATCCTTACCTA |
| w_crRNA_22 | TTGAAAAGTAATAGGTCAAGGAATGCCGCTGGTTGCCCACCCTAGTCAT |
| | TG |
| SEQ ID NO: 178 | GTCCACTTTAATAAGTGGTGCCTTCCAAAGCTATATGCTGAGGGAGGAT |
| increase_interaction_ | GGGCGCTGTTGCGGCGTCTGCCCACCTCAGAGTGGGTATCCTTACCTAT |
| w_crRNA_23 | TGAAAAGTAATAGGTCAAGGAACGCAACTGGTTGCCCACCCTAGTCATT |
| | G |
| SEQ ID NO: 179 | GTCCACTTTAATAAGTGGTGCCTTCCAAAGCTATATGCTGAGGGAGGAT |
| increase_interaction_ | GGGCGCTGTTGTAGCGTCTGCCCACCTCAGAGTGGGTATCCTTACCTAT |
| w_crRNA_24 | TGAAAAGTAATAGGTCAAGGAATACAACTGGTTGCCCACCCTAGTCATT |
| | G |
| SEQ ID NO: 180 | GTCCACTTTAATAAGTGGTGCCTTCCAAAGCTATATGCTGAGGGAGGAT |
| increase_interaction_ | GGGCGCTGCCGCAGCGTCTGCCCACCTCAGAGTGGGTATCCTTACCTAT |
| w_crRNA_25 | TGAAAAGTAATAGGTCAAGGAATGCGGCTGGTTGCCCACCCTAGTCATT |
| | G |
| SEQ ID NO: 181 | GTCCACTTTAATAAGTGGTGCCTTCCAAAGCTATATGCTGAGGGAGGAT |
| increase_interaction_ | GGGCGCTGCGGCAGCGTCTGCCCACCTCAGAGTGGGTATCCTTACCTAT |
| w_crRNA_26 | TGAAAAGTAATAGGTCAAGGAATGCCGCTGGTTGCCCACCCTAGTCATT |
| | G |

SL4 modification

| | |
|---|---|
| SEQ ID NO: 182 | GCTCCACTTTAATAAGTGGTGCCTTCCAAAGCTATATGCTGAGGGAGGA |
| increase_interaction_ | TGGGCGCTGTTGCAGCGTCTGCCCACGCTAGACGTGGGTATCCTTACCT |
| of_SL4_3 | ATTGAAAAGTAATAGGTCAAGGAATGCAACTGGTTGCCCACCCTAGTC |
| | ATTG |
| SEQ ID NO: 183 | GCTCCACTTTAATAAGTGGTGCCTTCCAAAGCTATATGCTGAGGGAGGA |
| increase_interaction_ | TGGGCGCTGTTGCAGCGTCTGCCCACTGCTAGACAGTGGGTATCCTTAC |
| of_SL4_4 | CTATTGAAAAGTAATAGGTCAAGGAATGCAACTGGTTGCCCACCCTAGT |
| | CATTG |
| SEQ ID NO: 184 | GCTCCACTTTAATAAGTGGTGCCTTCCAAAGCTATATGCTGAGGGAGGA |
| increase_interaction_ | TGGGCGCTGTTGCAGCGTCTGCCCACCTGCTAGACAGGTGGGTATCCTT |
| of_SL4_5 | ACCTATTGAAAAGTAATAGGTCAAGGAATGCAACTGGTTGCCCACCCTA |
| | GTCATTG |
| SEQ ID NO: 185 | GCTCCACTTTAATAAGTGGTGCCTTCCAAAGCTATATGCTGAGGGAGGA |
| increase_interaction_ | TGGGCGCTGTTGCAGCGTCTGCCCACGCTCAGACGTGGGTATCCTTACC |
| of_SL4_6 | TATTGAAAAGTAATAGGTCAAGGAATGCAACTGGTTGCCCACCCTAGTC |
| | ATTG |
| SEQ ID NO: 186 | GCTCCACTTTAATAAGTGGTGCCTTCCAAAGCTATATGCTGAGGGAGGA |
| increase_interaction_ | TGGGCGCTGTTGCAGCGTCTGCCCACTGCTCAGACAGTGGGTATCCTTA |
| of_SL4_7 | CCTATTGAAAAGTAATAGGTCAAGGAATGCAACTGGTTGCCCACCCTAG |
| | TCATTG |
| SEQ ID NO: 287 | GCTCCACTTTAATAAGTGGTGCCTTCCAAAGCTATATGCTGAGGGAGGA |
| increase_interaction_ | TGGGCGCTGTTGCAGCGTCTGCCCACCTGCTCAGACAGGTGGGTATCCT |
| of_SL4_8 | TACCTATTGAAAAGTAATAGGTCAAGGAATGCAACTGGTTGCCCACCCT |
| | AGTCATTG |
| SEQ ID NO: 187 | GCTCCACTTTAATAAGTGGTGCCTTCCAAAGCTATATGCTGAGGGAGGA |
| increase_interaction_ | TGGGCGCTGTTGCAGCGTCTGCCCACGCTGCTCAGACAGCGTGGGTATC |
| of_SL4_9 | CTTACCTATTGAAAAGTAATAGGTCAAGGAATGCAACTGGTTGCCCACC |
| | CTAGTCATTG |
| SEQ ID NO: 188 | GCTCCACTTTAATAAGTGGTGCCTTCCAAAGCTATATGCTGAGGGAGGA |
| increase_interaction_ | TGGGCGCTGTTGCAGCGTCTGCCCACTGCTGCTCAGACAGCAGTGGGTA |
| of_SL4_10 | TCCTTACCTATTGAAAAGTAATAGGTCAAGGAATGCAACTGGTTGCCCA |
| | CCCTAGTCATTG |
| SEQ ID NO: 189 | GCTCCACTTTAATAAGTGGTGCCTTCCAAAGCTATATGCTGAGGGAGGA |
| SL3_MS2_hp | TGGGCGCTGTTGCAGCGTCTGCCCACACATGAGGATCACCCATGTGTGG |
| | GTATCCTTACCTATTGAAAAGTAATAGGTCAAGGAATGCAACTGGTTGC |
| | CCACCCTAGTCATTG |

SL5 modification

| | |
|---|---|
| SEQ ID NO: 190 | GTCCACTTTAATAAGTGGTGCCTTCCAAAGCTATATGCTGAGGGAGGAT |
| increase_interaction_ | GGGCGCTGTTGCAGCGTCTGCCCACCTCAGAGTGGGTATCCTTACCTAT |
| of_SL5_4 | TAAAAGTAATAGGTCAAGGAATGCAACTGGTTGCCCACCCTAGTCATTG |
| SEQ ID NO: 191 | GTCCACTTTAATAAGTGGTGCCTTCCAAAGCTATATGCTGAGGGAGGAT |
| increase_interaction_ | GGGCGCTGTTGCAGCGTCTGCCCACCTCAGAGTGGGTATCCTTACCTAT |
| of_SL5_5 | TGGAAAAGCTAATAGGTCAAGGAATGCAACTGGTTGCCCACCCTAGTC |
| | ATTG |
| SEQ ID NO: 192 | GTCCACTTTAATAAGTGGTGCCTTCCAAAGCTATATGCTGAGGGAGGAT |
| increase_interaction_ | GGGCGCTGTTGCAGCGTCTGCCCACCTCAGAGTGGGTATCCTTACCTAT |
| of_SL5_6 | TGCTAAAAGAGTAATAGGTCAAGGAATGCAACTGGTTGCCCACCCTAG |
| | TCATTG |
| SEQ ID NO: 193 | GTCCACTTTAATAAGTGGTGCCTTCCAAAGCTATATGCTGAGGGAGGAT |
| increase_interaction_ | GGGCGCTGTTGCAGCGTCTGCCCACCTCAGAGTGGGTATCCTTACCTAT |
| of_SL5_7 | TGTGAAAAGCATAATAGGTCAAGGAATGCAACTGGTTGCCCACCCTAG |
| | TCATTG |
| SEQ ID NO: 194 | GTCCACTTTAATAAGTGGTGCCTTCCAAAGCTATATGCTGAGGGAGGAT |
| increase_interaction_ | GGGCGCTGTTGCAGCGTCTGCCCACCTCAGAGTGGGTATCCTTACCTAT |
| of_SL5_8 | TGCTGAAAAGCAGTAATAGGTCAAGGAATGCAACTGGTTGCCCACCCT |
| | AGTCATTG |
| SEQ ID NO: 195 | GTCCACTTTAATAAGTGGTGCCTTCCAAAGCTATATGCTGAGGGAGGAT |
| increase_interaction_ | GGGCGCTGTTGCAGCGTCTGCCCACCTCAGAGTGGGTATCCTTACCTAT |
| of_SL5_9 | TGGCTGAAAAGCAGCTAATAGGTCAAGGAATGCAACTGGTTGCCCACC |
| | CTAGTCATTG |
| SEQ ID NO: 196 | GTCCACTTTAATAAGTGGTGCCTTCCAAAGCTATATGCTGAGGGAGGAT |
| increase_interaction_ | GGGCGCTGTTGCAGCGTCTGCCCACCTCAGAGTGGGTATCCTTACCTAT |
| of_SL5_10 | TGTGCTGAAAAGCAGCATAATAGGTCAAGGAATGCAACTGGTTGCCCA |
| | CCCTAGTCATTG |
| SEQ ID NO: 197 | GTCCACTTTAATAAGTGGTGCCTTCCAAAGCTATATGCTGAGGGAGGAT |
| SL4_MS2_hp | GGGCGCTGTTGCAGCGTCTGCCCACCTCAGAGTGGGTATCCTTACCTAT |
| | TACATGAGGATCACCCATGTAATAGGTCAAGGAATGCAACTGGTTGCCC |
| | ACCCTAGTCATTG |
| SEQ ID NO: 198 | GCTCCACTTGCTAATGCAAGTGGTGCCTTCCAAAGCTATATGCTGAGGG |
| sgRNA version3.2 | AGGATGGGCGCTGCGGCATGCGTCTGCCCACCTCAGAGTGGGTATCCTT |
| | ACCTATTGAAAAGTAATAGGTCAAGGAATGCCGCTGGTTGCCCACCCTA |
| | GTCATTG |

TABLE 15

| | Location of N-termini |
|---|---|
| PsaCas12f construct name | (amino acid position) |
| cpPsaCas12f_1 | I77 |
| cpPsaCas12f_2 | N104 |
| cpPsaCas12f_3 | P146 |
| cpPsaCas12f_4 | E224 |
| cpPsaCas12f_5 | N266 |
| cpPsaCas12f_6 | D375 |
| cpPsaCas12f_7 | K349 |
| cpPsaCas12f_8 | K55 |
| cpPsaCas12f_9 | 537K |
| cpPsaCas12f_10 | A407 |
| cpPsaCas12f_11 | R216 |
| cpPsaCas12f_12 | N520 |

TABLE 16

| | |
|---|---|
| SEQ ID NO: 199 | GCTCCGCTTTAATAAGCGGTGCCTTCCAAAGCTATATGCTGAGGGAGGA |
| 5pr_trunc_7-B12 (= | TGGGCGCTGCCGCATGCGTCTGCCCACCTCAGAGTGGGTATCCTTACCT |
| increase interaction_w_ | ATTGAAAAGTAATAGGTCAAGGAATGCGGCTGGTTGCCCACCCTAGTC |
| crRNA 21) | ATTG |
| SEQ ID NO: 200 | GCACCACTTTAATAAGTGGTGCCTTCCAAAGCTATATGCTGAGGGAGGA |
| SL1_modification_1 + | TGGGCGCTGCCGCATGCGTCTGCCCACCTCAGAGTGGGTATCCTTACCT |
| increase_interaction_w_ | ATTGAAAAGTAATAGGTCAAGGAATGCGGCTGGTTGCCCACCCTAGTC |
| crRNA_21 | ATTG |
| SEQ ID NO: 201 | GCTCCACTGTAATCAGTGGTGCCTTCCAAAGCTATATGCTGAGGGAGGA |
| SL1_modification_3 + | TGGGCGCTGCCGCATGCGTCTGCCCACCTCAGAGTGGGTATCCTTACCT |
| increase_interaction_w_ | ATTGAAAAGTAATAGGTCAAGGAATGCGGCTGGTTGCCCACCCTAGTC |
| crRNA_21 | ATTG |
| SEQ ID NO: 202 | GCTCCACTTGCTAATGCAAGTGGTGCCTTCCAAAGCTATATGCTGAGGG |
| SL1_modification_5 + | AGGATGGGCGCTGCCGCATGCGTCTGCCCACCTCAGAGTGGGTATCCTT |
| increase_interaction_w_ | ACCTATTGAAAAGTAATAGGTCAAGGAATGCGGCTGGTTGCCCACCCTA |
| crRNA_21 | GTCATTG |
| SEQ ID NO: 203 | GCTCCACTTACATGAGGATCACCCATGTAAGTGGTGCCTTCCAAAGCTA |
| SL1_modification_8 + | TATGCTGAGGGAGGATGGGCGCTGCCGCATGCGTCTGCCCACCTCAGA |
| increase_interaction_w_ | GTGGGTATCCTTACCTATTGAAAAGTAATAGGTCAAGGAATGCGGCTGG |
| crRNA_21_sgRNA | TTGCCCACCCTAGTCATTG |
| 3.1 | |
| SEQ ID NO: 204 | GTCCACTTTAATAAGTGGTGCCTTCCAAAGCTATATGCTGAGGGAGGAT |
| SL1_MS2_hp + | GGGCGCTGCCGCATGCGTCTGCCCACCTCAGAGTGGGTATCCTTACCTA |
| increase_interaction_w_ | TTGAAAAGTAATAGGTCAAGGAATGCGGCTGGTTGCCCACCCTAGTCAT |
| crRNA_21 | TG |
| SEQ ID NO: 205 | GCTCCGCTTTAATAAGCGGTGCCTTCCAAAGCTATATGCTGAGGGAGGA |
| 5pr_trunc_7 + | TGGGCGCTGCGGCATGCGTCTGCCCACCTCAGAGTGGGTATCCTTACCT |
| increase_interaction_w_ | ATTGAAAAGTAATAGGTCAAGGAATGCCGCTGGTTGCCCACCCTAGTCA |
| crRNA_22 | TTG |
| SEQ ID NO: 206 | GCACCACTTTAATAAGTGGTGCCTTCCAAAGCTATATGCTGAGGGAGGA |
| SL1_modification_1 + | TGGGCGCTGCGGCATGCGTCTGCCCACCTCAGAGTGGGTATCCTTACCT |
| increase_interaction_w_ | ATTGAAAAGTAATAGGTCAAGGAATGCCGCTGGTTGCCCACCCTAGTCA |
| crRNA_22 | TTG |
| SEQ ID NO: 207 | GCTCCACTGTAATCAGTGGTGCCTTCCAAAGCTATATGCTGAGGGAGGA |
| SL1_modification_3 + | TGGGCGCTGCGGCATGCGTCTGCCCACCTCAGAGTGGGTATCCTTACCT |
| increase_interaction_w_ | ATTGAAAAGTAATAGGTCAAGGAATGCCGCTGGTTGCCCACCCTAGTCA |
| crRNA_22 | TTG |
| SEQ ID NO: 198 | GCTCCACTTGCTAATGCAAGTGGTGCCTTCCAAAGCTATATGCTGAGGG |
| SL1_modification_5 + | AGGATGGGCGCTGCGGCATGCGTCTGCCCACCTCAGAGTGGGTATCCTT |
| increase_interaction_w_ | ACCTATTGAAAAGTAATAGGTCAAGGAATGCCGCTGGTTGCCCACCCTA |
| crRNA_22 | GTCATTG |
| SEQ ID NO: 208 | GCTCCACTTACATGAGGATCACCCATGTAAGTGGTGCCTTCCAAAGCTA |
| SL1_modification_8 + | TATGCTGAGGGAGGATGGGCGCTGCGGCATGCGTCTGCCCACCTCAGA |
| increase_interaction_w_ | GTGGGTATCCTTACCTATTGAAAAGTAATAGGTCAAGGAATGCCGCTGG |
| crRNA_22 | TTGCCCACCCTAGTCATTG |
| SEQ ID NO: 209 | GTCCACTTTAATAAGTGGTGCCTTCCAAAGCTATATGCTGAGGGAGGAT |
| SL1_MS2_hp + | GGGCGCTGCGGCATGCGTCTGCCCACCTCAGAGTGGGTATCCTTACCTA |
| increase_interaction_w_ | TTGAAAAGTAATAGGTCAAGGAATGCCGCTGGTTGCCCACCCTAGTCAT |
| crRNA_22 | TG |
| SEQ ID NO: 210 | GCTCCGCTTTAATAAGCGGTGCCTTCCAAAGCTATATGCTGAGGGAGGA |
| 5pr_trunc_7 + | TGGGCGCTGCCGCAGCGTCTGCCCACCTCAGAGTGGGTATCCTTACCTA |
| increase_interaction_w_ | TTGAAAAGTAATAGGTCAAGGAATGCGGCTGGTTGCCCACCCTAGTCAT |
| crRNA_25 | TG |
| SEQ ID NO: 211 | GCACCACTTTAATAAGTGGTGCCTTCCAAAGCTATATGCTGAGGGAGGA |
| SL1_modification_1 + | TGGGCGCTGCCGCAGCGTCTGCCCACCTCAGAGTGGGTATCCTTACCTA |
| increase_interaction_w_ | TTGAAAAGTAATAGGTCAAGGAATGCGGCTGGTTGCCCACCCTAGTCAT |
| crRNA_25 | TG |
| SEQ ID NO: 212 | GCTCCACTGTAATCAGTGGTGCCTTCCAAAGCTATATGCTGAGGGAGGA |
| SL1_modification_3 + | TGGGCGCTGCCGCAGCGTCTGCCCACCTCAGAGTGGGTATCCTTACCTA |
| increase_interaction_w_ | TTGAAAAGTAATAGGTCAAGGAATGCGGCTGGTTGCCCACCCTAGTCAT |
| crRNA_25 | TG |
| SEQ ID NO: 213 | GCTCCACTTGCTAATGCAAGTGGTGCCTTCCAAAGCTATATGCTGAGGG |
| SL1_modification_5 + | AGGATGGGCGCTGCCGCAGCGTCTGCCCACCTCAGAGTGGGTATCCTTA |
| increase_interaction_w | CCTATTGAAAAGTAATAGGTCAAGGAATGCGGCTGGTTGCCCACCCTAG |
| crRNA_25 | TCATTG |
| SEQ ID NO: 214 | GCTCCACTTACATGAGGATCACCCATGTAAGTGGTGCCTTCCAAAGCTA |
| SL1_modification_8 + | TATGCTGAGGGAGGATGGGCGCTGCCGCAGCGTCTGCCCACCTCAGAG |
| increase_interaction_w_ | TGGGTATCCTTACCTATTGAAAAGTAATAGGTCAAGGAATGCGGCTGGT |
| crRNA_25 | TGCCCACCCTAGTCATTG |
| SEQ ID NO: 215 | GTCCACTTTAATAAGTGGTGCCTTCCAAAGCTATATGCTGAGGGAGGAT |
| SL1_MS2_hp + | GGGCGCTGCCGCAGCGTCTGCCCACCTCAGAGTGGGTATCCTTACCTAT |
| increase_interaction_w_ | TGAAAAGTAATAGGTCAAGGAATGCGGCTGGTTGCCCACCCTAGTCATT |
| crRNA_25 | G |
| SEQ ID NO: 216 | GCTCCGCTTTAATAAGCGGTGCCTTCCAAAGCTATATGCTGAGGGAGGA |
| 5pr_trunc_7 + | TGGGCGCTGCGGCAGCGTCTGCCCACCTCAGAGTGGGTATCCTTACCTA |
| increase_interaction_w_ | TTGAAAAGTAATAGGTCAAGGAATGCCGCTGGTTGCCCACCCTAGTCAT |
| crRNA_26 | TG |
| SEQ ID NO: 217 | GCACCACTTTAATAAGTGGTGCCTTCCAAAGCTATATGCTGAGGGAGGA |
| SL1_modification_1 + | TGGGCGCTGCGGCAGCGTCTGCCCACCTCAGAGTGGGTATCCTTACCTA |
| increase_interaction_w_ | TTGAAAAGTAATAGGTCAAGGAATGCCGCTGGTTGCCCACCCTAGTCAT |
| crRNA_26 | TG |
| SEQ ID NO: 218 | GCTCCACTGTAATCAGTGGTGCCTTCCAAAGCTATATGCTGAGGGAGGA |
| SL1_modification_3 + | TGGGCGCTGCGGCAGCGTCTGCCCACCTCAGAGTGGGTATCCTTACCTA |
| increase_interaction_w_ | TTGAAAAGTAATAGGTCAAGGAATGCCGCTGGTTGCCCACCCTAGTCAT |
| crRNA_26 | TG |
| SEQ ID NO: 219 | GCTCCACTTGCTAATGCAAGTGGTGCCTTCCAAAGCTATATGCTGAGGG |
| SL1_modification_5 + | AGGATGGGCGCTGCGGCAGCGTCTGCCCACCTCAGAGTGGGTATCCTTA |
| increase_interaction_w_ | CCTATTGAAAAGTAATAGGTCAAGGAATGCCGCTGGTTGCCCACCCTAG |
| crRNA_26 | TCATTG |
| SEQ ID NO: 220 | GCTCCACTTACATGAGGATCACCCATGTAAGTGGTGCCTTCCAAAGCTA |
| SL1_modification_8 + | TATGCTGAGGGAGGATGGGCGCTGCGGCAGCGTCTGCCCACCTCAGAG |
| increase_interaction_w_ | TGGGTATCCTTACCTATTGAAAAGTAATAGGTCAAGGAATGCCGCTGGT |
| crRNA_26 | TGCCCACCCTAGTCATTG |
| SEQ ID NO: 221 | GTCCACTTTAATAAGTGGTGCCTTCCAAAGCTATATGCTGAGGGAGGAT |
| SL1_MS2_hp + | GGGCGCTGCGGCAGCGTCTGCCCACCTCAGAGTGGGTATCCTTACCTAT |
| increase_interaction_w_ | TGAAAAGTAATAGGTCAAGGAATGCCGCTGGTTGCCCACCCTAGTCATT |
| crRNA_26 | G |
| SEQ ID NO: 222 | GCTCCACTTTAATAAGTGGTGCCTTCCAAAGCTATATGCTGAGGGAGGA |
| best_guide_v2 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |

TABLE 17

| | |
|---|---|
| SEQ ID NO: 223 | TCCACTTTAATAAGTGGTGCCTTCCAAAGCTATATGCTGAGGGAGGATG |
| EMX_Cas12f_g_2 | GGCGCTGTTGCAGCGTCTGCCCACCTCAGAGTGGGTATCCTTACCTATT |
| | GAAAAGTAATAGGTCAAGGAATGCAACTGGTTGCCCACCCTAGTCATT |
| | G |
| SEQ ID NO: 224 | GCCTTCCAAAGCTATATGCTGAGGGAGGATGGGCGCTGTTGCAGCGTCT |
| EMX_Cas12f_g_3 | GCCCACCTCAGAGTGGGTATCCTTACCTATTGAAAAGTAATAGGTCAAG |
| | GAATGCAACTGGTTGCCCACCCTAGTCATTG |
| SEQ ID NO: 225 | GCTCCACTTTAATAAGTGGTGCCTTCCAAAGCTATATGCTGAGGGAGGA |
| EMX1-stagger_25 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TGGAG |
| SEQ ID NO: 226 | GCTCCACTTTAATAAGTGGTGCCTTCCAAAGCTATATGCTGAGGGAGGA |
| EMX1-stagger_24 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TGGA |
| SEQ ID NO: 227 | GCTCCACTTTAATAAGTGGTGCCTTCCAAAGCTATATGCTGAGGGAGGA |
| EMX1-stagger 23 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TGG |
| SEQ ID NO: 228 | GCTCCACTTTAATAAGTGGTGCCTTCCAAAGCTATATGCTGAGGGAGGA |
| EMX1-stagger_22 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | TG |
| SEQ ID NO: 229 | GCTCCACTTTAATAAGTGGTGCCTTCCAAAGCTATATGCTGAGGGAGGA |
| EMX1-stagger_21 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| | T |
| SEQ ID NO: 230 | GCTCCACTTTAATAAGTGGTGCCTTCCAAAGCTATATGCTGAGGGAGGA |
| EMX1-stagger_20 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCAT |
| SEQ ID NO: 231 | GCTCCACTTTAATAAGTGGTGCCTTCCAAAGCTATATGCTGAGGGAGGA |
| EMX1-stagger_19 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTCA |
| SEQ ID NO: 232 | GCTCCACTTTAATAAGTGGTGCCTTCCAAAGCTATATGCTGAGGGAGGA |
| EMX1-stagger_18 | TGGGCGCTGTTGCAGCGTCTGCCCACCTCAGAGTGGGTATCCTTACCTA |
| | TTGAAAAGTAATAGGTCAAGGAATGCAACTGGTTGCCCACCCTAGTC |

TABLE 18

| | |
|---|---|
| SEQ ID NO: 233 | MPSETYITKTLSLKLIPSDEEKQALENYFITFQRAVNFAIDRIVDIRSSFRYLN |
| Cas12f_intraprotein_ | KNEQFPAVCDCCGKKEKIMYVNIGSPKKKRKVSGVWLDGVNIFSVSILLVS |
| NLS_1_orange | AWLEFKGFVRAHICKTCYSGVAGNMFIRKQMYPNDKEGWKVSRSYNIKV |
| | NAPGLTGTEYAMAIRKAISILRSFEKRRRNAERRIIEYEKSKKEYLELIDDVE |
| | KGKTNKIVVLEKEGHQRVKRYKHKNWPEKWQGISLNKAKSKVKDIEKRIK |
| | KLKEWKHPTLNRPYVELHKNNVRIVGYETVELKLGNKMYTIHFASISNLR |
| | KPFRKQKKKSIEYLKHLLTLALKRNLETYPSIIKRGKNFFLQYPVRVTVKVP |
| | KLTKNFKAFGIDRGVNRLAVGCIISKDGKLTNKNIFFFHGKEAWAKENRYK |
| | KIRDRLYAMAKKLRGDKTKKIRLYHEIRKKFRHKVKYFRRNYLHNISKQIV |
| | EIAKENTPTVIVLEDLRYLRERTYRGKGRSKKAKKTNYKLNTFTYRMLIDM |
| | IKYKAEEAGVPVMIIDPRNTSRKCSKCGYVDENNRKQASFKCLKCGYSLN |
| | ADLNAAVNIAKAFYECPTFRWEEKLHAYVCSEPDK |
| SEQ ID NO: 234 | MPSETYITKTLSLKLIPSDEEKQALENYFITFQRAVNFAIDRIVDIRSSFRYLN |
| Cas12f_intraprotein_ | KNEQFPAVCDCCGKKEKIMYVNIVWLDGVNIFSVSILLVSAWLEFKGFVRG |
| NLS_2_orange | SPKKKRKVSGAHICKTCYSGVAGNMFIRKQMYPNDKEGWKVSRSYNIKV |
| | NAPGLTGTEYAMAIRKAISILRSFEKRRRNAERRIIEYEKSKKEYLELIDDVE |
| | KGKTNKIVVLEKEGHQRVKRYKHKNWPEKWQGISLNKAKSKVKDIEKRIK |
| | KLKEWKHPTLNRPYVELHKNNVRIVGYETVELKLGNKMYTIHFASISNLR |
| | KPFRKQKKKSIEYLKHLLTLALKRNLETYPSIIKRGKNFFLQYPVRVTVKVP |
| | KLTKNFKAFGIDRGVNRLAVGCIISKDGKLTNKNIFFFHGKEAWAKENRYK |
| | KIRDRLYAMAKKLRGDKTKKIRLYHEIRKKFRHKVKYFRRNYLHNISKQIV |
| | EIAKENTPTVIVLEDLRYLRERTYRGKGRSKKAKKTNYKLNTFTYRMLIDM |
| | IKYKAEEAGVPVMIIDPRNTSRKCSKCGYVDENNRKQASFKCLKCGYSLN |
| | ADLNAAVNIAKAFYECPTFRWEEKLHAYVCSEPDK |
| SEQ ID NO: 235 | MPSETYITKTLSLKLIPSDEEKQALENYFITFQRAVNFAIDRIVDIRSSFRYLN |
| Cas12f_intraprotein_ | KNEQFPAVCDCCGKKEKIMYVNIVWLDGVNIFSVSILLVSAWLEFKGFVRA |
| NLS_3_orange | HICKTCYSGVAGNMFIRKQMYPNDKEGWKVSRSYNIKVNAPGSPKKKRK |
| | VSGGLTGTEYAMAIRKAISILRSFEKRRRNAERRIIEYEKSKKEYLELIDDVE |
| | KGKTNKIVVLEKEGHQRVKRYKHKNWPEKWQGISLNKAKSKVKDIEKRIK |
| | KLKEWKHPTLNRPYVELHKNNVRIVGYETVELKLGNKMYTIHFASISNLR |
| | KPFRKQKKKSIEYLKHLLTLALKRNLETYPSIIKRGKNFFLQYPVRVTVKVP |
| | KLTKNFKAFGIDRGVNRLAVGCIISKDGKLTNKNIFFFHGKEAWAKENRYK |
| | KIRDRLYAMAKKLRGDKTKKIRLYHEIRKKFRHKVKYFRRNYLHNISKQIV |
| | EIAKENTPTVIVLEDLRYLRERTYRGKGRSKKAKKTNYKLNTFTYRMLIDM |
| | IKYKAEEAGVPVMIIDPRNTSRKCSKCGYVDENNRKQASFKCLKCGYSLN |
| | ADLNAAVNIAKAFYECPTFRWEEKLHAYVCSEPDK |
| SEQ ID NO: 236 | MPSETYITKTLSLKLIPSDEEKQALENYFITFQRAVNFAIDRIVDIRSSFRYLN |
| Cas12f_intraprotein_ | KNEQFPAVCDCCGKKEKIMYVNIVWLDGVNIFSVSILLVSAWLEFKGFVRA |
| NLS_4_orange | HICKTCYSGVAGNMFIRKQMYPNDKEGWKVSRSYNIKVNAPGLTGTEYA |
| | MAIRKAISILRSFEKRRRNAERRIIEYEKSKKEYLELIDDVEKGKTNKIVVLE |
| | KEGHQRVKRYKHKNWPEGSPKKKRKVSGKWQGISLNKAKSKVKDIEKRI |
| | KKLKEWKHPTLNRPYVELHKNNVRIVGYETVELKLGNKMYTIHFASISNL |
| | RKPFRKQKKKSIEYLKHLLTLALKRNLETYPSIIKRGKNFFLQYPVRVTVKV |
| | PKLTKNFKAFGIDRGVNRLAVGCIISKDGKLTNKNIFFFHGKEAWAKENRY |
| | KKIRDRLYAMAKKLRGDKTKKIRLYHEIRKKFRHKVKYFRRNYLHNISKQI |
| | VEIAKENTPTVIVLEDLRYLRERTYRGKGRSKKAKKTNYKLNTFTYRMLID |
| | MIKYKAEEAGVPVMIIDPRNTSRKCSKCGYVDENNRKQASFKCLKCGYSL |
| | NADLNAAVNIAKAFYECPTFRWEEKLHAYVCSEPDK |
| SEQ ID NO: 237 | MPSETYITKTLSLKLIPSDEEKQALENYFITFQRAVNFAIDRIVDIRSSFRYLN |
| Cas12f_intraprotein_ | KNEQFPAVCDCCGKKEKIMYVNIVWLDGVNIFSVSILLVSAWLEFKGFVRA |
| NLS_5_orange | HICKTCYSGVAGNMFIRKQMYPNDKEGWKVSRSYNIKVNAPGLTGTEYA |
| | MAIRKAISILRSFEKRRRNAERRIIEYEKSKKEYLELIDDVEKGKTNKIVVLE |
| | KEGHQRVKRYKHKNWPEKWQGISLNKAKSKVKDIEKRIKKLKEWKHPTL |
| | NRPYVELHKNGSPKKKRKVSGNVRIVGYETVELKLGNKMYTIHFASISNLR |
| | KPFRKQKKKSIEYLKHLLTLALKRNLETYPSIIKRGKNFFLQYPVRVTVKVP |
| | KLTKNFKAFGIDRGVNRLAVGCIISKDGKLTNKNIFFFHGKEAWAKENRYK |
| | KIRDRLYAMAKKLRGDKTKKIRLYHEIRKKFRHKVKYFRRNYLHNISKQIV |
| | EIAKENTPTVIVLEDLRYLRERTYRGKGRSKKAKKTNYKLNTFTYRMLIDM |
| | IKYKAEEAGVPVMIIDPRNTSRKCSKCGYVDENNRKQASFKCLKCGYSLN |
| | ADLNAAVNIAKAFYECPTFRWEEKLHAYVCSEPDK |
| SEQ ID NO: 238 | MPSETYITKTLSLKLIPSDEEKQALENYFITFQRAVNFAIDRIVDIRSSFRYLN |
| Cas12f_intraprotein_ | KNEQFPAVCDCCGKKEKIMYVNIVWLDGVNIFSVSILLVSAWLEFKGFVRA |
| NLS_6_orange | HICKTCYSGVAGNMFIRKQMYPNDKEGWKVSRSYNIKVNAPGLTGTEYA |
| | MAIRKAISILRSFEKRRRNAERRIIEYEKSKKEYLELIDDVEKGKTNKIVVLE |
| | KEGHQRVKRYKHKNWPEKWQGISLNKAKSKVKDIEKRIKKLKEWKHPTL |
| | NRPYVELHKNNVRIVGYETVELKLGNKMYTIHFASISNLRKPFRKQKKKSI |
| | EYLKHLLTLALKRNLETYPSIIKRGKNFFLQYPVRVTVKVPKLTKNFKAFGI |
| | DRGVNRLAVGCIISKDGSPKKKRKVSGGKLTNKNIFFFHGKEAWAKENRY |
| | KKIRDRLYAMAKKLRGDKTKKIRLYHEIRKKFRHKVKYFRRNYLHNISKQI |
| | VEIAKENTPTVIVLEDLRYLRERTYRGKGRSKKAKKTNYKLNTFTYRMLID |
| | MIKYKAEEAGVPVMIIDPRNTSRKCSKCGYVDENNRKQASFKCLKCGYSL |
| | NADLNAAVNIAKAFYECPTFRWEEKLHAYVCSEPDK |
| SEQ ID NO: 239 | MKRTADGSEFESPKKKRKVMPSETYITKTLSLKLIPSDEEKQALENYFITFQ |
| Cas12f_intraprotein_ | RAVNFAIDRIVDIRSSFRYLNKNEQFPAVCDCCGKKEKIMYVNIGSPKKKR |
| and_flanking_NLS_1_ | KVSGVWLDGVNIFSVSILLVSAWLEFKGFVRAHICKTCYSGVAGNMFIRKQ |
| grey | MYPNDKEGWKVSRSYNIKVNAPGLTGTEYAMAIRKAISILRSFEKRRRNAE |
| | RRIIEYEKSKKEYLELIDDVEKGKTNKIVVLEKEGHQRVKRYKHKNWPEK |
| | WQGISLNKAKSKVKDIEKRIKKLKEWKHPTLNRPYVELHKNNVRIVGYET |
| | VELKLGNKMYTIHFASISNLRKPFRKQKKKSIEYLKHLLTLALKRNLETYPS |
| | IIKRGKNFFLQYPVRVTVKVPKLTKNFKAFGIDRGVNRLAVGCIISKDGKLT |
| | NKNIFFFHGKEAWAKENRYKKIRDRLYAMAKKLRGDKTKKIRLYHEIRKK |
| | FRHKVKYFRRNYLHNISKQIVEIAKENTPTVIVLEDLRYLRERTYRGKGRSK |
| | KAKKTNYKLNTFTYRMLIDMIKYKAEEAGVPVMIIDPRNTSRKCSKCGYV |
| | DENNRKQASFKCLKCGYSLNADLNAAVNIAKAFYECPTFRWEEKLHAYV |
| | CSEPDKSGGSKRTADGSEFEPKKKRKV |
| SEQ ID NO: 240 | MKRTADGSEFESPKKKRKVMPSETYITKTLSLKLIPSDEEKQALENYFITFQ |
| Cas12f_intraprotein_ | RAVNFAIDRIVDIRSSFRYLNKNEQFPAVCDCCGKKEKIMYVNIVWLDGVN |
| and_flanking_NLS_2_ | IFSVSILLVSAWLEFKGFVRGSPKKKRKVSGAHICKTCYSGVAGNMFIRKQ |
| grey | MYPNDKEGWKVSRSYNIKVNAPGLTGTEYAMAIRKAISILRSFEKRRRNAE |
| | RRIIEYEKSKKEYLELIDDVEKGKTNKIVVLEKEGHQRVKRYKHKNWPEK |
| | WQGISLNKAKSKVKDIEKRIKKLKEWKHPTLNRPYVELHKNNVRIVGYET |
| | VELKLGNKMYTIHFASISNLRKPFRKQKKKSIEYLKHLLTLALKRNLETYPS |
| | IIKRGKNFFLQYPVRVTVKVPKLTKNFKAFGIDRGVNRLAVGCIISKDGKLT |
| | NKNIFFFHGKEAWAKENRYKKIRDRLYAMAKKLRGDKTKKIRLYHEIRKK |
| | FRHKVKYFRRNYLHNISKQIVEIAKENTPTVIVLEDLRYLRERTYRGKGRSK |
| | KAKKTNYKLNTFTYRMLIDMIKYKAEEAGVPVMIIDPRNTSRKCSKCGYV |
| | DENNRKQASFKCLKCGYSLNADLNAAVNIAKAFYECPTFRWEEKLHAYV |
| | CSEPDKSGGSKRTADGSEFEPKKKRKV |
| SEQ ID NO: 241 | MKRTADGSEFESPKKKRKVMPSETYITKTLSLKLIPSDEEKQALENYFITFQ |
| Cas12f_intraprotein_ | RAVNFAIDRIVDIRSSFRYLNKNEQFPAVCDCCGKKEKIMYVNIVWLDGVN |
| and_flanking_NLS_3_ | IFSVSILLVSAWLEFKGFVRAHICKTCYSGVAGNMFIRKQMYPNDKEGWK |
| grey | VSRSYNIKVNAPGSPKKKRKVSGGLTGTEYAMAIRKAISILRSFEKRRRNAE |
| | RRIIEYEKSKKEYLELIDDVEKGKTNKIVVLEKEGHQRVKRYKHKNWPEK |
| | WQGISLNKAKSKVKDIEKRIKKLKEWKHPTLNRPYVELHKNNVRIVGYET |
| | VELKLGNKMYTIHFASISNLRKPFRKQKKKSIEYLKHLLTLALKRNLETYPS |
| | IIKRGKNFFLQYPVRVTVKVPKLTKNFKAFGIDRGVNRLAVGCIISKDGKLT |
| | NKNIFFFHGKEAWAKENRYKKIRDRLYAMAKKLRGDKTKKIRLYHEIRKK |
| | FRHKVKYFRRNYLHNISKQIVEIAKENTPTVIVLEDLRYLRERTYRGKGRSK |
| | KAKKTNYKLNTFTYRMLIDMIKYKAEEAGVPVMIIDPRNTSRKCSKCGYV |
| | DENNRKQASFKCLKCGYSLNADLNAAVNIAKAFYECPTFRWEEKLHAYV |
| | CSEPDKSGGSKRTADGSEFEPKKKRKV |
| SEQ ID NO: 242 | MKRTADGSEFESPKKKRKVMPSETYITKTLSLKLIPSDEEKQALENYFITFQ |
| Cas12f_intraprotein_ | RAVNFAIDRIVDIRSSFRYLNKNEQFPAVCDCCGKKEKIMYVNIVWLDGVN |
| and_flanking_NLS_4_ | IFSVSILLVSAWLEFKGFVRAHICKTCYSGVAGNMFIRKQMYPNDKEGWK |
| grey | VSRSYNIKVNAPGLTGTEYAMAIRKAISILRSFEKRRRNAERRIIEYEKSKKE |
| | YLELIDDVEKGKTNKIVVLEKEGHQRVKRYKHKNWPEGSPKKKRKVSGK |
| | WQGISLNKAKSKVKDIEKRIKKLKEWKHPTLNRPYVELHKNNVRIVGYET |
| | VELKLGNKMYTIHFASISNLRKPFRKQKKKSIEYLKHLLTLALKRNLETYPS |
| | IIKRGKNFFLQYPVRVTVKVPKLTKNFKAFGIDRGVNRLAVGCIISKDGKLT |
| | NKNIFFFHGKEAWAKENRYKKIRDRLYAMAKKLRGDKTKKIRLYHEIRKK |
| | FRHKVKYFRRNYLHNISKQIVEIAKENTPTVIVLEDLRYLRERTYRGKGRSK |
| | KAKKTNYKLNTFTYRMLIDMIKYKAEEAGVPVMIIDPRNTSRKCSKCGYV |
| | DENNRKQASFKCLKCGYSLNADLNAAVNIAKAFYECPTFRWEEKLHAYV |
| | CSEPDKSGGSKRTADGSEFEPKKKRKV |
| SEQ ID NO: 243 | MKRTADGSEFESPKKKRKVMPSETYITKTLSLKLIPSDEEKQALENYFITFQ |
| Cas12f_intraprotein_ | RAVNFAIDRIVDIRSSFRYLNKNEQFPAVCDCCGKKEKIMYVNIVWLDGVN |
| and_flanking_NLS_5_ | IFSVSILLVSAWLEFKGFVRAHICKTCYSGVAGNMFIRKQMYPNDKEGWK |
| grey | VSRSYNIKVNAPGLTGTEYAMAIRKAISILRSFEKRRRNAERRIIEYEKSKKE |
| | YLELIDDVEKGKTNKIVVLEKEGHQRVKRYKHKNWPEKWQGISLNKAKS |
| | KVKDIEKRIKKLKEWKHPTLNRPYVELHKNGSPKKKRKVSGNVRIVGYET |
| | VELKLGNKMYTIHFASISNLRKPFRKQKKKSIEYLKHLLTLALKRNLETYPS |
| | IIKRGKNFFLQYPVRVTVKVPKLTKNFKAFGIDRGVNRLAVGCIISKDGKLT |
| | NKNIFFFHGKEAWAKENRYKKIRDRLYAMAKKLRGDKTKKIRLYHEIRKK |
| | FRHKVKYFRRNYLHNISKQIVEIAKENTPTVIVLEDLRYLRERTYRGKGRSK |
| | KAKKTNYKLNTFTYRMLIDMIKYKAEEAGVPVMIIDPRNTSRKCSKCGYV |
| | DENNRKQASFKCLKCGYSLNADLNAAVNIAKAFYECPTFRWEEKLHAYV |
| | CSEPDKSGGSKRTADGSEFEPKKKRKV |
| SEQ ID NO: 244 | MKRTADGSEFESPKKKRKVMPSETYITKTLSLKLIPSDEEKQALENYFITFQ |
| Cas12f_intraprotein_ | RAVNFAIDRIVDIRSSFRYLNKNEQFPAVCDCCGKKEKIMYVNIVWLDGVN |
| and_flanking_NLS_6_ | IFSVSILLVSAWLEFKGFVRAHICKTCYSGVAGNMFIRKQMYPNDKEGWK |
| grey | VSRSYNIKVNAPGLTGTEYAMAIRKAISILRSFEKRRRNAERRIIEYEKSKKE |
| | YLELIDDVEKGKTNKIVVLEKEGHQRVKRYKHKNWPEKWQGISLNKAKS |
| | KVKDIEKRIKKLKEWKHPTLNRPYVELHKNNVRIVGYETVELKLGNKMYT |
| | IHFASISNLRKPFRKQKKKSIEYLKHLLTLALKRNLETYPSIIKRGKNFFLQY |
| | PVRVTVKVPKLTKNFKAFGIDRGVNRLAVGCIISKDGSPKKKRKVSGGKLT |
| | NKNIFFFHGKEAWAKENRYKKIRDRLYAMAKKLRGDKTKKIRLYHEIRKK |
| | FRHKVKYFRRNYLHNISKQIVEIAKENTPTVIVLEDLRYLRERTYRGKGRSK |
| | KAKKTNYKLNTFTYRMLIDMIKYKAEEAGVPVMIIDPRNTSRKCSKCGYV |
| | DENNRKQASFKCLKCGYSLNADLNAAVNIAKAFYECPTFRWEEKLHAYV |
| | CSEPDKSGGSKRTADGSEFEPKKKRKV |

TABLE 19

| | |
|---|---|
| SEQ ID NO: 245 | GCTCCACTTGCTAATGCAAGTGGTGCCTTCCAAAGCTATATGCTGAGGG |
| RNF2_g8_PsaCas12f_ | AGGATGGGCGCTGCGGCATGCGTCTGCCCACCTCAGAGTGGGTATCCTT |
| targeting | ACCTATTGAAAAGTAATAGGTCAAGGAATGCCGCTATGAGTTACAACG |
| | AACACCTC |

TABLE 20

| | |
|---|---|
| SEQ ID NO: 246 | GCTCCACTTGCTAATGCAAGTGGTGCCTTCCAAACCAAAGCCTATATGG |
| SL5_4 + cr21 + SL2_3 + | CTGAGGGAGGATGGGCGCTGCCGCATGCGTCTGCCCACCTCAGAGTGG |
| SL1_8 | GTATCCTTACCTATTAAAAGTAATAGGTCAAGGAATGCGGCTGGTTGCC |
| | CACCCTAGTCATTG |
| SEQ ID NO: 247 | GCACCACTTTAATAAGTGGTGCCTTCCAAAGCTGTATATCAGCTGAGGG |
| SL5_4 + cr21 + SL2_4 + | AGGATGGGCGCTGCCGCATGCGTCTGCCCACCTCAGAGTGGGTATCCTT |
| SL1_3 | ACCTATTAAAAGTAATAGGTCAAGGAATGCGGCTGGTTGCCCACCCTAG |
| | TCATTG |
| SEQ ID NO: 248 | GCTCCACTTGCTAATGCAAGTGGTGCCTTCCAAAGCTGTATATCAGCTG |
| SL5_4 + cr21 + SL2_4 + | AGGGAGGATGGGCGCTGCCGCATGCGTCTGCCCACCTCAGAGTGGGTA |
| SL1_8 | TCCTTACCTATTAAAAGTAATAGGTCAAGGAATGCGGCTGGTTGCCCAC |
| | CCTAGTCATTG |
| SEQ ID NO: 249 | GCACCACTTTAATAAGTGGTGCCTTCCAAAGCTGCTATATGCAGCTGAG |
| SL5_4 + cr21 + SL2_5 + | GGAGGATGGGCGCTGCCGCATGCGTCTGCCCACCTCAGAGTGGGTATCC |
| SL1_3 | TTACCTATTAAAAGTAATAGGTCAAGGAATGCGGCTGGTTGCCCACCCT |
| | AGTCATTG |
| SEQ ID NO: 250 | GCTCCACTTGCTAATGCAAGTGGTGCCTTCCAAACCAAAGCCTATATGG |
| SL5_4 + cr22 + SL2_3 + | CTGAGGGAGGATGGGCGCTGCGGCATGCGTCTGCCCACCTCAGAGTGG |
| SL1_8 | GTATCCTTACCTATTAAAAGTAATAGGTCAAGGAATGCCGCTGGTTGCC |
| | CACCCTAGTCATTG |
| SEQ ID NO: 251 | GCACCACTTTAATAAGTGGTGCCTTCCAAAGCTGTATATCAGCTGAGGG |
| SL5_4 + cr22 + SL2_4 + | AGGATGGGCGCTGCGGCATGCGTCTGCCCACCTCAGAGTGGGTATCCTT |
| SL1_3 | ACCTATTAAAAGTAATAGGTCAAGGAATGCCGCTGGTTGCCCACCCTAG |
| | TCATTG |
| SEQ ID NO: 252 | GCTCCACTTGCTAATGCAAGTGGTGCCTTCCAAAGCTGTATATCAGCTG |
| SL5_4 + cr22 + SL2_4 + | AGGGAGGATGGGCGCTGCGGCATGCGTCTGCCCACCTCAGAGTGGGTA |
| SL1_8 | TCCTTACCTATTAAAAGTAATAGGTCAAGGAATGCCGCTGGTTGCCCAC |
| | CCTAGTCATTG |
| SEQ ID NO: 288 | GCACCACTTTAATAAGTGGTGCCTTCCAAAGCTGCTATATGCAGCTGAG |
| SL5_4 + cr22 + SL2_5 + | GGAGGATGGGCGCTGCGGCATGCGTCTGCCCACCTCAGAGTGGGTATC |
| SL1_3 | CTTACCTATTAAAAGTAATAGGTCAAGGAATGCCGCTGGTTGCCCACCC |
| | TAGTCATTG |
| SEQ ID NO: 253 | GCTCCACTTGCTAATGCAAGTGGTGCCTTCCAAACCAAAGCCTATATGG |
| SL5_5 + cr21 + SL2_3 + | CTGAGGGAGGATGGGCGCTGCCGCATGCGTCTGCCCACCTCAGAGTGG |
| SL1_8 | GTATCCTTACCTATTGGAAAAGCTAATAGGTCAAGGAATGCGGCTGGTT |
| | GCCCACCCTAGTCATTG |
| SEQ ID NO: 254 | GCACCACTTTAATAAGTGGTGCCTTCCAAAGCTGTATATCAGCTGAGGG |
| SL5_5 + cr21 + SL2_4 + | AGGATGGGCGCTGCCGCATGCGTCTGCCCACCTCAGAGTGGGTATCCTT |
| SL1_3 | ACCTATTGGAAAAGCTAATAGGTCAAGGAATGCGGCTGGTTGCCCACC |
| | CTAGTCATTG |
| SEQ ID NO: 255 | GCTCCACTTGCTAATGCAAGTGGTGCCTTCCAAAGCTGTATATCAGCTG |
| SL5_5 + cr21 + SL2_4 + | AGGGAGGATGGGCGCTGCCGCATGCGTCTGCCCACCTCAGAGTGGGTA |
| SL1_8 | TCCTTACCTATTGGAAAAGCTAATAGGTCAAGGAATGCGGCTGGTTGCC |
| | CACCCTAGTCATTG |
| SEQ ID NO: 256 | GCACCACTTTAATAAGTGGTGCCTTCCAAAGCTGCTATATGCAGCTGAG |
| SL5_5 + cr21 + SL2_5 + | GGAGGATGGGCGCTGCCGCATGCGTCTGCCCACCTCAGAGTGGGTATCC |
| SL1_3 | TTACCTATTGGAAAAGCTAATAGGTCAAGGAATGCGGCTGGTTGCCCAC |
| | CCTAGTCATTG |
| SEQ ID NO: 257 | GCTCCACTTGCTAATGCAAGTGGTGCCTTCCAAACCAAAGCCTATATGG |
| SL5_5 + cr22 + SL2_3 + | CTGAGGGAGGATGGGCGCTGCGGCATGCGTCTGCCCACCTCAGAGTGG |
| SL1_8 | GTATCCTTACCTATTGGAAAAGCTAATAGGTCAAGGAATGCCGCTGGTT |
| | GCCCACCCTAGTCATTG |
| SEQ ID NO: 258 | GCACCACTTTAATAAGTGGTGCCTTCCAAAGCTGTATATCAGCTGAGGG |
| SL5_5 + cr22 + SL2_4 + | AGGATGGGCGCTGCGGCATGCGTCTGCCCACCTCAGAGTGGGTATCCTT |
| SL1_3 | ACCTATTGGAAAAGCTAATAGGTCAAGGAATGCCGCTGGTTGCCCACCC |
| | TAGTCATTG |
| SEQ ID NO: 259 | GCTCCACTTGCTAATGCAAGTGGTGCCTTCCAAAGCTGTATATCAGCTG |
| SL5_5 + cr22 + SL2_4 + | AGGGAGGATGGGCGCTGCGGCATGCGTCTGCCCACCTCAGAGTGGGTA |
| SL1_8 | TCCTTACCTATTGGAAAAGCTAATAGGTCAAGGAATGCCGCTGGTTGCC |
| | CACCCTAGTCATTG |
| SEQ ID NO: 260 | GCACCACTTTAATAAGTGGTGCCTTCCAAAGCTGCTATATGCAGCTGAG |
| SL5_5 + cr22 + SL2_5 + | GGAGGATGGGCGCTGCGGCATGCGTCTGCCCACCTCAGAGTGGGTATC |
| SL1_3 | CTTACCTATTGGAAAAGCTAATAGGTCAAGGAATGCCGCTGGTTGCCCA |
| | CCCTAGTCATTG |
| SEQ ID NO: 261 | GCTCCACTTGCTAATGCAAGTGGTGCCTTCCAAACCAAAGCCTATATGG |
| SL5_7+ cr21 + SL2_3 + | CTGAGGGAGGATGGGCGCTGCCGCATGCGTCTGCCCACCTCAGAGTGG |
| SL1_8 | GTATCCTTACCTATTGTGAAAAGCATAATAGGTCAAGGAATGCGGCTGG |
| | TTGCCCACCCTAGTCATTG |
| SEQ ID NO: 262 | GCACCACTTTAATAAGTGGTGCCTTCCAAAGCTGTATATCAGCTGAGGG |
| SL5_7 + cr21 + SL2_4 + | AGGATGGGCGCTGCCGCATGCGTCTGCCCACCTCAGAGTGGGTATCCTT |
| SL1_3 | ACCTATTGTGAAAAGCATAATAGGTCAAGGAATGCGGCTGGTTGCCCA |
| | CCCTAGTCATTG |
| SEQ ID NO: 263 | GCTCCACTTGCTAATGCAAGTGGTGCCTTCCAAAGCTGTATATCAGCTG |
| SL5_7 + cr21 + SL2_4 + | AGGGAGGATGGGCGCTGCCGCATGCGTCTGCCCACCTCAGAGTGGGTA |
| SL1_8 | TCCTTACCTATTGTGAAAAGCATAATAGGTCAAGGAATGCGGCTGGTTG |
| | CCCACCCTAGTCATTG |
| SEQ ID NO: 264 | GCACCACTTTAATAAGTGGTGCCTTCCAAAGCTGCTATATGCAGCTGAG |
| SL5_7 + cr21 + SL2_5 + | GGAGGATGGGCGCTGCCGCATGCGTCTGCCCACCTCAGAGTGGGTATCC |
| SL1_3 | TTACCTATTGTGAAAAGCATAATAGGTCAAGGAATGCGGCTGGTTGCCC |
| | ACCCTAGTCATTG |
| SEQ ID NO: 265 | GCTCCACTTGCTAATGCAAGTGGTGCCTTCCAAACCAAAGCCTATATGG |
| SL5_7+ cr22 + SL2_3 + | CTGAGGGAGGATGGGCGCTGCGGCATGCGTCTGCCCACCTCAGAGTGG |
| SL1_8 | GTATCCTTACCTATTGTGAAAAGCATAATAGGTCAAGGAATGCCGCTGG |
| | TTGCCCACCCTAGTCATTG |
| SEQ ID NO: 266 | GCACCACTTTAATAAGTGGTGCCTTCCAAAGCTGTATATCAGCTGAGGG |
| SL5_7 + cr22 + SL2_4 + | AGGATGGGCGCTGCGGCATGCGTCTGCCCACCTCAGAGTGGGTATCCTT |
| SL1_3 | ACCTATTGTGAAAAGCATAATAGGTCAAGGAATGCCGCTGGTTGCCCAC |
| | CCTAGTCATTG |
| SEQ ID NO: 267 | GCTCCACTTGCTAATGCAAGTGGTGCCTTCCAAAGCTGTATATCAGCTG |
| SL5_7 + cr22 + SL2_4 + | AGGGAGGATGGGCGCTGCGGCATGCGTCTGCCCACCTCAGAGTGGGTA |
| SL1_8 | TCCTTACCTATTGTGAAAAGCATAATAGGTCAAGGAATGCCGCTGGTTG |
| | CCCACCCTAGTCATTG |
| SEQ ID NO: 268+ | GCACCACTTTAATAAGTGGTGCCTTCCAAAGCTGCTATATGCAGCTGAG |
| SL5_7+ cr22 + SL2_5 + | GGAGGATGGGCGCTGCGGCATGCGTCTGCCCACCTCAGAGTGGGTATC |
| SL1_3 | CTTACCTATTGTGAAAAGCATAATAGGTCAAGGAATGCCGCTGGTTGCC |
| | CACCCTAGTCATTG |
| SEQ ID NO: 269 | GCTCCGCTTTAATAAGCGGTGCCTTCCAAAGCTGTATATCAGCTGAGGG |
| SL2_4 + SL1_1 | AGGATGGGCGCTGTTGCAGCGTCTGCCCACCTCAGAGTGGGTATCCTTA |
| | CCTATTGAAAAGTAATAGGTCAAGGAATGCAACTGGTTGCCCACCCTAG |
| | TCATTG |
| SEQ ID NO: 270 | GCACCACTTTAATAAGTGGTGCCTTCCAAAGCTGTATATCAGCTGAGGG |
| SL2_4 + SL1_3 | AGGATGGGCGCTGTTGCAGCGTCTGCCCACCTCAGAGTGGGTATCCTTA |
| | CCTATTGAAAAGTAATAGGTCAAGGAATGCAACTGGTTGCCCACCCTAG |
| | TCATTG |
| SEQ ID NO: 271 | GCTCCACTGTAATCAGTGGTGCCTTCCAAAGCTGTATATCAGCTGAGGG |
| SL2_4 + SL1_5 | AGGATGGGCGCTGTTGCAGCGTCTGCCCACCTCAGAGTGGGTATCCTTA |
| | CCTATTGAAAAGTAATAGGTCAAGGAATGCAACTGGTTGCCCACCCTAG |
| | TCATTG |
| SEQ ID NO: 272 | GCTCCACTTGCTAATGCAAGTGGTGCCTTCCAAAGCTGTATATCAGCTG |
| SL2_4 + SL1_8 | AGGGAGGATGGGCGCTGTTGCAGCGTCTGCCCACCTCAGAGTGGGTAT |
| | CCTTACCTATTGAAAAGTAATAGGTCAAGGAATGCAACTGGTTGCCCAC |
| | CCTAGTCATTG |

TABLE 21

| | |
|---|---|
| SEQ ID NO: 273 | MKRTADGSEFESPKKKRKVSGGSISNKTFKFKPSRNQKDRYTKDIYTIKPN |
| cpPsaCas12f_1 | AHICKTCYSGVAGNMFIRKQMYPNDKEGWKVSRSYNIKVNAPGLTGTEY |
| | AMAIRKAISILRSFEKRRRNAERRIIEYEKSKKEYLELIDDVEKGKTNKIVVL |
| | EKEGHQRVKRYKHKNWPEKWQGISLNKAKSKVKDIEKRIKKLKEWKHPT |
| | LNRPYVELHKNNVRIVGYETVELKLGNKMYTIHFASISNLRKPFRKQKKKS |
| | IEYLKHLLTLALKRNLETYPSIIKRGKNFFLQYPVRVTVKVPKLTKNFKAFG |
| | IDRGVNRLAVGCIISKDGKLTNKNIFFFHGKEAWAKENRYKKIRDRLYAM |
| | AKKLRGDKTKKIRLYHEIRKKFRHKVKYFRRNYLHNISKQIVEIAKENTPT |
| | VIVLEDLRYLRERTYRGKGRSKKAKKTNYKLNTFTYRMLIDMIKYKAEEA |
| | GVPVMIIDPRNTSRKCSKCGYVDENNRKQASFKCLKCGYSLNADLNAAVN |
| | IAKAFYECPTFRWEEKLHAYVCSEPDKGGSGGSGGSGGSGGSGGSGGMPS |
| | ETYITKTLSLKLIPSDEEKQALENYFITFQRAVNFAIDRIVDIRSSFRYLNKNE |
| | QFPAVCDCCGKKEKIMYVNSGGSKRTADGSEFEPKKKRKV |
| SEQ ID NO: 274 | MKRTADGSEFESPKKKRKVSGGSNAHICKTCYSGVAGNMFIRKQMYPND |
| cpPsaCas12f_2 | KEGWKVSRSYNIKVNAPGLTGTEYAMAIRKAISILRSFEKRRRNAERRIIEY |
| | EKSKKEYLELIDDVEKGKTNKIVVLEKEGHQRVKRYKHKNWPEKWQGISL |
| | NKAKSKVKDIEKRIKKLKEWKHPTLNRPYVELHKNNVRIVGYETVELKLG |
| | NKMYTIHFASISNLRKPFRKQKKKSIEYLKHLLTLALKRNLETYPSIIKRGK |
| | NFFLQYPVRVTVKVPKLTKNFKAFGIDRGVNRLAVGCIISKDGKLTNKNIFF |
| | FHGKEAWAKENRYKKIRDRLYAMAKKLRGDKTKKIRLYHEIRKKFRHKV |
| | KYFRRNYLHNISKQIVEIAKENTPTVIVLEDLRYLRERTYRGKGRSKKAKK |
| | TNYKLNTFTYRMLIDMIKYKAEEAGVPVMIIDPRNTSRKCSKCGYVDENN |
| | RKQASFKCLKCGYSLNADLNAAVNIAKAFYECPTFRWEEKLHAYVCSEPD |
| | KGGSGGSGGSGGSGGSGGSGGMPSETYITKTLSLKLIPSDEEKQALENYFIT |
| | FQRAVNFAIDRIVDIRSSFRYLNKNEQFPAVCDCCGKKEKIMYVNISNKTFK |
| | FKPSRNQKDRYTKDIYTIKPSGGSKRTADGSEFEPKKKRKV |
| SEQ ID NO: 275 | MKRTADGSEFESPKKKRKVSGGSPGLTGTEYAMAIRKAISILRSFEKRRRN |
| cpPsaCas12f_3 | AERRIIEYEKSKKEYLELIDDVEKGKTNKIVVLEKEGHQRVKRYKHKNWPE |
| | KWQGISLNKAKSKVKDIEKRIKKLKEWKHPTLNRPYVELHKNNVRIVGYE |
| | TVELKLGNKMYTIHFASISNLRKPFRKQKKKSIEYLKHLLTLALKRNLETYP |
| | SIIKRGKNFFLQYPVRVTVKVPKLTKNFKAFGIDRGVNRLAVGCIISKDGKL |
| | TNKNIFFFHGKEAWAKENRYKKIRDRLYAMAKKLRGDKTKKIRLYHEIRK |
| | KFRHKVKYFRRNYLHNISKQIVEIAKENTPTVIVLEDLRYLRERTYRGKGRS |
| | KKAKKTNYKLNTFTYRMLIDMIKYKAEEAGVPVMIIDPRNTSRKCSKCGY |
| | VDENNRKQASFKCLKCGYSLNADLNAAVNIAKAFYECPTFRWEEKLHAY |
| | VCSEPDKGGSGGSGGSGGSGGSGGSGGMPSETYITKTLSLKLIPSDEEKQAL |
| | ENYFITFQRAVNFAIDRIVDIRSSFRYLNKNEQFPAVCDCCGKKEKIMYVNI |
| | SNKTFKFKPSRNQKDRYTKDIYTIKPNAHICKTCYSGVAGNMFIRKQMYPN |
| | DKEGWKVSRSYNIKVNASGGSKRTADGSEFEPKKKRKV |
| SEQ ID NO: 276 | MKRTADGSEFESPKKKRKVSGGSEKWQGISLNKAKSKVKDIEKRIKKLKE |
| cpPsaCas12f_4 | WKHPTLNRPYVELHKNNVRIVGYETVELKLGNKMYTIHFASISNLRKPFRK |
| | QKKKSIEYLKHLLTLALKRNLETYPSIIKRGKNFFLQYPVRVTVKVPKLTKN |
| | FKAFGIDRGVNRLAVGCIISKDGKLTNKNIFFFHGKEAWAKENRYKKIRDR |
| | LYAMAKKLRGDKTKKIRLYHEIRKKFRHKVKYFRRNYLHNISKQIVEIAKE |
| | NTPTVIVLEDLRYLRERTYRGKGRSKKAKKTNYKLNTFTYRMLIDMIKYK |
| | AEEAGVPVMIIDPRNTSRKCSKCGYVDENNRKQASFKCLKCGYSLNADLN |
| | AAVNIAKAFYECPTFRWEEKLHAYVCSEPDKGGSGGSGGSGGSGGSGGSG |
| | GMPSETYITKTLSLKLIPSDEEKQALENYFITFQRAVNFAIDRIVDIRSSFRYL |
| | NKNEQFPAVCDCCGKKEKIMYVNISNKTFKFKPSRNQKDRYTKDIYTIKPN |
| | AHICKTCYSGVAGNMFIRKQMYPNDKEGWKVSRSYNIKVNAPGLTGTEY |
| | AMAIRKAISILRSFEKRRRNAERRIIEYEKSKKEYLELIDDVEKGKTNKIVVL |
| | EKEGHQRVKRYKHKNWPSGGSKRTADGSEFEPKKKRKV |
| SEQ ID NO: 277 | MKRTADGSEFESPKKKRKVSGGSNNVRIVGYETVELKLGNKMYTIHFASIS |
| cpPsaCas12f_5 | NLRKPFRKQKKKSIEYLKHLLTLALKRNLETYPSIIKRGKNFFLQYPVRVTV |
| | KVPKLTKNFKAFGIDRGVNRLAVGCIISKDGKLTNKNIFFFHGKEAWAKEN |
| | RYKKIRDRLYAMAKKLRGDKTKKIRLYHEIRKKFRHKVKYFRRNYLHNIS |
| | KQIVEIAKENTPTVIVLEDLRYLRERTYRGKGRSKKAKKTNYKLNTFTYRM |
| | LIDMIKYKAEEAGVPVMIIDPRNTSRKCSKCGYVDENNRKQASFKCLKCGY |
| | SLNADLNAAVNIAKAFYECPTFRWEEKLHAYVCSEPDKGGSGGSGGSGGS |
| | GGSGGSGGMPSETYITKTLSLKLIPSDEEKQALENYFITFQRAVNFAIDRIVD |
| | IRSSFRYLNKNEQFPAVCDCCGKKEKIMYVNISNKTFKFKPSRNQKDRYTK |
| | DIYTIKPNAHICKTCYSGVAGNMFIRKQMYPNDKEGWKVSRSYNIKVNAP |
| | GLTGTEYAMAIRKAISILRSFEKRRRNAERRIIEYEKSKKEYLELIDDVEKGK |
| | TNKIVVLEKEGHQRVKRYKHKNWPEKWQGISLNKAKSKVKDIEKRIKKLK |
| | EWKHPTLNRPYVELHKSGGSKRTADGSEFEPKKKRKV |
| SEQ ID NO: 278 | MKRTADGSEFESPKKKRKVSGGSDGKLTNKNIFFFHGKEAWAKENRYKKI |
| cpPsaCas12f_6 | RDRLYAMAKKLRGDKTKKIRLYHEIRKKFRHKVKYFRRNYLHNISKQIVEI |
| | AKENTPTVIVLEDLRYLRERTYRGKGRSKKAKKTNYKLNTFTYRMLIDMI |
| | KYKAEEAGVPVMIIDPRNTSRKCSKCGYVDENNRKQASFKCLKCGYSLNA |
| | DLNAAVNIAKAFYECPTFRWEEKLHAYVCSEPDKGGSGGSGGSGGSGGSG |
| | GSGGMPSETYITKTLSLKLIPSDEEKQALENYFITFQRAVNFAIDRIVDIRSSF |
| | RYLNKNEQFPAVCDCCGKKEKIMYVNISNKTFKFKPSRNQKDRYTKDIYTI |
| | KPNAHICKTCYSGVAGNMFIRKQMYPNDKEGWKVSRSYNIKVNAPGLTGT |
| | EYAMAIRKAISILRSFEKRRRNAERRIIEYEKSKKEYLELIDDVEKGKTNKIV |
| | VLEKEGHQRVKRYKHKNWPEKWQGISLNKAKSKVKDIEKRIKKLKEWKH |
| | PTLNRPYVELHKNNVRIVGYETVELKLGNKMYTIHFASISNLRKPFRKQKK |
| | KSIEYLKHLLTLALKRNLETYPSIIKRGKNFFLQYPVRVTVKVPKLTKNFKA |
| | FGIDRGVNRLAVGCIISKSGGSKRTADGSEFEPKKKRKV |
| SEQ ID NO: 279 | MKRTADGSEFESPKKKRKVSGGSKLTKNFKAFGIDRGVNRLAVGCIISKDG |
| cpPsaCas12f_7 | KLTNKNIFFFHGKEAWAKENRYKKIRDRLYAMAKKLRGDKTKKIRLYHEI |
| | RKKFRHKVKYFRRNYLHNISKQIVEIAKENTPTVIVLEDLRYLRERTYRGK |
| | GRSKKAKKTNYKLNTFTYRMLIDMIKYKAEEAGVPVMIIDPRNTSRKCSKC |
| | GYVDENNRKQASFKCLKCGYSLNADLNAAVNIAKAFYECPTFRWEEKLH |
| | AYVCSEPDKGGSGGSGGSGGSGGSGGSGGMPSETYITKTLSLKLIPSDEEK |
| | QALENYFITFQRAVNFAIDRIVDIRSSFRYLNKNEQFPAVCDCCGKKEKIMY |
| | VNISNKTFKFKPSRNQKDRYTKDIYTIKPNAHICKTCYSGVAGNMFIRKQM |
| | YPNDKEGWKVSRSYNIKVNAPGLTGTEYAMAIRKAISILRSFEKRRRNAER |
| | RIIEYEKSKKEYLELIDDVEKGKTNKIVVLEKEGHQRVKRYKHKNWPEKW |
| | QGISLNKAKSKVKDIEKRIKKLKEWKHPTLNRPYVELHKNNVRIVGYETVE |
| | LKLGNKMYTIHFASISNLRKPFRKQKKKSIEYLKHLLTLALKRNLETYPSIIK |
| | RGKNFFLQYPVRVTVKVPSGGSKRTADGSEFEPKKKRKV |
| SEQ ID NO: 280 | MKRTADGSEFESPKKKRKVSGGSKNEQFPAVCDCCGKKEKIMYVNISNKT |
| cpPsaCas12f_8 | FKFKPSRNQKDRYTKDIYTIKPNAHICKTCYSGVAGNMFIRKQMYPNDKEG |
| | WKVSRSYNIKVNAPGLTGTEYAMAIRKAISILRSFEKRRRNAERRIIEYEKS |
| | KKEYLELIDDVEKGKTNKIVVLEKEGHQRVKRYKHKNWPEKWQGISLNK |
| | AKSKVKDIEKRIKKLKEWKHPTLNRPYVELHKNNVRIVGYETVELKLGNK |
| | MYTIHFASISNLRKPFRKQKKKSIEYLKHLLTLALKRNLETYPSIIKRGKNFF |
| | LQYPVRVTVKVPKLTKNFKAFGIDRGVNRLAVGCIISKDGKLTNKNIFFFH |
| | GKEAWAKENRYKKIRDRLYAMAKKLRGDKTKKIRLYHEIRKKFRHKVKY |
| | FRRNYLHNISKQIVEIAKENTPTVIVLEDLRYLRERTYRGKGRSKKAKKTN |
| | YKLNTFTYRMLIDMIKYKAEEAGVPVMIIDPRNTSRKCSKCGYVDENNRK |
| | QASFKCLKCGYSLNADLNAAVNIAKAFYECPTFRWEEKLHAYVCSEPDKG |
| | GSGGSGGSGGSGGSGGSGGMPSETYITKTLSLKLIPSDEEKQALENYFITFQ |
| | RAVNFAIDRIVDIRSSFRYLNSGGSKRTADGSEFEPKKKRKV |
| SEQ ID NO: 281 | MKRTADGSEFESPKKKRKVSGGSKQASFKCLKCGYSLNADLNAAVNIAKA |
| cpPsaCas12f_9 | FYECPTFRWEEKLHAYVCSEPDKGGSGGSGGSGGSGGSGGSGGMPSETYIT |
| | KTLSLKLIPSDEEKQALENYFITFQRAVNFAIDRIVDIRSSFRYLNKNEQFPA |
| | VCDCCGKKEKIMYVNISNKTFKFKPSRNQKDRYTKDIYTIKPNAHICKTCY |
| | SGVAGNMFIRKQMYPNDKEGWKVSRSYNIKVNAPGLTGTEYAMAIRKAIS |
| | ILRSFEKRRRNAERRIIEYEKSKKEYLELIDDVEKGKTNKIVVLEKEGHQRV |
| | KRYKHKNWPEKWQGISLNKAKSKVKDIEKRIKKLKEWKHPTLNRPYVEL |
| | HKNNVRIVGYETVELKLGNKMYTIHFASISNLRKPFRKQKKKSIEYLKHLL |
| | TLALKRNLETYPSIIKRGKNFFLQYPVRVTVKVPKLTKNFKAFGIDRGVNRL |
| | AVGCIISKDGKLTNKNIFFFHGKEAWAKENRYKKIRDRLYAMAKKLRGDK |
| | TKKIRLYHEIRKKFRHKVKYFRRNYLHNISKQIVEIAKENTPTVIVLEDLRY |
| | LRERTYRGKGRSKKAKKTNYKLNTFTYRMLIDMIKYKAEEAGVPVMIIDP |
| | RNTSRKCSKCGYVDENNRSGGSKRTADGSEFEPKKKRKV |
| SEQ ID NO: 282 | MKRTADGSEFESPKKKRKVSGGSAMAKKLRGDKTKKIRLYHEIRKKFRHK |
| cpPsaCas12f_10 | VKYFRRNYLHNISKQIVEIAKENTPTVIVLEDLRYLRERTYRGKGRSKKAK |
| | KTNYKLNTFTYRMLIDMIKYKAEEAGVPVMIIDPRNTSRKCSKCGYVDEN |
| | NRKQASFKCLKCGYSLNADLNAAVNIAKAFYECPTFRWEEKLHAYVCSEP |
| | DKGGSGGSGGSGGSGGSGGSGGMPSETYITKTLSLKLIPSDEEKQALENYFI |
| | TFQRAVNFAIDRIVDIRSSFRYLNKNEQFPAVCDCCGKKEKIMYVNISNKTF |
| | KFKPSRNQKDRYTKDIYTIKPNAHICKTCYSGVAGNMFIRKQMYPNDKEG |
| | WKVSRSYNIKVNAPGLTGTEYAMAIRKAISILRSFEKRRRNAERRIIEYEKS |
| | KKEYLELIDDVEKGKTNKIVVLEKEGHQRVKRYKHKNWPEKWQGISLNK |
| | AKSKVKDIEKRIKKLKEWKHPTLNRPYVELHKNNVRIVGYETVELKLGNK |
| | MYTIHFASISNLRKPFRKQKKKSIEYLKHLLTLALKRNLETYPSIIKRGKNFF |
| | LQYPVRVTVKVPKLTKNFKAFGIDRGVNRLAVGCIISKDGKLTNKNIFFFH |
| | GKEAWAKENRYKKIRDRLYSGGSKRTADGSEFEPKKKRKV |
| SEQ ID NO: 283 | MKRTADGSEFESPKKKRKVSGGSRYKHKNWPEKWQGISLNKAKSKVKDI |
| cpPsaCas12f_11 | EKRIKKLKEWKHPTLNRPYVELHKNNVRIVGYETVELKLGNKMYTIHFASI |
| | SNLRKPFRKQKKKSIEYLKHLLTLALKRNLETYPSIIKRGKNFFLQYPVRVT |
| | VKVPKLTKNFKAFGIDRGVNRLAVGCIISKDGKLTNKNIFFFHGKEAWAKE |
| | NRYKKIRDRLYAMAKKLRGDKTKKIRLYHEIRKKFRHKVKYFRRNYLHNI |
| | SKQIVEIAKENTPTVIVLEDLRYLRERTYRGKGRSKKAKKTNYKLNTFTYR |
| | MLIDMIKYKAEEAGVPVMIIDPRNTSRKCSKCGYVDENNRKQASFKCLKC |
| | GYSLNADLNAAVNIAKAFYECPTFRWEEKLHAYVCSEPDKGGSGGSGGSG |
| | GSGGSGGSGGMPSETYITKTLSLKLIPSDEEKQALENYFITFQRAVNFAIDRI |
| | VDIRSSFRYLNKNEQFPAVCDCCGKKEKIMYVNISNKTFKFKPSRNQKDRY |
| | TKDIYTIKPNAHICKTCYSGVAGNMFIRKQMYPNDKEGWKVSRSYNIKVN |
| | APGLTGTEYAMAIRKAISILRSFEKRRRNAERRIIEYEKSKKEYLELIDDVEK |
| | GKTNKIVVLEKEGHQRVKSGGSKRTADGSEFEPKKKRKV |
| SEQ ID NO: 284 | MKRTADGSEFESPKKKRKVSGGSNTSRKCSKCGYVDENNRKQASFKCLKC |
| cpPsaCas12f_12 | GYSLNADLNAAVNIAKAFYECPTFRWEEKLHAYVCSEPDKGGSGGSGGSG |
| | GSGGSGGSGGMPSETYITKTLSLKLIPSDEEKQALENYFITFQRAVNFAIDRI |
| | VDIRSSFRYLNKNEQFPAVCDCCGKKEKIMYVNISNKTFKFKPSRNQKDRY |
| | TKDIYTIKPNAHICKTCYSGVAGNMFIRKQMYPNDKEGWKVSRSYNIKVN |
| | APGLTGTEYAMAIRKAISILRSFEKRRRNAERRIIEYEKSKKEYLELIDDVEK |
| | GKTNKIVVLEKEGHQRVKRYKHKNWPEKWQGISLNKAKSKVKDIEKRIKK |
| | LKEWKHPTLNRPYVELHKNNVRIVGYETVELKLGNKMYTIHFASISNLRKP |
| | FRKQKKKSIEYLKHLLTLALKRNLETYPSIIKRGKNFFLQYPVRVTVKVPKL |
| | TKNFKAFGIDRGVNRLAVGCIISKDGKLTNKNIFFFHGKEAWAKENRYKKI |
| | RDRLYAMAKKLRGDKTKKIRLYHEIRKKFRHKVKYFRRNYLHNISKQIVEI |
| | AKENTPTVIVLEDLRYLRERTYRGKGRSKKAKKTNYKLNTFTYRMLIDMI |
| | KYKAEEAGVPVMIIDPRSGGSKRTADGSEFEPKKKRKV |

### EXAMPLES

While several experimental Examples are contemplated, these Examples are intended non-limiting.

### Example 1

### Computational Discovery of Miniature CRISPR Nucleases

The computational discovery of miniature CRISPR nucleases was performed (FIGS. 1A-1D).

Novel miniature CRISPR nucleases from metagenomic samples were identified by computer discovery (FIG. 1A). Initial panning for small CRISPR nucleases yielded orthologs, including 30 novel Cas12f orthologs, 20 novel Cas12j orthologs, and 45 novel Cas12m orthologs (FIG. 1B). These orthologs comprise a C-terminal RuvC domain indicative of Cas12 systems and CRISPR arrays of 2 or more spacers with direct repeats that fold with an appropriate secondary structure (FIG. 1E). The Cas12f and Cas 12m systems have readily identifiable putative tracrRNAs found by a homology search of the DR against the surrounding locus and a secondary structure modeling/prediction to identify the tracrRNA sequence with the best folding energy to the crRNA (FIG. 1F). The Cas12js systems do not have any identifiable tracrRNA and the Cas12m systems do have identifiable tracrRNAs. The new subclasses of Cas12s require or do not require tracrRNA.

FIG. 1C shows the size distribution of Cas12a and FIG. 1D shows the size distribution of CasM ortholog.

### Example 2—PsaCas12f sgRNA Constructs

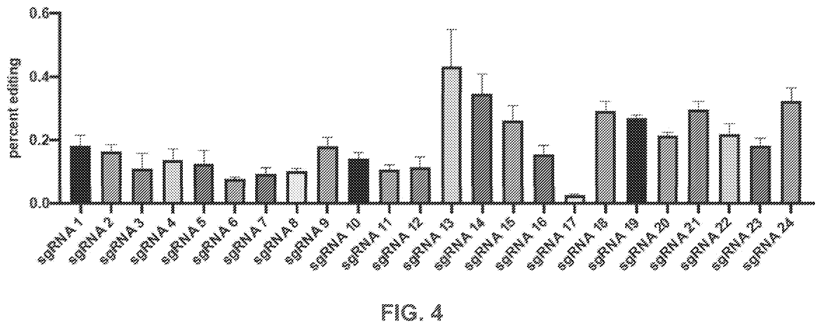

PsaCas12f sgRNA constructs were tested in human mammalian cells (FIG. 4).

A panel of 24 sgRNA designs against a pUC19 reported plasmid with PsaCas12f was tested. The sgRNA designs are disclosed in Table 1 and achieved up to about 0.5% editing. The experiments were performed with plasmid expression in HEK293FT for 48-72 hours.

### Example 3—PsaCas12f sgRNA Designs Based on sgRNA Secondary Structure

SgRNA's secondary structure is critical to enabling the specific and effective recognition between Cas9 and the target sequence. To further improve the cleavage efficiency of the PsaCas12f-sgRNA complex, sgRNA variants were designed to comprise genetic mutations which would impact the sgRNA's secondary structure as well as interactions with the sgRNA-protein complex.

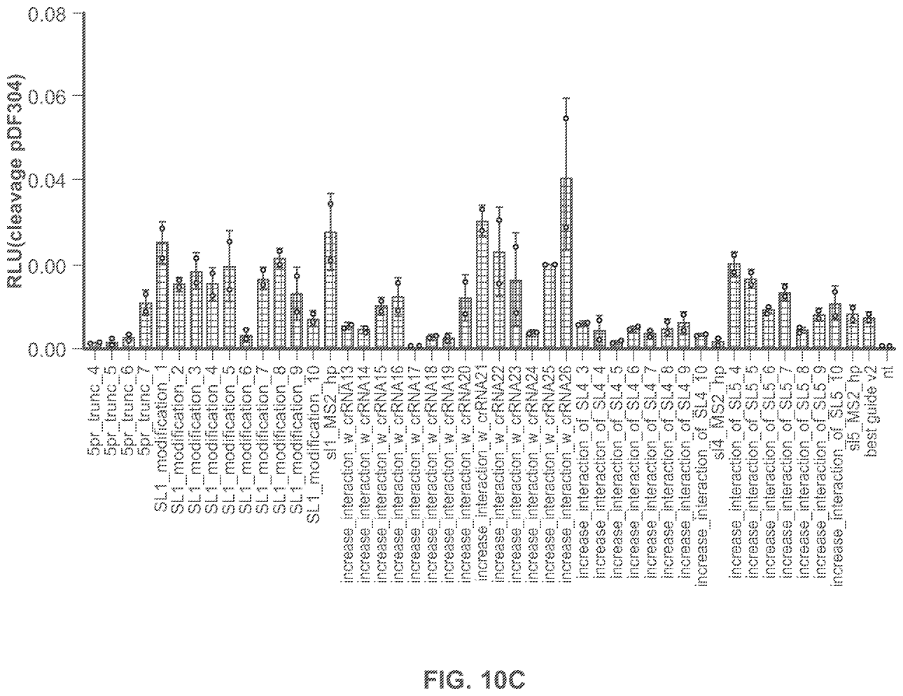

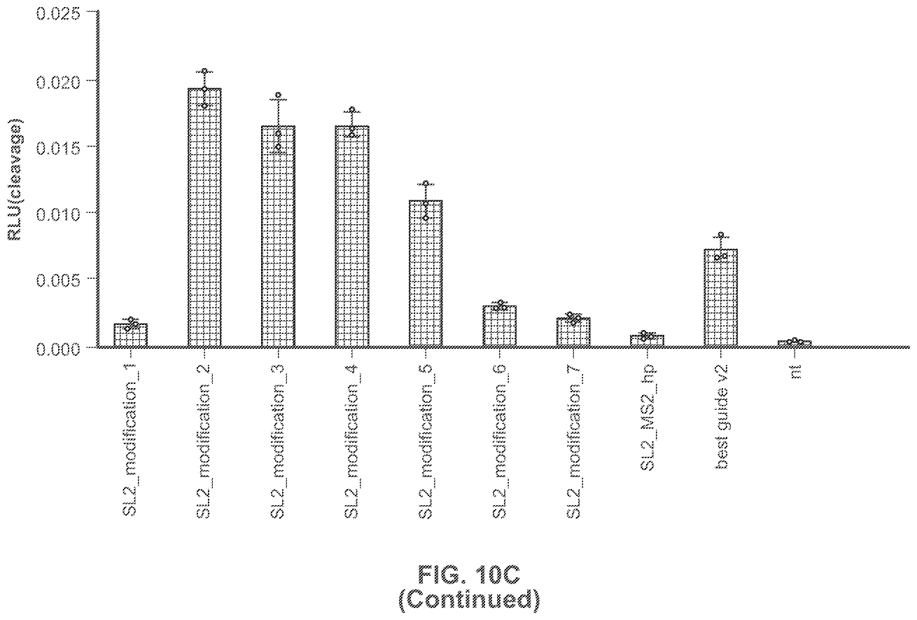

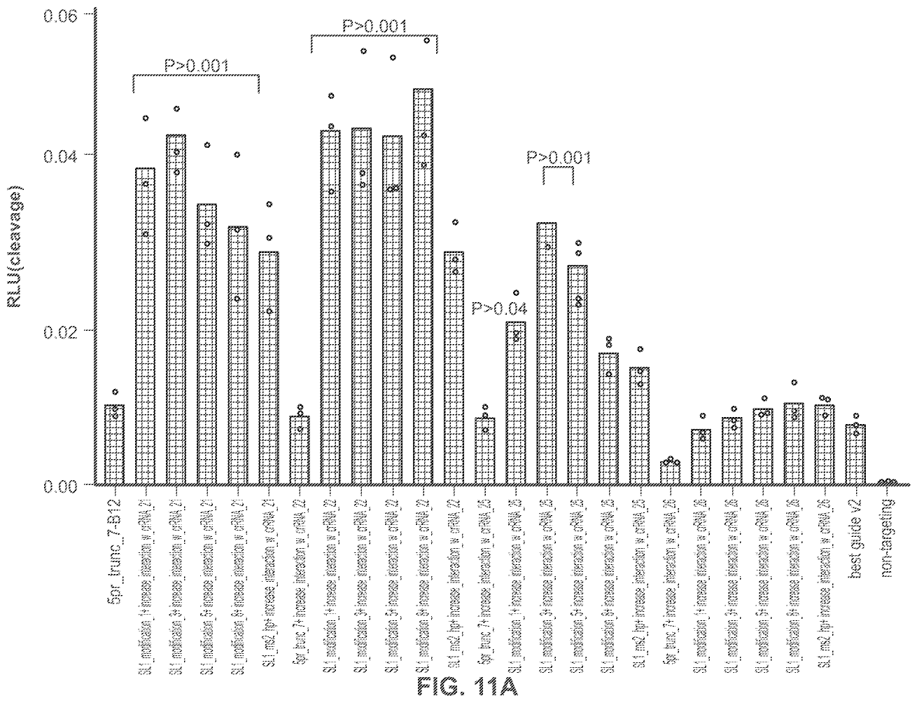

The predicted sgRNA secondary structure was obtained through use of in silico structure determination. Stem loop 1-3 (SL1-3) were predicted via http://rna.tbi.univie.ac.at/. Stem loop 4 (SL4, interacts with crRNA) and stem loop 5 (SL5) were informed by Takeda et al., Mol Cell, 81(3):558-570 (2021). FIG. 10A illustrates the resulting sgRNA secondary structure with SL1-SL3 marked by blue, red, and green boxes, respectively.

Using this predicted sgRNA secondary structure, genetic mutations were engineered into SLa, SL2, SL3, SL4, or SL5. FIG. 10B lists and annotates all the sgRNA variants designed (see also sequence listing in Table 14). Red denotes nucleobase changes that were introduced, orange denotes nucleobases that form stems, and violet denotes loops that were added to allow recruitment of MS2 coat/proteins.

Subsequently, using an in vitro luciferase reporter assay, the sgRNA variants were tested to assess whether secondary structure modifications of SL1-SL5 could impact cleavage efficiency. Briefly, HEK293T cells were seeded and transfected with 25 ng of a luciferase reporter, 100ng of different CRISPR guides annotated above, and 300ng of PsaCas12f-expressing plasmid. Seventy-two hours after transfection, media was harvested from cells and analyzed for luciferase expression.

The corresponding bar graph in FIG. 10C shows the results of the reporter assay. Notably, certain genetic modifications to SL1, SL2, SL3, SL4, or SL5 increased the cleavage efficiency over controls (control sgRNA constructs previously optimized using a different strategy, labeled “5pr_trunc4-7” and “best guide v2”).

### Example 4—PsaCas12f sgRNA Combination Mutant Stem-Loop Constructs

The sgRNA variants in Example 3 each targeted a different stem-loop regions (SL1, SL2, SL3, SL4, or SL5). It was hypothesized that each stem-loop region may impact a variety of functions (e.g., hairpin stability, transcription efficiency, protein interaction) and that combining the single stem-loop mutant variants designed in Example 3 would further improve cleavage efficiency. Accordingly, sgRNA variants which contained a combination of modifications from the sgRNA variants with single modifications at a particular stem-loop region was designed (also called, “combination constructs”). The aim of the sgRNA combination stem-loop variants was to increase folding and Cas12f interaction (e.g., GC content increase, sgRNA truncation/mismatch correction in stem loops, removal of premature termination signals).

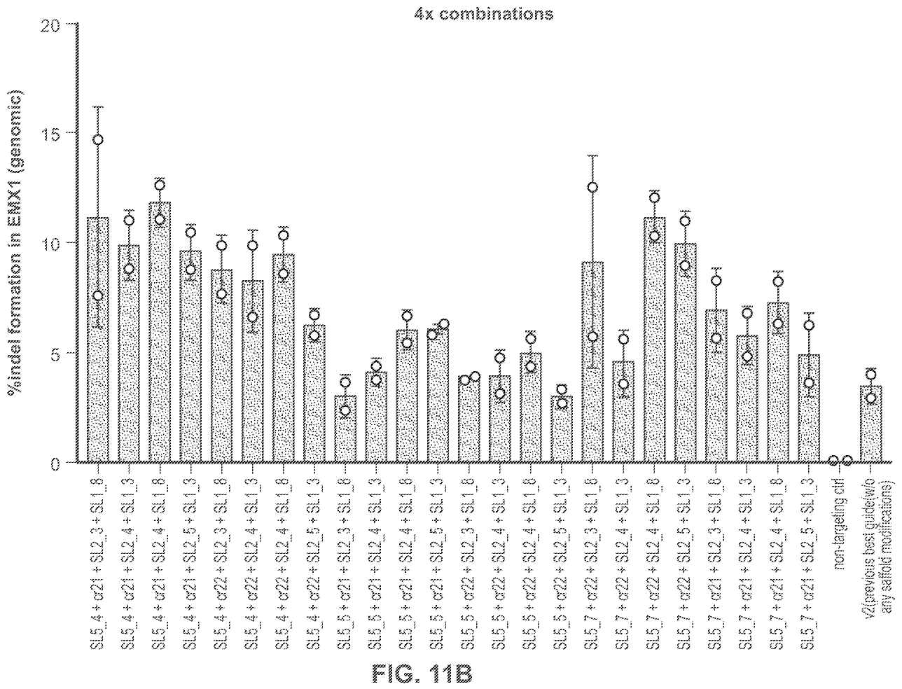

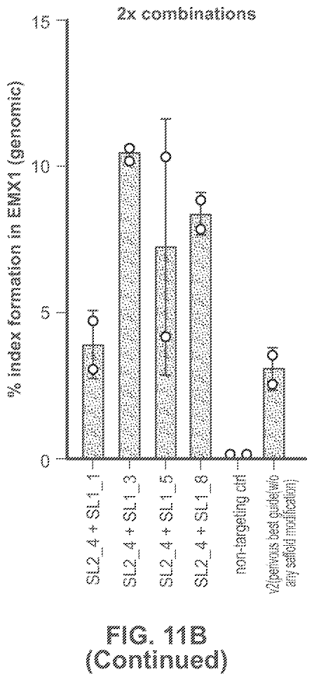

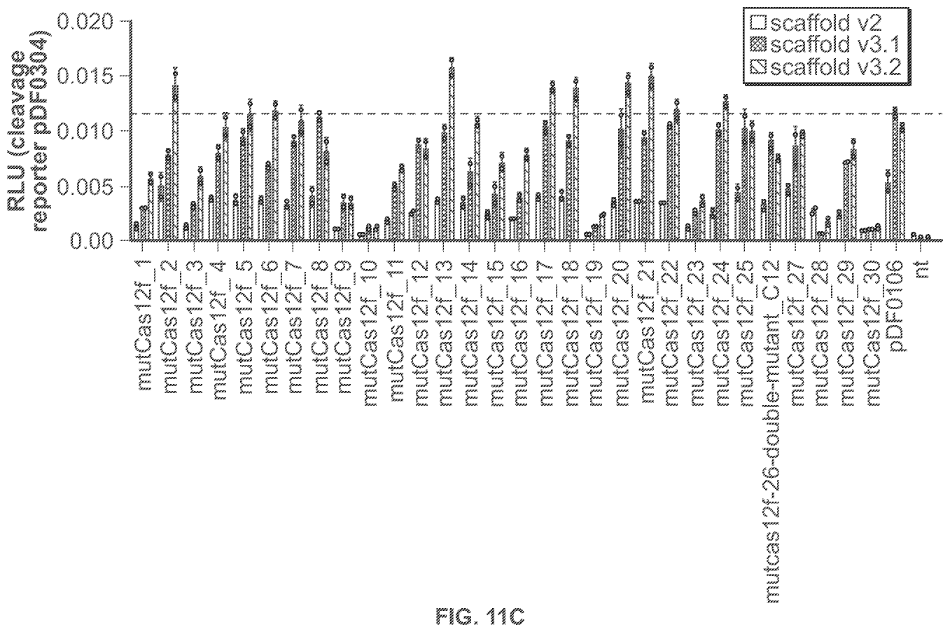

Combination constructs are presented in Table 16. FIG. 11A shows the resulting performance of the combination constructs relative to controls in the in vitro luciferase reporter assay. Surprisingly, certain combinations, such as, the construct labeled, “SL1_modification_1+increase_interaction_w_crRNA_22,” resulted in enhanced cleavage efficiency (about 0.035% RLU cleavage) relative to the single modification construct labeled, “SL1_modification_1,” (about 0.025% RLU cleavage), compare FIG. 10C to FIG. 11A).

Subsequently, combination constructs, either double variants with modifications of stem loop 1 and 2 (labeled, 2× combinations in FIG. 11B) or quadruple variants with modifications of stem loop 1, 2, 3, and 5 (labeled 4× combinations in FIG. 11B) were interrogated for cleavage efficiency at the EMX1 (empty spiracles-like protein 1) locus.

Briefly to measure cleavage efficiency at the EMX1 locus, 100ng of different CRISPR guides annotated above in Table 16 and 300ng of PsaCas12f-expressing plasmid were transfected into HEK293FT cells. Seventy-two hours after transfection, cells were harvested for their genomic DNA and primers amplifying EMX1 genomic locus were used to amplify the genomic region in the locus. Subsequently, next generation sequencing (NGS) was performed on these amplified gDNA and the insertion/deletion profile caused by Cas12f with the different guides was analyzed with CRISPResso.

FIG. 11B shows the result of the editing efficiencies at the EMX1 locus for the combination constructs noted above. Notably, for the 4× combination constructs tested, the construct labeled, “SL5_4+cr21+SL2_4+SL1_8,” had greater editing efficiency at the EMX1 locus than the control constructs with either a single stem-loop modification or no stem-loop modification. It is not entirely obvious why certain combination constructs work better than other combination. For example, compare the EMX1 editing efficiency of the 2× combinations “SL2_4+SL1_1” with “SL2_4+SL1_3.” One hypothesis is that certain base-pair combinations do not provide optimal sgRNA folding/sgRNA-protein interaction and these occurrences are difficult to predict in silico.

The best sgRNA combination mutant stem-loop constructs named (1) scaffold “version 2”, (2) “version 3.1, SL1_modification_8+increase_interaction_w_crRNA_21, or SEQ ID NO: 203”, and (3) “v. 3.2, SEQ ID NO: 198”) from FIGS. 11A and 11B were subsequently tested with 30 different PsaCas12f mutants relative to controls in the in vitro luciferase reporter assay the order to test the robustness of the sgRNA scaffold as shown in FIG. 11C. Notably, scaffold “v. 3.2” which includes the modification of mutant combination “SL1_8” and “interaction_w_cRNA_22” performed well across the panel of PsaCas12f mutants tested demonstrating the robustness of the “v.3.2” as a sgRNA scaffold.

### Example 5—Spacer Optimization for sgRNA Scaffold Version 3.2 for PsaCas12f

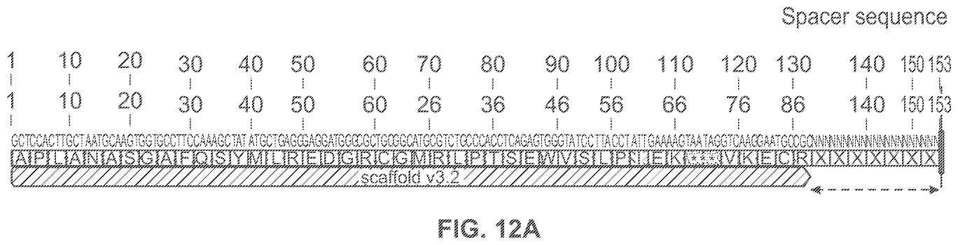

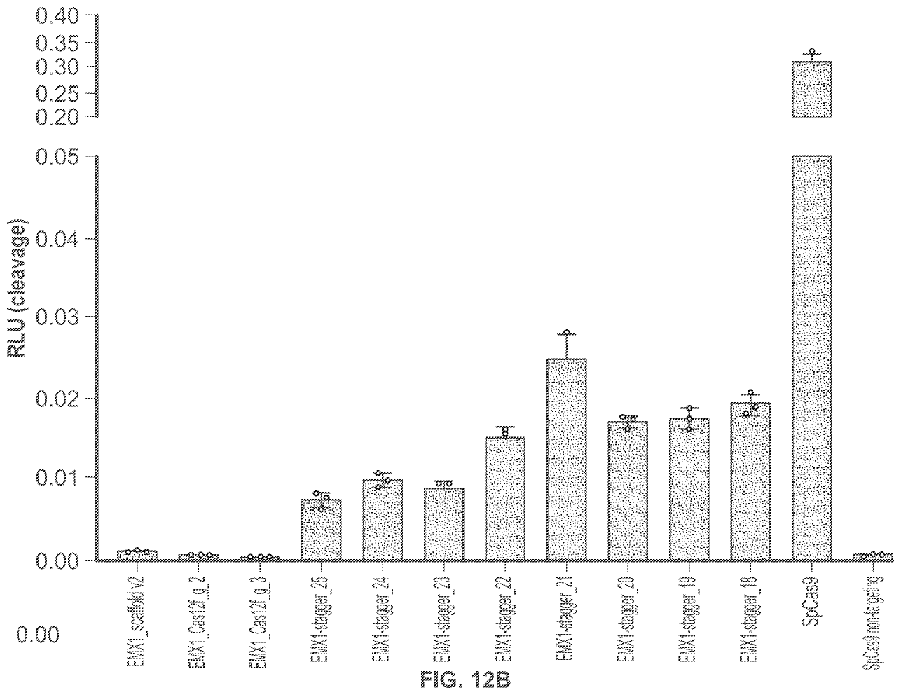

The sgRNA spacer sequence can impact target specificity and the degree of off-target activity. FIG. 12A is a schematic of the sgRNA scaffold version 3.2 which highlights the position of the spacer sequence at the 3′ end. This experiment was designed to test the cleavage efficiency of the sgRNA v. 3.2 scaffold from Example 4 by varying the nucleotide length of the sgRNA spacer sequence.

To test spacer length, the version 3.2 sgRNA scaffold was tested in the in vitro luciferase reporter assay at spacer sequence lengths of 2, 3, 18, 19, 20, 21, 22, 23, 24, and 25 base pairs relative to controls. FIG. 12B shows that using v3.2 sgRNA scaffold for PsaCas12f, the highest cleavage efficiency was achieved using a spacer sequence of 21 bp for this specific target. While 22 bp, 20 bp, 19 bp and even 18 bp still worked, 21 bp showed the highest gene editing. As such, for the PsaCas12f-version3.2 sgRNA 20 bp or 21 bp is enough to allow sufficient base-pairing before cleavage.

### Example 6—PsaCas12f with the sgRNA Scaffold Version 3.2 is More Efficacious than UnCas12f (Cas14a1)

PsaCas12f with the sgRNA scaffold version 3.2 described in Example 4 was then compared to a different Cas12f protein which is similarly small and has good on-target efficiency called, Un1Cas12f1 (also called Cas14a1) at either the HBB (hemoglobin subunit beta) or the RNF2 (ring finger protein 2) genomic locus. UnlCas12f1 is a protein identified from an uncultured archaeon (Un1).

Briefly, 100ng of different CRISPR guides based on scaffold version 2 with different spacer lengths according to their descriptions (e.g., stagger_24 denotes a spacer length of 24 nt) annotated in Table 17 and 300ng of PsaCas12f-expressing plasmid are transfected into HEK293FT cells. Two spacer sequences targeting either RNF2 or HBB genomic locus were designed with sgRNA v3.2 scaffold. Seventy-two hours after transfection, cells were harvested for their genomic DNA and primers amplifying the corresponding genomic locus were used to amplify the gDNA in the locus. Subsequently, next generation sequencing (NGS) was performed on these amplified gDNA, and insertion/deletion profile caused by Cas12f with different guide was analyzed with CRISPResso.

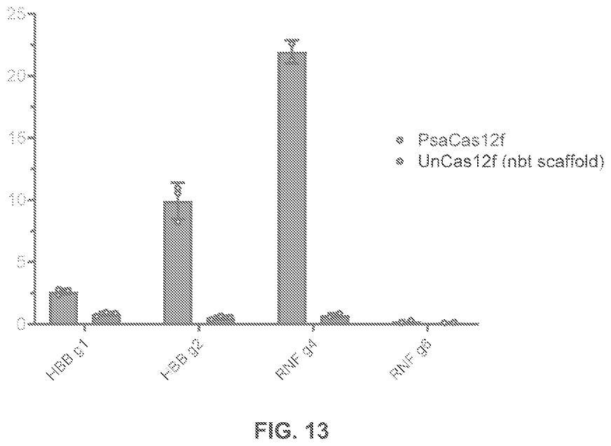

FIG. 13 shows that PsaCas12f with the sgRNA scaffold version 3.2 outperformed Un1Cas12f1 with the nbt scaffold in terms of indel activity (insertion/deletion formation) at both sites tested in the Hbb locus (g1 and g2) as well as one a site in the RNF locus (g4). As such, PsaCas12f with the sgRNA scaffold version 3.2 allows efficient indel formation and may be a useful tool for broad genome engineering applications.

### Example 7—PsaCas12f NLS Constructs

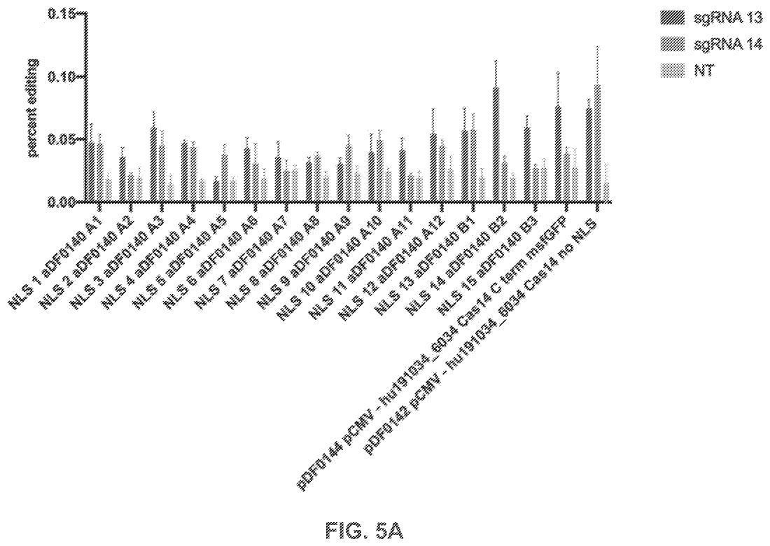

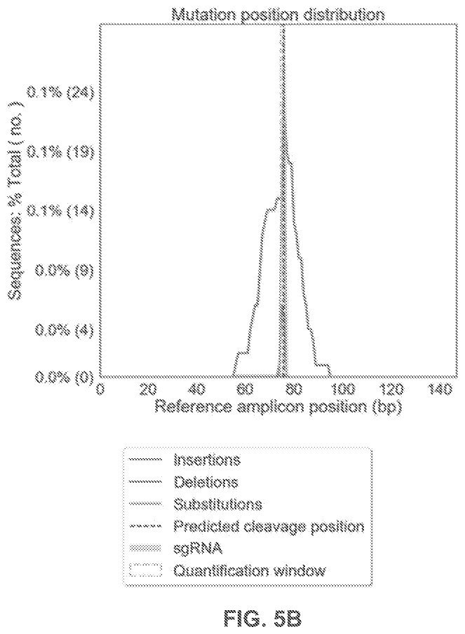

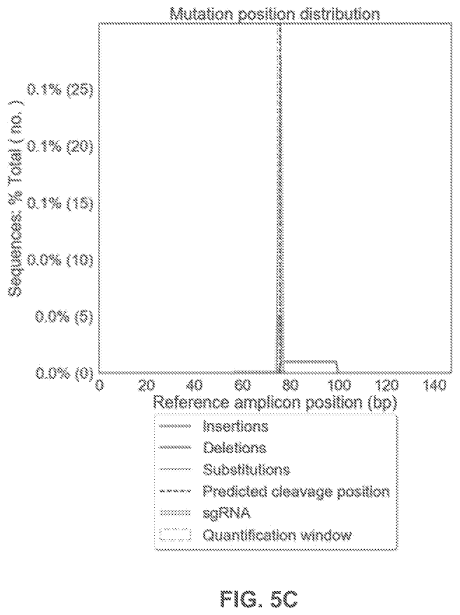

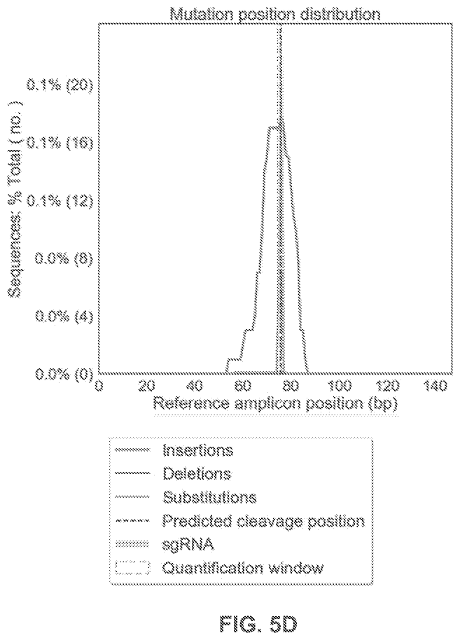

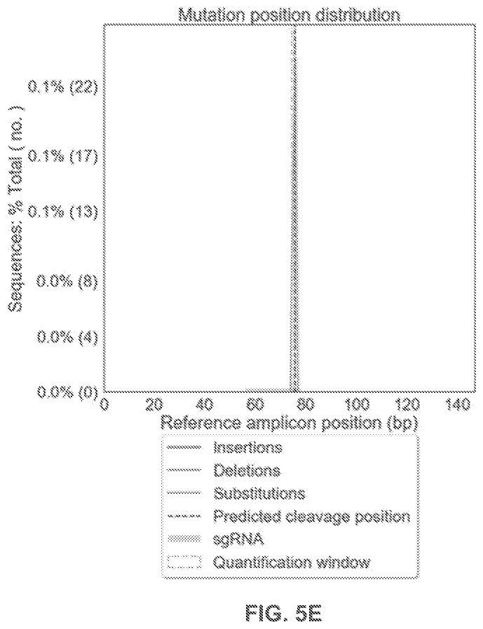

PsaCas12f Nuclear Localization Signals (NLS) constructs were tested in HEK293FT human mammalian cells (FIG. 5A-5D).

A panel of 15 NLS designs fused to PsaCas12f against a pUC19 reported plasmid using the top two guide sequences from Example 2 was tested. The NLS designs are disclosed in Table 1 and achieve up to about 0.1% editing (FIG. 5A). The experiments were performed with plasmid expression in HEK293FT for 48-72 hours. The sequencing traces show bona-fide editing as illustrated in FIGS. 5B-5E. Editing with PsaCas12f (NLS14) with sgRNA (FIG. 5B) or non-targeting guide (FIG. 5C) shows clear deletions (purple) and insertions (red). Editing with PsaCas12f (no NLS) with sgRNA (FIG. 5D) or non-targeting target guide (FIG. 5E) also shows clear deletion (purple) and insertions (red).

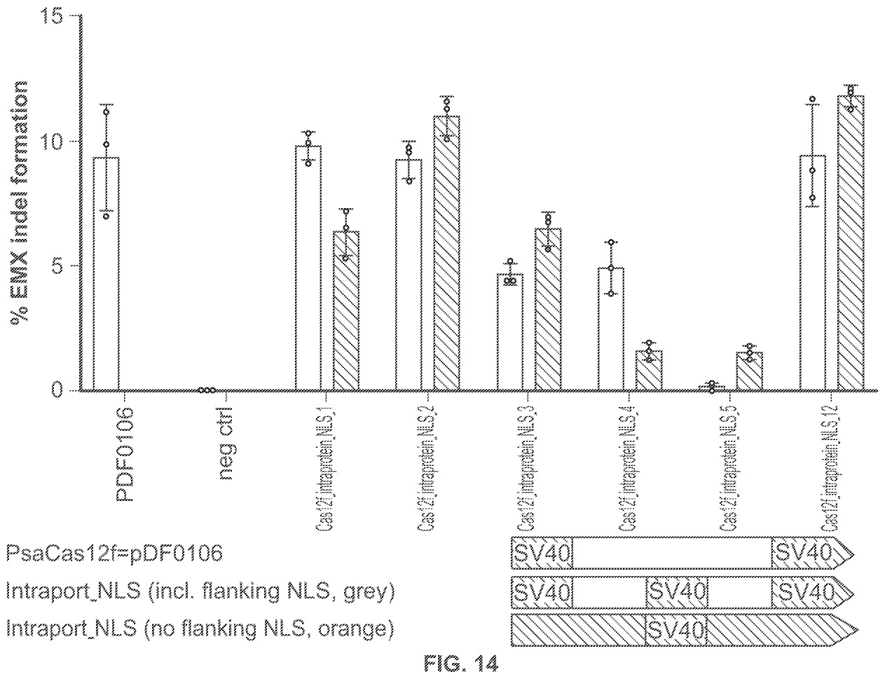

Intra NLS signals could allow better design of proteins delivered via viral-like particles, Banskota et al., Cell, 185(2):250-265 (2022), or enable inducible NLS signals following conformational change, Saleh et al., Exp Cell Res, 260(1):105-115 (2000). As such, an intra-protein NLS sequence derived from SV40 (simian virus 40) was fused at random positions into PsaCas12f as shown in FIG. 14 and annotated in Table 18. These constructs were tested for indel activity at the EMX genomic locus.

Briefly, seventy-two hours after transfection, cells were harvested for their genomic DNA and primers amplifying the corresponding EMX genomic locus was used to amplify the gDNA in the locus. Subsequently, next generation sequencing (NGS) is performed on these amplified gDNA, and insertion/deletion profile was analyzed with CRISPResso.

Intra NLS signals, labeled “NLS_2”, “NLS_3”, “NLS-5”, and “NLS_6,” had higher indel activity at the EMX locus than wild-type PsaCas12f which was flanked by two NLS sequences on the N- and C-terminus (labeled, “pDF0106”) as shown in FIG. 14. Therefore, intra NLS signals could provide alternative localization to flanking NLS signals while still maintaining optimal gene editing activity. Intra NLS signals could be advantageous for example, when the N- or C-terminal NLS fusions interfere with protein function.

### Example 8—CRISPR Editing with PsaCas12f and Guide RNA Delivered by Adeno-Associated Virus (AAV)

Adeno associated virus (AAV) is a US Food and Drug administration approved safe vehicle for gene therapies and for this reason AAV-loadable CRISPR tools are advantageous. AAV has a limited payload size of <4.7 kb which hampers clinical applications of most CRISPR tools. Therefore, this Example validates AAV delivery of PsaCas12f-sgRNA.

Briefly, PsaCas12f with the best NLS configuration (flanking SV40NLS) was cloned into AAV ITR along with a guide targeting RUNX1 (runt-related transcription factor 1) genomic locus. Subsequently, the plasmid was transfected into HEK293FT cells with AAV helper plasmid to make AAV particles. AAV particles in the media from the producer cell line was collected and subsequently added to HEK293FT cells. Four days after transduction, the indel profile at the RUNX1 locus was analyzed with NGS.

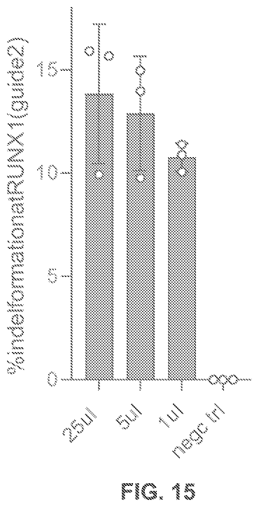

As shown in FIG. 15, the AAV-loaded with PsaCas12f plus guide had indel frequencies of about 10-14% at the RUNX1 genomic locus increasing commensurately with the amount transduced into HEK293 cells (1, 5, or 25 μl). This experiment demonstrates that PsaCas12f can be effectively expressed from AAV particles while maintaining the ability to induce cleavage at a genomic target.

### Example 9—PsaCas12f with Guide CrRNA/TracrRNA

PsaCas12f with CrRNA/tracrRNA guide was screened at different free-energy local minima (FIG. 6).

Results from PsaCas12f show that many crRNA/tracrRNA designs must be screened at a variety of free-energy local minima to find optimal combinations for activity in bacterial or mammalian protein lysate. A 20-nt DR and 90-nt tracrRNA were found to provide optimal activity for dsDNA cleavage and that they can be combined for a sgRNA. These designs showed that the computational and experimental RNA screening can yield optimal designs and that sgRNA has a significant effect on activity.

### Example 10—Genome Editing by Cas12f Family Members

Cas12f family members were tested for genome editing (FIG. 7). These tests from Cas12f family members for indel generation at EMX1 result in editing efficiencies above background.

### Example 11—Screening of a Panel of 12 Cas12f Orthologs

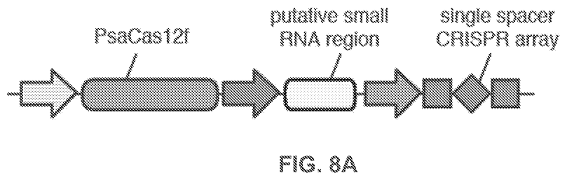

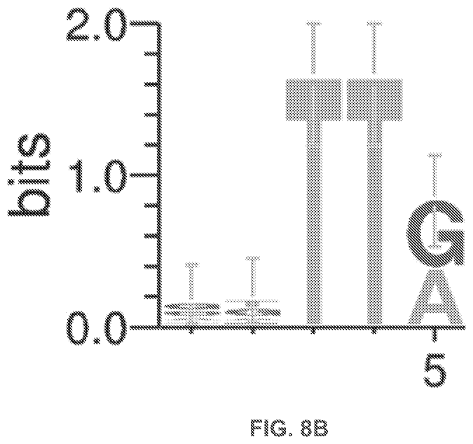

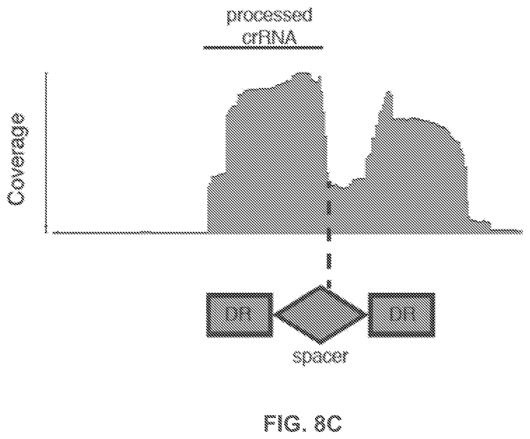

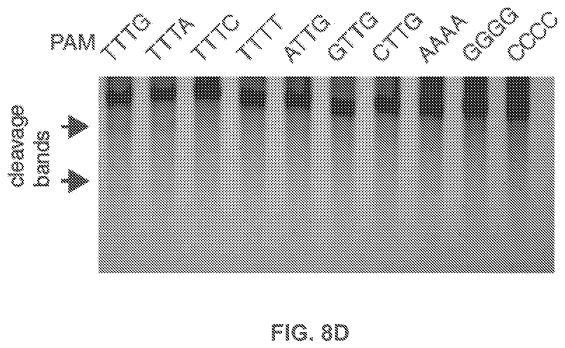

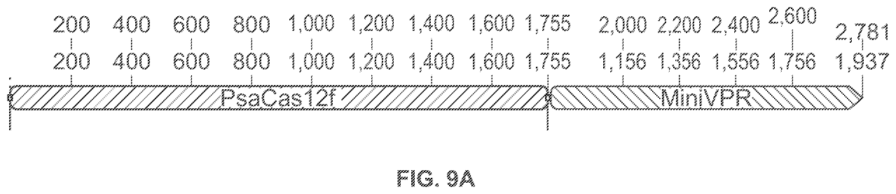

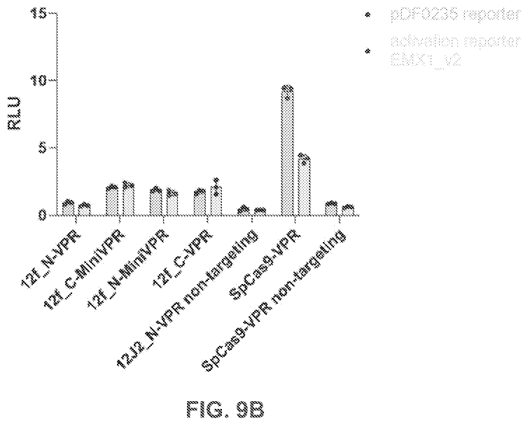

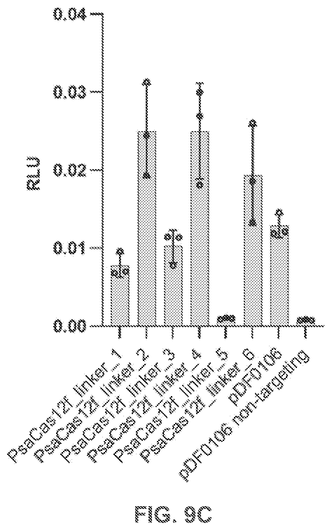

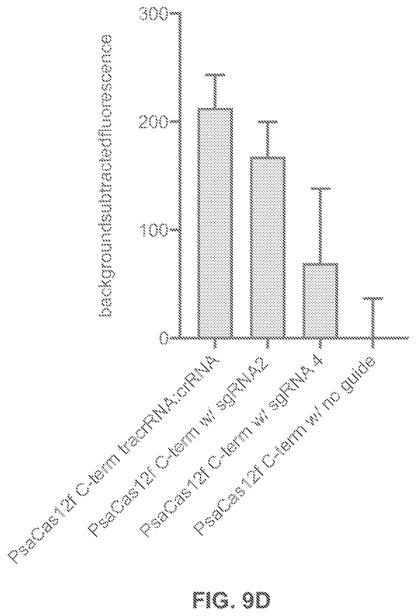

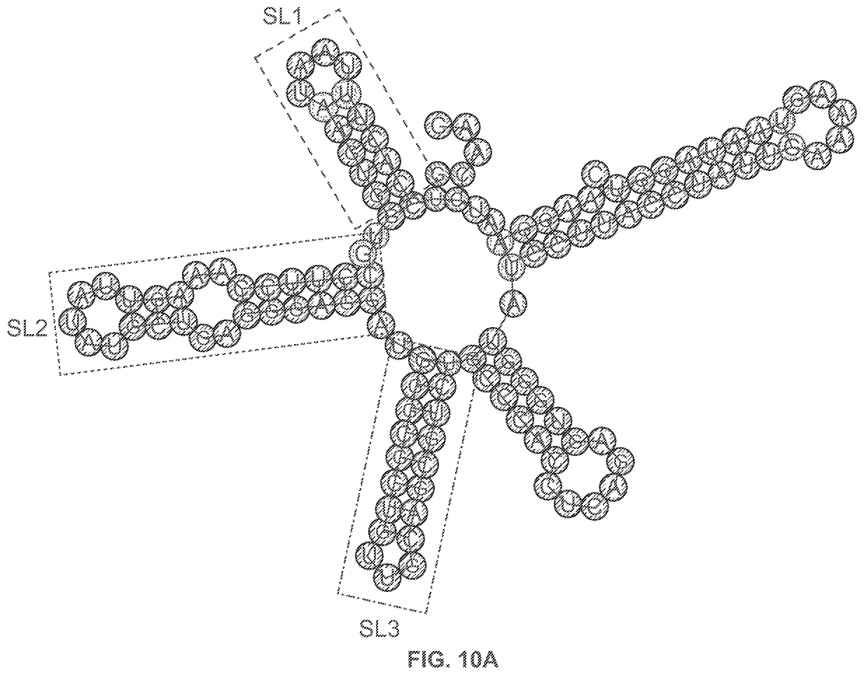

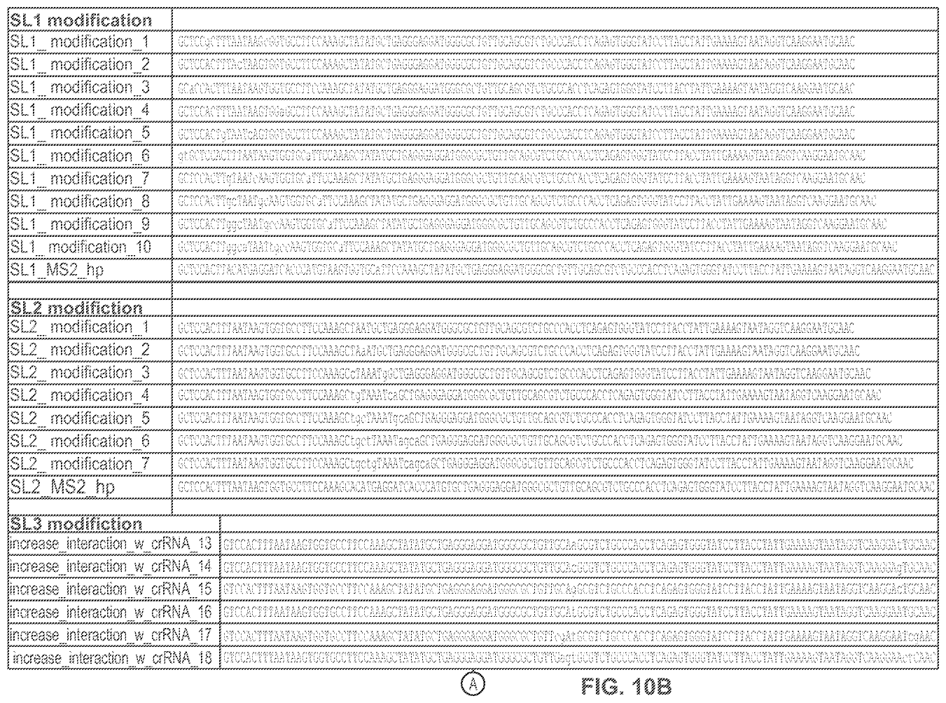

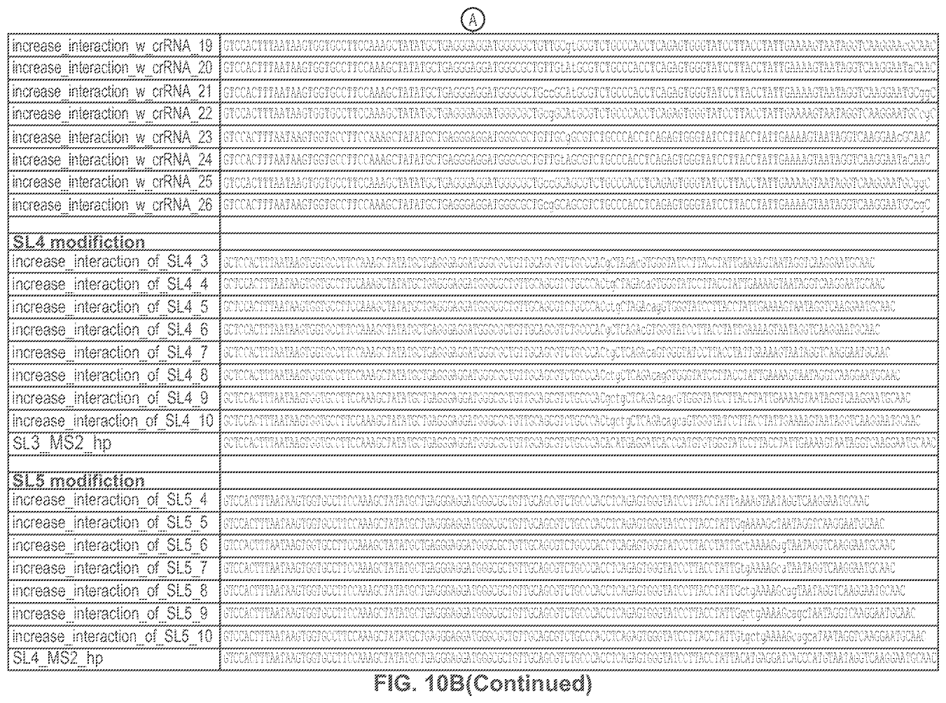

A panel of 12 novel Cas12f orthologs ranging in size between 400-800 amino acids was screened. In order to maintain the correct small RNA species from these orthologs, non-coding regions from the surrounding loci along with the Cas12f genes were cloned (FIG. 8A). Purification of lysate from these samples enabled testing of in vitro cleavage on degenerate PAM libraries, where cleaved fragments can be enriched to determine the PAM. Of all 12 proteins, one of the orthologs, the Cas12f from Pseudomonas aeruginosa (g-proteobacteria) (PsaCas12f), a 586-residue protein, had substantial cleavage activity determined by this high-throughput PAM screen. PAM characterization had determined the motif of PsaCas12f to be TTR (FIG. 8B). Additionally, small RNA sequencing of these purified proteins can determine the mature isoforms of the processed crRNA and tracrRNA (FIG. 8C), yielding a natural DR length of 31 nt and tracrRNA length of 97 nt. Lastly, the PAM of PsaCas12f on fixed sequence targets was validated to demonstrate detectable in vitro cleavage by gel readouts (FIG. 8D). The characterization of PsaCas12f and the corresponding RNA species, as well as other effectors selected from the high-throughput screening can be optimized for activity by guide RNA engineering.

### Example 12—PsaCas12f Circular Permutation

While Cas nucleases did not evolve to function as a modular DNA-binding scaffold optimizing Cas nucleases by fusion to functional protein domains using linkers may enable controlled nuclease activity and broaden the use of Cas nuclease as a genetic tool. Oakes et al. Cell, 176(2): 254-267 (2019). One way to change the CRISPR architecture to enable fusion to other protein domains is by protein circular permutation (CP). Id. CP is the topological rearrangement of a protein's primary sequence, connecting its N- and C-terminus with a peptide linker, while concurrently splitting its sequence at a different position to create new, adjacent N and C termini. Yu and Lutz, Trends Biotechnol, 28: 18-25 (2011).

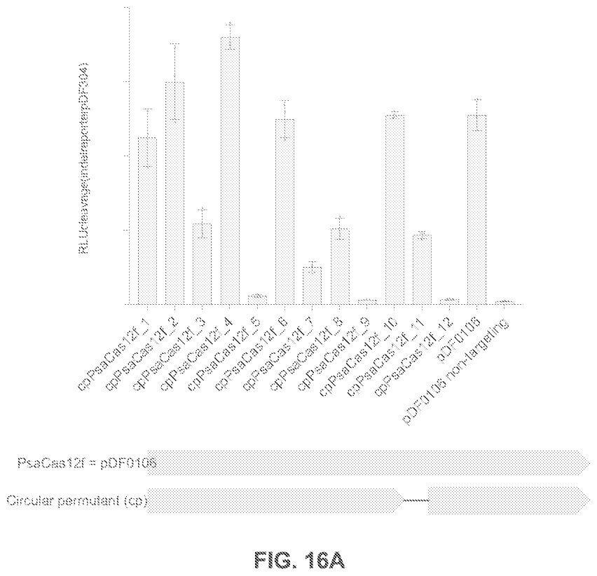

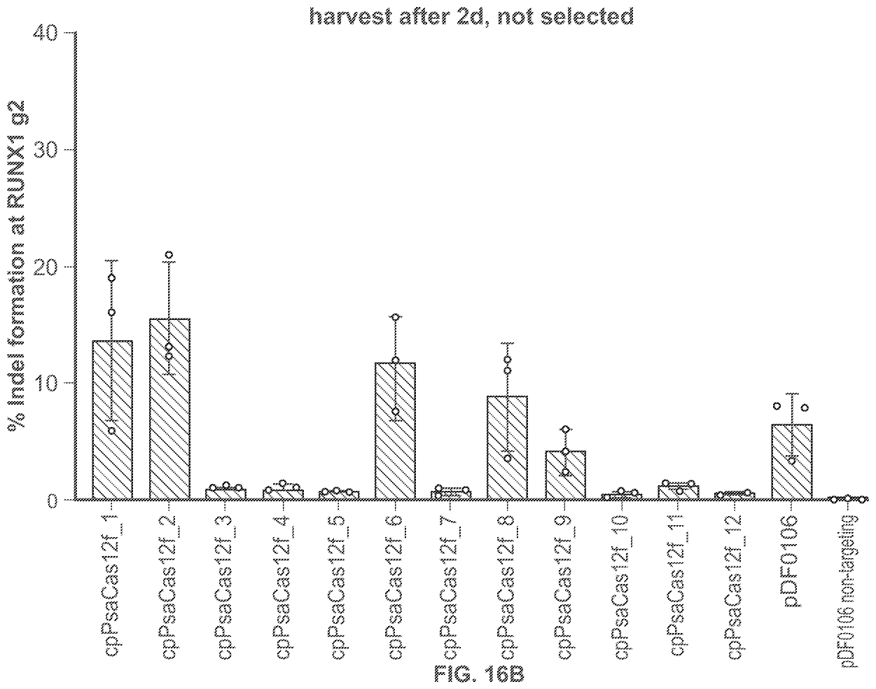

To test whether PsaCas12f proteins as described above could undergo circular permutation without impacting functional activity, the PsaCas12f sequence was split at different positions to create new adjacent N- and C-termini using a (GGS)₆ peptide linker (SEO ID NO: 286) as shown in Table 15 (see also, bottom schematic in FIG. 16A).

Circular permutation constructs listed in Table 21 were then tested for editing efficiency either using the in vitro luciferase reporter assay described above or by testing indel formation at the RUNX1 genomic locus as shown in FIG. 16A and FIG. 16B, respectively.

Briefly, for the in vitro luciferase reporter assay 25ng of Gluc reporter, 100ng of the CRISPR guide, and 300ng of either regular PsaCas12f-expressing plasmid (control, labeled pDF0106) or different circular permutation of the protein encoding plasmids were transfected into HEK293FT cells. Seventy-two hours after transfection, media is harvested from cells and analyzed for luciferase expression. For assessment of indel formation at the RUNX1 genomic locus, the same panel of circular permutations of PsaCas12f proteins were tested with guides targeting genomic RUNX1 locus. Cell transfection conditions were the same as for the in vitro luciferase, PCR was used to amplify the genomic locus at RUNX1 and indel efficiency estimated by CRISPResso.

Notably, some circular permutations of PsaCas12f are functional and allow for different positioned N- and C-termini. Interestingly, the editing efficiency changes depending on the guide that is used (compare editing efficiencies from FIG. 16A and FIG. 16B).

### Example 13—PsaCas12f Sequence Optimization via Machine Learning

The wild-type PsaCas12f sequences was sent to a machine learning model (Facebook Evolutionary Scale Modeling (ESM), https://github.com/facebookresearch/esm) for prediction of point mutations on the protein that could result in higher editing efficiencies. Namely, the original WT sequence was used as input in the ESM model. The output of the ESM model was a single vector (1×1280), and this vector was subsequently used as an input in a linear regression model to predict the output which is the indel formation rate. New mutations made on the protein were sent through the model in a similar fashion to predict the indel and subsequently tested in vitro.

Forty-eight different point mutations were compared with one unifying best guide, v3.2 scaffold described above and a spacer targeting RNF2 (tatgagttacaacgaacacctc (SEO ID NO: 3171) (see Table 18) targeting the genomic RNF2 locus. Seventy-two hours after transfection of the panel of PsaCas12f variants containing a single point mutation (plus the sgRNA), genomic locus at RNF2 was PCR amplified and subjected to NGS. Indel profile is quantified by CRISPResso for all the mutants.

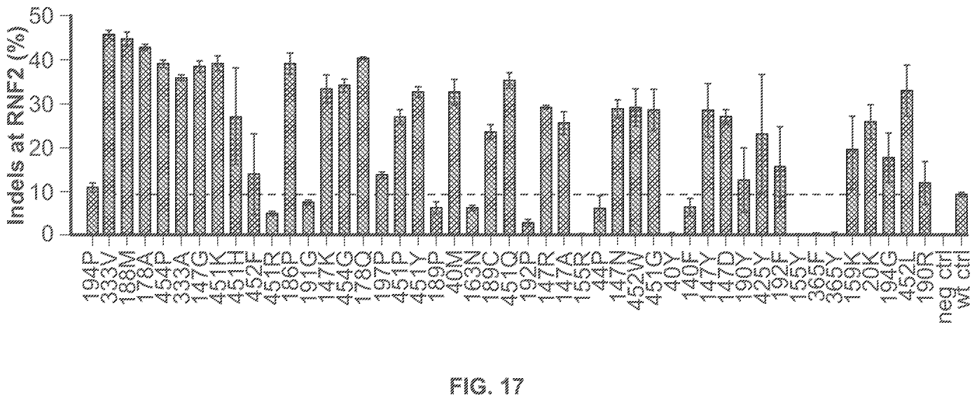

Of the panel of point mutations tested, the point mutation at position 333 of PsaCas12f to Valine from Lysine dramatically increased the cleavage efficacy of PsaCas12f as shown in FIG. 17.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A composition comprising:
(a) a target specific nuclease comprising an amino acid sequence 70% identical to an amino acid sequence selected from the group consisting of SEQ ID NOs: 1-19; and
(b) a guide RNA (gRNA)
wherein a target comprises a DNA target.

2. The composition of claim 1, wherein the DNA target is a single stranded DNA.

3. The composition of claim 1, wherein the DNA target is a double stranded DNA.

4. The composition of claim 1, wherein the target specific nuclease has a length less than about 1000 amino acids.

5. The composition of claim 4, wherein the target specific nuclease has a length less than about 900 amino acids.

6. The composition of claim 5, wherein the target specific nuclease has a length less than about 800 amino acids.

7. The composition of claim 1, wherein the amino acid sequence is SEQ ID NO: 1.

8. The composition of claim 1 wherein the target specific nuclease comprises an amino acid sequence 90% identical to the amino acid sequence of SEQ ID NO: 1.

9. The composition of claim 1, wherein the target specific nuclease comprises an amino acid sequence 95% identical to the amino acid sequence of SEQ ID NO: 1.

10. The composition of claim 1, wherein the target specific nuclease comprises an amino acid sequence 98% identical to the amino acid sequence of SEQ ID NO: 1.

11. The composition of claim 1, wherein the target specific nuclease comprises an amino acid sequence 99% identical to the amino acid sequence of SEQ ID NO: 1.

12. The composition of claim 1, wherein the nuclease is the amino acid sequence of SEQ ID NO. 1.

13. The composition of any one of the previous claims, wherein the target specific nuclease is selected from the group consisting of Cas12f, Cas12m, and any variants thereof; and optionally wherein the target specific nuclease is PsaCas12f.

14. The composition of any one of the previous claims, wherein the gRNA is a single guide RNA (sgRNA) or a dual guide (dgRNA).

15. The composition of any one of the previous claims, wherein the gRNA is a sgRNA comprising a nucleic acid sequence 70% identical to a nucleic acid sequence selected from the group consisting of SEQ ID NOs: 20-43, 61-79, 145-198.

16. The composition of anyone one of the previous claims, wherein the gRNA has a spacer region with a sequence comprising a length of about 17 to about 53 nucleotides (nt), optionally wherein the sequence comprises a length of about 29 to about 53 nt, optionally wherein the sequence comprises a length of about 40 to about 50 nt; or optionally wherein the sequence comprises a length of about 21 to 22 nt.

17. The composition of anyone one of the previous claims, wherein the gRNA has a direct repeat region with a sequence having a length of from about 20 to about 29 nt.

18. The composition of anyone of the previous claims, wherein the gRNA has a tracrRNA region with a sequence having a length of from about 27 to about 35 nt.

19. The composition of anyone one of the previous claims, wherein the target is in a cell.

20. The composition of claim 19, wherein the cell is a prokaryotic cell.

21. The composition of claim 19, wherein the cell is a eukaryotic cell.

22. The composition of claim 21, wherein the eukaryotic cell is a mammalian cell.

23. The composition of claim 22, wherein the mammalian cell is a human cell.

24. The composition of anyone one of the previous claims, wherein the amino acid sequence specifically binds to a protospacer-adjacent motif (PAM).

25. The composition of claim 24, wherein the PAM is selected from the group consisting of NNNNGATT, NNNNGNNN, NNG, NG, NGAN, NGNG, NGAG, NGCG, NAAG, NGN, NRN, NNGRRN, NNNRRT, TTTN, TTTV, TYCV, TATV, TYCV, TATV, TTN, KYTV, TYCV, TATV, TBN, any variants thereof, and any combinations thereof.

26. A nucleic acid molecule encoding the target specific nuclease of any of the preceding claims.

27. A nucleic acid molecule encoding the gRNA of any of the preceding claims.

28. One or more vectors comprising the nucleic acid molecule of claims 26-27.

29. A cell comprising the composition of claims 1-25, the nucleic acid molecule of claims 26-27 or the one or more vectors of claim 28.

30. The cell of claim 29, wherein the cell is a prokaryotic cell.

31. The cell of claim 29, wherein the cell is a eukaryotic cell.

32. The cell of claim 31, wherein the eukaryotic cell is a mammalian cell.

33. The cell of claim 32, wherein the mammalian cell is a human cell.

34. A method of inserting or deleting one or more base pairs in a DNA, the method comprising
(a) cleaving the DNA at a target site with a target specific nuclease, wherein the cleavage results in overhangs on both DNA ends;
(b) inserting a nucleotide complementary to the overhanging nucleotide on both of the DNA ends, or removing the overhanging nucleotide on both of the DNA ends; and
(c) ligating the DNA ends together, thereby inserting or deleting one or more base pairs in the DNA,
wherein the nuclease comprising an amino acid sequence 70% identical to an amino acid sequence selected from the group consisting of SEQ ID NOs: 1-19, and
wherein the target specificity of the target specific nuclease is provided by a guide RNA (gRNA).

35. The method of claim 34, wherein the target specific nuclease has a length less than about 1000 amino acids.

36. The method of claim 35, wherein the target specific nuclease has a length less than about 900 amino acids.

37. The method of claim 36, wherein the target specific nuclease has a length less than about 800 amino acids.

38. The method of claim 34, wherein the amino acid sequence is SEQ ID NO: 1.

39. The method of claim 38, wherein the target specific nuclease comprises an amino acid sequence 90% identical to the amino acid sequence of SEQ ID NO: 1.

40. The method of claim 38, wherein the target specific nuclease comprises an amino acid sequence 95% identical to the amino acid sequence of SEQ ID NO: 1.

41. The method of claim 38, wherein the target specific nuclease comprises an amino acid sequence 98% identical to the amino acid sequence of SEQ ID NO: 1.

42. The method of claim 38, wherein the target specific nuclease comprises an amino acid sequence 99% identical to the amino acid sequence of SEQ TD NO: 1.

43. The method of claim 34, wherein the nuclease is the amino acid sequence of SEQ ID NO: 1.

44. The method of any one of claims 34-43 wherein the target specific nuclease is selected from the group consisting of Cas12f, Cas12m, and any variants thereof; and optionally wherein the target specific nuclease is PsaCas12f.

45. The composition of any one of claims 34-44, wherein the gRNA is a single guide RNA (sgRNA) or a dual guide RNA (dgRNA).

46. The method of claim 45, wherein the gRNA is a sgRNA comprising a nucleic acid sequence 70% identical to a nucleic acid sequence selected from the group consisting of SEQ ID NOs: 20-43, 61-79, and 145-198.

47. The method of any one of claims 34-46, wherein the gRNA has a spacer region with a sequence having a length of from about 17 to about 30 nucleotides (nit), about 22 nt: or wherein the gRNA has a spacer region with a sequence having a length of from about 20 to about 53 nt, from about 29 to about 53 nt or from about 40 to about 50 nt.

48. The method of any one of claims 34-47, wherein the DNA target is in a cell.

49. The method of claim 48, wherein the cell is a prokaryotic cell.

50. The method of claim 49, wherein the cell is a eukaryotic cell.

51. The method of claim 50, wherein the eukaryotic cell is a mammalian cell.

52. The method of claim 51, wherein the mammalian cell is a human cell.

53. The method of any one of claims 34-52, wherein the amino acid sequence specifically binds to a protospacer-adjacent motif (PAM).

54. The method of claim 53, wherein the PAM is selected from the group consisting of NNNNGATT, NNNNGNNN, NNG, NG, NGAN, NGNG, NGAG, NGCG, NAAG, NGN, NRN, NNGRRN, NNNRRT, TTTN, TTTV, TYCV, TATV, TYCV, TATV, TTN, KYTV, TYCV, TATV, TBN, any variants thereof, and any combinations thereof.

55. A method of detecting a DNA target, the method comprising:
coupling the DNA target with a reporter to form a DNA-reporter complex;
mixing the DNA-reporter complex with a target specific nuclease and a guide RNA (gRNA);
cleaving the DNA-reporter complex; and
measuring a signal from the reporter, thereby detecting the DNA target.

56. The method of claim 55, wherein the target specific nuclease is selected from the group consisting of Cas12f, Cas12m, and any variants thereof; and optionally wherein the target specific nuclease is PsaCas12f.

57. The method of claim 55 wherein the target specific nuclease is complexed with a crRNA.

58. The method of claim 55, wherein the reporter is a fluorescent reporter.

59. A method for activating or inhibiting the expression of a gene, the method comprising mixing the composition of claim 1 with one or more transcription factors, wherein the target specific nuclease lacks endonuclease ability, wherein the target DNA comprises the gene, thereby activating the gene.

60. A method for nucleic acid base editing, the method comprising mixing the composition of claim 1, wherein the target specific nuclease is a nickase or a nuclease coupled to a deaminase, thereby editing the nucleic acid base from the target DNA.

61. A method for activating or inhibiting the expression of a gene, the method comprising mixing the composition of claim 1 with one or more epigenetic modifiers, wherein the target specific nuclease lacks endonuclease activity, wherein the target DNA comprises the gene, and modifying the target DNA or one or more histones associated to the target DNA, thereby activating or inhibiting the gene.

62. The method of claim 68, wherein the epigenetic modifier comprises KRAB, DNMT3a, DNMT1, DNMT3b, DNMT3L, TET1, p300, any variants thereof, or any combinations thereof.

63. The composition of any one of claims 1-25, wherein the gRNA comprises a nucleic acid sequence 70% identical to a nucleic acid sequence from the group consisting of SEQ ID NO: 246-272.

64. The composition of any one of claims 1-25, wherein the target specific nuclease is fused to a nuclear localization signal (NLS).

65. The composition of claim 64, wherein the NLS signal is at the 5′ or 3′ termini of the target specific nuclease nucleic acid sequence.

66. The composition of claim 64, wherein the NLS signal is in an intra-protein region.

67. The composition of any one of claims 63-65, wherein the NLS is derived from SV40.

68. The composition of any one of claims 63-66, wherein the target specific nuclease comprises a nucleic acid sequence 70% identical to a nucleic acid sequence from the group consisting of SEQ ID NO: 233-244.

69. The composition of any one of claims 1-25 or 63-68, wherein the target specific nuclease and the gRNA are delivered to the cell containing the DNA target in one or more adeno-associated viral (AAV) vectors.

70. The composition of any one of claims 1-25 or 63-69, wherein the target specific nuclease has been circular permutated.

71. The composition of claim 70, wherein the target specific nuclease is PasCas12f.

72. The composition of claim 70 or 71, wherein the target specific nuclease comprises a nucleic acid sequence 70% identical to a nucleic acid sequence from the group consisting of SEQ ID NO: 273-285.

73. The composition of any one of claims 1-25 or 63-72, wherein the target specific nuclease has a point mutation at amino acid position 333 encoding a valine.

74. The composition of claim 73, wherein the point mutation at amino acid position 333 is mutated to a lysine.


SEQUENCE LISTING (318 sequences)

SEQ ID NO: 1 - protein, 586 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 2 - protein, 534 aa, Artificial Sequence
  Description of Artificial Sequence: Synthetic polypeptide
MEEENFDNAEVTTGIKFKLKLNSETREKLNNYFNEYGKAINFAVRIIQKQLADDRFAGKAKLDENKKQLLDEDGKKIWDFPSESCSCGKQVVRYVNGKPFCQECYRNKFSENGIRKRMYSAKGRKAEYDINIKNSTNRISKTHFNYAIREAFILDKSIKKQRKERFRRLNDMMRKLQEFIDIREGKRLVCPKIERQKVERYIHPAWINKEKKIEEFRGYSLSVVNSKIKALDRNIKREEKSLKEKGQINFKARRLMLDKSVKFTDTNKVSFTISKSLPKEYELDLPKKEKRLNWLKEKIEIIKNQKPKYAYLLRRGDDFYLQYTLQTKPEIKTTHSGAVGIDRGISHIAVYTFVSNDGKNERPLFLSSSEILRLKNLQKERDKFLRRKHNKIRKKSNMRNIEDKIQLILHNYSKQIVDFAKEKNAFIVFEKLEKPKKSRSKMSKKEQYKLSLFTFKKLSDLVDYKAKREGIKVIYIEPAYTSKECSHCGEKVNTQRPFNGNYSLFKCNKCGIILNSDYNASINIAKKGLNIFNI

SEQ ID NO: 3 - protein, 449 aa, Artificial Sequence
  Description of Artificial Sequence: Synthetic polypeptide
MAKGEKNNDVLYRAVKFEIRPTLNQETILQRISSNLRLIWNEAWKERQDRYEIFFKPIYERIYNAKKKALEKGFTDLWEKEVAKFSQQVLVKRGFPLQLVLEQKSLFAELKKAFEEHGITLYDQINALTAKRSLNTEFGLIPRNWQEETLDALDGSFKSFFALRKRGDKDAKPPSERTNEDSFYKIPGRSGFKVTDDGKVIVSFGKLSETLVGRIPEYQQEKLSHAKNLKKFEIVRDERDMAKSGCFWISIAYEIPKPPELPFNPSKAVFLAIGASWIGIISPRGEFCWRMPRPDFHWKPKINAVDERLKRVSKGSIKWKRLIFARSKMFAIMARQQKQHGQYEVIKRLLELGVCFVVTDLKVRSKEGSLADSSKAERGGSPFGANWSAQNTGNIANLVAKLTDHVSALGGMVIKRKSPELLVEEKRLPQEKRKILLAQKLKDEFLSLN

SEQ ID NO: 4 - protein, 482 aa, Artificial Sequence
  Description of Artificial Sequence: Synthetic polypeptide
MKNTKEEKWMQTYCFDLTDEEFGAENIRLATHISDSLVPLFNEVLLQVLKGDETIKELKQEVKLRGRALKQKAKEAQMEDLWDRENNEIDDEEWLERGYDQEVIKEHRDYVDEIAKLYSENKVTAFDHNYHYAQENLEAIGCTAPYVNISAGLRRGAIKNCHGAVDSWRKHLATGDYKSKPPGQQEVGKFYMLRCEPGCAVTKDRKNVRISLGDRKSSPVFELPGLDNSKNKPLHMLLRSDAKVKSFTLSRRSARNPDKKESDLQKPGVWRISINFELPLPEKKPATEYNTVALVIGSNYLGVALHDSERNFPLNLPLPHKHWFPIIGDIEGRANVPWRKKGSKKWRRKMFGVQKKHSGGRQACYRYMARQQKQGEYETIADHLIGCGVHFVVSKPSINHPKGLADASAPDRGGDTGPNRIISSTGVNSLVLKLKQKVKEFGGSVTEMEAPPLPERFRFWDSGPKKVIVAQLLRNQYLAQKK

SEQ ID NO: 5 - protein, 591 aa, Artificial Sequence
  Description of Artificial Sequence: Synthetic polypeptide
MVKQTTFFCKECNKNINIPRNIIKKLESNHISQDQAIKKAKERHNKKKHSLILGIKFKLYVKNKEDKEKLNSYFEEYAKAVTFAAQIIDKIKSGYLPQWKKDKKLKRIIFPKGKCDFCGTKTEIGWISKRGKKICKNCYSKEYGENGIRKKLYATRGRKVNPSYNIFNATKKLAATHYNYAIREAFQLLEANRKQRQERIRRLLRDKKRLREFEDLIEKPDRRIELPMKTRQREKRYIHISQKDKINELRGYTLHKIKEKIRILRRNTEREERALRKKTPIIFKGNRIMLFPQGIKFDKENNKVKITIAKNLPKEFIFSGTNVANKHGRRFFKEKLNLISQQKPKYAYLIRKQTKNSKKITDYDYYLQYTIETVYKIRKNYDGIIGIDRGINNLACLVLLEKNQEKPCGVKFYKGKEINALKIKRRKQLYFLRRKHNRKQKQKRIRRIEPKINQILHIISKEIVELAKEKNFAIGLEQLEKPKKSRFRQRRKERYFLSLFNFKTLSTFIEYKAKKEGIRVIYIPPERTSQICSHCAIKGDVHTNTIRPYRKPNAKKSSSSLFKCKKCGVELNADYNAAFNIAQKSLKILST

SEQ ID NO: 6 - protein, 373 aa, Artificial Sequence
  Description of Artificial Sequence: Synthetic polypeptide
MKIKEQSEVRELLKAYKYRIYPNKEQRLYLAKTFGCTRFIYNKMLSDRIKVYEENKDLDIKKVKYPTPAQYKKEFTWLKEVDSLALANAQMNLDKAYKNFFRDKSMGFPKFKSKKVNYYSYTTNNQKGTVYIEDGYIKLPKLKTMIKIKQHRKFNGLIKSCTISKTPSNKYYISILVYTENKQLPKVDKKVGIDVGLKEFAITSNGEFFSNPKWLRKSEKRLRKLQKDLSRKQKGSNNRCKARLKVAKLHEKITNQRKNFLHKLSIKLIRENQSIVIEDLKVKNMLQNHKLAKAISEVSWYEFRTMLEYKADWYGRELIIAPSNYASSQICSNCGYKNKEVKNLELREWVCPKCGIHHHRDINASKNLLKLAI

SEQ ID NO: 7 - protein, 402 aa, Artificial Sequence
  Description of Artificial Sequence: Synthetic polypeptide
MLVFEAKLRGTKEQYERLDEAIRTARFVRNSCLRYWMDNKGEKVGRYELSAYCAVLAKEFPWAKKLNSMARQASAERAWTAIARFYDNCKKKVSGKKGFPKFKKYKTRDSVEYKTSGWKLSEDRRTITFTDGFKAGSFKTWGTRDLHFYQLKQIKRVRVVRRADGYYVQFCIDQDRVEKREPTGTAIGLDVGLNHFYTDSDGQTVENPRHLRKSEKALKRLQRRLAKTQKGSKNRQKARNRLGRKHLKVSRQRKDFAVKTALCVVQSNDLVAYEDLKVRNMVKNHNLAKSISDAAWSTFRQWMEYFGKVFGVATVAVPPQYTSQNCSNCGEKIQKSLSTRTHRCPHCGFVADRDHNAAINILELGLSTVGHTETHASGDIDLCLGGETPQSKSSRRKRKPHQ

SEQ ID NO: 8 - protein, 387 aa, Artificial Sequence
  Description of Artificial Sequence: Synthetic polypeptide
MDQIIKGVKLRLYPNRGQKDKLWQMFGNDRFVWNQMLSMAKTRYQNNPRASFINGYGMDTLLKVLKNEYPFLKESDSTSLQVVNHKLNQSFQMLFKHRGGYPRFKSRKATKQAYTGKSKVSVVAKRCLKLPKIGYIKTSKTNQLVDTKIKRYTVSYDATGRYYLSLQVEVPAPELLPKTGKVVGLDVGLADLAISSDGVKYGTFNAKWLDKQVNKWQSAYAKRKYRATIAVRQWNHNHKTVKEELNDYQNWQRARRYKARYQAKVANKRQDNLQKLTTELVKQYDVIVIEDLKTKNLQKNHHLAKSIANASWYQLRTMLEYKCAWYGRQLIIVKPNYTSQICSSCGYHNGPKPLKIREWTCSKCGVHHDRDINAAINILHKGLKANG

SEQ ID NO: 9 - protein, 360 aa, Artificial Sequence
  Description of Artificial Sequence: Synthetic polypeptide
MTSNKCAEEGQKKVSVTPITFNFWLTKVKDRIFELEDQTTVLLKDVSVDLSRQVLKMLAGAWQSYFELRKRGDTEARPPSPKKEGWFQTMAWSNFTVRQGSIFVPGYQKNRIEIKLGDYLKRMVEDKEVAYVTLYRDRFSGEFNLSVVVKNPAPKHIEHPKVIRAIDLGAGDIAVSDSSGAEYLIPARRPDKHWMPLIAQVEHRAERCIKGSRAYKRRMKARRVMHEKSGNQKDSYQRKLARALFSGEVEAIVIGKGKTRLGLAQSESGTPDQHYGAQNTGYLFRQLLYIKEKAKERGIPVVEFPDPQRKGELEDSQKKFFASRELLSLGCKKFKIEVPNSFVQGEFIFNQGKGGKPKVA

SEQ ID NO: 10 - protein, 603 aa, Artificial Sequence
  Description of Artificial Sequence: Synthetic polypeptide
MAITVHTAGVHYRWTDNPPEQLMRQLRLAHDLREDLVTLQLDYETAKAGIWSSYPAVAAAETELADAESAAEQAAAAVSEERTKLRTKRITGPLAQKLTAARKRVREARSTRRAAISEVHEEAKGRLVDASDALKAQQKALYKTYCQDGDLFWATFNDVLDHHKAAVKRIGQMRAAGQPAQLRHHRFDGTGSIAVQLQRQAGQPQRTPELIADVDGKYGRVLSVPWVQPDRWERIPRRERRMIGRVTVRMRAGQLSGEPQWLDIPVQQHRMLPLDADITGARLTVTRTAGTLRAQISVTAKIPDPEPVTDGPDVAVHLGWRNTDTGVRVARWRSTEPIEVPFDFRDTLTVDPGGRSGEIFVPEAVPRRVERAHLIASHRADRMNELRARLVDYLAETGPRPHPSREGEELGAGNVRMWKSPNRFAWLARVWADDESVSTDIREALAQWRHQDWISWHHQEGGRRRSAAQRLDVYRQVAAVLVSQAGRLVLDDTSYADIAQRSATTKTEELPNETAARINRRRAHAAPGELRQTLVAAADRDAVPVDTVSHTGVSVVHAKCGHENPSDGRFMSVVVACDGCGEKYDQDESALTHMLTRAVQSAA

SEQ ID NO: 11 - protein, 596 aa, Artificial Sequence
  Description of Artificial Sequence: Synthetic polypeptide
MTTMTVHTMGVHYKWQIPEVLRQQLWLAHNLREDLVSLQLAYDDDLKAIWSSYPDVAQAEDTMAAAEADAVALSERVKQARIEARSKKISTELTQQLRDAKKRLKDARQARRDAIAVVKDDAAERRKARSDQLAADQKALYGQYCRDGDLYWASFNTVLDHHKTAVKRIAAQRASGKPATLRHHRFDGSGTIAVQLQRQAGAPPRTPMVLADEAGKYRNVLHIPGWTDPDVWEQMTRSQCRQSGRVTVRMRCGSTDGQPQWIDLPVQVHRWLPADADITGAELVVTRVAGIYRAKLCVTARIGDTEPVTSGPTVALHLGWRSTEEGTAVATWRSDAPLDIPFGLRTVMRVDAAGTSGIIVVPATIERRLTRTENIASSRSLALDALRDKVVGWLSDNDAPTYRDAPLEAATVKQWKSPQRFASLAHAWKDNGTEISDILWAWFSLDRKQWAQQENGRRKALGHRDDLYRQIAAVISDQAGHVLVDDTSVAELSARAMERTELPTEVQQKIDRRRDHAAPGGLRASVVAAMTRDGVPVTIVAAADFTRTHSRCGHVNPADDRYLSNPVRCDGCGAMYDQDRSFVTLMLRAATAPSNP

SEQ ID NO: 12 - protein, 591 aa, Artificial Sequence
  Description of Artificial Sequence: Synthetic polypeptide
MPDQLTQQLRLAHDLREDLVTLEYEYEDAVKAVWSSYPAVAALEAQVAELDERASELASTVKEEKSRQRTKRPSHPAVAQLAETRAQLKAAKASRREAIASVRDEATERLRTISDERYAAQKQLYRDYCTDGLLYWATFNAVLDHHKTAVKRIAAHRKQGRAAQLRHHRWDGTGTISVQLQRQATDPARTPAIIADADTGKWRSSLIVPWVNPDVWDTMDRASRRKAGRVVIRMRCGSSRNPDGTKTSEWIDVPVQQHRMLPADADITAAQLTVRREGADLRATIGITAKIPDQGEVDEGPTIAVHLGWRSSDHGTVVATWRSTEPLDIPETLRGVITTQSAERTVGSIVVPHRIEQRVHHHATVASHRDLAVDSIRDTLVAWLTEHGPQPHPYDGDPITAASVQRWKAPRRFAWLALQWRDTPPPEGADIAETLEAWRRADKKLWLESEHGRGRALRHRTDLHRQVAAYFAGVAGRIVVDDSDIAQIAGTAKHSELLTDVDRQIARRRAIAAPGMLRAAIVAAATRDEVPTTTVSHTGLSRVHAACGHENPADDRYLMQPVLCDGCGRTYDTDLSATILMLQRASAATSN

SEQ ID NO: 13 - protein, 415 aa, Artificial Sequence
  Description of Artificial Sequence: Synthetic polypeptide
MLRAYKYRIYPTDEQKVLFAKTFGCCRFVYNWALNLKITAYKERKETLGNVYLTNLMKSELKVEHEWLSEVNSQSLQSSLRNLDTAYTNFFRNTKAVGFPRFKSRKDKQSFLCPQHCRVDFEKGTITIPKAKDIPAVLHRRFKGTVKTVTISMTPSGRYFASVLVDTSMQEMKPSEPMRDTTVGIDLGIKSLAVCSDGRTFANPKNLQRSLDRLKLLQKRLSRKQKGSANRNKARIRVARLQEHIANSRKDSLHKITHALTHDSQVRTICMEDLNVKGMQRNHHLAQAVGDASFGMFLTLLEYKCSWYGVNLIKIDRFAPSSKTCGKCGHVYKGLNLSERSWTCPECGTHHDRDFNAACNIKEFGLKALPTERGKVKPVDCPLVDDRPRVLKSNGRKKQEKRGGIGISEAAKSLV

SEQ ID NO: 14 - protein, 300 aa, Artificial Sequence
  Description of Artificial Sequence: Synthetic polypeptide
PQGIKFDKENNKVKITIAKNLPKEFIFSGTNVANKHGRRFFKEKLNLISQQKPKYAYLIRKQTKNSKKITDYDYYLQYTIETVYKIRKNYDGIIGIDRGINNLACLVLLEKNQEKPCGVKFYKGKEINALKIKRRKQLYFLRRKHNRKQKQKRIRRIEPKINQILHIISKEIVELAKEKNFAIGLEQLEKPKKSRFRQRRKERYFLSLFNFKTLSTFIEYKAKKEGIRVIYIPPERTSQICSHCAIKGDVHTNTIRPYRKPNAKKSSSSLFKCKKCGVELNADYNAAFNIAQKSLKILST

SEQ ID NO: 15 - protein, 204 aa, Artificial Sequence
  Description of Artificial Sequence: Synthetic polypeptide
DRGINNLACLVLLEKNQEKPCGVKFYKGKEINALKIKRRKQLYFLRRKHNRKQKQKRIRRIEPKINQILHIISKEIVELAKEKNFAIGLEQLEKPKKSRFRQRRKERYFLSLFNFKTLSTFIEYKAKKEGIRVIYIPPERTSQICSHCAIKGDVHTNTIRPYRKPNAKKSSSSLFKCKKCGVELNADYNAAFNIAQKSLKILST

SEQ ID NO: 16 - protein, 193 aa, Artificial Sequence
  Description of Artificial Sequence: Synthetic polypeptide
LLEKNQEKPCGVKFYKGKEINALKIKRRKQLYFLRRKHNRKQKQKRIRRIEPKINQILHIISKEIVELAKEKNFAIGLEQLEKPKKSRFRQRRKERYFLSLFNFKTLSTFIEYKAKKEGIRVIYIPPERTSQICSHCAIKGDVHTNTIRPYRKPNAKKSSSSLFKCKKCGVELNADYNAAFNIAQKSLKILST

SEQ ID NO: 17 - protein, 370 aa, Artificial Sequence
  Description of Artificial Sequence: Synthetic polypeptide
MEYSYKFRVYPTAAQAEQIQRTFGCCRFVWNHYLALRKDLYEQDGKTMNYNACSGDMTQLKKTLLWLREVDATALQSSLRDLDTAYQNFFRRVKKGEKPGYPKFKSKHHSKKSYKSKCVGTNIKVLDKAVQLPKLGLVKCRISKEVKGRILSATISQNPSGKYFVAICCTDVELEPLTSTGAVAGIDMGLKAFAITSDGVEYPNHKYLTKSQKKLAKLQRQLSRKSKGSKRREKARIQVARLHEHVANQRQDMLHKLSTDLVRNYDLIAIEDLAPSNMVKNHMLAKAISDASWGEFPRQLKYKAEWHGKKVVTVGRFFPSSQLCSNCGAQWSGTKDLSVRQWTCPVCGAIHDRDMNAARNILNEGLRLMA

SEQ ID NO: 18 - protein, 371 aa, Artificial Sequence
  Description of Artificial Sequence: Synthetic polypeptide
VYNYFLSQRKEQYRLTGKSDNYYAQAKTLTALKKQEETAWLKEVNAQTLQFAIKSLESAYTNFFKKSAKFPKFKSKHSKNSFTVPQSASVAGGRLFIPKFTEGIKCSVHREIKGKIGKVTITKSPSGKYFVSVFTEEEYITQLEKTGKSIGLDMGLKDLLITSEGEIFNNNRYTRRYECKLAKAQRHLSRKKKGSRGFENQRLKVARLHEKIVNSRTDYLHKCSISLVRRYDIICIEDLNVKGMTKNHHLAKSITDASWGKFVSMLTYKAEWNNKKVVDVDRYFPSSQTCNVCGYVNKQIKDLSVREWECPHCHTHHDRDKNAAINILRIGLNNNISAGTVDYTGGEEVRTDLLESHSSVKPEANEPLVHG

SEQ ID NO: 19 - protein, 382 aa, Artificial Sequence
  Description of Artificial Sequence: Synthetic polypeptide
MLAKHFGCSRFVYNYFLSQRKEQYRLTGKSDNYYAQAKTLTALKKQEETAWLKEVNAQTLQFAIKSLESAYTNFFKKSAKFPKFKSKHSKNSFTVPQSASVAGGRLFIPKFTEGIKCSVHREIKGKIGKVTITKSPSGKYFVSVFTEEEYITQLEKTGKSIGLDMGLKDLLITSEGEIFNNNRYTRRYECKLAKAQRHLSRKKKGSRGFENQRLKVARLHEKIVNSRTDYLHKCSISLVRRYDIICIEDLNVKGMTKNHHLAKSITDASWGKFVSMLTYKAEWNNKKVVDVDRYFPSSQTCNVCGYVNKQIKDLSVREWECPHCHTHHDRDKNAAINILRIGLNNNISAGTVDYTGGEEVRTDLLESHSSVKPEANEPLVHG

SEQ ID NO: 20 - DNA, 144 bp, Artificial Sequence
  Description of Artificial Sequence: Synthetic polynucleotide
gattgtattatgctccactttaataagtggtgccttccaaagctatatgctgagggaggatgggcgctgttgcagcgtctgcccacctcagagtgggtatccttacctattgaaaacccaaagtaataggtcaaggaatgcaac

SEQ ID NO: 21 - DNA, 138 bp, Artificial Sequence
  Description of Artificial Sequence: Synthetic polynucleotide
gattgtattatgctccactttaataagtggtgccttccaaagctatatgctgagggaggatgggcgctgttgcagcgtctgcccacctcagagtgggtatccttacctattgaaaagtaataggtcaaggaatgcaac

SEQ ID NO: 22 - DNA, 135 bp, Artificial Sequence
  Description of Artificial Sequence: Synthetic polynucleotide
gattgtattatgctccactttaataagtggtgccttccaaagctatatgctgagggaggatgggcgctgttgcagcgtctgcccacctcagagtgggtatccttacctattgaaaaataggtcaaggaatgcaac

SEQ ID NO: 23 - DNA, 136 bp, Artificial Sequence
  Description of Artificial Sequence: Synthetic polynucleotide
gattgtattatgctccactttaataagtggtgccttccaaagctatatgctgagggaggatgggcgctgttgcagcgtctgcccacctcagagtgggtatccttacctattgaaataataggtcaaggaatgcaac

SEQ ID NO: 24 - DNA, 128 bp, Artificial Sequence
  Description of Artificial Sequence: Synthetic polynucleotide
gctttaataagtggtgccttccaaagctatatgctgagggaggatgggcgctgttgcagcgtctgcccacctcagagtgggtatccttacctattgaaaacccaaagtaataggtcaaggaatgcaac

SEQ ID NO: 25 - DNA, 122 bp, Artificial Sequence
  Description of Artificial Sequence: Synthetic polynucleotide
gctttaataagtggtgccttccaaagctatatgctgagggaggatgggcgctgttgcagcgtctgcccacctcagagtgggtatccttacctattgaaaagtaataggtcaaggaatgcaac

SEQ ID NO: 26 - DNA, 119 bp, Artificial Sequence
  Description of Artificial Sequence: Synthetic polynucleotide
gctttaataagtggtgccttccaaagctatatgctgagggaggatgggcgctgttgcagcgtctgcccacctcagagtgggtatccttacctattgaaaaataggtcaaggaatgcaac

SEQ ID NO: 27 - DNA, 120 bp, Artificial Sequence
  Description of Artificial Sequence: Synthetic polynucleotide
gctttaataagtggtgccttccaaagctatatgctgagggaggatgggcgctgttgcagcgtctgcccacctcagagtgggtatccttacctattgaaataataggtcaaggaatgcaac

SEQ ID NO: 28 - DNA, 116 bp, Artificial Sequence
  Description of Artificial Sequence: Synthetic polynucleotide
ggtgccttccaaagctatatgctgagggaggatgggcgctgttgcagcgtctgcccacctcagagtgggtatccttacctattgaaaacccaaagtaataggtcaaggaatgcaac

SEQ ID NO: 29 - DNA, 110 bp, Artificial Sequence
  Description of Artificial Sequence: Synthetic polynucleotide
ggtgccttccaaagctatatgctgagggaggatgggcgctgttgcagcgtctgcccacctcagagtgggtatccttacctattgaaaagtaataggtcaaggaatgcaac

SEQ ID NO: 30 - DNA, 107 bp, Artificial Sequence
  Description of Artificial Sequence: Synthetic polynucleotide
ggtgccttccaaagctatatgctgagggaggatgggcgctgttgcagcgtctgcccacctcagagtgggtatccttacctattgaaaaataggtcaaggaatgcaac

SEQ ID NO: 31 - DNA, 108 bp, Artificial Sequence
  Description of Artificial Sequence: Synthetic polynucleotide
ggtgccttccaaagctatatgctgagggaggatgggcgctgttgcagcgtctgcccacctcagagtgggtatccttacctattgaaataataggtcaaggaatgcaac

SEQ ID NO: 32 - DNA, 113 bp, Artificial Sequence
  Description of Artificial Sequence: Synthetic polynucleotide
gccttccaaagctatatgctgagggaggatgggcgctgttgcagcgtctgcccacctcagagtgggtatccttacctattgaaaacccaaagtaataggtcaaggaatgcaac

SEQ ID NO: 33 - DNA, 107 bp, Artificial Sequence
  Description of Artificial Sequence: Synthetic polynucleotide
gccttccaaagctatatgctgagggaggatgggcgctgttgcagcgtctgcccacctcagagtgggtatccttacctattgaaaagtaataggtcaaggaatgcaac

SEQ ID NO: 34 - DNA, 105 bp, Artificial Sequence
  Description of Artificial Sequence: Synthetic polynucleotide
gccttccaaagctatatgctgagggaggatgggcgctgttgcagcgtcttgcccacctcagagtgggtatccttacctattgaaaaataggtcaaggaatgcaac

SEQ ID NO: 35 - DNA, 107 bp, Artificial Sequence
  Description of Artificial Sequence: Synthetic polynucleotide
gccttccaaagctatatgctgagggaggatgggcgctgttgcagcgtctgccccacctcagagtgggtatccttacctattgaaataataggtcaaggaattgcaac

SEQ ID NO: 36 - DNA, 115 bp, Artificial Sequence
  Description of Artificial Sequence: Synthetic polynucleotide
gtgccttccaaagctatatgctgagggaggatgggcgctgttgcagcgtctgcccacctcagagtgggtatccttacctattgaaaacccaaagtaataggtcaaggaatgcaac

SEQ ID NO: 37 - DNA, 109 bp, Artificial Sequence
  Description of Artificial Sequence: Synthetic polynucleotide
gtgccttccaaagctatatgctgagggaggatgggcgctgttgcagcgtctgcccacctcagagtgggtatccttacctattgaaaagtaataggtcaaggaatgcaac

SEQ ID NO: 38 - DNA, 106 bp, Artificial Sequence
  Description of Artificial Sequence: Synthetic polynucleotide
gtgccttccaaagctatatgctgagggaggatgggcgctgttgcagcgtctgcccacctcagagtgggtatccttacctattgaaaaataggtcaaggaatgcaac

SEQ ID NO: 39 - DNA, 107 bp, Artificial Sequence
  Description of Artificial Sequence: Synthetic polynucleotide
gtgccttccaaagctatatgctgagggaggatgggcgctgttgcagcgtctgcccacctcagagtgggtatccttacctattgaaataataggtcaaggaatgcaac

SEQ ID NO: 40 - DNA, 132 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgaaaacccaaagtaataggtcaaggaatgcaac

SEQ ID NO: 41 - DNA, 126 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgaaaagtaataggtcaaggaatgcaac

SEQ ID NO: 42 - DNA, 123 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgaaaaataggtcaaggaatgcaac

SEQ ID NO: 43 - DNA, 124 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgaaataataggtcaaggaatgcaac

SEQ ID NO: 44 - protein, 599 aa, Artificial Sequence
  Description of Artificial Sequence: Synthetic polypeptide
EGAPKKKRKVGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 45 - protein, 596 aa, Artificial Sequence
  Description of Artificial Sequence: Synthetic polypeptide
PKKKRKVGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 46 - protein, 598 aa, Artificial Sequence
  Description of Artificial Sequence: Synthetic polypeptide
PAAKRVKLDGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 47 - protein, 598 aa, Artificial Sequence
  Description of Artificial Sequence: Synthetic polypeptide
KLKIKRPVKGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 48 - protein, 609 aa, Artificial Sequence
  Description of Artificial Sequence: Synthetic polypeptide
AVKRPAATKKAGQAKKKKLDGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 49 - protein, 599 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSEGAPKKKRKV

SEQ ID NO: 50 - protein, 596 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSPKKKRKV

SEQ ID NO: 51 - protein, 598 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSPAAKRVKLD

SEQ ID NO: 52 - protein, 598 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSKLKIKRPVK

SEQ ID NO: 53 - protein, 609 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSAVKRPAATKKAGQAKKKKLD

SEQ ID NO: 54 - protein, 612 aa, Artificial Sequence
  Description of Artificial Sequence: Synthetic polypeptide
EGAPKKKRKVGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSEGAPKKKRKV

SEQ ID NO: 55 - protein, 605 aa, Artificial Sequence
  Description of Artificial Sequence: Synthetic polypeptide
PKKKRKVGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSPKKKRK

SEQ ID NO: 56 - protein, 610 aa, Artificial Sequence
  Description of Artificial Sequence: Synthetic polypeptide
PAAKRVKLDGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSPAAKRVKLD

SEQ ID NO: 57 - protein, 610 aa, Artificial Sequence
  Description of Artificial Sequence: Synthetic polypeptide
KLKIKRPVKGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSKLKIKRPVK

SEQ ID NO: 58 - protein, 632 aa, Artificial Sequence
  Description of Artificial Sequence: Synthetic polypeptide
AVKRPAATKKAGQAKKKKLDGGSMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSAVKRPAATKKAGQAKKKKLD

SEQ ID NO: 59 - protein, 827 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYK

SEQ ID NO: 60 - protein, 586 aa, Artificial Sequence; identical to SEQ ID NO: 1
  Description of Artificial Sequence: Synthetic polypeptide

SEQ ID NO: 61 - DNA, 128 bp, Artificial Sequence; identical to SEQ ID NO: 24
  Description of Artificial Sequence: Synthetic polynucleotide

SEQ ID NO: 62 - DNA, 135 bp, Artificial Sequence; identical to SEQ ID NO: 22
  Description of Artificial Sequence: Synthetic polynucleotide

SEQ ID NO: 63 - DNA, 122 bp, Artificial Sequence; identical to SEQ ID NO: 25
  Description of Artificial Sequence: Synthetic polynucleotide

SEQ ID NO: 64 - DNA, 119 bp, Artificial Sequence; identical to SEQ ID NO: 26
  Description of Artificial Sequence: Synthetic polynucleotide

SEQ ID NO: 65 - DNA, 120 bp, Artificial Sequence; identical to SEQ ID NO: 27
  Description of Artificial Sequence: Synthetic polynucleotide

SEQ ID NO: 66 - DNA, 116 bp, Artificial Sequence; identical to SEQ ID NO: 28
  Description of Artificial Sequence: Synthetic polynucleotide

SEQ ID NO: 67 - DNA, 110 bp, Artificial Sequence; identical to SEQ ID NO: 29
  Description of Artificial Sequence: Synthetic polynucleotide

SEQ ID NO: 68 - DNA, 107 bp, Artificial Sequence; identical to SEQ ID NO: 30
  Description of Artificial Sequence: Synthetic polynucleotide

SEQ ID NO: 69 - DNA, 108 bp, Artificial Sequence; identical to SEQ ID NO: 31
  Description of Artificial Sequence: Synthetic polynucleotide

SEQ ID NO: 70 - DNA, 144 bp, Artificial Sequence; identical to SEQ ID NO: 20
  Description of Artificial Sequence: Synthetic polynucleotide

SEQ ID NO: 71 - DNA, 137 bp, Artificial Sequence
  Description of Artificial Sequence: Synthetic polynucleotide
gattgtattatgctccactttaataagtggtgccttccaaagctatatgctgagggaggatgggcgctgttgcagcgtctgcccacctcagatgggtatccttacctattgaaaagtaataggtcaaggaatgcaac

SEQ ID NO: 72 - DNA, 136 bp, Artificial Sequence; identical to SEQ ID NO: 23
  Description of Artificial Sequence: Synthetic polynucleotide

SEQ ID NO: 73 - DNA, 113 bp, Artificial Sequence; identical to SEQ ID NO: 32
  Description of Artificial Sequence: Synthetic polynucleotide

SEQ ID NO: 74 - DNA, 113 bp, Artificial Sequence; identical to SEQ ID NO: 32
  Description of Artificial Sequence: Synthetic polynucleotide

SEQ ID NO: 75 - DNA, 105 bp, Artificial Sequence; identical to SEQ ID NO: 34
  Description of Artificial Sequence: Synthetic polynucleotide

SEQ ID NO: 76 - DNA, 106 bp, Artificial Sequence; identical to SEQ ID NO: 38
  Description of Artificial Sequence: Synthetic polynucleotide

SEQ ID NO: 77 - DNA, 107 bp, Artificial Sequence; identical to SEQ ID NO: 39
  Description of Artificial Sequence: Synthetic polynucleotide

SEQ ID NO: 78 - DNA, 133 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgaaaaacccaaagtaataggtcaaggaatgcaac

SEQ ID NO: 79 - DNA, 104 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgaaaa

SEQ ID NO: 80 - DNA, 52 bp, Artificial Sequence
  Description of Artificial Sequence: Synthetic oligonucleotide
gacccaaagtaataggtcaaggaatgcaactggttgcccaccctagtcattg

SEQ ID NO: 81 - DNA, 46 bp, Artificial Sequence
  Description of Artificial Sequence: Synthetic oligonucleotide
gagtaataggtcaaggaatgcaactggttgcccaccctagtcattg

SEQ ID NO: 82 - DNA, 43 bp, Artificial Sequence
  Description of Artificial Sequence: Synthetic oligonucleotide
gaataggtcaaggaatgcaactggttgcccaccctagtcattg

SEQ ID NO: 83 - DNA, 44 bp, Artificial Sequence
  Description of Artificial Sequence: Synthetic oligonucleotide
gtaataggtcaaggaatgcaactggttgcccaccctagtcattg

SEQ ID NO: 84 - DNA, 109 bp, Artificial Sequence
  Description of Artificial Sequence: Synthetic polynucleotide
gattgtattatgctccactttaataagtggtgccttccaaagctatatgctgagggaggatgggcgctgttgcagcgtctgcccacctcagagtgggtatccttaccta

SEQ ID NO: 85 - DNA, 93 bp, Artificial Sequence
  Description of Artificial Sequence: Synthetic oligonucleotide
gctttaataagtggtgccttccaaagctatatgctgagggaggatgggcgctgttgcagcgtctgcccacctcagagtgggtatccttaccta

SEQ ID NO: 86 - DNA, 81 bp, Artificial Sequence
  Description of Artificial Sequence: Synthetic oligonucleotide
ggtgccttccaaagctatatgctgagggaggatgggcgctgttgcagcgtctgcccacctcagagtgggtatccttaccta

SEQ ID NO: 87 - DNA, 78 bp, Artificial Sequence
  Description of Artificial Sequence: Synthetic oligonucleotide
gccttccaaagctatatgctgagggaggatgggcgctgttgcagcgtctgcccacctcagagtgggtatccttaccta

SEQ ID NO: 88 - DNA, 109 bp, Artificial Sequence; identical to SEQ ID NO: 84
  Description of Artificial Sequence: Synthetic polynucleotide

SEQ ID NO: 89 - DNA, 97 bp, Artificial Sequence
  Description of Artificial Sequence: Synthetic oligonucleotide
gtccactttaataagtggtgccttccaaagctatatgctgagggaggatgggcgctgttgcagcgtctgcccacctcagagtgggtatccttaccta

SEQ ID NO: 90 - DNA, 80 bp, Artificial Sequence
  Description of Artificial Sequence: Synthetic oligonucleotide
gtgccttccaaagctatatgctgagggaggatgggcgctgttgcagcgtctgcccacctcagagtgggtatccttaccta

SEQ ID NO: 91 - DNA, 132 bp, Artificial Sequence
  Description of Artificial Sequence: Synthetic polynucleotide
gttatgctccactttaataagtggtgccttccaaagctatatgctgagggaggatgggcgctgttgcagcgtctgcccacctcagagtgggtatccttacctattgaaaagtaataggtcaaggaatgcaac

SEQ ID NO: 92 - DNA, 131 bp, Artificial Sequence
  Description of Artificial Sequence: Synthetic polynucleotide
gtatgctccactttaataagtggtgccttccaaagctatatgctgagggaggatgggcgctgttgcagcgtctgcccacctcagagtgggtatccttacctattgaaaagtaataggtcaaggaatgcaac

SEQ ID NO: 93 - DNA, 130 bp, Artificial Sequence
  Description of Artificial Sequence: Synthetic polynucleotide
gatgctccactttaataagtggtgccttccaaagctatatgctgagggaggatgggcgctgttgcagcgtctgcccacctcagagtgggtatccttacctattgaaaagtaataggtcaaggaatgcaac

SEQ ID NO: 94 - DNA, 126 bp, Artificial Sequence; identical to SEQ ID NO: 41
  Description of Artificial Sequence: Synthetic polynucleotide

SEQ ID NO: 95 - DNA, 127 bp, Artificial Sequence
  Description of Artificial Sequence: Synthetic polynucleotide
gctccgctttaataagcggtgccttccaaagctatatgctgagggaggatgggcgctgttgcagcgtctgcccacctcagagtgggtatccttacctattgaaaagtaataggtcaaggaatgcaac

SEQ ID NO: 96 - DNA, 127 bp, Artificial Sequence
  Description of Artificial Sequence: Synthetic polynucleotide
gctccactttactaagtggtgccttccaaagctatatgctgagggaggatgggcgctgttgcagcgtctgcccacctcagagtgggtatccttacctattgaaaagtaataggtcaaggaatgcaac

SEQ ID NO: 97 - DNA, 127 bp, Artificial Sequence
  Description of Artificial Sequence: Synthetic polynucleotide
gcaccactttaataagtggtgccttccaaagctatatgctgagggaggatgggcgctgttgcagcgtctgcccacctcagagtgggtatccttacctattgaaaagtaataggtcaaggaatgcaac

SEQ ID NO: 98 - DNA, 127 bp, Artificial Sequence
  Description of Artificial Sequence: Synthetic polynucleotide
gctccactttaataagtggagccttccaaagctatatgctgagggaggatgggcgctgttgcagcgtctgcccacctcagagtgggtatccttacctattgaaaagtaataggtcaaggaatgcaac

SEQ ID NO: 99 - DNA, 127 bp, Artificial Sequence
  Description of Artificial Sequence: Synthetic polynucleotide
gctccactgtaatcagtggtgccttccaaagctatatgctgagggaggatgggcgctgttgcagcgtctgcccacctcagagtgggtatccttacctattgaaaagtaataggtcaaggaatgcaac

SEQ ID NO: 100 - DNA, 129 bp, Artificial Sequence
  Description of Artificial Sequence: Synthetic polynucleotide
gtgctccactttaataagtggtgcattccaaagctatatgctgagggaggatgggcgctgttgcagcgtctgcccacctcagagtgggtatccttacctattgaaaagtaataggtcaaggaatgcaac

SEQ ID NO: 101 - DNA, 129 bp, Artificial Sequence
  Description of Artificial Sequence: Synthetic polynucleotide
gctccacttgtaatcaagtggtgccttccaaagctatatgctgagggaggatgggcgctgttgcagcgtctgcccacctcagagtgggtatccttacctattgaaaagtaataggtcaaggaatgcaac

SEQ ID NO: 102 - DNA, 131 bp, Artificial Sequence
  Description of Artificial Sequence: Synthetic polynucleotide
gctccacttgctaatgcaagtggtgccttccaaagctatatgctgagggaggatgggcgctgttgcagcgtctgcccacctcagagtgggtatccttacctattgaaaagtaataggtcaaggaatgcaac

SEQ ID NO: 103 - DNA, 133 bp, Artificial Sequence
  Description of Artificial Sequence: Synthetic polynucleotide
gctccacttggctaatgccaagtggtgccttccaaagctatatgctgagggaggatgggcgctgttgcagcgtctgcccacctcagagtgggtatccttacctattgaaaagtaataggtcaaggaatgcaac

SEQ ID NO: 104 - DNA, 135 bp, Artificial Sequence
  Description of Artificial Sequence: Synthetic polynucleotide
gctccacttggcataattgccaagtggtgccttccaaagctatatgctgagggaggatgggcgctgttgcagcgtctgcccacctcagagtgggtatccttacctattgaaaagtaataggtcaaggaatgcaac

SEQ ID NO: 105 - DNA, 142 bp, Artificial Sequence
  Description of Artificial Sequence: Synthetic polynucleotide
gctccacttacatgaggatcacccatgtaagtggtgccttccaaagctatatgctgagggaggatgggcgctgttgcagcgtctgcccacctcagagtgggtatccttacctattgaaaagtaataggtcaaggaatgcaac

SEQ ID NO: 106 - DNA, 126 bp, Artificial Sequence
  Description of Artificial Sequence: Synthetic polynucleotide
gctccactttaataagtggtgccttccaaagctaatgctgagggaggatgggcgctgttgcagcgtctgcccacctcagagtgggtatccttacctattgaaaagtaataggtcaaggaatgcaac

SEQ ID NO: 107 - DNA, 127 bp, Artificial Sequence
  Description of Artificial Sequence: Synthetic polynucleotide
gctccactttaataagtggtgccttccaaagctaaatgctgagggaggatgggcgctgttgcagcgtctgcccacctcagagtgggtatccttacctattgaaaagtaataggtcaaggaatgcaac

SEQ ID NO: 108 - DNA, 129 bp, Artificial Sequence
  Description of Artificial Sequence: Synthetic polynucleotide
gctccactttaataagtggtgccttccaaagcctatatggctgagggaggatgggcgctgttgcagcgtctgcccacctcagagtgggtatccttacctattgaaaagtaataggtcaaggaatgcaac

SEQ ID NO: 109 - DNA, 131 bp, Artificial Sequence
  Description of Artificial Sequence: Synthetic polynucleotide
gctccactttaataagtggtgccttccaaagctgtatatcagctgagggaggatgggcgctgttgcagcgtctgcccacctcagagtgggtatccttacctattgaaaagtaataggtcaaggaatgcaac

SEQ ID NO: 110 - DNA, 133 bp, Artificial Sequence
  Description of Artificial Sequence: Synthetic polynucleotide
gctccactttaataagtggtgccttccaaagctgctatatgcagctgagggaggatgggcgctgttgcagcgtctgcccacctcagagtgggtatccttacctattgaaaagtaataggtcaaggaatgcaac

SEQ ID NO: 111 - DNA, 135 bp, Artificial Sequence
  Description of Artificial Sequence: Synthetic polynucleotide
gctccactttaataagtggtgccttccaaagctgcttatatagcagctgagggaggatgggcgctgttgcagcgtctgcccacctcagagtgggtatccttacctattgaaaagtaataggtcaaggaatgcaac

SEQ ID NO: 112 - DNA, 137 bp, Artificial Sequence
  Description of Artificial Sequence: Synthetic polynucleotide
gctccactttaataagtggtgccttccaaagctgctgtatatcagcagctgagggaggatgggcgctgttgcagcgtctgcccacctcagagtgggtatccttacctattgaaaagtaataggtcaaggaatgcaac

SEQ ID NO: 113 - DNA, 141 bp, Artificial Sequence
  Description of Artificial Sequence: Synthetic polynucleotide
gctccactttaataagtggtgccttccaaagcacatgaggatcacccatgtgctgagggaggatgggcgctgttgcagcgtctgcccacctcagagtgggtatccttacctattgaaaagtaataggtcaaggaatgcaac

SEQ ID NO: 114 - DNA, 127 bp, Artificial Sequence
  Description of Artificial Sequence: Synthetic polynucleotide
gtccactttaataagtggtgccttccaaagctatatgctgagggaggatgggcgctgttgcaagcgtctgcccacctcagagtgggtatccttacctattgaaaagtaataggtcaaggattgcaac

SEQ ID NO: 115 - DNA, 127 bp, Artificial Sequence
  Description of Artificial Sequence: Synthetic polynucleotide
gtccactttaataagtggtgccttccaaagctatatgctgagggaggatgggcgctgttgcacgcgtctgcccacctcagagtgggtatccttacctattgaaaagtaataggtcaaggagtgcaac

SEQ ID NO: 116 - DNA, 127 bp, Artificial Sequence
  Description of Artificial Sequence: Synthetic polynucleotide
gtccactttaataagtggtgccttccaaagctatatgctgagggaggatgggcgctgttgcaggcgtctgcccacctcagagtgggtatccttacctattgaaaagtaataggtcaaggactgcaac

SEQ ID NO: 117 - DNA, 127 bp, Artificial Sequence
  Description of Artificial Sequence: Synthetic polynucleotide
gtccactttaataagtggtgccttccaaagctatatgctgagggaggatgggcgctgttgcatgcgtctgcccacctcagagtgggtatccttacctattgaaaagtaataggtcaaggaatgcaac

SEQ ID NO: 118 - DNA, 127 bp, Artificial Sequence
  Description of Artificial Sequence: Synthetic polynucleotide
gtccactttaataagtggtgccttccaaagctatatgctgagggaggatgggcgctgttcgatgcgtctgcccacctcagagtgggtatccttacctattgaaaagtaataggtcaaggaatcgaac

SEQ ID NO: 119 - DNA, 127 bp, Artificial Sequence
  Description of Artificial Sequence: Synthetic polynucleotide
gtccactttaataagtggtgccttccaaagctatatgctgagggaggatgggcgctgttgagtgcgtctgcccacctcagagtgggtatccttacctattgaaaagtaataggtcaaggaactcaac

SEQ ID NO: 120 - DNA, 127 bp, Artificial Sequence
  Description of Artificial Sequence: Synthetic polynucleotide
gtccactttaataagtggtgccttccaaagctatatgctgagggaggatgggcgctgttgcgtgcgtctgcccacctcagagtgggtatccttacctattgaaaagtaataggtcaaggaacgcaac

SEQ ID NO: 121 - DNA, 127 bp, Artificial Sequence
  Description of Artificial Sequence: Synthetic polynucleotide
gtccactttaataagtggtgccttccaaagctatatgctgagggaggatgggcgctgttgtatgcgtctgcccacctcagagtgggtatccttacctattgaaaagtaataggtcaaggaatacaac

SEQ ID NO: 122 - DNA, 127 bp, Artificial Sequence
  Description of Artificial Sequence: Synthetic polynucleotide
gtccactttaataagtggtgccttccaaagctatatgctgagggaggatgggcgctgccgcatgcgtctgcccacctcagagtgggtatccttacctattgaaaagtaataggtcaaggaatgcggc

SEQ ID NO: 123 - DNA, 127 bp, Artificial Sequence
  Description of Artificial Sequence: Synthetic polynucleotide
gtccactttaataagtggtgccttccaaagctatatgctgagggaggatgggcgctgcggcatgcgtctgcccacctcagagtgggtatccttacctattgaaaagtaataggtcaaggaatgccgc

SEQ ID NO: 124 - DNA, 126 bp, Artificial Sequence
  Description of Artificial Sequence: Synthetic polynucleotide
gtccactttaataagtggtgccttccaaagctatatgctgagggaggatgggcgctgttgcggcgtctgcccacctcagagtgggtatccttacctattgaaaagtaataggtcaaggaacgcaac

SEQ ID NO: 125 - DNA, 126 bp, Artificial Sequence
  Description of Artificial Sequence: Synthetic polynucleotide
gtccactttaataagtggtgccttccaaagctatatgctgagggaggatgggcgctgttgtagcgtctgcccacctcagagtgggtatccttacctattgaaaagtaataggtcaaggaatacaac

SEQ ID NO: 126 - DNA, 126 bp, Artificial Sequence
  Description of Artificial Sequence: Synthetic polynucleotide
gtccactttaataagtggtgccttccaaagctatatgctgagggaggatgggcgctgccgcagcgtctgcccacctcagagtgggtatccttacctattgaaaagtaataggtcaaggaatgcggc

SEQ ID NO: 127 - DNA, 126 bp, Artificial Sequence
  Description of Artificial Sequence: Synthetic polynucleotide
gtccactttaataagtggtgccttccaaagctatatgctgagggaggatgggcgctgcggcagcgtctgcccacctcagagtgggtatccttacctattgaaaagtaataggtcaaggaatgccgc

SEQ ID NO: 128 - DNA, 128 bp, Artificial Sequence
  Description of Artificial Sequence: Synthetic polynucleotide
gctccactttaataagtggtgccttccaaagctatatgctgagggaggatgggcgctgttgcagcgtctgcccacgctagacgtgggtatccttacctattgaaaagtaataggtcaaggaatgcaac

SEQ ID NO: 129 - DNA, 130 bp, Artificial Sequence
  Description of Artificial Sequence: Synthetic polynucleotide
gctccactttaataagtggtgccttccaaagctatatgctgagggaggatgggcgctgttgcagcgtctgcccactgctagacagtgggtatccttacctattgaaaagtaataggtcaaggaatgcaac

SEQ ID NO: 130 - DNA, 132 bp, Artificial Sequence
  Description of Artificial Sequence: Synthetic polynucleotide
gctccactttaataagtggtgccttccaaagctatatgctgagggaggatgggcgctgttgcagcgtctgcccacctgctagacaggtgggtatccttacctattgaaaagtaataggtcaaggaatgcaac

SEQ ID NO: 131 - DNA, 129 bp, Artificial Sequence
  Description of Artificial Sequence: Synthetic polynucleotide
gctccactttaataagtggtgccttccaaagctatatgctgagggaggatgggcgctgttgcagcgtctgcccacgctcagacgtgggtatccttacctattgaaaagtaataggtcaaggaatgcaac

SEQ ID NO: 132 - DNA, 131 bp, Artificial Sequence
  Description of Artificial Sequence: Synthetic polynucleotide
gctccactttaataagtggtgccttccaaagctatatgctgagggaggatgggcgctgttgcagcgtctgcccactgctcagacagtgggtatccttacctattgaaaagtaataggtcaaggaatgcaac

SEQ ID NO: 133 - DNA, 133 bp, Artificial Sequence
  Description of Artificial Sequence: Synthetic polynucleotide
gctccactttaataagtggtgccttccaaagctatatgctgagggaggatgggcgctgttgcagcgtctgcccacctgctcagacaggtgggtatccttacctattgaaaagtaataggtcaaggaatgcaac

SEQ ID NO: 134 - DNA, 135 bp, Artificial Sequence
  Description of Artificial Sequence: Synthetic polynucleotide
gctccactttaataagtggtgccttccaaagctatatgctgagggaggatgggcgctgttgcagcgtctgcccacgctgctcagacagcgtgggtatccttacctattgaaaagtaataggtcaaggaatgcaac

SEQ ID NO: 135 - DNA, 137 bp, Artificial Sequence
  Description of Artificial Sequence: Synthetic polynucleotide
gctccactttaataagtggtgccttccaaagctatatgctgagggaggatgggcgctgttgcagcgtctgcccactgctgctcagacagcagtgggtatccttacctattgaaaagtaataggtcaaggaatgcaac

SEQ ID NO: 136 - DNA, 140 bp, Artificial Sequence
  Description of Artificial Sequence: Synthetic polynucleotide
gctccactttaataagtggtgccttccaaagctatatgctgagggaggatgggcgctgttgcagcgtctgcccacacatgaggatcacccatgtgtgggtatccttacctattgaaaagtaataggtcaaggaatgcaac

SEQ ID NO: 137 - DNA, 125 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattaaaagtaataggtcaaggaatgcaac

SEQ ID NO: 138 - DNA, 128 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattggaaaagctaataggtcaaggaatgcaac

SEQ ID NO: 139 - DNA, 130 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgctaaaagagtaataggtcaaggaatgcaac

SEQ ID NO: 140 - DNA, 130 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgtgaaaagcataataggtcaaggaatgcaac

SEQ ID NO: 141 - DNA, 132 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgctgaaaagcagtaataggtcaaggaatgcaac

SEQ ID NO: 142 - DNA, 134 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattggctgaaaagcagctaataggtcaaggaatgcaac

SEQ ID NO: 143 - DNA, 136 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgtgctgaaaagcagcataataggtcaaggaatgcaac

SEQ ID NO: 144 - DNA, 138 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattacatgaggatcacccatgtaataggtcaaggaatgcaac

SEQ ID NO: 145 - DNA, 154 bp, Artificial Sequence
  Description of Artificial Sequence: Synthetic polynucleotide
gttatgctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 146 - DNA, 153 bp, Artificial Sequence
  Description of Artificial Sequence: Synthetic polynucleotide
gtatgctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 147 - DNA, 152 bp, Artificial Sequence
  Description of Artificial Sequence: Synthetic polynucleotide
gatgctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 148 - DNA, 148 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 149 - DNA, 149 bp, Artificial Sequence
  Description of Artificial Sequence: Synthetic polynucleotide
gctccgctttaataagcggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 150 - DNA, 149 bp, Artificial Sequence
  Description of Artificial Sequence: Synthetic polynucleotide
gctccactttactaagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 151 - DNA, 149 bp, Artificial Sequence
  Description of Artificial Sequence: Synthetic polynucleotide
gcaccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 152 - DNA, 149 bp, Artificial Sequence
  Description of Artificial Sequence: Synthetic polynucleotide
gctccactttaataagtggagccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 153 - DNA, 149 bp, Artificial Sequence
  Description of Artificial Sequence: Synthetic polynucleotide
gctccactgtaatcagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 154 - DNA, 151 bp, Artificial Sequence
  Description of Artificial Sequence: Synthetic polynucleotide
gtgctccactttaataagtggtgcattccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 155 - DNA, 151 bp, Artificial Sequence
  Description of Artificial Sequence: Synthetic polynucleotide
gctccacttgtaatcaagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 156 - DNA, 153 bp, Artificial Sequence
  Description of Artificial Sequence: Synthetic polynucleotide
gctccacttgctaatgcaagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 157 - DNA, 155 bp, Artificial Sequence
  Description of Artificial Sequence: Synthetic polynucleotide
gctccacttggctaatgccaagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 158 - DNA, 157 bp, Artificial Sequence
  Description of Artificial Sequence: Synthetic polynucleotide
gctccacttggcataattgccaagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 159 - DNA, 164 bp, Artificial Sequence
  Description of Artificial Sequence: Synthetic polynucleotide
gctccacttacatgaggatcacccatgtaagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 160 - DNA, 148 bp, Artificial Sequence
  Description of Artificial Sequence: Synthetic polynucleotide
gctccactttaataagtggtgccttccaaagctaatgctgagggaggatgggcgctgttgcagcgtctgcccacctcagagtgggtatccttacctattgaaaagtaataggtcaaggaatgcaactggttgcccaccctagtcattg

SEQ ID NO: 161 - DNA, 149 bp, Artificial Sequence
  Description of Artificial Sequence: Synthetic polynucleotide
gctccactttaataagtggtgccttccaaagctaaatgctgagggaggatgggcgctgttgcagcgtctgcccacctcagagtgggtatccttacctattgaaaagtaataggtcaaggaatgcaactggttgcccaccctagtcattg

SEQ ID NO: 162 - DNA, 151 bp, Artificial Sequence
  Description of Artificial Sequence: Synthetic polynucleotide
gctccactttaataagtggtgccttccaaagcctatatggctgagggaggatgggcgctgttgcagcgtctgcccacctcagagtgggtatccttacctattgaaaagtaataggtcaaggaatgcaactggttgcccaccctagtcattg

SEQ ID NO: 163 - DNA, 153 bp, Artificial Sequence
  Description of Artificial Sequence: Synthetic polynucleotide
gctccactttaataagtggtgccttccaaagctgtatatcagctgagggaggatgggcgctgttgcagcgtctgcccacctcagagtgggtatccttacctattgaaaagtaataggtcaaggaatgcaactggttgcccaccctagtcattg

SEQ ID NO: 164 - DNA, 155 bp, Artificial Sequence
  Description of Artificial Sequence: Synthetic polynucleotide
gctccactttaataagtggtgccttccaaagctgctatatgcagctgagggaggatgggcgctgttgcagcgtctgcccacctcagagtgggtatccttacctattgaaaagtaataggtcaaggaatgcaactggttgcccaccctagtcattg

SEQ ID NO: 165 - DNA, 157 bp, Artificial Sequence
  Description of Artificial Sequence: Synthetic polynucleotide
gctccactttaataagtggtgccttccaaagctgcttatatagcagctgagggaggatgggcgctgttgcagcgtctgcccacctcagagtgggtatccttacctattgaaaagtaataggtcaaggaatgcaactggttgcccaccctagtcattg

SEQ ID NO: 166 - DNA, 159 bp, Artificial Sequence
  Description of Artificial Sequence: Synthetic polynucleotide
gctccactttaataagtggtgccttccaaagctgctgtatatcagcagctgagggaggatgggcgctgttgcagcgtctgcccacctcagagtgggtatccttacctattgaaaagtaataggtcaaggaatgcaactggttgcccaccctagtcattg

SEQ ID NO: 167 - DNA, 163 bp, Artificial Sequence
  Description of Artificial Sequence: Synthetic polynucleotide
gctccactttaataagtggtgccttccaaagcacatgaggatcacccatgtgctgagggaggatgggcgctgttgcagcgtctgcccacctcagagtgggtatccttacctattgaaaagtaataggtcaaggaatgcaactggttgcccaccctagtcattg

SEQ ID NO: 168 - DNA, 149 bp, Artificial Sequence
  Description of Artificial Sequence: Synthetic polynucleotide
gtccactttaataagtggtgccttccaaagctatatgctgagggaggatgggcgctgttgcaagcgtctgcccacctcagagtgggtatccttacctattgaaaagtaataggtcaaggattgcaactggttgcccaccctagtcattg

SEQ ID NO: 169 - DNA, 149 bp, Artificial Sequence
  Description of Artificial Sequence: Synthetic polynucleotide
gtccactttaataagtggtgccttccaaagctatatgctgagggaggatgggcgctgttgcacgcgtctgcccacctcagagtgggtatccttacctattgaaaagtaataggtcaaggagtgcaactggttgcccaccctagtcattg

SEQ ID NO: 170 - DNA, 149 bp, Artificial Sequence
  Description of Artificial Sequence: Synthetic polynucleotide
gtccactttaataagtggtgccttccaaagctatatgctgagggaggatgggcgctgttgcaggcgtctgcccacctcagagtgggtatccttacctattgaaaagtaataggtcaaggactgcaactggttgcccaccctagtcattg

SEQ ID NO: 171 - DNA, 149 bp, Artificial Sequence
  Description of Artificial Sequence: Synthetic polynucleotide
gtccactttaataagtggtgccttccaaagctatatgctgagggaggatgggcgctgttgcatgcgtctgcccacctcagagtgggtatccttacctattgaaaagtaataggtcaaggaatgcaactggttgcccaccctagtcattg

SEQ ID NO: 172 - DNA, 149 bp, Artificial Sequence
  Description of Artificial Sequence: Synthetic polynucleotide
gtccactttaataagtggtgccttccaaagctatatgctgagggaggatgggcgctgttcgatgcgtctgcccacctcagagtgggtatccttacctattgaaaagtaataggtcaaggaatcgaactggttgcccaccctagtcattg

SEQ ID NO: 173 - DNA, 149 bp, Artificial Sequence
  Description of Artificial Sequence: Synthetic polynucleotide
gtccactttaataagtggtgccttccaaagctatatgctgagggaggatgggcgctgttgagtgcgtctgcccacctcagagtgggtatccttacctattgaaaagtaataggtcaaggaactcaactggttgcccaccctagtcattg

SEQ ID NO: 174 - DNA, 149 bp, Artificial Sequence
  Description of Artificial Sequence: Synthetic polynucleotide
gtccactttaataagtggtgccttccaaagctatatgctgagggaggatgggcgctgttgcgtgcgtctgcccacctcagagtgggtatccttacctattgaaaagtaataggtcaaggaacgcaactggttgcccaccctagtcattg

SEQ ID NO: 175 - DNA, 149 bp, Artificial Sequence
  Description of Artificial Sequence: Synthetic polynucleotide
gtccactttaataagtggtgccttccaaagctatatgctgagggaggatgggcgctgttgtatgcgtctgcccacctcagagtgggtatccttacctattgaaaagtaataggtcaaggaatacaactggttgcccaccctagtcattg

SEQ ID NO: 176 - DNA, 149 bp, Artificial Sequence
  Description of Artificial Sequence: Synthetic polynucleotide
gtccactttaataagtggtgccttccaaagctatatgctgagggaggatgggcgctgccgcatgcgtctgcccacctcagagtgggtatccttacctattgaaaagtaataggtcaaggaatgcggctggttgcccaccctagtcattg

SEQ ID NO: 177 - DNA, 149 bp, Artificial Sequence
  Description of Artificial Sequence: Synthetic polynucleotide
gtccactttaataagtggtgccttccaaagctatatgctgagggaggatgggcgctgcggcatgcgtctgcccacctcagagtgggtatccttacctattgaaaagtaataggtcaaggaatgccgctggttgcccaccctagtcattg

SEQ ID NO: 178 - DNA, 148 bp, Artificial Sequence
  Description of Artificial Sequence: Synthetic polynucleotide
gtccactttaataagtggtgccttccaaagctatatgctgagggaggatgggcgctgttgcggcgtctgcccacctcagagtgggtatccttacctattgaaaagtaataggtcaaggaacgcaactggttgcccaccctagtcattg

SEQ ID NO: 179 - DNA, 148 bp, Artificial Sequence
  Description of Artificial Sequence: Synthetic polynucleotide
gtccactttaataagtggtgccttccaaagctatatgctgagggaggatgggcgctgttgtagcgtctgcccacctcagagtgggtatccttacctattgaaaagtaataggtcaaggaatacaactggttgcccaccctagtcattg

SEQ ID NO: 180 - DNA, 148 bp, Artificial Sequence
  Description of Artificial Sequence: Synthetic polynucleotide
gtccactttaataagtggtgccttccaaagctatatgctgagggaggatgggcgctgccgcagcgtctgcccacctcagagtgggtatccttacctattgaaaagtaataggtcaaggaatgcggctggttgcccaccctagtcattg

SEQ ID NO: 181 - DNA, 148 bp, Artificial Sequence
  Description of Artificial Sequence: Synthetic polynucleotide
gtccactttaataagtggtgccttccaaagctatatgctgagggaggatgggcgctgcggcagcgtctgcccacctcagagtgggtatccttacctattgaaaagtaataggtcaaggaatgccgctggttgcccaccctagtcattg

SEQ ID NO: 182 - DNA, 150 bp, Artificial Sequence
  Description of Artificial Sequence: Synthetic polynucleotide
gctccactttaataagtggtgccttccaaagctatatgctgagggaggatgggcgctgttgcagcgtctgcccacgctagacgtgggtatccttacctattgaaaagtaataggtcaaggaatgcaactggttgcccaccctagtcattg

SEQ ID NO: 183 - DNA, 152 bp, Artificial Sequence
  Description of Artificial Sequence: Synthetic polynucleotide
gctccactttaataagtggtgccttccaaagctatatgctgagggaggatgggcgctgttgcagcgtctgcccactgctagacagtgggtatccttacctattgaaaagtaataggtcaaggaatgcaactggttgcccaccctagtcattg

SEQ ID NO: 184 - DNA, 154 bp, Artificial Sequence
  Description of Artificial Sequence: Synthetic polynucleotide
gctccactttaataagtggtgccttccaaagctatatgctgagggaggatgggcgctgttgcagcgtctgcccacctgctagacaggtgggtatccttacctattgaaaagtaataggtcaaggaatgcaactggttgcccaccctagtcattg

SEQ ID NO: 185 - DNA, 151 bp, Artificial Sequence
  Description of Artificial Sequence: Synthetic polynucleotide
gctccactttaataagtggtgccttccaaagctatatgctgagggaggatgggcgctgttgcagcgtctgcccacgctcagacgtgggtatccttacctattgaaaagtaataggtcaaggaatgcaactggttgcccaccctagtcattg

SEQ ID NO: 186 - DNA, 153 bp, Artificial Sequence
  Description of Artificial Sequence: Synthetic polynucleotide
gctccactttaataagtggtgccttccaaagctatatgctgagggaggatgggcgctgttgcagcgtctgcccactgctcagacagtgggtatccttacctattgaaaagtaataggtcaaggaatgcaactggttgcccaccctagtcattg

SEQ ID NO: 187 - DNA, 157 bp, Artificial Sequence
  Description of Artificial Sequence: Synthetic polynucleotide
gctccactttaataagtggtgccttccaaagctatatgctgagggaggatgggcgctgttgcagcgtctgcccacgctgctcagacagcgtgggtatccttacctattgaaaagtaataggtcaaggaatgcaactggttgcccaccctagtcattg

SEQ ID NO: 188 - DNA, 159 bp, Artificial Sequence
  Description of Artificial Sequence: Synthetic polynucleotide
gctccactttaataagtggtgccttccaaagctatatgctgagggaggatgggcgctgttgcagcgtctgcccactgctgctcagacagcagtgggtatccttacctattgaaaagtaataggtcaaggaatgcaactggttgcccaccctagtcattg

SEQ ID NO: 189 - DNA, 162 bp, Artificial Sequence
  Description of Artificial Sequence: Synthetic polynucleotide
gctccactttaataagtggtgccttccaaagctatatgctgagggaggatgggcgctgttgcagcgtctgcccacacatgaggatcacccatgtgtgggtatccttacctattgaaaagtaataggtcaaggaatgcaactggttgcccaccctagtcattg

SEQ ID NO: 190 - DNA, 147 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattaaaagtaataggtcaaggaatgcaactggttgcccaccctagtcattg

SEQ ID NO: 191 - DNA, 150 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattggaaaagctaataggtcaaggaatgcaactggttgcccaccctagtcattg

SEQ ID NO: 192 - DNA, 152 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgctaaaagagtaataggtcaaggaatgcaactggttgcccaccctagtcattg

SEQ ID NO: 193 - DNA, 152 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgtgaaaagcataataggtcaaggaatgcaactggttgcccaccctagtcattg

SEQ ID NO: 194 - DNA, 154 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgctgaaaagcagtaataggtcaaggaatgcaactggttgcccaccctagtcattg

SEQ ID NO: 195 - DNA, 156 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattggctgaaaagcagctaataggtcaaggaatgcaactggttgcccaccctagtcattg

SEQ ID NO: 196 - DNA, 158 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattgtgctgaaaagcagcataataggtcaaggaatgcaactggttgcccaccctagtcattg

SEQ ID NO: 197 - DNA, 160 bp, Artificial Sequence
  Description of Artificial Sequence: Synthetic polynucleotide
gtccactttaataagtggtgccttccaaagctatatgctgagggaggatgggcgctgttgcagcgtctgcccacctcagagtgggtatccttacctattacatgaggatcacccatgtaataggtcaaggaatgcaactggttgcccaccctagtcattg

SEQ ID NO: 198 - DNA, 154 bp, Artificial Sequence
  Description of Artificial Sequence: Synthetic polynucleotide
gctccacttgctaatgcaagtggtgccttccaaagctatatgctgagggaggatgggcgctgcggcatgcgtctgcccacctcagagtgggtatccttacctattgaaaagtaataggtcaaggaatgccgctggttgcccaccctagtcattg

SEQ ID NO: 199 - DNA, 150 bp, Artificial Sequence
  Description of Artificial Sequence: Synthetic polynucleotide
gctccgctttaataagcggtgccttccaaagctatatgctgagggaggatgggcgctgccgcatgcgtctgcccacctcagagtgggtatccttacctattgaaaagtaataggtcaaggaatgcggctggttgcccaccctagtcattg

SEQ ID NO: 200 - DNA, 150 bp, Artificial Sequence
  Description of Artificial Sequence: Synthetic polynucleotide
gcaccactttaataagtggtgccttccaaagctatatgctgagggaggatgggcgctgccgcatgcgtctgcccacctcagagtgggtatccttacctattgaaaagtaataggtcaaggaatgcggctggttgcccaccctagtcattg

SEQ ID NO: 201 - DNA, 150 bp, Artificial Sequence
  Description of Artificial Sequence: Synthetic polynucleotide
gctccactgtaatcagtggtgccttccaaagctatatgctgagggaggatgggcgctgccgcatgcgtctgcccacctcagagtgggtatccttacctattgaaaagtaataggtcaaggaatgcggctggttgcccaccctagtcattg

SEQ ID NO: 202 - DNA, 154 bp, Artificial Sequence
  Description of Artificial Sequence: Synthetic polynucleotide
gctccacttgctaatgcaagtggtgccttccaaagctatatgctgagggaggatgggcgctgccgcatgcgtctgcccacctcagagtgggtatccttacctattgaaaagtaataggtcaaggaatgcggctggttgcccaccctagtcattg

SEQ ID NO: 203 - DNA, 165 bp, Artificial Sequence
  Description of Artificial Sequence: Synthetic polynucleotide
gctccacttacatgaggatcacccatgtaagtggtgccttccaaagctatatgctgagggaggatgggcgctgccgcatgcgtctgcccacctcagagtgggtatccttacctattgaaaagtaataggtcaaggaatgcggctggttgcccaccctagtcattg

SEQ ID NO: 204 - DNA, 149 bp, Artificial Sequence; identical to SEQ ID NO: 176
  Description of Artificial Sequence: Synthetic polynucleotide

SEQ ID NO: 205 - DNA, 150 bp, Artificial Sequence
  Description of Artificial Sequence: Synthetic polynucleotide
gctccgctttaataagcggtgccttccaaagctatatgctgagggaggatgggcgctgcggcatgcgtctgcccacctcagagtgggtatccttacctattgaaaagtaataggtcaaggaatgccgctggttgcccaccctagtcattg

SEQ ID NO: 206 - DNA, 150 bp, Artificial Sequence
  Description of Artificial Sequence: Synthetic polynucleotide
gcaccactttaataagtggtgccttccaaagctatatgctgagggaggatgggcgctgcggcatgcgtctgcccacctcagagtgggtatccttacctattgaaaagtaataggtcaaggaatgccgctggttgcccaccctagtcattg

SEQ ID NO: 207 - DNA, 150 bp, Artificial Sequence
  Description of Artificial Sequence: Synthetic polynucleotide
gctccactgtaatcagtggtgccttccaaagctatatgctgagggaggatgggcgctgcggcatgcgtctgcccacctcagagtgggtatccttacctattgaaaagtaataggtcaaggaatgccgctggttgcccaccctagtcattg

SEQ ID NO: 208 - DNA, 165 bp, Artificial Sequence
  Description of Artificial Sequence: Synthetic polynucleotide
gctccacttacatgaggatcacccatgtaagtggtgccttccaaagctatatgctgagggaggatgggcgctgcggcatgcgtctgcccacctcagagtgggtatccttacctattgaaaagtaataggtcaaggaatgccgctggttgcccaccctagtcattg

SEQ ID NO: 209 - DNA, 149 bp, Artificial Sequence; identical to SEQ ID NO: 177
  Description of Artificial Sequence: Synthetic polynucleotide

SEQ ID NO: 210 - DNA, 149 bp, Artificial Sequence
  Description of Artificial Sequence: Synthetic polynucleotide
gctccgctttaataagcggtgccttccaaagctatatgctgagggaggatgggcgctgccgcagcgtctgcccacctcagagtgggtatccttacctattgaaaagtaataggtcaaggaatgcggctggttgcccaccctagtcattg

SEQ ID NO: 211 - DNA, 149 bp, Artificial Sequence
  Description of Artificial Sequence: Synthetic polynucleotide
gcaccactttaataagtggtgccttccaaagctatatgctgagggaggatgggcgctgccgcagcgtctgcccacctcagagtgggtatccttacctattgaaaagtaataggtcaaggaatgcggctggttgcccaccctagtcattg

SEQ ID NO: 212 - DNA, 149 bp, Artificial Sequence
  Description of Artificial Sequence: Synthetic polynucleotide
gctccactgtaatcagtggtgccttccaaagctatatgctgagggaggatgggcgctgccgcagcgtctgcccacctcagagtgggtatccttacctattgaaaagtaataggtcaaggaatgcggctggttgcccaccctagtcattg

SEQ ID NO: 213 - DNA, 153 bp, Artificial Sequence
  Description of Artificial Sequence: Synthetic polynucleotide
gctccacttgctaatgcaagtggtgccttccaaagctatatgctgagggaggatgggcgctgccgcagcgtctgcccacctcagagtgggtatccttacctattgaaaagtaataggtcaaggaatgcggctggttgcccaccctagtcattg

SEQ ID NO: 214 - DNA, 164 bp, Artificial Sequence
  Description of Artificial Sequence: Synthetic polynucleotide
gctccacttacatgaggatcacccatgtaagtggtgccttccaaagctatatgctgagggaggatgggcgctgccgcagcgtctgcccacctcagagtgggtatccttacctattgaaaagtaataggtcaaggaatgcggctggttgcccaccctagtcattg

SEQ ID NO: 215 - DNA, 148 bp, Artificial Sequence; identical to SEQ ID NO: 180
  Description of Artificial Sequence: Synthetic polynucleotide

SEQ ID NO: 216 - DNA, 149 bp, Artificial Sequence
  Description of Artificial Sequence: Synthetic polynucleotide
gctccgctttaataagcggtgccttccaaagctatatgctgagggaggatgggcgctgcggcagcgtctgcccacctcagagtgggtatccttacctattgaaaagtaataggtcaaggaatgccgctggttgcccaccctagtcattg

SEQ ID NO: 217 - DNA, 149 bp, Artificial Sequence
  Description of Artificial Sequence: Synthetic polynucleotide
gcaccactttaataagtggtgccttccaaagctatatgctgagggaggatgggcgctgcggcagcgtctgcccacctcagagtgggtatccttacctattgaaaagtaataggtcaaggaatgccgctggttgcccaccctagtcattg

SEQ ID NO: 218 - DNA, 149 bp, Artificial Sequence
  Description of Artificial Sequence: Synthetic polynucleotide
gctccactgtaatcagtggtgccttccaaagctatatgctgagggaggatgggcgctgcggcagcgtctgcccacctcagagtgggtatccttacctattgaaaagtaataggtcaaggaatgccgctggttgcccaccctagtcattg

SEQ ID NO: 219 - DNA, 153 bp, Artificial Sequence
  Description of Artificial Sequence: Synthetic polynucleotide
gctccacttgctaatgcaagtggtgccttccaaagctatatgctgagggaggatgggcgctgcggcagcgtctgcccacctcagagtgggtatccttacctattgaaaagtaataggtcaaggaatgccgctggttgcccaccctagtcattg

SEQ ID NO: 220 - DNA, 164 bp, Artificial Sequence
  Description of Artificial Sequence: Synthetic polynucleotide
gctccacttacatgaggatcacccatgtaagtggtgccttccaaagctatatgctgagggaggatgggcgctgcggcagcgtctgcccacctcagagtgggtatccttacctattgaaaagtaataggtcaaggaatgccgctggttgcccaccctagtcattg

SEQ ID NO: 221 - DNA, 148 bp, Artificial Sequence; identical to SEQ ID NO: 181
  Description of Artificial Sequence: Synthetic polynucleotide

SEQ ID NO: 222 - DNA, 149 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 223 - DNA, 147 bp, Artificial Sequence
  Description of Artificial Sequence: Synthetic polynucleotide
tccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 224 - DNA, 129 bp, Artificial Sequence
  Description of Artificial Sequence: Synthetic polynucleotide
gccttccaaagctatatgctgagggaggatgggcgctgttgcagcgtctgcccacctcagagtgggtatccttacctattgaaaagtaataggtcaaggaatgcaactggttgcccaccctagtcattg

SEQ ID NO: 225 - DNA, 152 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattggag

SEQ ID NO: 226 - DNA, 151 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattgga

SEQ ID NO: 227 - DNA, 150 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcattgg

SEQ ID NO: 228 - DNA, 149 bp, Artificial Sequence; identical to SEQ ID NO: 222
  Description of Artificial Sequence: Synthetic polynucleotide

SEQ ID NO: 229 - DNA, 148 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcatt

SEQ ID NO: 230 - DNA, 147 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtcat

SEQ ID NO: 231 - DNA, 146 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtca

SEQ ID NO: 232 - DNA, 145 bp, Artificial Sequence
  Description of Artificial Sequence: Synthetic polynucleotide
gctccactttaataagtggtgccttccaaagctatatgctgagggaggatgggcgctgttgcagcgtctgcccacctcagagtgggtatccttacctattgaaaagtaataggtcaaggaatgcaactggttgcccaccctagtc

SEQ ID NO: 233 - protein, 597 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNIGSPKKKRKVSGVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 234 - protein, 597 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRGSPKKKRKVSGAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 235 - protein, 597 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGSPKKKRKVSGGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 236 - protein, 597 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEGSPKKKRKVSGKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 237 - protein, 597 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNGSPKKKRKVSGNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 238 - protein, 597 aa, Artificial Sequence
  Description of Artificial Sequence: Synthetic polypeptide
MPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGSPKKKRKVSGGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDK

SEQ ID NO: 239 - protein, 637 aa, Artificial Sequence
  Description of Artificial Sequence: Synthetic polypeptide
MKRTADGSEFESPKKKRKVMPSETYITKTLSLKLIPSDEEKQALENYFITFQRAVNFAIDRIVDIRSSFRYLNKNEQFPAVCDCCGKKEKIMYVNIGSPKKKRKVSGVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKSGGSKRTADGSEFEPKKKRKV

SEQ ID NO: 240 - protein, 637 aa, Artificial Sequence
  Description of Artificial Sequence: Synthetic polypeptide
MKRTADGSEFESPKKKRKVMPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRGSPKKKRKVSGAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKSGGSKRTADGSEFEPKKKRKV

SEQ ID NO: 241 - protein, 637 aa, Artificial Sequence
  Description of Artificial Sequence: Synthetic polypeptide
MKRTADGSEFESPKKKRKVMPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGSPKKKRKVSGGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKSGGSKRTADGSEFEPKKKRKV

SEQ ID NO: 242 - protein, 637 aa, Artificial Sequence
  Description of Artificial Sequence: Synthetic polypeptide
MKRTADGSEFESPKKKRKVMPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEGSPKKKRKVSGKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKSGGSKRTADGSEFEPKKKRKV

SEQ ID NO: 243 - protein, 637 aa, Artificial Sequence
  Description of Artificial Sequence: Synthetic polypeptide
MKRTADGSEFESPKKKRKVMPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNGSPKKKRKVSGNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKSGGSKRTADGSEFEPKKKRKV

SEQ ID NO: 244 - protein, 637 aa, Artificial Sequence
  Description of Artificial Sequence: Synthetic polypeptide
MKRTADGSEFESPKKKRKVMPSETYITKTLSLKLIPSDEEKQALENYFITFQRAVNFAIDRIVDIRSSFRYLNKNEQFPAVCDCCGKKEKIMYVNIVWLDGVNIFSVSILLVSAWLEFKGFVRAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGSPKKKRKVSGGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKSGGSKRTADGSEFEPKKKRKV

SEQ ID NO: 245 - DNA, 154 bp, Artificial Sequence
  Description of Artificial Sequence: Synthetic polynucleotide
gctccacttgctaatgcaagtggtgccttccaaagctatatgctgagggaggatgggcgctgcggcatgcgtctgcccacctcagagtgggtatccttacctattgaaaagtaataggtcaaggaatgccgctatgagttacaacgaacacctc

SEQ ID NO: 246 - DNA, 160 bp, Artificial Sequence
  Description of Artificial Sequence: Synthetic polynucleotide
gctccacttgctaatgcaagtggtgccttccaaaccaaagcctatatggctgagggaggatgggcgctgccgcatgcgtctgcccacctcagagtgggtatccttacctattaaaagtaataggtcaaggaatgcggctggttgcccaccctagtcattg

SEQ ID NO: 247 - DNA, 153 bp, Artificial Sequence
  Description of Artificial Sequence: Synthetic polynucleotide
gcaccactttaataagtggtgccttccaaagctgtatatcagctgagggaggatgggcgctgccgcatgcgtctgcccacctcagagtgggtatccttacctattaaaagtaataggtcaaggaatgcggctggttgcccaccctagtcattg

SEQ ID NO: 248 - DNA, 157 bp, Artificial Sequence
  Description of Artificial Sequence: Synthetic polynucleotide
gctccacttgctaatgcaagtggtgccttccaaagctgtatatcagctgagggaggatgggcgctgccgcatgcgtctgcccacctcagagtgggtatccttacctattaaaagtaataggtcaaggaatgcggctggttgcccaccctagtcattg

SEQ ID NO: 249 - DNA, 155 bp, Artificial Sequence
  Description of Artificial Sequence: Synthetic polynucleotide
gcaccactttaataagtggtgccttccaaagctgctatatgcagctgagggaggatgggcgctgccgcatgcgtctgcccacctcagagtgggtatccttacctattaaaagtaataggtcaaggaatgcggctggttgcccaccctagtcattg

SEQ ID NO: 250 - DNA, 160 bp, Artificial Sequence
  Description of Artificial Sequence: Synthetic polynucleotide
gctccacttgctaatgcaagtggtgccttccaaaccaaagcctatatggctgagggaggatgggcgctgcggcatgcgtctgcccacctcagagtgggtatccttacctattaaaagtaataggtcaaggaatgccgctggttgcccaccctagtcattg

SEQ ID NO: 251 - DNA, 153 bp, Artificial Sequence
  Description of Artificial Sequence: Synthetic polynucleotide
gcaccactttaataagtggtgccttccaaagctgtatatcagctgagggaggatgggcgctgcggcatgcgtctgcccacctcagagtgggtatccttacctattaaaagtaataggtcaaggaatgccgctggttgcccaccctagtcattg

SEQ ID NO: 252 - DNA, 157 bp, Artificial Sequence
  Description of Artificial Sequence: Synthetic polynucleotide
gctccacttgctaatgcaagtggtgccttccaaagctgtatatcagctgagggaggatgggcgctgcggcatgcgtctgcccacctcagagtgggtatccttacctattaaaagtaataggtcaaggaatgccgctggttgcccaccctagtcattg

SEQ ID NO: 253 - DNA, 163 bp, Artificial Sequence
  Description of Artificial Sequence: Synthetic polynucleotide
gctccacttgctaatgcaagtggtgccttccaaaccaaagcctatatggctgagggaggatgggcgctgccgcatgcgtctgcccacctcagagtgggtatccttacctattggaaaagctaataggtcaaggaatgcggctggttgcccaccctagtcattg

SEQ ID NO: 254 - DNA, 156 bp, Artificial Sequence
  Description of Artificial Sequence: Synthetic polynucleotide
gcaccactttaataagtggtgccttccaaagctgtatatcagctgagggaggatgggcgctgccgcatgcgtctgcccacctcagagtgggtatccttacctattggaaaagctaataggtcaaggaatgcggctggttgcccaccctagtcattg

SEQ ID NO: 255 - DNA, 160 bp, Artificial Sequence
  Description of Artificial Sequence: Synthetic polynucleotide
gctccacttgctaatgcaagtggtgccttccaaagctgtatatcagctgagggaggatgggcgctgccgcatgcgtctgcccacctcagagtgggtatccttacctattggaaaagctaataggtcaaggaatgcggctggttgcccaccctagtcattg

SEQ ID NO: 256 - DNA, 158 bp, Artificial Sequence
  Description of Artificial Sequence: Synthetic polynucleotide
gcaccactttaataagtggtgccttccaaagctgctatatgcagctgagggaggatgggcgctgccgcatgcgtctgcccacctcagagtgggtatccttacctattggaaaagctaataggtcaaggaatgcggctggttgcccaccctagtcattg

SEQ ID NO: 257 - DNA, 163 bp, Artificial Sequence
  Description of Artificial Sequence: Synthetic polynucleotide
gctccacttgctaatgcaagtggtgccttccaaaccaaagcctatatggctgagggaggatgggcgctgcggcatgcgtctgcccacctcagagtgggtatccttacctattggaaaagctaataggtcaaggaatgccgctggttgcccaccctagtcattg

SEQ ID NO: 258 - DNA, 156 bp, Artificial Sequence
  Description of Artificial Sequence: Synthetic polynucleotide
gcaccactttaataagtggtgccttccaaagctgtatatcagctgagggaggatgggcgctgcggcatgcgtctgcccacctcagagtgggtatccttacctattggaaaagctaataggtcaaggaatgccgctggttgcccaccctagtcattg

SEQ ID NO: 259 - DNA, 160 bp, Artificial Sequence
  Description of Artificial Sequence: Synthetic polynucleotide
gctccacttgctaatgcaagtggtgccttccaaagctgtatatcagctgagggaggatgggcgctgcggcatgcgtctgcccacctcagagtgggtatccttacctattggaaaagctaataggtcaaggaatgccgctggttgcccaccctagtcattg

SEQ ID NO: 260 - DNA, 158 bp, Artificial Sequence
  Description of Artificial Sequence: Synthetic polynucleotide
gcaccactttaataagtggtgccttccaaagctgctatatgcagctgagggaggatgggcgctgcggcatgcgtctgcccacctcagagtgggtatccttacctattggaaaagctaataggtcaaggaatgccgctggttgcccaccctagtcattg

SEQ ID NO: 261 - DNA, 165 bp, Artificial Sequence
  Description of Artificial Sequence: Synthetic polynucleotide
gctccacttgctaatgcaagtggtgccttccaaaccaaagcctatatggctgagggaggatgggcgctgccgcatgcgtctgcccacctcagagtgggtatccttacctattgtgaaaagcataataggtcaaggaatgcggctggttgcccaccctagtcattg

SEQ ID NO: 262 - DNA, 158 bp, Artificial Sequence
  Description of Artificial Sequence: Synthetic polynucleotide
gcaccactttaataagtggtgccttccaaagctgtatatcagctgagggaggatgggcgctgccgcatgcgtctgcccacctcagagtgggtatccttacctattgtgaaaagcataataggtcaaggaatgcggctggttgcccaccctagtcattg

SEQ ID NO: 263 - DNA, 162 bp, Artificial Sequence
  Description of Artificial Sequence: Synthetic polynucleotide
gctccacttgctaatgcaagtggtgccttccaaagctgtatatcagctgagggaggatgggcgctgccgcatgcgtctgcccacctcagagtgggtatccttacctattgtgaaaagcataataggtcaaggaatgcggctggttgcccaccctagtcattg

SEQ ID NO: 264 - DNA, 160 bp, Artificial Sequence
  Description of Artificial Sequence: Synthetic polynucleotide
gcaccactttaataagtggtgccttccaaagctgctatatgcagctgagggaggatgggcgctgccgcatgcgtctgcccacctcagagtgggtatccttacctattgtgaaaagcataataggtcaaggaatgcggctggttgcccaccctagtcattg

SEQ ID NO: 265 - DNA, 165 bp, Artificial Sequence
  Description of Artificial Sequence: Synthetic polynucleotide
gctccacttgctaatgcaagtggtgccttccaaaccaaagcctatatggctgagggaggatgggcgctgcggcatgcgtctgcccacctcagagtgggtatccttacctattgtgaaaagcataataggtcaaggaatgccgctggttgcccaccctagtcattg

SEQ ID NO: 266 - DNA, 158 bp, Artificial Sequence
  Description of Artificial Sequence: Synthetic polynucleotide
gcaccactttaataagtggtgccttccaaagctgtatatcagctgagggaggatgggcgctgcggcatgcgtctgcccacctcagagtgggtatccttacctattgtgaaaagcataataggtcaaggaatgccgctggttgcccaccctagtcattg

SEQ ID NO: 267 - DNA, 162 bp, Artificial Sequence
  Description of Artificial Sequence: Synthetic polynucleotide
gctccacttgctaatgcaagtggtgccttccaaagctgtatatcagctgagggaggatgggcgctgcggcatgcgtctgcccacctcagagtgggtatccttacctattgtgaaaagcataataggtcaaggaatgccgctggttgcccaccctagtcattg

SEQ ID NO: 268 - DNA, 160 bp, Artificial Sequence
  Description of Artificial Sequence: Synthetic polynucleotide
gcaccactttaataagtggtgccttccaaagctgctatatgcagctgagggaggatgggcgctgcggcatgcgtctgcccacctcagagtgggtatccttacctattgtgaaaagcataataggtcaaggaatgccgctggttgcccaccctagtcattg

SEQ ID NO: 269 - DNA, 153 bp, Artificial Sequence
  Description of Artificial Sequence: Synthetic polynucleotide
gctccgctttaataagcggtgccttccaaagctgtatatcagctgagggaggatgggcgctgttgcagcgtctgcccacctcagagtgggtatccttacctattgaaaagtaataggtcaaggaatgcaactggttgcccaccctagtcattg

SEQ ID NO: 270 - DNA, 153 bp, Artificial Sequence
  Description of Artificial Sequence: Synthetic polynucleotide
gcaccactttaataagtggtgccttccaaagctgtatatcagctgagggaggatgggcgctgttgcagcgtctgcccacctcagagtgggtatccttacctattgaaaagtaataggtcaaggaatgcaactggttgcccaccctagtcattg

SEQ ID NO: 271 - DNA, 153 bp, Artificial Sequence
  Description of Artificial Sequence: Synthetic polynucleotide
gctccactgtaatcagtggtgccttccaaagctgtatatcagctgagggaggatgggcgctgttgcagcgtctgcccacctcagagtgggtatccttacctattgaaaagtaataggtcaaggaatgcaactggttgcccaccctagtcattg

SEQ ID NO: 272 - DNA, 157 bp, Artificial Sequence
  Description of Artificial Sequence: Synthetic polynucleotide
gctccacttgctaatgcaagtggtgccttccaaagctgtatatcagctgagggaggatgggcgctgttgcagcgtctgcccacctcagagtgggtatccttacctattgaaaagtaataggtcaaggaatgcaactggttgcccaccctagtcattg

SEQ ID NO: 273 - protein, 650 aa, Artificial Sequence
  Description of Artificial Sequence: Synthetic polypeptide
MKRTADGSEFESPKKKRKVSGGSISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNSGGSKRTADGSEFEPKKKRKV

SEQ ID NO: 274 - protein, 650 aa, Artificial Sequence
  Description of Artificial Sequence: Synthetic polypeptide
MKRTADGSEFESPKKKRKVSGGSNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPSGGSKRTADGSEFEPKKKRKV

SEQ ID NO: 275 - protein, 650 aa, Artificial Sequence
  Description of Artificial Sequence: Synthetic polypeptide
MKRTADGSEFESPKKKRKVSGGSPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNASGGSKRTADGSEFEPKKKRKV

SEQ ID NO: 276 - protein, 650 aa, Artificial Sequence
  Description of Artificial Sequence: Synthetic polypeptide
MKRTADGSEFESPKKKRKVSGGSEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPSGGSKRTADGSEFEPKKKRKV

SEQ ID NO: 277 - protein, 650 aa, Artificial Sequence
  Description of Artificial Sequence: Synthetic polypeptide
MKRTADGSEFESPKKKRKVSGGSNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKSGGSKRTADGSEFEPKKKRKV

SEQ ID NO: 278 - protein, 650 aa, Artificial Sequence
  Description of Artificial Sequence: Synthetic polypeptide
MKRTADGSEFESPKKKRKVSGGSDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKSGGSKRTADGSEFEPKKKRKV

SEQ ID NO: 279 - protein, 650 aa, Artificial Sequence
  Description of Artificial Sequence: Synthetic polypeptide
MKRTADGSEFESPKKKRKVSGGSKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPSGGSKRTADGSEFEPKKKRKV

SEQ ID NO: 280 - protein, 650 aa, Artificial Sequence
  Description of Artificial Sequence: Synthetic polypeptide
MKRTADGSEFESPKKKRKVSGGSKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNSGGSKRTADGSEFEPKKKRKV

SEQ ID NO: 281 - protein, 650 aa, Artificial Sequence
  Description of Artificial Sequence: Synthetic polypeptide
MKRTADGSEFESPKKKRKVSGGSKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRSGGSKRTADGSEFEPKKKRKV

SEQ ID NO: 282 - protein, 650 aa, Artificial Sequence
  Description of Artificial Sequence: Synthetic polypeptide
MKRTADGSEFESPKKKRKVSGGSAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYSGGSKRTADGSEFEPKKKRKV

SEQ ID NO: 283 - protein, 650 aa, Artificial Sequence
  Description of Artificial Sequence: Synthetic polypeptide
MKRTADGSEFESPKKKRKVSGGSRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKSGGSKRTADGSEFEPKKKRKV

SEQ ID NO: 284 - protein, 650 aa, Artificial Sequence
  Description of Artificial Sequence: Synthetic polypeptide
MKRTADGSEFESPKKKRKVSGGSNTSRKCSKCGYVDENNRKQASFKCLKCGYSLNADLNAAVNIAKAFYECPTFRWEEKLHAYVCSEPDKGGSGGSGGSGGSGGSGGSGGMPSETYITKTLSLKLIPSDEEKQALENYFITFQRAVNFAIDRIVDIRSSFRYLNKNEQFPAVCDCCGKKEKIMYVNISNKTFKFKPSRNQKDRYTKDIYTIKPNAHICKTCYSGVAGNMFIRKQMYPNDKEGWKVSRSYNIKVNAPGLTGTEYAMAIRKAISILRSFEKRRRNAERRIIEYEKSKKEYLELIDDVEKGKTNKIVVLEKEGHQRVKRYKHKNWPEKWQGISLNKAKSKVKDIEKRIKKLKEWKHPTLNRPYVELHKNNVRIVGYETVELKLGNKMYTIHFASISNLRKPFRKQKKKSIEYLKHLLTLALKRNLETYPSIIKRGKNFFLQYPVRVTVKVPKLTKNFKAFGIDRGVNRLAVGCIISKDGKLTNKNIFFFHGKEAWAKENRYKKIRDRLYAMAKKLRGDKTKKIRLYHEIRKKFRHKVKYFRRNYLHNISKQIVEIAKENTPTVIVLEDLRYLRERTYRGKGRSKKAKKTNYKLNTFTYRMLIDMIKYKAEEAGVPVMIIDPRSGGSKRTADGSEFEPKKKRKV

SEQ ID NO: 285 - skipped

SEQ ID NO: 286 - protein, 18 aa, Artificial Sequence
  Description of Artificial Sequence: Synthetic peptide
GGSGGSGGSGGSGGSGGS

SEQ ID NO: 287 - DNA, 155 bp, Artificial Sequence
  Description of Artificial Sequence: Synthetic polynucleotide
gctccactttaataagtggtgccttccaaagctatatgctgagggaggatgggcgctgttgcagcgtctgcccacctgctcagacaggtgggtatccttacctattgaaaagtaataggtcaaggaatgcaactggttgcccaccctagtcattg

SEQ ID NO: 288 - DNA, 155 bp, Artificial Sequence
  Description of Artificial Sequence: Synthetic polynucleotide
gcaccactttaataagtggtgccttccaaagctgctatatgcagctgagggaggatgggcgctgcggcatgcgtctgcccacctcagagtgggtatccttacctattaaaagtaataggtcaaggaatgccgctggttgcccaccctagtcattg

SEQ ID NO: 289 - DNA, 61 bp, Artificial Sequence
  Description of Artificial Sequence: Synthetic oligonucleotide
  modified_base (13)..(41): a, c, t, g, unknown or other
atggggaggacannnnnnnnnnnnnnnnnnnnnnnnnnnnncaaacccacgagggcagagt

SEQ ID NO: 290 - DNA, 67 bp, Unknown
  Description of Unknown: EMX1 sequence
atggggaggacatcgatgtcacctccaatgactagggtgggcaaccacaaacccacgagggcagagt

SEQ ID NO: 291 - DNA, 41 bp, Artificial Sequence
  Description of Artificial Sequence: Synthetic oligonucleotide
atggggaggacatcgatgtcacaaacccacgagggcagagt

SEQ ID NO: 292 - DNA, 53 bp, Artificial Sequence
  Description of Artificial Sequence: Synthetic oligonucleotide
atggggaggacatcgatgtcacgtgggcaaccacaaacccacgagggcagagt

SEQ ID NO: 293 - DNA, 44 bp, Artificial Sequence
  Description of Artificial Sequence: Synthetic oligonucleotide
atggggaggacatcgatgtcaccacaaacccacgagggcagagt

SEQ ID NO: 294 - DNA, 37 bp, Artificial Sequence
  Description of Artificial Sequence: Synthetic oligonucleotide
atggggaggacatcgatgtcacccacgagggcagagt

SEQ ID NO: 295 - DNA, 41 bp, Artificial Sequence
  Description of Artificial Sequence: Synthetic oligonucleotide
atggggaggacatcgatgccacaaacccacgagggcagagt

SEQ ID NO: 296 - DNA, 33 bp, Artificial Sequence
  Description of Artificial Sequence: Synthetic oligonucleotide
atggggaggacatcgatgtcacgagggcagagt

SEQ ID NO: 297 - DNA, 48 bp, Artificial Sequence
  Description of Artificial Sequence: Synthetic oligonucleotide
atggggaggacatcgatgtgggcaaccacaaacccacgagggcagagt

SEQ ID NO: 298 - DNA, 33 bp, Artificial Sequence
  Description of Artificial Sequence: Synthetic oligonucleotide
atggggaggaccacaaacccacgagggcagagt

SEQ ID NO: 299 - DNA, 54 bp, Artificial Sequence
  Description of Artificial Sequence: Synthetic oligonucleotide
atggggaggacatcgatgtcacggtgggcaaccacaaacccacgagggcagagt

SEQ ID NO: 300 - DNA, 61 bp, Artificial Sequence
  Description of Artificial Sequence: Synthetic oligonucleotide
atggggaggacatcgatgtcactgactagggtgggcaaccacaaacccacgagggcagagt

SEQ ID NO: 301 - DNA, 52 bp, Artificial Sequence
  Description of Artificial Sequence: Synthetic oligonucleotide
atggggaggacatcgatgtcactgggcaaccacaaacccacgagggcagagt

SEQ ID NO: 302 - DNA, 57 bp, Artificial Sequence
  Description of Artificial Sequence: Synthetic oligonucleotide
atggggaggacatcgatgtcactagggtgggcaaccacaaacccacgagggcagagt

SEQ ID NO: 303 - DNA, 45 bp, Artificial Sequence
  Description of Artificial Sequence: Synthetic oligonucleotide
atggggaggacatcgtgggcaaccacaaacccacgagggcagagt

SEQ ID NO: 304 - DNA, 60 bp, Artificial Sequence
  Description of Artificial Sequence: Synthetic oligonucleotide
atggggaggacatcgatgtcacgactagggtgggcaaccacaaacccacgagggcagagt

SEQ ID NO: 305 - DNA, 42 bp, Artificial Sequence
  Description of Artificial Sequence: Synthetic oligonucleotide
atggggaggacgtgggcaaccacaaacccacgagggcagagt

SEQ ID NO: 306 - DNA, 51 bp, Artificial Sequence
  Description of Artificial Sequence: Synthetic oligonucleotide
atggggaggacatcgatgtcacgggcaaccacaaacccacgagggcagagt

SEQ ID NO: 307 - DNA, 32 bp, Artificial Sequence
  Description of Artificial Sequence: Synthetic oligonucleotide
atggggaggacacaaacccacgagggcagagt

SEQ ID NO: 308 - DNA, 46 bp, Artificial Sequence
  Description of Artificial Sequence: Synthetic oligonucleotide
atggggaggacatcgatgggcaaccacaaacccacgagggcagagt

SEQ ID NO: 309 - DNA, 46 bp, Artificial Sequence
  Description of Artificial Sequence: Synthetic oligonucleotide
atggggaggacatcggtgggcaaccacaaacccacgagggcagagt

SEQ ID NO: 310 - DNA, 59 bp, Artificial Sequence
  Description of Artificial Sequence: Synthetic oligonucleotide
atggggaggacatcgatgtcacactagggtgggcaaccacaaacccacgagggcagagt

SEQ ID NO: 311 - DNA, 58 bp, Artificial Sequence
  Description of Artificial Sequence: Synthetic oligonucleotide
atggggaggacatcgatgtcacctagggtgggcaaccacaaacccacgagggcagagt

SEQ ID NO: 312 - DNA, 30 bp, Artificial Sequence
  Description of Artificial Sequence: Synthetic oligonucleotide
atggggaggacaaacccacgagggcagagt

SEQ ID NO: 313 - DNA, 45 bp, Artificial Sequence
  Description of Artificial Sequence: Synthetic oligonucleotide
atggggaggacatcgatgtcacccacaaacccacgagggcagagt

SEQ ID NO: 314 - RNA, 126 nt, Artificial Sequence
  Description of Artificial Sequence: Synthetic polynucleotide
guccacuuuaauaaguggugccuuccaaagcuauaugcugagggaggaugggcgcuguugcagcgucuccccaccucagaguggguauccuuaccuauugaaaaguaauaggucaaggaaugcaac

SEQ ID NO: 315 - DNA, 153 bp, Artificial Sequence
  Description of Artificial Sequence: Synthetic polynucleotide
  modified_base (133)..(153): a, c, t, g, unknown or other
gctccacttgctaatgcaagtggtgccttccaaagctatatgctgagggaggatgggcgctgcggcatgcgtctgcccacctcagagtgggtatccttacctattgaaaagtaataggtcaaggaatgccgcnnnnnnnnnnnnnnnnnnnnn

SEQ ID NO: 316 - protein, 37 aa, Artificial Sequence
  Description of Artificial Sequence: Synthetic polypeptide
APLANASGAFQSYMLREDGRCGMRLPTSEWVSLPIEK

SEQ ID NO: 317 - DNA, 22 bp, Artificial Sequence
  Description of Artificial Sequence: Synthetic oligonucleotide
tatgagttacaacgaacacctc

SEQ ID NO: 318 - protein, 12 aa, Artificial Sequence
  Description of Artificial Sequence: Synthetic peptide
  MOD_RES (6)..(12): Any amino acid
VKECRXXXXXXX